(19) United States
(12) Patent Application Publication
(10) Pub. No.: US 2025/0195536 A1
(43) Pub. Date: Jun. 19, 2025

(54) BICYCLOOCTANE KRAS INHIBITORS

(71) Applicant: Incyte Corporation, Wilmington, DE (US)

(72) Inventors: Yannik Boni, Wilmington, DE (US); Chengtsung Lai, Wilmington, DE (US); Rocco Policarpo, III, Wilmington, DE (US); Alexander Sokolsky, Wilmington, DE (US); Xiaozhao Wang, Mt. Laurel, NJ (US); Haolin Yin, Wilmington, DE (US); Wenhao Zhang, Wilmington, DE (US); Gencheng Li, Wilmington, DE (US); Nathaniel Greenwood, Wilmington, DE (US)

(21) Appl. No.: 18/980,424

(22) Filed: Dec. 13, 2024

Related U.S. Application Data
(60) Provisional application No. 63/609,753, filed on Dec. 13, 2023.
(60) Provisional application No. 63/572,705, filed on Apr. 1, 2024.

(51) Int. Cl. A61K 31/551; C07D 519/00

(52) U.S. Cl. CPC A61K 31/551; C07D 519/00

(57) ABSTRACT

Disclosed are compounds of Formula (I), methods of using the compounds for inhibiting KRAS activity and pharmaceutical compositions comprising such compounds. The compounds are useful in treating, preventing or ameliorating diseases or disorders associated with KRAS activity such as cancer.

### RELATED APPLICATIONS

This application claims priority to U.S. Provisional Application No. 63/609,753 filed on Dec. 13, 2023, and U.S. Provisional Application No. 63/572,705 filed on Apr. 1, 2024, the entire contents of which are incorporated by reference.

### FIELD OF THE INVENTION

This disclosure provides compounds as well as their compositions and methods of use. The compounds modulate KRAS activity and are useful in the treatment of various diseases including cancer.

### BACKGROUND OF THE INVENTION

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al. Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al. Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

### SUMMARY

The present disclosure provides, inter alia, a compound of Formula (I):

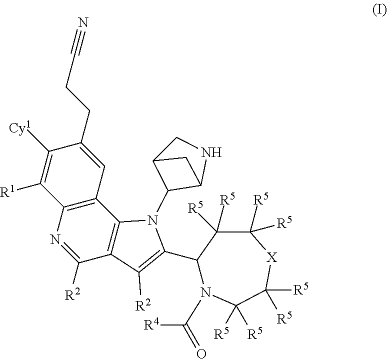

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure further provides methods of inhibiting KRAS activity, which comprises administering to an individual a compound of the disclosure, or a pharmaceutically acceptable salt thereof. The present disclosure also provides uses of the compounds described herein in the manufacture of a medicament for use in therapy. The present disclosure also provides the compounds described herein for use in therapy.

The present disclosure further provides methods of treating a disease or disorder in a patient comprising administering to the patient a therapeutically effective amount of a compound of the disclosure, or a pharmaceutically acceptable salt thereof.

The details of one or more embodiments are set forth in the description below. Other features, objects, and advantages will be apparent from the description and from the claims.

### DETAILED DESCRIPTION

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

### I. Compounds

In an aspect, provided herein is a compound having Formula (I):

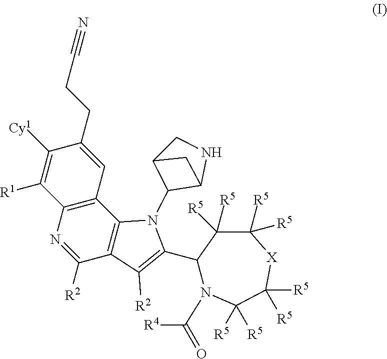

or a pharmaceutically acceptable salt thereof, wherein:
Cy¹ is C₆₋₁₀ aryl or 6-10 membered heteroaryl; wherein the C₆₋₁₀ aryl and 6-10 membered heteroaryl forming Cy¹ are each optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, C(O)R^{bCy1}, C(O)NR^{cCy1}R^{dCy1}, C(O)OR^{aCy1}, NR^{cCy1}R^{dCy1}, and S(O)₂R^{bCy1},
each R^{aCy1}, R^{bCy1}, R^{cCy1}, and R^{dCy1} is independently selected from H, C₁₋₃ alkyl, and C₁₋₃ haloalkyl;
R¹ is selected from H, D, C₁₋₃ alkyl, C₁₋₃ haloalkyl, C₂₋₃ alkenyl, C₂₋₃ alkynyl, cyclopropyl, halo, CN, OH, C₁₋₃ alkoxy, and C₁₋₃ haloalkoxy; wherein the C₁₋₃ alkyl, C₂₋₃ alkenyl, and C₂₋₃ alkynyl forming R¹ are each optionally substituted with 1 or 2 substituents independently selected from R^{1A}; and wherein the cyclopropyl forming R¹ is optionally substituted with 1 or 2 substituents independently selected from C₁₋₃ alkyl, C₁₋₃ haloalkyl, C₂₋₃ alkenyl, C₂₋₃ alkynyl, and R^{1A}; and wherein the C₁₋₃ alkyl, C₂₋₃ alkenyl, and C₂₋₃ alkynyl forming R¹ is optionally substituted with 1 or 2 substituents independently selected from R^{1B};
each R^{1A} is independently selected from C₁₋₃ alkyl, C₂₋₃ alkenyl, C₂₋₃ alkynyl and R^{1B}, wherein the C₁₋₃ alkyl, C₂₋₃ alkenyl, and C₂₋₃ alkynyl forming R^{1A} is optionally substituted with 1 or 2 substituents independently selected from R^{1B};
each R^{1B} is independently selected from D, halo, CN, OH, C₁₋₃ alkoxy, C₁₋₃ haloalkoxy, amino, C₁₋₃ alkylamino, and di(C₁₋₃ alkyl)amino;
R² is selected from H, D, C₁₋₃ alkyl, C₂₋₃ alkenyl, C₂₋₃ alkynyl, C₁₋₃ haloalkyl, Cy^{R2}, halo, CN, OR^{a2}, C(O)R^{b2}, C(O)NR^{c2}R^{d2}, NR^{c2}R^{e2}, and NR^{c2}C(O)R^{b2}; wherein the C₁₋₃ alkyl, C₂₋₃ alkenyl, and C₂₋₃ alkynyl forming R² are each optionally substituted with Cy^{R2}, and are also optionally substituted with 1, 2, or 3 substituents independently selected from R^{2B};
Cy^{R2} is selected from C₃₋₅ cycloalkyl, 4-6 membered heterocycloalkyl, C₆₋₁₀ aryl, and 5-6 membered heteroaryl, wherein the C₃₋₅cycloalkyl, 4-6 membered heterocycloalkyl, C₆₋₁₀ aryl, and 5-6 membered heteroaryl forming Cy^{R2} are each optionally substituted with 1, 2, or 3 substituents independently selected from R^{2A};
each R^{2A} is independently selected from C₁₋₃ alkyl, C₂₋₃ alkenyl, C₂₋₃ alkynyl, C₁₋₃ haloalkyl, and R^{2B}, wherein the C₁₋₃ alkyl, C₂₋₃ alkenyl, and C₂₋₃ alkynyl, forming R^{2A} are each optionally substituted with 1, 2 or 3 substituents independently selected from R^{2B};
each R^{2B} is independently selected from C₃₋₆ cycloalkyl, 4-10 membered heterocycloalkyl, C₆₋₁₀ aryl, 5-6 membered heteroaryl, halo, D, CN, OR^{a2B}, C(O)R^{b2B}, C(O)NR^{c2B}R^{d2B}, C(O)OR^{a2B}, NR^{c2B}R^{d2B}, and S(O)₂R^{b2B}; wherein the C₁₋₃ alkyl, C₃₋₆cycloalkyl, 4-6 membered heterocycloalkyl, C₆₋₁₀ aryl, and 5-6 membered heteroaryl forming R^{2B} are each optionally substituted with 1, 2, or 3 substituents independently selected from R^{2C};
each R^{2C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, 5-6 membered heteroaryl, halo, D, CN, OR^{a2C}, C(O)R^{b2C}, C(O)NR^{c2C}R^{d2C}, C(O)OR^{a2C}, NR^{c2C}R^{d2C}, and S(O)₂R^{b2C};
each R^{a2} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming R^{a2} are each optionally substituted with 1, 2, or 3 substituents independently selected from R^{2A}; and wherein the C₁₋₆ alkyl, C₂₋₆ alkenyl, and C₂₋₆ alkynyl forming R^{a2} are each optionally substituted with 1, 2, or 3 substituents independently selected from R^{2B};
each R^{b2}, R^{c2}, and R^{d2} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming R^{b2}, R^{c2}, and R^{d2} are each optionally substituted with 1, 2, or 3 substituents independently selected from R^{2A}; and wherein the C₁₋₃ alkyl, C₂₋₃ alkenyl, and C₂₋₃ alkynyl forming R^{b2}, R^{c2}, and R^{d2} are each optionally substituted with 1, 2, or 3 substituents independently selected from R^{2B}; or
any R^{c2} and R^{d2} attached to the same N atom, together with the N atom to which they are attached, form a 4-, 5-, or 6-membered heterocycloalkyl group optionally substituted with 1, 2, or 3 substituents independently selected from R^{2B};
each R^{e2} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming R^{e2} are each optionally substituted with 1, 2, or 3 substituents independently selected from R^{2A}; and wherein the C₁₋₃ alkyl, C₂₋₃ alkenyl, and C₂₋₃ alkynyl, forming R^{e2} are each optionally substituted with 1, 2, or 3 substituents independently selected from R^{2B}; or
R^{c2} and R^{e2} attached to the same N atom, together with the N atom to which they are attached, form a 4-, 5-, or 6-membered heterocycloalkyl group optionally substituted with 1, 2, or 3 substituents independently selected from R^{2B};
each R^{a2B}, R^{b2B}, R^{c2B} and R^{d2B} is independently selected from H, C₁₋₃ alkyl, and C₁₋₃ haloalkyl;
each R^{a2C}, R^{b2C}, R^{c2C} and R^{d2C} is independently selected from H, C₁₋₃ alkyl, and C₁₋₃ haloalkyl;
R³ is selected from H, D, C₁₋₃ alkyl, C₁₋₃ haloalkyl, C₂₋₃ alkenyl, C₂₋₃ alkynyl, C₃₋₆ cycloalkyl, halo, CN, and OR^{a3}; wherein the C₃₋₆ cycloalkyl forming R³ is optionally substituted with 1, 2 or 3 substituents independently selected from R^{3A}; and wherein the C₁₋₃ alkyl, C₂₋₃ alkenyl and C₂₋₃ alkynyl forming R³ are each optionally substituted with 1, 2, or 3 substituents independently selected from R^{3B};
each R^{3A} is independently selected from C₁₋₃ alkyl, C₂₋₃ alkenyl, C₂₋₃ alkynyl and R^{3B}, wherein the C₁₋₃ alkyl, C₂₋₃ alkenyl, and C₂₋₃ alkynyl forming R^{3A} are each optionally substituted by 1, 2 or 3 substituents each independently selected from R^{3B};
each R^{3B} is independently selected from D, OH, CN, halo, C₁₋₃ alkoxy, C₁₋₃ haloalkoxy, amino, C₁₋₃ alkylamino, and di(C₁₋₃ alkyl)amino;
R^{a3} is selected from H, C₁₋₃ alkyl, C₁₋₃ haloalkyl, C₂₋₃ alkenyl, and C₂₋₃ alkynyl;
R⁴ is selected from C₁₋₃ alkyl, C₂₋₃ alkenyl, C₂₋₃ alkynyl, C₁₋₃ haloalkyl, Cy^{R4}, OR^{4A}, and NR^{4B}R^{4C}; wherein the C₁₋₃ alkyl, C₂₋₃ alkenyl, and C₂₋₃ alkynyl forming R⁴ are each optionally substituted with Cy^{R4} and also optionally substituted with 1, 2, or 3 substituents independently selected from R^{4E};
Cy^{R4} is selected from C₃₋₁₀ cycloalkyl, 4-10 membered heterocycloalkyl, C₆₋₁₀ aryl, 5-10 membered heteroaryl, each optionally substituted with 1, 2, 3, 4, or 5 substituents independently selected from R^{4D};
R^{4A} is selected from C₁₋₃ alkyl, C₂₋₃ alkenyl, C₂₋₃ alkynyl, and Cy^{R4}; wherein the C₁₋₃ alkyl, C₂₋₃ alkenyl, and C₂₋₃ alkynyl forming R^{4A} are each optionally substituted with Cy^{R4} and also optionally substituted with 1, 2, or 3 substituents independently selected from R^{4E};
R^{4B} is selected from H, C₁₋₃ alkyl, C₂₋₃ alkenyl, C₂₋₃ alkynyl, and Cy^{R4}; wherein the C₁₋₃ alkyl, C₂₋₃ alkenyl, and C₂₋₃ alkynyl forming R^{4B} are each optionally substituted with Cy^{R4} and also optionally substituted with 1, 2, or 3 substituents independently selected from R^{4E}R^{4C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{4C} are each optionally substituted with 1, 2, or 3 substituents independently selected from R^{4E}; or
R^{4B} and R^{4C}, together with the N atom to which they are both attached, optionally form a 4-, 5-, 6-, or 7-membered heterocycloalkyl group that is optionally substituted with 1, 2, or 3 substituents independently selected from independently selected from R^{4D};
each R^{4D} is independently selected from C₁₋₃ alkyl, C₁₋₃ haloalkyl, C₂₋₃ alkenyl, C₂₋₃ alkynyl, and R^{4E}; wherein each of said C₁₋₃ alkyl, C₂₋₃ alkenyl, and C₂₋₃ alkynyl forming R^{4D} is optionally substituted with 1, 2, or 3 substituents independently selected from R^{4E};
each R^{4E} is independently selected from D, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, and S(O)₂NR^{c4}R^{d4}; R^{a4}, R^{b4}, R^{C4}, and R^{d4} are each independently selected from H, C₁₋₃ alkyl, C₂₋₃ alkenyl, C₂₋₃ alkynyl, and Cy^{Ra4} wherein the C₁₋₃ alkyl, C₂₋₃ alkenyl, and C₂₋₃ alkynyl forming R^{a4}, R^{b4}, R^{c4}, and R^{d4} are each optionally substituted by Cy^{Ra4};
Cy^{Ra4} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₃ alkyl, C₂₋₃ alkynyl, C₁₋₃ haloalkyl, C₂₋₃ alkenyl, halo, CN, OR^{a4A}, SR^{a4A}, C(O)R^{b4A}, C(O)NR^{c4A}R^{d4A}, C(O)OR^{a4A}, OC(O)R^{b4A}, OC(O)NR^{c4A}R^{d4A}, NR^{c4A}R^{d4A}, NR^{c4A}C(O)R^{b4A}, NR^{c4A}C(O)NR^{c4A}R^{d4A}, NR^{c4A}C(O)OR^{a4A}, C(═NR^{e4A})NR^{c4A}R^{d4A}, NR^{c4A}C(═NR^{e4A})NR^{c4A}R^{d4A}, S(O)R^{b4A}, S(O)NR^{c4A}R^{d4A}, S(O)₂R^{b4A}, NR^{c4A}S(O)₂R^{b4A}, and S(O)₂NR^{c4A}R^{d4A}; or
R^{c4} and R^{d4} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₂₋₃ alkenyl, C₂₋₃ alkynyl, C₁₋₃ haloalkyl, halo, CN, OR^{a4A}, SR^{a4A}, C(O)R^{b4A}, C(O)NR^{c4A}R^{d4A}, C(O)OR^{a4A}, OC(O)R^{b4A}, OC(O)NR^{c4A}R^{d4A}, NR^{c4A}R^{d4A}, NR^{c4A}C(O)R^{b4A}, NR^{c4A}C(O)NR^{c4A}R^{d4A}, NR^{c4A}C(O)OR^{a4A}, C(═NR^{e4A})NR^{c4A}R^{d4A}, NR^{c4A}C(═NR^{e4A})NR^{c4A}R^{d4A}, S(O)R^{b4A}, S(O)NR^{c4A}R^{d4A}, S(O)₂R^{b4A}, NR^{c4A}S(O)₂R^{b4A}, and S(O)₂NR^{c4A}R^{d4A};
R^{a4A}, R^{b4A}, R^{c4A}, and R^{d4A} are each independently selected from H, C₁₋₃ alkyl, C₂₋₃ alkenyl, C₂₋₃ alkynyl, C₁₋₃ haloalkyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a4A}, R^{b4A}, R^{c4A}, and R^{d4A} are each optionally substituted with 1, 2, or 3 substituents independently selected from OH, CN, amino, NH(C₁₋₆ alkyl), N(C₁₋₆ alkyl)₂, halo, C₁₋₆alkyl, C₂₋₆ alkenyl, C₂₋₆ alkynyl, C₁₋₆ alkoxy, C₁₋₆ haloalkyl, and C₁₋₆ haloalkoxy;
R^{c4A} and R^{d4A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy;
R^{e4} and R^{e4A} are each, independently, H, CN or NO₂;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl;
R⁶ is selected from H, C₁₋₆ alkyl, C₂₋₆ alkenyl, C₂₋₆ alkynyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from C₃₋₆cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is selected from H, C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6C} are each optionally substituted with Cy^{R6} and also optionally substituted with 1, 2, 3, or 4 substituents independently selected from R^{6F};
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E}; or
R^{6C} and R^{6D}, together with the N atom to which they are both attached, optionally form a 4-, 5-, 6-, or 7-membered heterocycloalkyl group or 5-membered heteroaryl group, that is optionally substituted with 1, 2, 3, or 4 substituents independently selected from independently selected from R^{6E};
each R^{6E} is independently selected from C₁₋₆ alkyl, C₁₋₆ haloalkyl, C₂₋₆ alkenyl, C₂₋₆ alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR⁶R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6};
Cy^{Ra6} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₆ alkyl, C₁₋₆ haloalkyl, C₂₋₆ alkenyl, C₂₋₆ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A}; or
R^{c6} and R^{d6} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₆ alkyl, C₁₋₆ haloalkyl, C₂₋₆ alkenyl, C₂₋₆ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A} NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A};
R^{a6A}, R^{b6A}, R^{c6A}, and R^{d6A} are each independently selected from H, C₁₋₆ alkyl, C₁₋₆ haloalkyl, C₂₋₆ alkenyl, C₂₋₆ alkynyl, aryl, C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl; wherein the C₁₋₆ alkyl, C₁₋₆ haloalkyl, C₂₋₆ alkenyl, C₂₋₆ alkynyl C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy; or
R^{c6A} and R^{d6A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy; and
R^{e6} and R^{e6A} are each, independently, H, CN or NO₂.

In an embodiment, the compound of Formula (I) is a compound of any one of the Formulae (I-A), (I-B), (I-C), (I-D), (I-E), (I-F), (I-G), (I-H), (I-I), and (I-J):

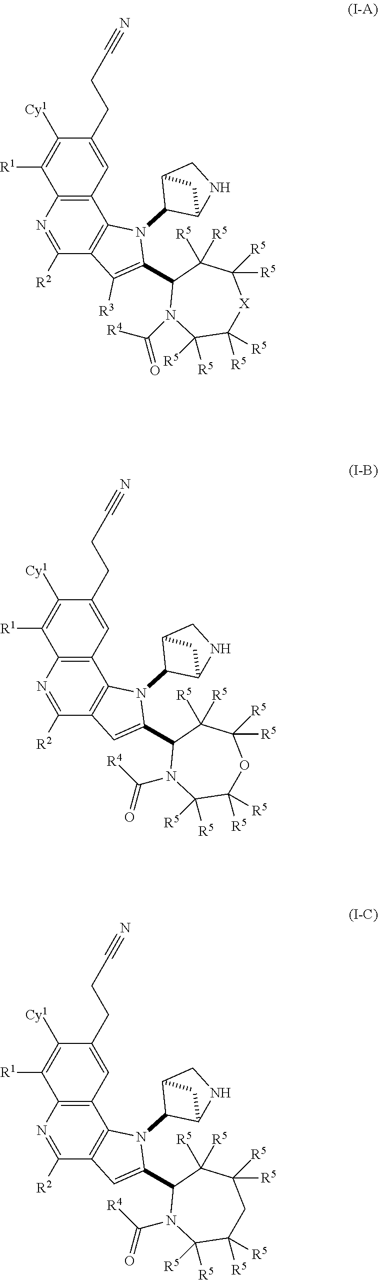

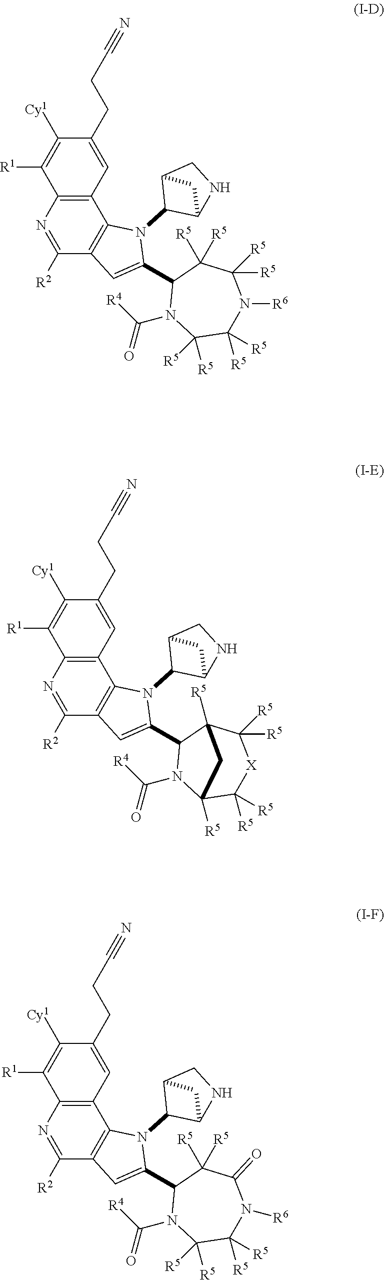

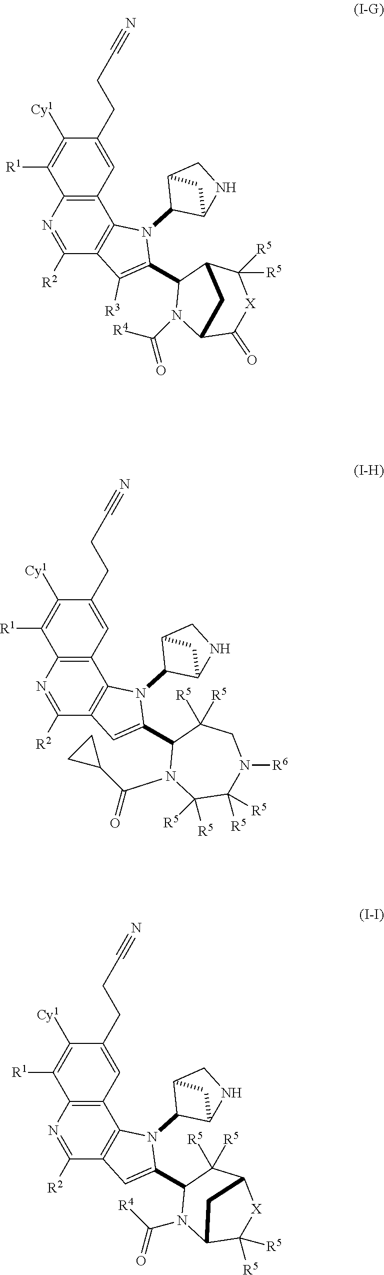

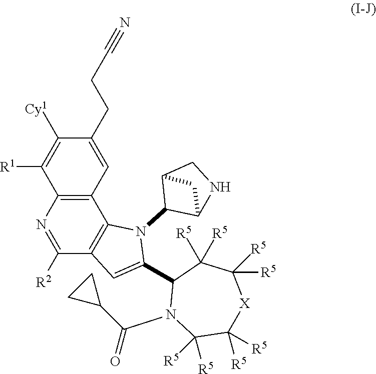

or a pharmaceutically acceptable salt thereof.

In another embodiment,
Cy¹ is C₆₋₁₀ aryl optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, C(O)R^{bCy1}, C(O)NR^{cCy1}R^{aCy1}, C(O)OR^{aCy1}, NR^{cCy1}R^{aCy1}, and S(O)₂R^{bCy1},
each R^{aCy1}, R^{bCy1}, R^{cCy1}, and R^{dCy1} is independently selected from H, C₁₋₃ alkyl, and C₁₋₃ haloalkyl;
R¹ is selected from H, D, C₁₋₃ alkyl, C₁₋₃ haloalkyl, C₂₋₃ alkenyl, C₂₋₃ alkynyl, cyclopropyl, halo, CN, OH, C₁₋₃ alkoxy, and C₁₋₃ haloalkoxy;
R² is selected from H, D, C₁₋₃ alkyl, C₂₋₃ alkenyl, C₂₋₃ alkynyl, C₁₋₃ haloalkyl, halo, and CN;
R³ is selected from H, D, C₁₋₃ alkyl, C₁₋₃ haloalkyl, C₂₋₃ alkenyl, C₂₋₃ alkynyl, C₃₋₆ cycloalkyl, halo, CN, and OR^{a3};
R^{a3} is H or C₁₋₃ alkyl;
R⁴ is selected from C₁₋₃ alkyl, C₂₋₃ alkenyl, C₂₋₃ alkynyl, C₁₋₃ haloalkyl, and Cy^{R4};
Cy^{R4} is selected from C₃₋₁₀ cycloalkyl and 4-10 membered heterocycloalkyl;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl; R⁶ is selected from H, C₁₋₆ alkyl, C₂₋₆ alkenyl, C₂₋₆ alkynyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from 3-6 cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is selected from H, C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6C} are each optionally substituted with Cy^{R6} and also optionally substituted with 1, 2, 3, or 4 substituents independently selected from R^{6F};
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E}; or
R^{6C} and R^{6D}, together with the N atom to which they are both attached, optionally form a 4-, 5-, 6-, or 7-membered heterocycloalkyl group or 5-membered heteroaryl group, that is optionally substituted with 1, 2, 3, or 4 substituents independently selected from independently selected from R^{6E};
each R^{6E} is independently selected from C₁₋₆ alkyl, C₁₋₆ haloalkyl, C₂₋₆ alkenyl, C₂₋₆ alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6};
Cy^{Ra6} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₆ alkyl, C₁₋₆ haloalkyl, C₂₋₆ alkenyl, C₂₋₆ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, S(O)R^{b6A}, S(O)NR^{c6A}R^{d6A}S(O)₂R^{b6A}, NR^{c6A}S(O)₂R^{b6A}, and S(O)₂NR^{c6A}R^{d6A}; and
R^{a6A}, R^{b6A}, R^{c6A}, and R^{d6A} are each independently selected from H, C₁₋₆ alkyl, C₁₋₆ haloalkyl, C₂₋₆ alkenyl, C₂₋₆ alkynyl, aryl, C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl; wherein the C₁₋₆ alkyl, C₁₋₆ haloalkyl, C₂₋₆ alkenyl, C₂₋₆ alkynyl C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy.

In yet another embodiment,
Cy¹ is C₆₋₁₀ aryl optionally substituted with 1, 2, 3, or 4 substituents independently selected from halo;
R¹ is halo;
R² is C₁₋₃ alkyl;
R³ is H;
R⁴ is Cy^{R4};
Cy^{R4} is C₃₋₁₀ cycloalkyl;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl;
R⁶ is selected from H, C₁₋₆ alkyl, C₂₋₆ alkenyl, C₂₋₆ alkynyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from C₃₋₆cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is H;
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E};
each R^{6E} is independently selected from C₁₋₆ alkyl, C₁₋₆ haloalkyl, C₂₋₆ alkenyl, C₂₋₆ alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6}; and
Cy^{Ra6} is selected from C₆₋₁₀ aryl, C₃₋₇ cycloalkyl, 5-10 membered heteroaryl, 4-10 membered heterocycloalkyl.

In still another embodiment,
Cy¹ is C₆₋₁₀ aryl optionally substituted with 1, 2, or 3 substituents independently selected from halo;
R¹ is halo;
R² is C₁₋₃ alkyl;
R³ is H;
R⁴ is Cy^{R4};
Cy^{R4} is C₃₋₁₀ cycloalkyl;
X is C(R⁵)₂, O, or N—R⁶;
each R⁵ is independently selected from H, D, halo, C₁₋₃ alkyl, and C₁₋₃ haloalkyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
R⁶ is selected from C₁₋₆ alkyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl forming R⁶ is optionally substituted with Cy^{R6}, and also optionally substituted with 1, 2, or 3 substituents independently selected from R^{6E};
Cy^{R6} is selected from C₃₋₆cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₁₋₆ haloalkyl, and Cy^{R6}; wherein the C₁₋₆ alkyl forming R^{6B} is optionally substituted with Cy^{R6} and also optionally substituted with 1 or 2 substituents independently selected from R^{6F};
R^{6C} is H;
R^{6D} is selected from H, C₁₋₆ alkyl, and C₁₋₆ haloalkyl; wherein the C₁₋₆ alkyl forming R^{6D} is optionally substituted with 1 or 2 substituents independently selected from R^{6E};
each R^{6E} is independently selected from C₁₋₆ alkyl, C₁₋₆ haloalkyl, and R^{6F}; wherein each of said C₁₋₆ alkyl forming R^{6E} is optionally substituted with 1, 2, or 3 substituents independently selected from R^{6F};
each R^{6F} is independently selected from D, halo, CN, OR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, and Cy^{Ra6} wherein the C₁₋₆ alkyl forming R^{a6}, R^{b6}, R^{c6}, and R^{d6} is optionally substituted by Cy^{Ra6}; and
Cy^{Ra6} is C₃₋₇ cycloalkyl.

In an embodiment, X is N—R⁶. In another embodiment, X is C(R⁵)₂. In yet another embodiment, X is O.

In still another embodiment, Cy¹ is phenyl optionally substituted with 1, 2, or 3 substituents independently selected from R^{Cy1}. In an embodiment, Cy¹ is phenyl optionally substituted with 1, 2, or 3 substituents independently selected from halo. In another embodiment, Cy¹ is phenyl optionally substituted with 1 or 2 chloro. In yet another embodiment, Cy¹ is 2,3-dichlorophenyl.

In an embodiment, R¹ is halo. In another embodiment, R¹ is fluoro.

In yet another embodiment, R² is C₁₋₃ alkyl. In still another embodiment, R² is methyl.

In an embodiment, R³ is H.

In another embodiment, R⁴ is Cy^{R4}.

In yet another embodiment, Cy^{R4} is C₃₋₆cycloalkyl. In still another embodiment, Cy^{R4} is cyclopropyl.

In an embodiment, each R⁵ is selected from H; optionally, two R⁵ attached to the same carbon atom, together with the carbon atom to which they are both attached, form a carbonyl; or, optionally, two R⁵ together form a bridged ring when both R⁵ forming the C₁₋₄ alkylene are attached to different, non-adjacent carbon atoms. In another embodiment, each R⁵ is selected from H; optionally, two R⁵ attached to the same carbon atom, together with the carbon atom to which they are both attached, form a carbonyl; or, optionally, two R⁵ together form a bridged ring when both R⁵ forming the C₁ alkylene are attached to different, non-adjacent carbon atoms.

In yet another embodiment, R⁶ is selected from Cy^{R6}, C(O)R^{6A}, C(O)OR^{6B}, C(O)NR^{6C}R^{6D}, and SO₂R^{6A}.

In still another embodiment, R^{6A} is Cy^{R6}.

In an embodiment, Cy^{R6} is selected from C₃₋₆ cycloalkyl, 4-6 membered heterocycloalkyl, C₆₋₁₀ aryl, and 5-10 membered heteroaryl, wherein the C₃₋₅ cycloalkyl, 4-6 membered heterocycloalkyl, C₆₋₁₀ aryl, and 5-10 membered heteroaryl forming Cy^{R6} are each optionally substituted with 1, 2, or 3 substituents independently selected from R^{6E}.

In another embodiment, Cy^{R6} is selected from phenyl, 2-fluorophenyl, 3-fluorophenyl, 4-fluorophenyl, 4-(trifluoromethoxy)phenyl, 4-cyanophenyl, 6-(trifluoromethyl)pyridin-3-yl, 5-naphthyridin-3-yl, 2,2-difluorobenzo[d][1,3]dioxol-5-yl, 1-(difluoromethyl)-1H-pyrazol-4-yl, pyridin-2-yl, pyridin-3-yl, pyridin-4-yl, 3-(trifluoromethyl)phenyl, 1-methyl-1H-pyrazol-4-yl, 4-(trifluoromethyl)phenyl, 6-methylpyridin-3-yl, 3,4-dichlorophenyl, 2-(trifluoromethyl)phenyl, 3-cyanophenyl, 3,5-bis(trifluoromethyl)phenyl, 3-chlorophenyl, 3-(trifluoromethoxy)phenyl, 5-(trifluoromethyl)pyridin-3-yl, 6-(difluoromethyl)pyridin-3-yl, 6-methoxypyridin-3-yl, 4-(difluoromethoxy)phenyl, 3-(difluoromethoxy)phenyl, 1-(trifluoromethyl)-1H-pyrazol-4-yl, 2-fluoro-4-(trifluoromethoxy)phenyl, 1-methyl-3-(trifluoromethyl)-1H-pyrazol-4-yl, 5-(trifluoromethyl)pyridin-2-yl, 5-fluoro-6-methylpyrimidin-4-yl, 5-isopropylthiazol-2-yl, pyrimidin-2-yl, 6-(trifluoromethyl)pyridazin-3-yl, 1-methyl-2-oxo-1,2-dihydropyridin-4-yl, 2-(trifluoromethyl)pyridin-4-yl, 4-(trifluoromethyl)pyridin-2-yl, pyrazin-2-yl, 5-(trifluoromethyl)pyrazin-2-yl, 5-(trifluoromethyl)pyrimidin-2-yl, 4-(trifluoromethyl)pyrimidin-2-yl, 4-methoxypyrimidin-2-yl, 5-(difluoromethoxy)pyrimidin-2-yl, 3-fluoropyridin-2-yl, 5-cyanopyridin-2-yl, 3-cyanopyridin-4-yl, 3-methylpyridin-2-yl, 3-methoxypyridin-2-yl, 3-fluoro-4-(trifluoromethyl)pyridin-2-yl, 3-fluoro-4-methylpyridin-2-yl, 3-fluoro-6-methylpyridin-2-yl, 3-fluoro-5-methylpyridin-2-yl, 4-(difluoromethyl)-3-fluoropyridin-2-yl, 3-fluoro-4-methoxypyridin-2-yl, 1-(difluoromethyl)-2-oxo-1,2-dihydropyridin-4-yl, 1-methyl-6-oxo-1,6-dihydropyridin-2-yl, 1-isobutyl-2-oxo-1,2-dihydropyridin-4-yl, 2-methoxypyridin-4-yl, 2-(difluoromethyl)pyridin-4-yl, imidazo[1,2-a]pyridin-7-yl, 2-hydroxypropan-2-yl)pyridin-4-yl, N,N-dimethyl-3-amidopyridin-4yl, 1-methyl-6-oxo-1,6-dihydropyridazin-4-yl, 1,6-dimethyl-2-oxo-1,2-dihydropyridin-4-yl, 1-ethyl-2-oxo-1,2-dihydropyridin-4-yl, 1-isopropyl-2-oxo-1,2-dihydropyridin-4-yl, 1-methyl-6-oxo-1,6-dihydropyrimidin-4-yl, 2-(trifluoromethyl)imidazo[1,2-a]pyridin-7-yl, pyrazolo[1,5-a]pyridin-5-yl, [1,2,4]triazolo[1,5-a]pyridin-7-yl, 2-oxo-2H-pyran-4-yl, 2-bromopyridin-4-yl, 4-fluoro-3-methylpyridin-2-yl, 4-fluoro-3-methylpyridin-4-yl, 2-methoxy-3-methylpyridin-4-yl, 2-methoxy-6-methylpyridin-4-yl, 2-fluoro-6-methylpyridin-4-yl, 2-ethoxypyridin-4-yl, 2-(cyclopropylmethoxy)pyridin-4-yl, 2-(2-methoxyethoxy)pyridin-4-yl, 2-(difluoromethyl)-6-methylpyridin-4-yl, 4-(difluoromethyl)-5-fluoropyridin-2-yl, 6-(difluoromethoxy)pyridin-2-yl, 4-(difluoromethyl)pyrimidin-2-yl, 6-(difluoromethyl)pyridin-2-yl, 4-(difluoromethyl)pyridin-2-yl, 4-methylthiazol-2-yl, 5-methylthiazol-2-yl, 1-methyl-1H-1,2,4-triazol-5-yl, thiazol-4-yl, 4-(trifluoromethyl)thiazol-2-yl, 4,5-dimethylthiazol-2-yl, 4-cyanothiazol-2-yl, 3-methyl-4-cyanothiazol-2-yl, 3-cyano-4-methylthiazol-2-yl, 5-ethylthiazol-2-yl, isoxazol-3-yl, 1,5-dimethyl-1H-imidazol-2-yl, 5-(difluoromethyl)thiazol-2-yl, 1,1-difluoroethyl-5-methylthiazol-2-yl, isothiazol-4-yl, 1,5-dimethyl-1H-pyrazol-3-yl, and 5-cyclopropylthiazol-2-yl, and cyclohexyl.

In yet another embodiment, Cy^{R6} is selected from cyclopropanecarbonyl, methylcarboxylate, 2,2,2-trifluoroethylcarboxamide, 3,5-dimethylisoxazol-4-yl)sulfonyl, 3-morpholinosulfonyl, methyl, and benzoyl.

In still another embodiment, R^{6B} is C₁₋₆ alkyl.

In an embodiment, R^{6C} is H. In another embodiment, R^{6D} is selected from H, C₁₋₄ alkyl, and C₁₋₄ haloalkyl. In yet another embodiment, R^{6C} is H; and R^{6D} is selected from H, C₁₋₄ alkyl, and C₁₋₄ haloalkyl.

In still another embodiment, each R^{6E} is independently selected from C₁₋₆ alkyl, C₁₋₆ haloalkyl, halo, CN, OR^{a6}, and C(O)NR^{c6}R^{d6}.

In an embodiment, each R^{a6}, R^{b6}, R^{c6}, and R^{d6} are each independently selected from H, C₁₋₆ alkyl, C₁₋₆ alkyl-O—C₁₋₆ alkyl, and C₁₋₆ haloalkyl, wherein alkyl is optionally substituted with C₃₋₇ cycloalkyl.

In other embodiments, the compound of Formula (I) is selected from a compound in Table 1, and pharmaceutically acceptable salts thereof.

TABLE 1

| Entry | Compound Name |
|---|---|
| 1 | 3-(1-(2-azabicyclo[2.1.1]hexan-5-yl)-2-(6-(cyclopropanecarbonyl)-3-phenyl-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 2 | 3-(1-(2-azabicyclo[2.1.1]hexan-5-yl)-2-(6-(cyclopropanecarbonyl)-3-(2- |
| | fluorophenyl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 3 | 3-(1-(2-azabicyclo[2.1.1]hexan-5-yl)-2-(6-(cyclopropanecarbonyl)-3-(4- |
| | (trifluoromethoxy)phenyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 4 | 4-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)benzonitrile |
| 5 | 3-(1-(2-azabicyclo[2.1.1]hexan-5-yl)-2-(6-(cyclopropanecarbonyl)-3-(6- |
| | (trifluoromethyl)pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 6 | 3-(1-(2-azabicyclo[2.1.1]hexan-5-yl)-2-(6-(cyclopropanecarbonyl)-3-(1,5- |
| | naphthyridin-3-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 7 | 3-(1-(2-azabicyclo[2.1.1]hexan-5-yl)-2-(6-(cyclopropanecarbonyl)-3-(2,2- |
| | difluorobenzo[d][1,3]dioxol-5-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 8 | 3-(1-(2-azabicyclo[2.1.1]hexan-5-yl)-2-(6-(cyclopropanecarbonyl)-3-(1- |
| | (difluoromethyl)-1H-pyrazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 9 | 3-(1-(2-azabicyclo[2.1.1]hexan-5-yl)-2-(6-(cyclopropanecarbonyl)-3-(pyridin-3-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 10 | 3-(1-(2-azabicyclo[2.1.1]hexan-5-yl)-2-(6-(cyclopropanecarbonyl)-3-(3- |
| | (trifluoromethyl)phenyl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 11 | 3-(1-(2-azabicyclo[2.1.1]hexan-5-yl)-2-(6-(cyclopropanecarbonyl)-3-(1-methyl-1H- |
| | pyrazol-4-yl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 12 | 3-(1-(2-azabicyclo[2.1.1]hexan-5-yl)-2-(6-(cyclopropanecarbonyl)-3-(4- |
| | (trifluoromethyl)phenyl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 13 | 3-(1-(2-azabicyclo[2.1.1]hexan-5-yl)-2-(6-(cyclopropanecarbonyl)-3-(3- |
| | fluorophenyl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 14 | 3-(1-(2-azabicyclo[2.1.1]hexan-5-yl)-2-(6-(cyclopropanecarbonyl)-3-(6- |
| | methylpyridin-3-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 15 | 3-(1-(2-azabicyclo[2.1.1]hexan-5-yl)-2-(6-(cyclopropanecarbonyl)-3-(3,4- |
| | dichlorophenyl)-3,6-diazabicyclo[3.2.1]octan-7-yl)-7-(2,3-dichlorophenyl)-6-fluoro- |
| | 4-methyl-1H-pyrrolo[3,2-c]quinolin-8-yl)propanenitrile |
| 16 | 3-(1-(2-azabicyclo[2.1.1]hexan-5-yl)-2-(6-(cyclopropanecarbonyl)-3-(2- |
| | (trifluoromethyl)phenyl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 17 | 3-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)benzonitrile |
| 18 | 3-(1-(2-azabicyclo[2.1.1]hexan-5-yl)-2-(3-(3,5-bis(trifluoromethyl)phenyl)-6- |
| | (cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 19 | 3-(1-(2-azabicyclo[2.1.1]hexan-5-yl)-2-(3-(3-chlorophenyl)-6- |
| | (cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 20 | 3-(1-(2-azabicyclo[2.1.1]hexan-5-yl)-2-(6-(cyclopropanecarbonyl)-3-(3- |
| | (trifluoromethoxy)phenyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 21 | 3-(1-(2-azabicyclo[2.1.1]hexan-5-yl)-2-(6-(cyclopropanecarbonyl)-3-(5- |
| | (trifluoromethyl)pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 22 | 3-(1-(2-azabicyclo[2.1.1]hexan-5-yl)-2-(6-(cyclopropanecarbonyl)-3-(6- |
| | (difluoromethyl)pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 23 | 3-(1-(2-azabicyclo[2.1.1]hexan-5-yl)-2-(6-(cyclopropanecarbonyl)-3-(4- |
| | fluorophenyl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 24 | 3-(1-(2-azabicyclo[2.1.1]hexan-5-yl)-2-(6-(cyclopropanecarbonyl)-3-(6- |
| | methoxypyridin-3-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 25 | 3-(1-(2-azabicyclo[2.1.1]hexan-5-yl)-2-(6-(cyclopropanecarbonyl)-3-(4- |
| | (difluoromethoxy)phenyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 26 | 3-(1-(2-azabicyclo[2.1.1]hexan-5-yl)-2-(6-(cyclopropanecarbonyl)-3-(3- |
| | (difluoromethoxy)phenyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 27 | 3-(1-(2-azabicyclo[2.1.1]hexan-5-yl)-2-(6-(cyclopropanecarbonyl)-3-(1- |
| | (trifluoromethyl)-1H-pyrazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 28 | 3-(1-(2-azabicyclo[2.1.1]hexan-5-yl)-2-(6-(cyclopropanecarbonyl)-3-(2-fluoro-4- |
| | (trifluoromethoxy)phenyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 29 | 3-(1-(2-azabicyclo[2.1.1]hexan-5-yl)-2-(6-(cyclopropanecarbonyl)-3-(1-methyl-3- |
| | (trifluoromethyl)-1H-pyrazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 30 | 3-(1-(2-azabicyclo[2.1.1]hexan-5-yl)-2-(6-(cyclopropanecarbonyl)-3-(5- |
| | (trifluoromethyl)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 31 | 3-(1-(2-azabicyclo[2.1.1]hexan-5-yl)-2-(6-(cyclopropanecarbonyl)-3-(5-fluoro-6- |
| | methylpyrimidin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 32 | 3-(1-(2-azabicyclo[2.1.1]hexan-5-yl)-2-(6-(cyclopropanecarbonyl)-3-(5- |
| | isopropylthiazol-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 33 | 3-(1-(2-azabicyclo[2.1.1]hexan-5-yl)-2-(6-(cyclopropanecarbonyl)-3-(pyrimidin-2- |
| | yl)-3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 34 | 3-(1-(2-azabicyclo[2.1.1]hexan-5-yl)-2-(6-(cyclopropanecarbonyl)-3-(6- |
| | (trifluoromethyl)pyridazin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 35 | 3-(1-(2-azabicyclo[2.1.1]hexan-5-yl)-2-(6-(cyclopropanecarbonyl)-3-(1-methyl-2- |
| | oxo-1,2-dihydropyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 36 | 3-(1-(2-azabicyclo[2.1.1]hexan-5-yl)-2-(6-(cyclopropanecarbonyl)-3-(pyridin-2-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 37 | 3-(1-(2-azabicyclo[2.1.1]hexan-5-yl)-2-(6-(cyclopropanecarbonyl)-3-(pyridin-4-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 38 | 3-(1-(2-azabicyclo[2.1.1]hexan-5-yl)-2-(6-(cyclopropanecarbonyl)-3-(2- |
| | (trifluoromethyl)pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 39 | 3-(1-(2-azabicyclo[2.1.1]hexan-5-yl)-2-(6-(cyclopropanecarbonyl)-3-(4- |
| | (trifluoromethyl)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 40 | 3-(1-(2-azabicyclo[2.1.1]hexan-5-yl)-2-(6-(cyclopropanecarbonyl)-3-(pyrazin-2-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 41 | 3-(1-(2-azabicyclo[2.1.1]hexan-5-yl)-2-(6-(cyclopropanecarbonyl)-3-(5- |
| | (trifluoromethyl)pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 42 | 3-(1-(2-azabicyclo[2.1.1]hexan-5-yl)-2-(6-(cyclopropanecarbonyl)-3-(4- |
| | methoxypyrimidin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 43 | 3-(1-(2-azabicyclo[2.1.1]hexan-5-yl)-2-(6-(cyclopropanecarbonyl)-3-(4- |
| | (trifluoromethyl)pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 44 | 3-(1-(2-azabicyclo[2.1.1]hexan-5-yl)-2-(6-(cyclopropanecarbonyl)-3-(5- |
| | (trifluoromethyl)pyrazin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 45 | 3-(1-(2-azabicyclo[2.1.1]hexan-5-yl)-2-(6-(cyclopropanecarbonyl)-3-(5- |
| | (difluoromethoxy)pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 46 | 3-(1-(2-azabicyclo[2.1.1]hexan-5-yl)-2-(6-(cyclopropanecarbonyl)-3-(3- |
| | fluoropyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 47 | 2-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)nicotinonitrile |
| 48 | 3-(1-(2-azabicyclo[2.1.1]hexan-5-yl)-2-(6-(cyclopropanecarbonyl)-3-(3- |
| | methylpyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 49 | 3-(1-(2-azabicyclo[2.1.1]hexan-5-yl)-2-(6-(cyclopropanecarbonyl)-3-(3- |
| | methoxypyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 50 | 3-(1-(2-azabicyclo[2.1.1]hexan-5-yl)-2-(6-(cyclopropanecarbonyl)-3-(3-fluoro-4- |
| | (trifluoromethyl)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 51 | 3-(1-(2-azabicyclo[2.1.1]hexan-5-yl)-2-(6-(cyclopropanecarbonyl)-3-(3-fluoro-4- |
| | methylpyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 52 | 3-(1-(2-azabicyclo[2.1.1]hexan-5-yl)-2-(6-(cyclopropanecarbonyl)-3-(3-fluoro-6- |
| | methylpyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 53 | 3-(1-(2-azabicyclo[2.1.1]hexan-5-yl)-2-(6-(cyclopropanecarbonyl)-3-(3-fluoro-5- |
| | methylpyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 54 | 3-(1-(2-azabicyclo[2.1.1]hexan-5-yl)-2-(6-(cyclopropanecarbonyl)-3-(4- |
| | (difluoromethyl)-3-fluoropyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 55 | 3-(1-(2-azabicyclo[2.1.1]hexan-5-yl)-2-(6-(cyclopropanecarbonyl)-3-(3-fluoro-4- |
| | methoxypyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 56 | 3-(1-(2-azabicyclo[2.1.1]hexan-5-yl)-2-(6-(cyclopropanecarbonyl)-3-(1- |
| | (difluoromethyl)-2-oxo-1,2-dihydropyridin-4-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 57 | 3-(1-(2-azabicyclo[2.1.1]hexan-5-yl)-2-(6-(cyclopropanecarbonyl)-3-(1-methyl-6- |
| | oxo-1,6-dihydropyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 58 | 3-(1-(2-azabicyclo[2.1.1]hexan-5-yl)-2-(6-(cyclopropanecarbonyl)-3-(1-isobutyl-2- |
| | oxo-1,2-dihydropyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 59 | 3-(1-(2-azabicyclo[2.1.1]hexan-5-yl)-2-(6-(cyclopropanecarbonyl)-3-(2- |
| | methoxypyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 60 | 3-(1-(2-azabicyclo[2.1.1]hexan-5-yl)-2-(6-(cyclopropanecarbonyl)-3-(2- |
| | (difluoromethyl)pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 61 | 3-(1-(2-azabicyclo[2.1.1]hexan-5-yl)-2-(6-(cyclopropanecarbonyl)-3-(imidazo[1,2- |
| | a]pyridin-7-yl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 62 | 3-(1-(2-azabicyclo[2.1.1]hexan-5-yl)-2-(6-(cyclopropanecarbonyl)-3-(2-(2- |
| | hydroxypropan-2-yl)pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 63 | 4-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)picolinonitrile |
| 64 | 4-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)-N,N-dimethylpicolinamide |
| 65 | 3-((R)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridazin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 66 | 3-(1-(2-azabicyclo[2.1.1]hexan-5-yl)-2-(6-(cyclopropanecarbonyl)-3-(1,6-dimethyl- |
| | 2-oxo-1,2-dihydropyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 67 | 3-(1-(2-azabicyclo[2.1.1]hexan-5-yl)-2-(6-(cyclopropanecarbonyl)-3-(1-ethyl-2-oxo- |
| | 1,2-dihydropyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 68 | 3-(1-(2-azabicyclo[2.1.1]hexan-5-yl)-2-(6-(cyclopropanecarbonyl)-3-(1-isopropyl-2- |
| | oxo-1,2-dihydropyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 69 | 3-(1-(2-azabicyclo[2.1.1]hexan-5-yl)-2-(6-(cyclopropanecarbonyl)-3-(1-methyl-6- |
| | oxo-1,6-dihydropyrimidin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 70 | 3-(1-(2-azabicyclo[2.1.1]hexan-5-yl)-2-(6-(cyclopropanecarbonyl)-3-(2- |
| | (trifluoromethyl)imidazo[1,2-a]pyridin-7-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 71 | 3-(1-(2-azabicyclo[2.1.1]hexan-5-yl)-2-(6-(cyclopropanecarbonyl)-3-(pyrazolo[1,5- |
| | a]pyridin-5-yl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 72 | 3-(2-(3-([1,2,4]triazolo[1,5-a]pyridin-7-yl)-6-(cyclopropanecarbonyl)-3,6- |
| | diazabicyclo[3.2.1]octan-7-yl)-1-(2-azabicyclo[2.1.1]hexan-5-yl)-7-(2,3- |
| | dichlorophenyl)-6-fluoro-4-methyl-1H-pyrrolo[3,2-c]quinolin-8-yl)propanenitrile |
| 73 | 3-(1-(2-azabicyclo[2.1.1]hexan-5-yl)-2-(6-(cyclopropanecarbonyl)-3-(2-oxo-2H- |
| | pyran-4-yl)-3,6-diazabicyclo[3.2.1]octan-7-yl)-7-(2,3-dichlorophenyl)-6-fluoro-4- |
| | methyl-1H-pyrrolo[3,2-c]quinolin-8-yl)propanenitrile |
| 74 | 3-(1-(2-azabicyclo[2.1.1]hexan-5-yl)-2-(3-(2-bromopyridin-4-yl)-6- |
| | (cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 75 | 3-(1-(2-azabicyclo[2.1.1]hexan-5-yl)-2-(6-(cyclopropanecarbonyl)-3-(4-fluoro-3- |
| | methylpyridin-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 76 | 3-(1-(2-azabicyclo[2.1.1]hexan-5-yl)-2-(6-(cyclopropanecarbonyl)-3-(2-fluoro-3- |
| | methylpyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 77 | 3-(1-(2-azabicyclo[2.1.1]hexan-5-yl)-2-(6-(cyclopropanecarbonyl)-3-(2-methoxy-3- |
| | methylpyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 78 | 3-(1-(2-azabicyclo[2.1.1]hexan-5-yl)-2-(6-(cyclopropanecarbonyl)-3-(2-methoxy-6- |
| | methylpyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 79 | 3-(1-(2-azabicyclo[2.1.1]hexan-5-yl)-2-(6-(cyclopropanecarbonyl)-3-(2-fluoro-6- |
| | methylpyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 80 | 3-(1-(2-azabicyclo[2.1.1]hexan-5-yl)-2-(6-(cyclopropanecarbonyl)-3-(2-fluoro-6- |
| | methylpyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 81 | 3-(1-(2-azabicyclo[2.1.1]hexan-5-yl)-2-(6-(cyclopropanecarbonyl)-3-(2- |
| | ethoxypyridin-4-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 82 | 3-(1-(2-azabicyclo[2.1.1]hexan-5-yl)-2-(6-(cyclopropanecarbonyl)-3-(2- |
| | (cyclopropylmethoxy)pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 83 | 3-(1-(2-azabicyclo[2.1.1]hexan-5-yl)-2-(6-(cyclopropanecarbonyl)-3-(2-(2- |
| | methoxyethoxy)pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 84 | 3-(1-(2-azabicyclo[2.1.1]hexan-5-yl)-2-(6-(cyclopropanecarbonyl)-3-(2- |
| | (difluoromethyl)-6-methylpyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 85 | 3-(1-(2-azabicyclo[2.1.1]hexan-5-yl)-2-(6-(cyclopropanecarbonyl)-3-(4- |
| | (difluoromethyl)-5-fluoropyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 86 | 3-(1-(2-azabicyclo[2.1.1]hexan-5-yl)-2-(6-(cyclopropanecarbonyl)-3-(6- |
| | (difluoromethoxy)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 87 | 3-(1-(2-azabicyclo[2.1.1]hexan-5-yl)-2-(6-(cyclopropanecarbonyl)-3-(4- |
| | (difluoromethyl)pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 88 | 3-(1-(2-azabicyclo[2.1.1]hexan-5-yl)-2-(6-(cyclopropanecarbonyl)-3-(6- |
| | (difluoromethyl)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 89 | 3-(1-(2-azabicyclo[2.1.1]hexan-5-yl)-2-(6-(cyclopropanecarbonyl)-3-(4- |
| | (difluoromethyl)pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 90 | 3-(1-(2-azabicyclo[2.1.1]hexan-5-yl)-2-(6-(cyclopropanecarbonyl)-3-(4- |
| | methylthiazol-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 91 | 3-(1-(2-azabicyclo[2.1.1]hexan-5-yl)-2-(6-(cyclopropanecarbonyl)-3-(5- |
| | methylthiazol-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 92 | 3-(1-(2-azabicyclo[2.1.1]hexan-5-yl)-2-(6-(cyclopropanecarbonyl)-3-(1-methyl-1H- |
| | 1,2,4-triazol-5-yl)-3,6-diazabicyclo[3.2.1]octan-7-yl)-7-(2,3-dichlorophenyl)-6-fluoro- |
| | 4-methyl-1H-pyrrolo[3,2-c]quinolin-8-yl)propanenitrile |
| 93 | 3-(1-(2-azabicyclo[2.1.1]hexan-5-yl)-2-(6-(cyclopropanecarbonyl)-3-(thiazol-4-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 94 | 3-(1-(2-azabicyclo[2.1.1]hexan-5-yl)-2-(6-(cyclopropanecarbonyl)-3-(4- |
| | (trifluoromethyl)thiazol-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 95 | 3-(1-(2-azabicyclo[2.1.1]hexan-5-yl)-2-(6-(cyclopropanecarbonyl)-3-(4,5- |
| | dimethylthiazol-2-yl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 96 | 2-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)thiazole-4-carbonitrile |
| 97 | 2-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)-5-methylthiazole-4-carbonitrile |
| 98 | 2-(7-(1-(2-azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-3,6- |
| | diazabicyclo[3.2.1]octan-3-yl)-4-methylthiazole-5-carbonitrile |
| 99 | 3-(1-(2-azabicyclo[2.1.1]hexan-5-yl)-2-(6-(cyclopropanecarbonyl)-3-(5-ethylthiazol- |
| | 2-yl)-3,6-diazabicyclo[3.2.1]octan-7-yl)-7-(2,3-dichlorophenyl)-6-fluoro-4-methyl- |
| | 1H-pyrrolo[3,2-c]quinolin-8-yl)propanenitrile |
| 100 | 3-(1-(2-azabicyclo[2.1.1]hexan-5-yl)-2-(6-(cyclopropanecarbonyl)-3-(isoxazol-3-yl)- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 101 | 3-(1-(2-azabicyclo[2.1.1]hexan-5-yl)-2-(6-(cyclopropanecarbonyl)-3-(1,5-dimethyl- |
| | 1H-imidazol-2-yl)-3,6-diazabicyclo[3.2.1]octan-7-yl)-7-(2,3-dichlorophenyl)-6-fluoro- |
| | 4-methyl-1H-pyrrolo[3,2-c]quinolin-8-yl)propanenitrile |
| 102 | 3-(1-(2-azabicyclo[2.1.1]hexan-5-yl)-2-(6-(cyclopropanecarbonyl)-3-(5- |
| | (difluoromethyl)thiazol-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 103 | 3-(1-(2-azabicyclo[2.1.1]hexan-5-yl)-2-(6-(cyclopropanecarbonyl)-3-(4-(1,1- |
| | difluoroethyl)-5-methylthiazol-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 104 | 3-(1-(2-azabicyclo[2.1.1]hexan-5-yl)-2-(6-(cyclopropanecarbonyl)-3-(isothiazol-4- |
| | yl)-3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 105 | 3-(1-(2-azabicyclo[2.1.1]hexan-5-yl)-2-(6-(cyclopropanecarbonyl)-3-(1,5-dimethyl- |
| | 1H-pyrazol-3-yl)-3,6-diazabicyclo[3.2.1]octan-7-yl)-7-(2,3-dichlorophenyl)-6-fluoro- |
| | 4-methyl-1H-pyrrolo[3,2-c]quinolin-8-yl)propanenitrile |
| 106 | 3-(1-(2-azabicyclo[2.1.1]hexan-5-yl)-2-(6-(cyclopropanecarbonyl)-3-(5- |
| | cyclopropylthiazol-2-yl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 107 | 3-(1-(2-Azabicyclo[2.1.1]hexan-5-yl)-2-(3,6-di(cyclopropanecarbonyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 108 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octane-3-carboxylate |
| 109 | 7-(1-(2-Azabicyclo[2.1.1]hexan-5-yl)-8-(2-cyanoethyl)-7-(2,3-dichlorophenyl)-6- |
| | fluoro-4-methyl-1H-pyrrolo[3,2-c]quinolin-2-yl)-6-(cyclopropanecarbonyl)-N-(2,2,2- |
| | trifluoroethyl)-3,6-diazabicyclo[3.2.1]octane-3-carboxamide |
| 110 | 3-(1-(2-Azabicyclo[2.1.1]hexan-5-yl)-2-(6-(cyclopropanecarbonyl)-3-((3,5- |
| | dimethylisoxazol-4-yl)sulfonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 111 | 3-(1-(2-Azabicyclo[2.1.1]hexan-5-yl)-2-(6-(cyclopropanecarbonyl)-3- |
| | (morpholinosulfonyl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 112 | 3-(1-(2-Azabicyclo[2.1.1]hexan-5-yl)-2-(6-(cyclopropanecarbonyl)-3-methyl-2-oxo- |
| | 3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 113 | 3-(1-(2-Azabicyclo[2.1.1]hexan-5-yl)-2-(3-cyclohexyl-6-(cyclopropanecarbonyl)-2- |
| | oxo-3,6-diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 114 | 3-(1-(2-Azabicyclo[2.1.1]hexan-5-yl)-2-(3-cyclohexyl-6-(cyclopropanecarbonyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 115 | 3-(1-(2-Azabicyclo[2.1.1]hexan-5-yl)-2-(6-(cyclopropanecarbonyl)-3-oxa-6- |
| | azabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 116 | 3-(1-(2-Azabicyclo[2.1.1]hexan-5-yl)-2-(6-(cyclopropanecarbonyl)-4-oxo-3-oxa-6- |
| | azabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 117 | 3-(1-(2-Azabicyclo[2.1.1]hexan-5-yl)-2-(4-(cyclopropanecarbonyl)-1-phenyl-1,4- |
| | diazepan-5-yl)-7-(2,3-dichlorophenyl)-6-fluoro-4-methyl-1H-pyrrolo[3,2-c]quinolin- |
| | 8-yl)propanenitrile |
| 118 | 3-(1-(2-Azabicyclo[2.1.1]hexan-5-yl)-2-(2-(cyclopropanecarbonyl)-6-(pyridin-2-yl)- |
| | 2,6-diazabicyclo[3.2.1]octan-3-yl)-7-(2,3-dichlorophenyl)-6-fluoro-4-methyl-1H- |
| | pyrrolo[3,2-c]quinolin-8-yl)propanenitrile |
| 119 | 3-(2-(6-Benzoyl-2-(cyclopropanecarbonyl)-2,6-diazabicyclo[3.2.1]octan-3-yl)-1-(2- |
| | azabicyclo[2.1.1]hexan-5-yl)-7-(2,3-dichlorophenyl)-6-fluoro-4-methyl-1H- |
| | pyrrolo[3,2-c]quinolin-8-yl)propanenitrile |
| 120a | 3-(1-(2-Azabicyclo[2.1.1]hexan-5-yl)-2-(6-(cyclopropanecarbonyl)-2-oxo-3-(4- |
| and | (trifluoromethyl)phenyl)-3,6-diazabicyclo[3.2.1]octan-7-yl)-7-(2,3-dichlorophenyl)-6- |
| 120b | fluoro-4-methyl-1H-pyrrolo[3,2-c]quinolin-8-yl)propanenitrile and |
| | 3-(1-(2-Azabicyclo[2.1.1]hexan-5-yl)-2-(6-(cyclopropanecarbonyl)-4-oxo-3-(4- |
| | (trifluoromethyl)phenyl)-3,6-diazabicyclo[3.2.1]octan-7-yl)-7-(2,3-dichlorophenyl)-6- |
| | fluoro-4-methyl-1H-pyrrolo[3,2-c]quinolin-8-yl)propanenitrile |
| 121 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-3-(6-(1,1- |
| | difluoroethyl)pyrimidin-4-yl)-2-methyl-3,6-diazabicyclo[3.2.1]octane-6-carboxylate |
| 122 | 3-(1-(2-Azabicyclo[2.1.1]hexan-5-yl)-2-(6-(cyclopropanecarbonyl)-4-oxo-3-(pyridin- |
| | 2-yl)-3,6-diazabicyclo[3.2.1]octan-7-yl)-7-(2,3-dichlorophenyl)-6-fluoro-4-methyl- |
| | 1H-pyrrolo[3,2-c]quinolin-8-yl)propanenitrile |
| 123 | 3-(1-(2-Azabicyclo[2.1.1]hexan-5-yl)-2-(6-(cyclopropanecarbonyl)-3-(5- |
| | isopropylthiazol-2-yl)-3,6-diazabicyclo[3.2.1]octan-7-yl-2,2-d2)-7-(2,3- |
| | dichlorophenyl)-6-fluoro-4-methyl-1H-pyrrolo[3,2-c]quinolin-8-yl)propanenitrile |
| 124 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-3-(6-(1,1- |
| | difluoroethyl)pyrimidin-4-yl)-3,6-diazabicyclo[3.2.1]octane-6-carboxylate |
| 125 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-3-(3- |
| | methoxypyrazin-2-yl)-3,6-diazabicyclo[3.2.1]octane-6-carboxylate |
| 126 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-3-(5- |
| | (cyclopropylmethoxy)pyrimidin-2-yl)-3,6-diazabicyclo[3.2.1]octane-6-carboxylate |
| 127 | Methyl 7-(1-(2-azabicyclo[2.1.1]hexan-5-yl)-8-(2-cyanoethyl)-7-(2,3- |
| | dichlorophenyl)-6-fluoro-4-methyl-1H-pyrrolo[3,2-c]quinolin-2-yl)-3-(5- |
| | cyanopyrazin-2-yl)-3,6-diazabicyclo[3.2.1]octane-6-carboxylate |
| 128 | 3-(1-(2-azabicyclo[2.1.1]hexan-5-yl)-7-(2,3-dichlorophenyl)-2-(3-(6-(1,1- |
| | difluoroethyl)pyrimidin-4-yl)-6-(1-fluorocyclopropane-1-carbonyl)-3,6- |
| | diazabicyclo[3.2.1]octan-7-yl)-6-fluoro-4-methyl-1H-pyrrolo[3,2-c]quinolin-8- |
| | yl)propanenitrile |
| 129 | 3-(1-(2-azabicyclo[2.1.1]hexan-5-yl)-7-(2,3-dichlorophenyl)-2-(3-(6-(1,1- |
| | difluoroethyl)pyrimidin-4-yl)-6-(2-hydroxyacetyl)-3,6-diazabicyclo[3.2.1]octan-7-yl)- |
| | 6-fluoro-4-methyl-1H-pyrrolo[3,2-c]quinolin-8-yl)propanenitrile |

In other embodiments, the compound of Formula (I) is selected from a compound in Table 2, and pharmaceutically acceptable salts thereof.

TABLE 2

| Entry | Compound Name |
|---|---|
| 1 | 3-(1-((1R,4R,5S)-2-azabicyclo[2.1.1]hexan-5-yl)-2-((1S,5R,7R)-6- |
| | (cyclopropanecarbonyl)-3-phenyl-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 2 | 3-((Rₐ)-1-((1R,4R,5S)-2-azabicyclo[2.1.1]hexan-5-yl)-2-((1S,5R,7R)-6- |
| | (cyclopropanecarbonyl)-3-phenyl-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 3 | 3-((Sₐ)-1-((1R,4R,5S)-2-azabicyclo[2.1.1]hexan-5-yl)-2-((1S,5R,7R)-6- |
| | (cyclopropanecarbonyl)-3-phenyl-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 4 | 3-(1-((1R,4R,5S)-2-azabicyclo[2.1.1]hexan-5-yl)-2-((1S,5R,7R)-6- |
| | (cyclopropanecarbonyl)-3-(2-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 5 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 6 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 7 | 3-(1-((1R,4R,5S)-2-azabicyclo[2.1.1]hexan-5-yl)-2-((1S,5R,7R)-6- |
| | (cyclopropanecarbonyl)-3-(4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 8 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 9 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 10 | 4-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)benzonitrile |
| 11 | 4-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)benzonitrile |
| 12 | 4-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)benzonitrile |
| 13 | 3-(1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 14 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 15 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 16 | 3-(1-((1R,4R,5S)-2-azabicyclo[2.1.1]hexan-5-yl)-2-((1S,5R,7R)-6- |
| | (cyclopropanecarbonyl)-3-(1,5-naphthyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 17 | 3-((Rₐ)-1-((1R,4R,5S)-2-azabicyclo[2.1.1]hexan-5-yl)-2-((1S,5R,7R)-6- |
| | (cyclopropanecarbonyl)-3-(1,5-naphthyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 18 | 3-((Sₐ)-1-((1R,4R,5S)-2-azabicyclo[2.1.1]hexan-5-yl)-2-((1S,5R,7R)-6- |
| | (cyclopropanecarbonyl)-3-(1,5-naphthyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 19 | 3-(1-((1R,4R,5S)-2-azabicyclo[2.1.1]hexan-5-yl)-2-((1S,5R,7R)-6- |
| | (cyclopropanecarbonyl)-3-(2,2-difluorobenzo[d][1,3]dioxol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 20 | 3-((Rₐ)-1-((1R,4R,5S)-2-azabicyclo[2.1.1]hexan-5-yl)-2-((1S,5R,7R)-6- |
| | (cyclopropanecarbonyl)-3-(2,2-difluorobenzo[d][1,3]dioxol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 21 | 3-((Sₐ)-1-((1R,4R,5S)-2-azabicyclo[2.1.1]hexan-5-yl)-2-((1S,5R,7R)-6- |
| | (cyclopropanecarbonyl)-3-(2,2-difluorobenzo[d][1,3]dioxol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 22 | 3-(1-((1R,4R,5S)-2-azabicyclo[2.1.1]hexan-5-yl)-2-((1S,5R,7R)-6- |
| | (cyclopropanecarbonyl)-3-(1-(difluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 23 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-(difluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 24 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-(difluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 25 | 3-(1-((1R,4R,5S)-2-azabicyclo[2.1.1]hexan-5-yl)-2-((1S,5R,7R)-6- |
| | (cyclopropanecarbonyl)-3-(pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 26 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 27 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyridin-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 28 | 3-(1-((1R,4R,5S)-2-azabicyclo[2.1.1]hexan-5-yl)-2-((1S,5R,7R)-6- |
| | (cyclopropanecarbonyl)-3-(3-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 29 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 30 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 31 | 3-(1-((1R,4R,5S)-2-azabicyclo[2.1.1]hexan-5-yl)-2-((1S,5R,7R)-6- |
| | (cyclopropanecarbonyl)-3-(1-methyl-1H-pyrazol-4-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 32 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-1H-pyrazol-4-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 33 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-1H-pyrazol-4-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 34 | 3-(1-((1R,4R,5S)-2-azabicyclo[2.1.1]hexan-5-yl)-2-((1S,5R,7R)-6- |
| | (cyclopropanecarbonyl)-3-(4-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 35 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 36 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 37 | 3-(1-((1R,4R,5S)-2-azabicyclo[2.1.1]hexan-5-yl)-2-((1S,5R,7R)-6- |
| | (cyclopropanecarbonyl)-3-(3-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 38 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 39 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 40 | 3-(1-((1R,4R,5S)-2-azabicyclo[2.1.1]hexan-5-yl)-2-((1S,5R,7R)-6- |
| | (cyclopropanecarbonyl)-3-(6-methylpyridin-3-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 41 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-methylpyridin-3-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 42 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-methylpyridin-3-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 43 | 3-(1-((1R,4R,5S)-2-azabicyclo[2.1.1]hexan-5-yl)-2-((1S,5R,7R)-6- |
| | (cyclopropanecarbonyl)-3-(3,4-dichlorophenyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 44 | 3-((Rₐ)-1-((1R,4R,5S)-2-azabicyclo[2.1.1]hexan-5-yl)-2-((1S,5R,7R)-6- |
| | (cyclopropanecarbonyl)-3-(3,4-dichlorophenyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 45 | 3-((Sₐ)-1-((1R,4R,5S)-2-azabicyclo[2.1.1]hexan-5-yl)-2-((1S,5R,7R)-6- |
| | (cyclopropanecarbonyl)-3-(3,4-dichlorophenyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 46 | 3-(1-((1R,4R,5S)-2-azabicyclo[2.1.1]hexan-5-yl)-2-((1S,5R,7R)-6- |
| | (cyclopropanecarbonyl)-3-(2-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 47 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 48 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)phenyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 49 | 3-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)benzonitrile |
| 50 | 3-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)benzonitrile |
| 51 | 3-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)benzonitrile |
| 52 | 3-(1-((1R,4R,5S)-2-azabicyclo[2.1.1]hexan-5-yl)-2-((1S,5R,7R)-3-(3,5- |
| | bis(trifluoromethyl)phenyl)-6-(cyclopropanecarbonyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 53 | 3-((Rₐ)-1-((1R,4R,5S)-2-azabicyclo[2.1.1]hexan-5-yl)-2-((1S,5R,7R)-3-(3,5- |
| | bis(trifluoromethyl)phenyl)-6-(cyclopropanecarbonyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 54 | 3-((Sₐ)-1-((1R,4R,5S)-2-azabicyclo[2.1.1]hexan-5-yl)-2-((1S,5R,7R)-3-(3,5- |
| | bis(trifluoromethyl)phenyl)-6-(cyclopropanecarbonyl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 55 | 3-(1-((1R,4R,5S)-2-azabicyclo[2.1.1]hexan-5-yl)-2-((1S,5R,7R)-3-(3-chlorophenyl)- |
| | 6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 56 | 3-((Rₐ)-1-((1R,4R,5S)-2-azabicyclo[2.1.1]hexan-5-yl)-2-((1S,5R,7R)-3-(3- |
| | chlorophenyl)-6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 57 | 3-((Sₐ)-1-((1R,4R,5S)-2-azabicyclo[2.1.1]hexan-5-yl)-2-((1S,5R,7R)-3-(3- |
| | chlorophenyl)-6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 58 | 3-(1-((1R,4R,5S)-2-azabicyclo[2.1.1]hexan-5-yl)-2-((1S,5R,7R)-6- |
| | (cyclopropanecarbonyl)-3-(3-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 59 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 60 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 61 | 3-(1-((1R,4R,5S)-2-azabicyclo[2.1.1]hexan-5-yl)-2-((1S,5R,7R)-6- |
| | (cyclopropanecarbonyl)-3-(5-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 62 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 63 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 64 | 3-(1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 65 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 66 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 67 | 3-(1-((1R,4R,5S)-2-azabicyclo[2.1.1]hexan-5-yl)-2-((1S,5R,7R)-6- |
| | (cyclopropanecarbonyl)-3-(4-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 68 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 69 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-fluorophenyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 70 | 3-(1-((1R,4R,5S)-2-azabicyclo[2.1.1]hexan-5-yl)-2-((1S,5R,7R)-6- |
| | (cyclopropanecarbonyl)-3-(6-methoxypyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 71 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-methoxypyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 72 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-methoxypyridin-3-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 73 | 3-(1-((1R,4R,5S)-2-azabicyclo[2.1.1]hexan-5-yl)-2-((1S,5R,7R)-6- |
| | (cyclopropanecarbonyl)-3-(4-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 74 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 75 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 76 | 3-(1-((1R,4R,5S)-2-azabicyclo[2.1.1]hexan-5-yl)-2-((1S,5R,7R)-6- |
| | (cyclopropanecarbonyl)-3-(3-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 77 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 78 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-(difluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 79 | 3-(1-((1R,4R,5S)-2-azabicyclo[2.1.1]hexan-5-yl)-2-((1S,5R,7R)-6- |
| | (cyclopropanecarbonyl)-3-(1-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 80 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 81 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 82 | 3-(1-((1R,4R,5S)-2-azabicyclo[2.1.1]hexan-5-yl)-2-((1S,5R,7R)-6- |
| | (cyclopropanecarbonyl)-3-(2-fluoro-4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 83 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-fluoro-4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 84 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-fluoro-4-(trifluoromethoxy)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 85 | 3-(1-((1R,4R,5S)-2-azabicyclo[2.1.1]hexan-5-yl)-2-((1S,5R,7R)-6- |
| | (cyclopropanecarbonyl)-3-(1-methyl-3-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 86 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-3-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 87 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-3-(trifluoromethyl)-1H-pyrazol-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 88 | 3-(1-((1R,4R,5S)-2-Azabicyclo[2.1.1]hexan-5-yl)-2-((1S,5R,7R)-6- |
| | (cyclopropanecarbonyl)-3-(5-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 89 | 3-((Rₐ)-1-((1R,4R,5S)-2-Azabicyclo[2.1.1]hexan-5-yl)-2-((1S,5R,7R)-6- |
| | (cyclopropanecarbonyl)-3-(5-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 90 | 3-((Sₐ)-1-((1R,4R,5S)-2-Azabicyclo[2.1.1]hexan-5-yl)-2-((1S,5R,7R)-6- |
| | (cyclopropanecarbonyl)-3-(5-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 91 | 3-(1-((1R,4R,5S)-2-Azabicyclo[2.1.1]hexan-5-yl)-2-((1S,5R,7R)-6- |
| | (cyclopropanecarbonyl)-3-(5-fluoro-6-methylpyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 92 | 3-((Rₐ)-1-((1R,4R,5S)-2-Azabicyclo[2.1.1]hexan-5-yl)-2-((1S,5R,7R)-6- |
| | (cyclopropanecarbonyl)-3-(5-fluoro-6-methylpyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 93 | 3-((Sₐ)-1-((1R,4R,5S)-2-Azabicyclo[2.1.1]hexan-5-yl)-2-((1S,5R,7R)-6- |
| | (cyclopropanecarbonyl)-3-(5-fluoro-6-methylpyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 94 | 3-(1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 95 | 3-((Rₐ)-1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 96 | 3-((Sₐ)-1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 97 | 3-(1-((1R,4R,5S)-2-azabicyclo[2.1.1]hexan-5-yl)-2-((1S,5R,7R)-6- |
| | (cyclopropanecarbonyl)-3-(pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 98 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 99 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyrimidin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 100 | 3-(1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridazin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 101 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridazin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 102 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-(trifluoromethyl)pyridazin-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 103 | 3-(1-((1R,4R,5S)-2-azabicyclo[2.1.1]hexan-5-yl)-2-((1S,5R,7R)-6- |
| | (cyclopropanecarbonyl)-3-(1-methyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 104 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 105 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 106 | 3-(1-((1R,4R,5S)-2-azabicyclo[2.1.1]hexan-5-yl)-2-((1S,5R,7R)-6- |
| | (cyclopropanecarbonyl)-3-(pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 107 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 108 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyridin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 109 | 3-(1-((1R,4R,5S)-2-azabicyclo[2.1.1]hexan-5-yl)-2-((1S,5R,7R)-6- |
| | (cyclopropanecarbonyl)-3-(pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 110 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 111 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyridin-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 112 | 3-(1-((1R,4R,5S)-2-azabicyclo[2.1.1]hexan-5-yl)-2-((1S,5R,7R)-6- |
| | (cyclopropanecarbonyl)-3-(2-(trifluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 113 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 114 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 115 | 3-(1-((1R,4R,5S)-2-azabicyclo[2.1.1]hexan-5-yl)-2-((1S,5R,7R)-6- |
| | (cyclopropanecarbonyl)-3-(4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 116 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 117 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 118 | 3-(1-((1R,4R,5S)-2-azabicyclo[2.1.1]hexan-5-yl)-2-((1S,5R,7R)-6- |
| | (cyclopropanecarbonyl)-3-(pyrazin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 119 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyrazin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 120 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyrazin-2-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 121 | 3-(1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 122 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 123 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 124 | 3-(1-((1R,4R,5S)-2-azabicyclo[2.1.1]hexan-5-yl)-2-((1S,5R,7R)-6- |
| | (cyclopropanecarbonyl)-3-(4-methoxypyrimidin-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 125 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-methoxypyrimidin-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 126 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-methoxypyrimidin-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 127 | 3-(1-((1R,4R,5S)-2-azabicyclo[2.1.1]hexan-5-yl)-2-((1S,5R,7R)-6- |
| | (cyclopropanecarbonyl)-3-(4-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 128 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 129 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 130 | 3-(1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrazin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 131 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrazin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 132 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-(trifluoromethyl)pyrazin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 133 | 3-(1-((1R,4R,5S)-2-azabicyclo[2.1.1]hexan-5-yl)-2-((1S,5R,7R)-6- |
| | (cyclopropanecarbonyl)-3-(5-(difluoromethoxy)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 134 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-(difluoromethoxy)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 135 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-(difluoromethoxy)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 136 | 3-(1-((1R,4R,5S)-2-azabicyclo[2.1.1]hexan-5-yl)-2-((1S,5R,7R)-6- |
| | (cyclopropanecarbonyl)-3-(3-fluoropyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 137 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoropyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 138 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoropyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 139 | 2-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)nicotinonitrile |
| 140 | 2-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)nicotinonitrile |
| 141 | 2-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)nicotinonitrile |
| 142 | 3-(1-((1R,4R,5S)-2-azabicyclo[2.1.1]hexan-5-yl)-2-((1S,5R,7R)-6- |
| | (cyclopropanecarbonyl)-3-(3-methylpyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 143 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-methylpyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 144 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-methylpyridin-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 145 | 3-(1-((1R,4R,5S)-2-azabicyclo[2.1.1]hexan-5-yl)-2-((1S,5R,7R)-6- |
| | (cyclopropanecarbonyl)-3-(3-methoxypyridin-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 146 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-methoxypyridin-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 147 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-methoxypyridin-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 148 | 3-(1-((1R,4R,5S)-2-azabicyclo[2.1.1]hexan-5-yl)-2-((1S,5R,7R)-6- |
| | (cyclopropanecarbonyl)-3-(3-fluoro-4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 149 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoro-4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 150 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoro-4-(trifluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 151 | 3-(1-((1R,4R,5S)-2-azabicyclo[2.1.1]hexan-5-yl)-2-((1S,5R,7R)-6- |
| | (cyclopropanecarbonyl)-3-(3-fluoro-4-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 152 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoro-4-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 153 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoro-4-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 154 | 3-(1-((1R,4R,5S)-2-azabicyclo[2.1.1]hexan-5-yl)-2-((1S,5R,7R)-6- |
| | (cyclopropanecarbonyl)-3-(3-fluoro-6-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 155 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoro-6-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 156 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoro-6-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 157 | 3-(1-((1R,4R,5S)-2-azabicyclo[2.1.1]hexan-5-yl)-2-((1S,5R,7R)-6- |
| | (cyclopropanecarbonyl)-3-(3-fluoro-5-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 158 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoro-5-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 159 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoro-5-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 160 | 3-(1-((1R,4R,5S)-2-azabicyclo[2.1.1]hexan-5-yl)-2-((1S,5R,7R)-6- |
| | (cyclopropanecarbonyl)-3-(4-(difluoromethyl)-3-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 161 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)-3-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 162 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)-3-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 163 | 3-(1-((1R,4R,5S)-2-azabicyclo[2.1.1]hexan-5-yl)-2-((1S,5R,7R)-6- |
| | (cyclopropanecarbonyl)-3-(3-fluoro-4-methoxypyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 164 | 3-((Rₐ)-1-((1R,4R,5S)-2-azabicyclo[2.1.1]hexan-5-yl)-2-((1S,5R,7R)-6- |
| | (cyclopropanecarbonyl)-3-(3-fluoro-4-methoxypyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 165 | 3-((Sₐ)-1-((1R,4R,5S)-2-azabicyclo[2.1.1]hexan-5-yl)-2-((1S,5R,7R)-6- |
| | (cyclopropanecarbonyl)-3-(3-fluoro-4-methoxypyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 166 | 3-(1-((1R,4R,5S)-2-azabicyclo[2.1.1]hexan-5-yl)-2-((1S,5R,7R)-6- |
| | (cyclopropanecarbonyl)-3-(1-(difluoromethyl)-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 167 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-(difluoromethyl)-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 168 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-(difluoromethyl)-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 169 | 3-(1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 170 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 171 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 172 | 3-(1-((1R,4R,5S)-2-azabicyclo[2.1.1]hexan-5-yl)-2-((1S,5R,7R)-6- |
| | (cyclopropanecarbonyl)-3-(1-isobutyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 173 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-isobutyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 174 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-isobutyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 175 | 3-(1-((1R,4R,5S)-2-azabicyclo[2.1.1]hexan-5-yl)-2-((1S,5R,7R)-6- |
| | (cyclopropanecarbonyl)-3-(2-methoxypyridin-4-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 176 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-methoxypyridin-4-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 177 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-methoxypyridin-4-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 178 | 3-(1-((1R,4R,5S)-2-azabicyclo[2.1.1]hexan-5-yl)-2-((1S,5R,7R)-6- |
| | (cyclopropanecarbonyl)-3-(2-(difluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 179 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(difluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 180 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(difluoromethyl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 181 | 3-(1-((1R,4R,5S)-2-azabicyclo[2.1.1]hexan-5-yl)-2-((1S,5R,7R)-6- |
| | (cyclopropanecarbonyl)-3-(imidazo[1,2-a]pyridin-7-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 182 | 3-((Rₐ)-1-((1R,4R,5S)-2-azabicyclo[2.1.1]hexan-5-yl)-2-((1S,5R,7R)-6- |
| | (cyclopropanecarbonyl)-3-(imidazo[1,2-a]pyridin-7-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 183 | 3-((Sₐ)-1-((1R,4R,5S)-2-azabicyclo[2.1.1]hexan-5-yl)-2-((1S,5R,7R)-6- |
| | (cyclopropanecarbonyl)-3-(imidazo[1,2-a]pyridin-7-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 184 | 3-(1-((1R,4R,5S)-2-azabicyclo[2.1.1]hexan-5-yl)-2-((1S,5R,7R)-6- |
| | (cyclopropanecarbonyl)-3-(2-(2-hydroxypropan-2-yl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 185 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(2-hydroxypropan-2-yl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 186 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(2-hydroxypropan-2-yl)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 187 | 4-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)picolinonitrile |
| 188 | 4-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)picolinonitrile |
| 189 | 4-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)picolinonitrile |
| 190 | 4-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)-N,N- |
| | dimethylpicolinamide |
| 191 | 4-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-N,N- |
| | dimethylpicolinamide |
| 192 | 4-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-N,N- |
| | dimethylpicolinamide |
| 193 | 3-(1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridazin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 194 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridazin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 195 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyridazin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 196 | 3-(1-((1R,4R,5S)-2-azabicyclo[2.1.1]hexan-5-yl)-2-((1S,5R,7R)-6- |
| | (cyclopropanecarbonyl)-3-(1,6-dimethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 197 | 3-((Rₐ)-1-((1R,4R,5S)-2-azabicyclo[2.1.1]hexan-5-yl)-2-((1S,5R,7R)-6- |
| | (cyclopropanecarbonyl)-3-(1,6-dimethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 198 | 3-((Sₐ)-1-((1R,4R,5S)-2-azabicyclo[2.1.1]hexan-5-yl)-2-((1S,5R,7R)-6- |
| | (cyclopropanecarbonyl)-3-(1,6-dimethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 199 | 3-(1-((1R,4R,5S)-2-azabicyclo[2.1.1]hexan-5-yl)-2-((1S,5R,7R)-6- |
| | (cyclopropanecarbonyl)-3-(1-ethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 200 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-ethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 201 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-ethyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 202 | 3-(1-((1R,4R,5S)-2-azabicyclo[2.1.1]hexan-5-yl)-2-((1S,5R,7R)-6- |
| | (cyclopropanecarbonyl)-3-(1-isopropyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 203 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-isopropyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 204 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-isopropyl-2-oxo-1,2-dihydropyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 205 | 3-(1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 206 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 207 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-6-oxo-1,6-dihydropyrimidin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 208 | 3-(1-((1R,4R,5S)-2-azabicyclo[2.1.1]hexan-5-yl)-2-((1S,5R,7R)-6- |
| | (cyclopropanecarbonyl)-3-(2-(trifluoromethyl)imidazo[1,2-a]pyridin-7-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 209 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)imidazo[1,2-a]pyridin-7-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 210 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(trifluoromethyl)imidazo[1,2-a]pyridin-7-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 211 | 3-(1-((1R,4R,5S)-2-azabicyclo[2.1.1]hexan-5-yl)-2-((1S,5R,7R)-6- |
| | (cyclopropanecarbonyl)-3-(pyrazolo[1,5-a]pyridin-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 212 | 3-((Rₐ)-1-((1R,4R,5S)-2-azabicyclo[2.1.1]hexan-5-yl)-2-((1S,5R,7R)-6- |
| | (cyclopropanecarbonyl)-3-(pyrazolo[1,5-a]pyridin-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 213 | 3-((Sₐ)-1-((1R,4R,5S)-2-azabicyclo[2.1.1]hexan-5-yl)-2-((1S,5R,7R)-6- |
| | (cyclopropanecarbonyl)-3-(pyrazolo[1,5-a]pyridin-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 214 | 3-(2-((1S,5R,7R)-3-([1,2,4]triazolo[1,5-a]pyridin-7-yl)-6-(cyclopropanecarbonyl)- |
| | 3,6-diazabicyclo[3.2.1]octan-7-yl)-1-((1R,4R,5S)-2-azabicyclo[2.1.1]hexan-5-yl)-7- |
| | (2,3-dichlorophenyl)-6-fluoro-4-methyl-1H-pyrrolo[3,2-c]quinolin-8-yl)propanenitrile |
| 215 | 3-((Rₐ)-2-((1S,5R,7R)-3-([1,2,4]triazolo[1,5-a]pyridin-7-yl)-6- |
| | (cyclopropanecarbonyl)-3,6-diazabicyclo[3.2.1]octan-7-yl)-1-((1R,4R,5S)-2- |
| | azabicyclo[2.1.1]hexan-5-yl)-7-(2,3-dichlorophenyl)-6-fluoro-4-methyl-1H- |
| | pyrrolo[3,2-c]quinolin-8-yl)propanenitrile |
| 216 | 3-((Sₐ)-2-((1S,5R,7R)-3-([1,2,4]triazolo[1,5-a]pyridin-7-yl)-6- |
| | (cyclopropanecarbonyl)-3,6-diazabicyclo[3.2.1]octan-7-yl)-1-((1R,4R,5S)-2- |
| | azabicyclo[2.1.1]hexan-5-yl)-7-(2,3-dichlorophenyl)-6-fluoro-4-methyl-1H- |
| | pyrrolo[3,2-c]quinolin-8-yl)propanenitrile |
| 217 | 3-(1-((1R,4R,5S)-2-azabicyclo[2.1.1]hexan-5-yl)-2-((1S,5R,7R)-6- |
| | (cyclopropanecarbonyl)-3-(2-oxo-2H-pyran-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 218 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-oxo-2H-pyran-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 219 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-oxo-2H-pyran-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 220 | 3-(1-((1R,4R,5S)-2-azabicyclo[2.1.1]hexan-5-yl)-2-((1S,5R,7R)-3-(2-bromopyridin- |
| | 4-yl)-6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 221 | 3-((Rₐ)-1-((1R,4R,5S)-2-azabicyclo[2.1.1]hexan-5-yl)-2-((1S,5R,7R)-3-(2- |
| | bromopyridin-4-yl)-6-(cyclopropanecarbonyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 222 | 3-((Sₐ)-1-((1R,4R,5S)-2-azabicyclo[2.1.1]hexan-5-yl)-2-((1S,5R,7R)-3-(2- |
| | bromopyridin-4-yl)-6-(cyclopropanecarbonyl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 223 | 3-(1-((1R,4R,5S)-2-azabicyclo[2.1.1]hexan-5-yl)-2-((1S,5R,7R)-6- |
| | (cyclopropanecarbonyl)-3-(4-fluoro-3-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 224 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-fluoro-3-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 225 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-fluoro-3-methylpyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 226 | 3-(1-((1R,4R,5S)-2-azabicyclo[2.1.1]hexan-5-yl)-2-((1S,5R,7R)-6- |
| | (cyclopropanecarbonyl)-3-(2-fluoro-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 227 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-fluoro-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 228 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-fluoro-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 229 | 3-(1-((1R,4R,5S)-2-azabicyclo[2.1.1]hexan-5-yl)-2-((1S,5R,7R)-6- |
| | (cyclopropanecarbonyl)-3-(2-methoxy-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 230 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-methoxy-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 231 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-methoxy-3-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 232 | 3-(1-((1R,4R,5S)-2-azabicyclo[2.1.1]hexan-5-yl)-2-((1S,5R,7R)-6- |
| | (cyclopropanecarbonyl)-3-(2-methoxy-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 233 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-methoxy-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 234 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-methoxy-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 235 | 3-(1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 236 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 237 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 238 | 3-(1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 239 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 240 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-fluoro-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 241 | 3-(1-((1R,4R,5S)-2-azabicyclo[2.1.1]hexan-5-yl)-2-((1S,5R,7R)-6- |
| | (cyclopropanecarbonyl)-3-(2-ethoxypyridin-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 242 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-ethoxypyridin-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 243 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-ethoxypyridin-4-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 244 | 3-(1-((1R,4R,5S)-2-azabicyclo[2.1.1]hexan-5-yl)-2-((1S,5R,7R)-6- |
| | (cyclopropanecarbonyl)-3-(2-(cyclopropylmethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 245 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(cyclopropylmethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 246 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(cyclopropylmethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 247 | 3-(1-((1R,4R,5S)-2-azabicyclo[2.1.1]hexan-5-yl)-2-((1S,5R,7R)-6- |
| | (cyclopropanecarbonyl)-3-(2-(2-methoxyethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 248 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(2-methoxyethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 249 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(2-methoxyethoxy)pyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 250 | 3-(1-((1R,4R,5S)-2-azabicyclo[2.1.1]hexan-5-yl)-2-((1S,5R,7R)-6- |
| | (cyclopropanecarbonyl)-3-(2-(difluoromethyl)-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 251 | 3-((Rₐ)-1-((1R,4R,5S)-2-azabicyclo[2.1.1]hexan-5-yl)-2-((1S,5R,7R)-6- |
| | (cyclopropanecarbonyl)-3-(2-(difluoromethyl)-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 252 | 3-((Sₐ)-1-((1R,4R,5S)-2-azabicyclo[2.1.1]hexan-5-yl)-2-((1S,5R,7R)-6- |
| | (cyclopropanecarbonyl)-3-(2-(difluoromethyl)-6-methylpyridin-4-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 253 | 3-(1-((1R,4R,5S)-2-azabicyclo[2.1.1]hexan-5-yl)-2-((1S,5R,7R)-6- |
| | (cyclopropanecarbonyl)-3-(4-(difluoromethyl)-5-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 254 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)-5-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 255 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)-5-fluoropyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 256 | 3-(1-((1R,4R,5S)-2-azabicyclo[2.1.1]hexan-5-yl)-2-((1S,5R,7R)-6- |
| | (cyclopropanecarbonyl)-3-(6-(difluoromethoxy)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 257 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-(difluoromethoxy)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 258 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-(difluoromethoxy)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 259 | 3-(1-((1R,4R,5S)-2-azabicyclo[2.1.1]hexan-5-yl)-2-((1S,5R,7R)-6- |
| | (cyclopropanecarbonyl)-3-(4-(difluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 260 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 261 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)pyrimidin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 262 | 3-(1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 263 | 3-((Rₐ)-1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 264 | 3-((Sₐ)-1-((1R,4R,5S)-2-azabicyclo[2.1.1]hexan-5-yl)-2-((1S,5R,7R)-6- |
| | (cyclopropanecarbonyl)-3-(6-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 265 | 3-(1-((1R,4R,5S)-2-azabicyclo[2.1.1]hexan-5-yl)-2-((1S,5R,7R)-6- |
| | (cyclopropanecarbonyl)-3-(4-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 266 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 267 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(difluoromethyl)pyridin-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 268 | 3-(1-((1R,4R,5S)-2-azabicyclo[2.1.1]hexan-5-yl)-2-((1S,5R,7R)-6- |
| | (cyclopropanecarbonyl)-3-(4-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 269 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 270 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 271 | 3-(1-((1R,4R,5S)-2-azabicyclo[2.1.1]hexan-5-yl)-2-((1S,5R,7R)-6- |
| | (cyclopropanecarbonyl)-3-(5-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 272 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 273 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-methylthiazol-2-yl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 274 | 3-(1-((1R,4R,5S)-2-azabicyclo[2.1.1]hexan-5-yl)-2-((1S,5R,7R)-6- |
| | (cyclopropanecarbonyl)-3-(1-methyl-1H-1,2,4-triazol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 275 | 3-((Rₐ)-1-((1R,4R,5S)-2-azabicyclo[2.1.1]hexan-5-yl)-2-((1S,5R,7R)-6- |
| | (cyclopropanecarbonyl)-3-(1-methyl-1H-1,2,4-triazol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 276 | 3-((Sₐ)-1-((1R,4R,5S)-2-azabicyclo[2.1.1]hexan-5-yl)-2-((1S,5R,7R)-6- |
| | (cyclopropanecarbonyl)-3-(1-methyl-1H-1,2,4-triazol-5-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 277 | 3-(1-((1R,4R,5S)-2-azabicyclo[2.1.1]hexan-5-yl)-2-((1S,5R,7R)-6- |
| | (cyclopropanecarbonyl)-3-(thiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 278 | 3-((Rₐ)-1-((1R,4R,5S)-2-azabicyclo[2.1.1]hexan-5-yl)-2-((1S,5R,7R)-6- |
| | (cyclopropanecarbonyl)-3-(thiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 279 | 3-((Sₐ)-1-((1R,4R,5S)-2-azabicyclo[2.1.1]hexan-5-yl)-2-((1S,5R,7R)-6- |
| | (cyclopropanecarbonyl)-3-(thiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 280 | 3-(1-((1R,4R,5S)-2-azabicyclo[2.1.1]hexan-5-yl)-2-((1S,5R,7R)-6- |
| | (cyclopropanecarbonyl)-3-(4-(trifluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 281 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 282 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(trifluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 283 | 3-(1-((1R,4R,5S)-2-azabicyclo[2.1.1]hexan-5-yl)-2-((1S,5R,7R)-6- |
| | (cyclopropanecarbonyl)-3-(4,5-dimethylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 284 | 3-((Rₐ)-1-((1R,4R,5S)-2-azabicyclo[2.1.1]hexan-5-yl)-2-((1S,5R,7R)-6- |
| | (cyclopropanecarbonyl)-3-(4,5-dimethylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 285 | 3-((Sₐ)-1-((1R,4R,5S)-2-azabicyclo[2.1.1]hexan-5-yl)-2-((1S,5R,7R)-6- |
| | (cyclopropanecarbonyl)-3-(4,5-dimethylthiazol-2-yl)-3,6-diazabicyclo[3.2.1]octan-7- |
| | yl)-7-(2,3-dichlorophenyl)-6-fluoro-4-methyl-1H-pyrrolo[3,2-c]quinolin-8- |
| | yl)propanenitrile |
| 286 | 2-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)thiazole-4-carbonitrile |
| 287 | 2-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)thiazole-4- |
| | carbonitrile |
| 288 | 2-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)thiazole-4- |
| | carbonitrile |
| 289 | 2-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)-5-methylthiazole-4- |
| | carbonitrile |
| 290 | 2-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-5-methylthiazole- |
| | 4-carbonitrile |
| 291 | 2-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-5-methylthiazole- |
| | 4-carbonitrile |
| 292 | 2-((1S,5R,7R)-7-(1-((1R,4R,5S)-2-azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-3,6-diazabicyclo[3.2.1]octan-3-yl)-4-methylthiazole-5- |
| | carbonitrile |
| 293 | 2-((1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-4-methylthiazole-5- |
| | carbonitrile |
| 294 | 2-((1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octan-3-yl)-4-methylthiazole-5- |
| | carbonitrile |
| 295 | 3-(1-((1R,4R,5S)-2-azabicyclo[2.1.1]hexan-5-yl)-2-((1S,5R,7R)-6- |
| | (cyclopropanecarbonyl)-3-(5-ethylthiazol-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 296 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-ethylthiazol-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 297 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-ethylthiazol-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 298 | 3-(1-((1R,4R,5S)-2-azabicyclo[2.1.1]hexan-5-yl)-2-((1S,5R,7R)-6- |
| | (cyclopropanecarbonyl)-3-(isoxazol-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 299 | 3-((Rₐ)-1-((1R,4R,5S)-2-azabicyclo[2.1.1]hexan-5-yl)-2-((1S,5R,7R)-6- |
| | (cyclopropanecarbonyl)-3-(isoxazol-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 300 | 3-((Sₐ)-1-((1R,4R,5S)-2-azabicyclo[2.1.1]hexan-5-yl)-2-((1S,5R,7R)-6- |
| | (cyclopropanecarbonyl)-3-(isoxazol-3-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 301 | 3-(1-((1R,4R,5S)-2-azabicyclo[2.1.1]hexan-5-yl)-2-((1S,5R,7R)-6- |
| | (cyclopropanecarbonyl)-3-(1,5-dimethyl-1H-imidazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 302 | 3-((Rₐ)-1-((1R,4R,5S)-2-azabicyclo[2.1.1]hexan-5-yl)-2-((1S,5R,7R)-6- |
| | (cyclopropanecarbonyl)-3-(1,5-dimethyl-1H-imidazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 303 | 3-((Sₐ)-1-((1R,4R,5S)-2-azabicyclo[2.1.1]hexan-5-yl)-2-((1S,5R,7R)-6- |
| | (cyclopropanecarbonyl)-3-(1,5-dimethyl-1H-imidazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 304 | 3-(1-((1R,4R,5S)-2-azabicyclo[2.1.1]hexan-5-yl)-2-((1S,5R,7R)-6- |
| | (cyclopropanecarbonyl)-3-(5-(difluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 305 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-(difluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 306 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-(difluoromethyl)thiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 307 | 3-(1-((1R,4R,5S)-2-azabicyclo[2.1.1]hexan-5-yl)-2-((1S,5R,7R)-6- |
| | (cyclopropanecarbonyl)-3-(4-(1,1-difluoroethyl)-5-methylthiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 308 | 3-((Rₐ)-1-((1R,4R,5S)-2-azabicyclo[2.1.1]hexan-5-yl)-2-((1S,5R,7R)-6- |
| | (cyclopropanecarbonyl)-3-(4-(1,1-difluoroethyl)-5-methylthiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 309 | 3-((Sₐ)-1-((1R,4R,5S)-2-azabicyclo[2.1.1]hexan-5-yl)-2-((1S,5R,7R)-6- |
| | (cyclopropanecarbonyl)-3-(4-(1,1-difluoroethyl)-5-methylthiazol-2-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 310 | 3-(1-((1R,4R,5S)-2-azabicyclo[2.1.1]hexan-5-yl)-2-((1S,5R,7R)-6- |
| | (cyclopropanecarbonyl)-3-(isothiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 311 | 3-((Rₐ)-1-((1R,4R,5S)-2-azabicyclo[2.1.1]hexan-5-yl)-2-((1S,5R,7R)-6- |
| | (cyclopropanecarbonyl)-3-(isothiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 312 | 3-((Sₐ)-1-((1R,4R,5S)-2-azabicyclo[2.1.1]hexan-5-yl)-2-((1S,5R,7R)-6- |
| | (cyclopropanecarbonyl)-3-(isothiazol-4-yl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 313 | 3-(1-((1R,4R,5S)-2-azabicyclo[2.1.1]hexan-5-yl)-2-((1S,5R,7R)-6- |
| | (cyclopropanecarbonyl)-3-(1,5-dimethyl-1H-pyrazol-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 314 | 3-((Rₐ)-1-((1R,4R,5S)-2-azabicyclo[2.1.1]hexan-5-yl)-2-((1S,5R,7R)-6- |
| | (cyclopropanecarbonyl)-3-(1,5-dimethyl-1H-pyrazol-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 315 | 3-((Sₐ)-1-((1R,4R,5S)-2-azabicyclo[2.1.1]hexan-5-yl)-2-((1S,5R,7R)-6- |
| | (cyclopropanecarbonyl)-3-(1,5-dimethyl-1H-pyrazol-3-yl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 316 | 3-(1-((1R,4R,5S)-2-azabicyclo[2.1.1]hexan-5-yl)-2-((1S,5R,7R)-6- |
| | (cyclopropanecarbonyl)-3-(5-cyclopropylthiazol-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 317 | 3-((Rₐ)-1-((1R,4R,5S)-2-azabicyclo[2.1.1]hexan-5-yl)-2-((1S,5R,7R)-6- |
| | (cyclopropanecarbonyl)-3-(5-cyclopropylthiazol-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 318 | 3-((Sₐ)-1-((1R,4R,5S)-2-azabicyclo[2.1.1]hexan-5-yl)-2-((1S,5R,7R)-6- |
| | (cyclopropanecarbonyl)-3-(5-cyclopropylthiazol-2-yl)-3,6-diazabicyclo[3.2.1]octan- |
| | 7-yl)-7-(2,3-dichlorophenyl)-6-fluoro-4-methyl-1H-pyrrolo[3,2-c]quinolin-8- |
| | yl)propanenitrile |
| 319 | 3-(1-((1R,4R,5S)-2-Azabicyclo[2.1.1]hexan-5-yl)-2-((1S,5R,7R)-3,6- |
| | di(cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 320 | 3-((Rₐ)-1-((1R,4R,5S)-2-Azabicyclo[2.1.1]hexan-5-yl)-2-((1S,5R,7R)-3,6- |
| | di(cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 321 | 3-((Sₐ)-1-((1R,4R,5S)-2-Azabicyclo[2.1.1]hexan-5-yl)-2-((1S,5R,7R)-3,6- |
| | di(cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 322 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octane-3-carboxylate |
| 323 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octane-3-carboxylate |
| 324 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-6-(cyclopropanecarbonyl)-3,6-diazabicyclo[3.2.1]octane-3-carboxylate |
| 325 | (1S,5R,7R)-7-(1-((1R,4R,5S)-2-Azabicyclo[2.1.1]hexan-5-yl)-8-(2-cyanoethyl)-7- |
| | (2,3-dichlorophenyl)-6-fluoro-4-methyl-1H-pyrrolo[3,2-c]quinolin-2-yl)-6- |
| | (cyclopropanecarbonyl)-N-(2,2,2-trifluoroethyl)-3,6-diazabicyclo[3.2.1]octane-3- |
| | carboxamide |
| 326 | (1S,5R,7R)-7-((Rₐ)-1-((1R,4R,5S)-2-Azabicyclo[2.1.1]hexan-5-yl)-8-(2-cyanoethyl)- |
| | 7-(2,3-dichlorophenyl)-6-fluoro-4-methyl-1H-pyrrolo[3,2-c]quinolin-2-yl)-6- |
| | (cyclopropanecarbonyl)-N-(2,2,2-trifluoroethyl)-3,6-diazabicyclo[3.2.1]octane-3- |
| | carboxamide |
| 327 | (1S,5R,7R)-7-((Sₐ)-1-((1R,4R,5S)-2-Azabicyclo[2.1.1]hexan-5-yl)-8-(2-cyanoethyl)- |
| | 7-(2,3-dichlorophenyl)-6-fluoro-4-methyl-1H-pyrrolo[3,2-c]quinolin-2-yl)-6- |
| | (cyclopropanecarbonyl)-N-(2,2,2-trifluoroethyl)-3,6-diazabicyclo[3.2.1]octane-3- |
| | carboxamide |
| 328 | 3-(1-((1R,4R,5S)-2-Azabicyclo[2.1.1]hexan-5-yl)-2-((1S,5R,7R)-6- |
| | (cyclopropanecarbonyl)-3-((3,5-dimethylisoxazol-4-yl)sulfonyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 329 | 3-((Rₐ)-1-((1R,4R,5S)-2-Azabicyclo[2.1.1]hexan-5-yl)-2-((1S,5R,7R)-6- |
| | (cyclopropanecarbonyl)-3-((3,5-dimethylisoxazol-4-yl)sulfonyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 330 | 3-((Sₐ)-1-((1R,4R,5S)-2-Azabicyclo[2.1.1]hexan-5-yl)-2-((1S,5R,7R)-6- |
| | (cyclopropanecarbonyl)-3-((3,5-dimethylisoxazol-4-yl)sulfonyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 331 | 3-(1-((1R,4R,5S)-2-Azabicyclo[2.1.1]hexan-5-yl)-2-((1S,5R,7R)-6- |
| | (cyclopropanecarbonyl)-3-(morpholinosulfonyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 332 | 3-((Rₐ)-1-((1R,4R,5S)-2-Azabicyclo[2.1.1]hexan-5-yl)-2-((1S,5R,7R)-6- |
| | (cyclopropanecarbonyl)-3-(morpholinosulfonyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 333 | 3-((Sₐ)-1-((1R,4R,5S)-2-Azabicyclo[2.1.1]hexan-5-yl)-2-((1S,5R,7R)-6- |
| | (cyclopropanecarbonyl)-3-(morpholinosulfonyl)-3,6-diazabicyclo[3.2.1]octan-7-yl)- |
| | 7-(2,3-dichlorophenyl)-6-fluoro-4-methyl-1H-pyrrolo[3,2-c]quinolin-8- |
| | yl)propanenitrile |
| 334 | 3-(1-((1R,4R,5S)-2-Azabicyclo[2.1.1]hexan-5-yl)-2-((1R,5R,7R)-6- |
| | (cyclopropanecarbonyl)-3-methyl-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 335 | 3-((Rₐ)-1-((1R,4R,5S)-2-Azabicyclo[2.1.1]hexan-5-yl)-2-((1R,5R,7R)-6- |
| | (cyclopropanecarbonyl)-3-methyl-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 336 | 3-((Sₐ)-1-((1R,4R,5S)-2-Azabicyclo[2.1.1]hexan-5-yl)-2-((1R,5R,7R)-6- |
| | (cyclopropanecarbonyl)-3-methyl-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 337 | 3-(1-((1R,4R,5S)-2-Azabicyclo[2.1.1]hexan-5-yl)-2-((1R,5R,7R)-3-cyclohexyl-6- |
| | (cyclopropanecarbonyl)-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 338 | 3-((Rₐ)-1-((1R,4R,5S)-2-Azabicyclo[2.1.1]hexan-5-yl)-2-((1R,5R,7R)-3-cyclohexyl- |
| | 6-(cyclopropanecarbonyl)-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 339 | 3-((Sₐ)-1-((1R,4R,5S)-2-Azabicyclo[2.1.1]hexan-5-yl)-2-((1R,5R,7R)-3-cyclohexyl- |
| | 6-(cyclopropanecarbonyl)-2-oxo-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 340 | 3-(1-((1R,4R,5S)-2-Azabicyclo[2.1.1]hexan-5-yl)-2-((1R,5R,7R)-3-cyclohexyl-6- |
| | (cyclopropanecarbonyl)-3,6-diazabicyclo[3.2.1]octan-7-yl)-7-(2,3-dichlorophenyl)- |
| | 6-fluoro-4-methyl-1H-pyrrolo[3,2-c]quinolin-8-yl)propanenitrile |
| 341 | 3-((Rₐ)-1-((1R,4R,5S)-2-Azabicyclo[2.1.1]hexan-5-yl)-2-((1R,5R,7R)-3-cyclohexyl- |
| | 6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 342 | 3-((Sₐ)-1-((1R,4R,5S)-2-Azabicyclo[2.1.1]hexan-5-yl)-2-((1R,5R,7R)-3-cyclohexyl- |
| | 6-(cyclopropanecarbonyl)-3,6-diazabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 343 | 3-(1-((1R,4R,5S)-2-Azabicyclo[2.1.1]hexan-5-yl)-2-((1R,5R,7R)-6- |
| | (cyclopropanecarbonyl)-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 344 | 3-((Rₐ)-1-((1R,4R,5S)-2-Azabicyclo[2.1.1]hexan-5-yl)-2-((1R,5R,7R)-6- |
| | (cyclopropanecarbonyl)-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 345 | 3-((Sₐ)-1-((1R,4R,5S)-2-Azabicyclo[2.1.1]hexan-5-yl)-2-((1R,5R,7R)-6- |
| | (cyclopropanecarbonyl)-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 346 | 3-(1-((1R,4R,5S)-2-Azabicyclo[2.1.1]hexan-5-yl)-2-((1R,5R,7R)-6- |
| | (cyclopropanecarbonyl)-4-oxo-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 347 | 3-((Rₐ)-1-((1R,4R,5S)-2-Azabicyclo[2.1.1]hexan-5-yl)-2-((1R,5R,7R)-6- |
| | (cyclopropanecarbonyl)-4-oxo-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 348 | 3-((Sₐ)-1-((1R,4R,5S)-2-Azabicyclo[2.1.1]hexan-5-yl)-2-((1R,5R,7R)-6- |
| | (cyclopropanecarbonyl)-4-oxo-3-oxa-6-azabicyclo[3.2.1]octan-7-yl)-7-(2,3- |
| | dichlorophenyl)-6-fluoro-4-methyl-1H-pyrrolo[3,2-c]quinolin-8-yl)propanenitrile |
| 349 | 3-(1-((1R,4R,5S)-2-Azabicyclo[2.1.1]hexan-5-yl)-2-((R)-4-(cyclopropanecarbonyl)- |
| | 1-phenyl-1,4-diazepan-5-yl)-7-(2,3-dichlorophenyl)-6-fluoro-4-methyl-1H- |
| | pyrrolo[3,2-c]quinolin-8-yl)propanenitrile |
| 350 | 3-((Rₐ)-1-((1R,4R,5S)-2-Azabicyclo[2.1.1]hexan-5-yl)-2-((R)-4- |
| | (cyclopropanecarbonyl)-1-phenyl-1,4-diazepan-5-yl)-7-(2,3-dichlorophenyl)-6- |
| | fluoro-4-methyl-1H-pyrrolo[3,2-c]quinolin-8-yl)propanenitrile |
| 351 | 3-((Sₐ)-1-((1R,4R,5S)-2-Azabicyclo[2.1.1]hexan-5-yl)-2-((R)-4- |
| | (cyclopropanecarbonyl)-1-phenyl-1,4-diazepan-5-yl)-7-(2,3-dichlorophenyl)-6- |
| | fluoro-4-methyl-1H-pyrrolo[3,2-c]quinolin-8-yl)propanenitrile |
| 352 | 3-(1-((1R,4R,5S)-2-Azabicyclo[2.1.1]hexan-5-yl)-2-((1R,3R,5R)-2- |
| | (cyclopropanecarbonyl)-6-(pyridin-2-yl)-2,6-diazabicyclo[3.2.1]octan-3-yl)-7-(2,3- |
| | dichlorophenyl)-6-fluoro-4-methyl-1H-pyrrolo[3,2-c]quinolin-8-yl)propanenitrile |
| 353 | 3-((Rₐ)-1-((1R,4R,5S)-2-Azabicyclo[2.1.1]hexan-5-yl)-2-((1R,3R,5R)-2- |
| | (cyclopropanecarbonyl)-6-(pyridin-2-yl)-2,6-diazabicyclo[3.2.1]octan-3-yl)-7-(2,3- |
| | dichlorophenyl)-6-fluoro-4-methyl-1H-pyrrolo[3,2-c]quinolin-8-yl)propanenitrile |
| 354 | 3-((Sₐ)-1-((1R,4R,5S)-2-Azabicyclo[2.1.1]hexan-5-yl)-2-((1R,3R,5R)-2- |
| | (cyclopropanecarbonyl)-6-(pyridin-2-yl)-2,6-diazabicyclo[3.2.1]octan-3-yl)-7-(2,3- |
| | dichlorophenyl)-6-fluoro-4-methyl-1H-pyrrolo[3,2-c]quinolin-8-yl)propanenitrile |
| 355 | 3-(2-((1R,3R,5R)-6-Benzoyl-2-(cyclopropanecarbonyl)-2,6- |
| | diazabicyclo[3.2.1]octan-3-yl)-1-((1R,4R,5S)-2-azabicyclo[2.1.1]hexan-5-yl)-7-(2,3- |
| | dichlorophenyl)-6-fluoro-4-methyl-1H-pyrrolo[3,2-c]quinolin-8-yl)propanenitrile |
| 356 | 3-((Rₐ)-2-((1R,3R,5R)-6-Benzoyl-2-(cyclopropanecarbonyl)-2,6- |
| | diazabicyclo[3.2.1]octan-3-yl)-1-((1R,4R,5S)-2-azabicyclo[2.1.1]hexan-5-yl)-7-(2,3- |
| | dichlorophenyl)-6-fluoro-4-methyl-1H-pyrrolo[3,2-c]quinolin-8-yl)propanenitrile |
| 357 | 3-((Sₐ)-2-((1R,3R,5R)-6-Benzoyl-2-(cyclopropanecarbonyl)-2,6- |
| | diazabicyclo[3.2.1]octan-3-yl)-1-((1R,4R,5S)-2-azabicyclo[2.1.1]hexan-5-yl)-7-(2,3- |
| | dichlorophenyl)-6-fluoro-4-methyl-1H-pyrrolo[3,2-c]quinolin-8-yl)propanenitrile |
| 358 | 3-(1-((1R,4R,5S)-2-Azabicyclo[2.1.1]hexan-5-yl)-2-((1R,5R,7R)-6- |
| | (cyclopropanecarbonyl)-2-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 359 | 3-((Rₐ)-1-((1R,4R,5S)-2-Azabicyclo[2.1.1]hexan-5-yl)-2-((1R,5R,7R)-6- |
| | (cyclopropanecarbonyl)-2-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 360 | 3-((Sₐ)-1-((1R,4R,5S)-2-Azabicyclo[2.1.1]hexan-5-yl)-2-((1R,5R,7R)-6- |
| | (cyclopropanecarbonyl)-2-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 361 | 3-(1-((1R,4R,5S)-2-Azabicyclo[2.1.1]hexan-5-yl)-2-((1S,5R,7R)-6- |
| | (cyclopropanecarbonyl)-4-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 362 | 3-((Rₐ)-1-((1R,4R,5S)-2-Azabicyclo[2.1.1]hexan-5-yl)-2-((1S,5R,7R)-6- |
| | (cyclopropanecarbonyl)-4-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 363 | 3-((Sₐ)-1-((1R,4R,5S)-2-Azabicyclo[2.1.1]hexan-5-yl)-2-((1S,5R,7R)-6- |
| | (cyclopropanecarbonyl)-4-oxo-3-(4-(trifluoromethyl)phenyl)-3,6- |
| | diazabicyclo[3.2.1]octan-7-yl)-7-(2,3-dichlorophenyl)-6-fluoro-4-methyl-1H- |
| | pyrrolo[3,2-c]quinolin-8-yl)propanenitrile |
| 364 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-2-methyl-3,6-diazabicyclo[3.2.1]octane-6- |
| | carboxylate |
| 365 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-2-methyl-3,6-diazabicyclo[3.2.1]octane-6- |
| | carboxylate |
| 366 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-2-methyl-3,6-diazabicyclo[3.2.1]octane-6- |
| | carboxylate |
| 367 | 3-(1-((1R,4R,5S)-2-Azabicyclo[2.1.1]hexan-5-yl)-2-((1S,5R,7R)-6- |
| | (cyclopropanecarbonyl)-4-oxo-3-(pyridin-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 368 | 3-((Rₐ)-1-((1R,4R,5S)-2-Azabicyclo[2.1.1]hexan-5-yl)-2-((1S,5R,7R)-6- |
| | (cyclopropanecarbonyl)-4-oxo-3-(pyridin-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 369 | 3-((Sₐ)-1-((1R,4R,5S)-2-Azabicyclo[2.1.1]hexan-5-yl)-2-((1S,5R,7R)-6- |
| | (cyclopropanecarbonyl)-4-oxo-3-(pyridin-2-yl)-3,6-diazabicyclo[3.2.1]octan-7-yl)-7- |
| | (2,3-dichlorophenyl)-6-fluoro-4-methyl-1H-pyrrolo[3,2-c]quinolin-8-yl)propanenitrile |
| 370 | 3-(1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl-2,2-d2)-7-(2,3-dichlorophenyl)-6-fluoro-4-methyl-1H-pyrrolo[3,2-c]quinolin-8- |
| | yl)propanenitrile |
| 371 | 3-((Rₐ)-1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl-2,2-d2)-7-(2,3-dichlorophenyl)-6-fluoro-4-methyl-1H-pyrrolo[3,2-c]quinolin-8- |
| | yl)propanenitrile |
| 372 | 3-((Sₐ)-1-((1R,4R,5S)-2-Azabicyclo[2.1.1]hexan-5-yl)-2-((1S,5R,7R)-6- |
| | (cyclopropanecarbonyl)-3-(5-isopropylthiazol-2-yl)-3,6-diazabicyclo[3.2.1]octan-7- |
| | yl-2,2-d2)-7-(2,3-dichlorophenyl)-6-fluoro-4-methyl-1H-pyrrolo[3,2-c]quinolin-8- |
| | yl)propanenitrile |
| 373 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-3,6-diazabicyclo[3.2.1]octane-6- |
| | carboxylate |
| 374 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-3,6-diazabicyclo[3.2.1]octane-6- |
| | carboxylate |
| 375 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(6-(1,1-difluoroethyl)pyrimidin-4-yl)-3,6-diazabicyclo[3.2.1]octane-6- |
| | carboxylate |
| 376 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(3-methoxypyrazin-2-yl)-3,6-diazabicyclo[3.2.1]octane-6-carboxylate |
| 377 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(3-methoxypyrazin-2-yl)-3,6-diazabicyclo[3.2.1]octane-6-carboxylate |
| 378 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(3-methoxypyrazin-2-yl)-3,6-diazabicyclo[3.2.1]octane-6-carboxylate |
| 379 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-(cyclopropylmethoxy)pyrimidin-2-yl)-3,6-diazabicyclo[3.2.1]octane-6- |
| | carboxylate |
| 380 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-(cyclopropylmethoxy)pyrimidin-2-yl)-3,6-diazabicyclo[3.2.1]octane-6- |
| | carboxylate |
| 381 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-(cyclopropylmethoxy)pyrimidin-2-yl)-3,6-diazabicyclo[3.2.1]octane-6- |
| | carboxylate |
| 382 | Methyl (1S,5R,7R)-7-(1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-cyanopyrazin-2-yl)-3,6-diazabicyclo[3.2.1]octane-6-carboxylate |
| 383 | Methyl (1S,5R,7R)-7-((Rₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-cyanopyrazin-2-yl)-3,6-diazabicyclo[3.2.1]octane-6-carboxylate |
| 384 | Methyl (1S,5R,7R)-7-((Sₐ)-1-((1R,4R,5S)-2-azabicyclo[2.1.1]hexan-5-yl)-8-(2- |
| | cyanoethyl)-7-(2,3-dichlorophenyl)-6-fluoro-4-methyl-1H-pyrrolo[3,2-c]quinolin-2- |
| | yl)-3-(5-cyanopyrazin-2-yl)-3,6-diazabicyclo[3.2.1]octane-6-carboxylate |
| 385 | 3-(1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(1-fluorocyclopropane-1- |
| | carbonyl)-3,6-diazabicyclo[3.2.1]octan-7-yl)-6-fluoro-4-methyl-1H-pyrrolo[3,2- |
| | c]quinolin-8-yl)propanenitrile |
| 386 | 3-((Rₐ)-1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(1-fluorocyclopropane-1- |
| | carbonyl)-3,6-diazabicyclo[3.2.1]octan-7-yl)-6-fluoro-4-methyl-1H-pyrrolo[3,2- |
| | c]quinolin-8-yl)propanenitrile |
| 387 | 3-((Sₐ)-1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(1-fluorocyclopropane-1- |
| | carbonyl)-3,6-diazabicyclo[3.2.1]octan-7-yl)-6-fluoro-4-methyl-1H-pyrrolo[3,2- |
| | c]quinolin-8-yl)propanenitrile |
| 388 | 3-(1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(2-hydroxyacetyl)-3,6- |
| | diazabicyclo[3.2.1]octan-7-yl)-6-fluoro-4-methyl-1H-pyrrolo[3,2-c]quinolin-8- |
| | yl)propanenitrile |
| 389 | 3-((Rₐ)-1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(2-hydroxyacetyl)-3,6- |
| | diazabicyclo[3.2.1]octan-7-yl)-6-fluoro-4-methyl-1H-pyrrolo[3,2-c]quinolin-8- |
| | yl)propanenitrile |
| 390 | 3-((Sₐ)-1-((1R,4R,5S)-2-azabicyclo[2.1.1]hexan-5-yl)-7-(2,3-dichlorophenyl)-2- |
| | ((1S,5R,7R)-3-(6-(1,1-difluoroethyl)pyrimidin-4-yl)-6-(2-hydroxyacetyl)-3,6- |
| | diazabicyclo[3.2.1]octan-7-yl)-6-fluoro-4-methyl-1H-pyrrolo[3,2-c]quinolin-8- |
| | yl)propanenitrile |

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule.

The term “Cₙ₋ₘ” indicates a range which includes the endpoints, wherein n and m are integers and indicate the number of carbons present in a chemical moiety. The term is intended to include each and every member in the indicated range. Thus, Cₙ₋ₘ includes each member in the series Cₙ, Cₙ₊₁, . . . Cₘ₋₁, and Cₘ. Examples include C₁₋₄ (which includes C₁, C₂, C₃, and C₄), C₁₋₆ (which includes C₁, C₂, C₃, C₄, C₅, and C₆) and the like.

The term “alkyl” employed alone or in combination with other terms, refers to a saturated hydrocarbon group that may be straight-chained or branched. The term “Cn-m alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cn-m alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkylenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms.

Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

The term “carbonyl,” employed alone or in combination with other terms, refers to a —C(═O)— group.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” or “oxy” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl. In some embodiments, the cycloalkyl group is tetrahydronaphthalenyl (e.g., 1,2,3,4-tetrahydronaphthalenyl).

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazobicyclo[2.2.1]heptanyl; pyrrolidinyl; hexahydropyrrolo[3,4-b]pyrrol-1(2H)-yl; 1,6-dihydropyridinyl; morpholinyl; azetidinyl; piperazinyl; and 4,7-diazaspiro[2.5]octan-7-yl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

When the compounds described herein contain a chiral center, unless otherwise indicated, the compounds can be any of the possible stereoisomers. In some embodiments, the compounds provided herein have the (R)-configuration. In other embodiments, the compounds have the (S)-configuration. In compounds with more than one chiral centers, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

Compounds provided herein can exist in the form of atropisomers (i.e., conformational diastereoisomers) that can be stable at ambient temperature and separable, e.g., by chromatography. For example, compounds in which Cy¹ is 2,3-dichlorophenyl, or any of the embodiments thereof, can exist in the form of atropisomers in which the conformation of the phenyl relative to the remainder of the molecule is as shown by the partial formulae Formula (II-A) or Formula (II-B) below. Reference to the compounds described herein or any of the embodiments is understood to include all such atropisomeric forms of the compounds, and mixtures thereof, including, without limitation, the atropisomeric forms represented by Formula (II-A) or Formula (II-B) below. Atropisomeric forms may be isolable by methods such as chromatography. The stereochemistry of atropisomeric forms can be designated applying IUPAC rules of nomenclature for stereochemistry. G. P. Moss, Pure & Appl. Chem., 1996, 68(12), 2193-2222. Without being limited by any theory, it is understood that, for a given compound, the atropisomer represented by Formula (II-A) is generally more potent as an inhibitor of KRAS (including G12C, G12D or G12V mutated forms of KRAS) than the atropisomer represented by Formula (II-B). In some embodiments, an atropisomer can be least partially or substantially separated from the alternative atropisomer of the compound, for example containing about 40% or less, about 30% or less, about 20% or less, about 10% or less, about 5% or less, about 2% or less, or about 1% or less of the alternative atropisomer.

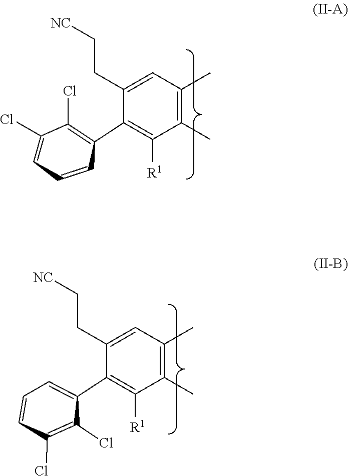

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. In the compounds provided herein, any atom not specifically designated as a particular isotope is meant to represent any stable isotope of that atom.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med. Chem. 2011, 54, 201-210; R. Xu et. al. J. Label Compd. Radiopharm. 2015, 58, 308-312). Unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium). In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “compound” is intended, unless otherwise specified, to include all stereoisomers, including without limitation, geometric isomers, configurational isomers, conformational isomers, rotational isomers, and atropisomers, of the structures depicted, including each of the embodiments thereof. The term is also intended to refer to compounds described herein regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds provided herein, or salts thereof, are substantially isolated. “Substantially isolated” means that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions “ambient temperature” and “room temperature” are understood in the art, and refer generally to a temperature, e.g., a reaction temperature, that is about the temperature of the room in which the reaction is carried out, e.g., a temperature from about 20° C. to about 30° C.

The present disclosure also includes pharmaceutically acceptable salts of the compounds described herein, including any of the embodiments thereof. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011). In some embodiments, the compounds described herein include the N-oxide forms.

### II. Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high-performance liquid chromatography (HPLC), liquid chromatography-mass spectroscopy (LCMS), or thin layer chromatography (TLC).

Compounds can be purified by those skilled in the art by a variety of methods, including high performance liquid chromatography (HPLC) (“Preparative LCMS Purification: Improved Compound Specific Method Optimization” Karl F. Blom, Brian Glass, Richard Sparks, Andrew P. Combs J. Combi. Chem. 2004, 6(6), 874-883) and normal phase silica chromatography.

The Schemes below provide general guidance in connection with preparing the compounds of the invention. One skilled in the art would understand that the preparations shown in the Schemes can be modified or optimized using general knowledge of organic chemistry to prepare various compounds of the invention.

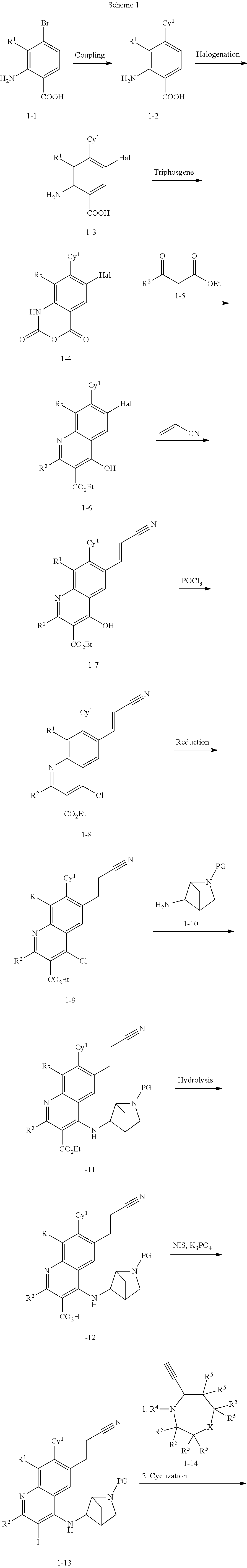

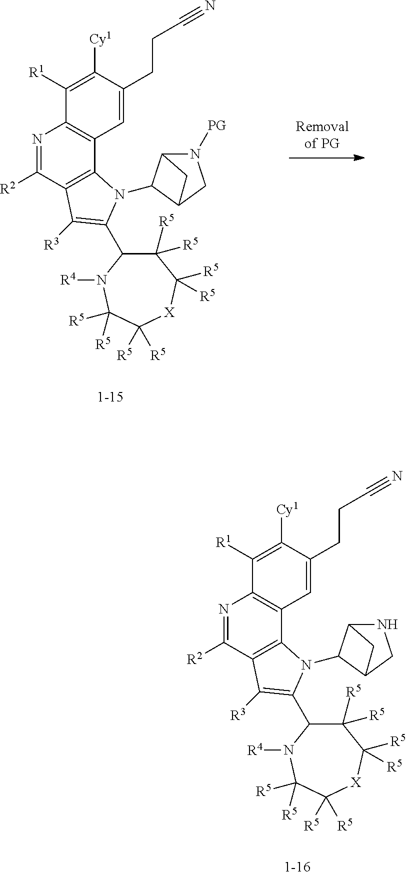

Compounds of formula 1-16 can be prepared via the synthetic route outlined in Scheme 1. The bromo of 1-1 can be converted to Cy¹ via standard Suzuki Cross-Coupling conditions (e.g., in the presence of a palladium catalyst and a suitable base), standard Negishi Cross-Coupling conditions, standard Stille Cross-Coupling conditions or other suitable methods to obtain 1-2. Halogenation of 1-2 with an appropriate reagent, such as N-iodosuccinimide (NIS), affords intermediate 1-3 (Hal is a halide, such as F, Cl, Br, or 1). Intermediate 1-3 can be treated with reagent such as triphosgene to afford compound 1-4. Intermediate 1-4 can then react with ester 1-5 to deliver compound 1-6. Coupling of halogen of 1-6 under standard Heck Cross-Coupling conditions or other suitable methods gives 1-7. Compound 1-8 can be prepared by treating 1-7 with an appropriate reagent such as POCl₃. Reduction of 1-8 under suitable conditions affords intermediate 1-9.

A S_{N}Ar reaction of intermediate 1-9 with amine 1-10 (PG is an appropriate protecting group, such as Boc) can be carried out to generate compound 1-11. Subsequent hydrolysis of ester of 1-11 affords 1-12, which can then be treated with NIS and an appropriate base (eg. K₃PO₄) to afford the corresponding iodide 2-13. Sonogashira coupling of 1-13 and compounds with formula of 1-14 followed by cyclization affords 1-15. Compound 1-15 can optionally undergo functionalization followed by deprotection of protecting group PG to afford compound 1-16. The order of the above described chemical reactions can be rearranged as appropriate to suite the preparation of different analogues.

For the synthesis of particular compounds, the general schemes described above and specific methods described herein for preparing particular compounds can be modified. For example, the products or intermediates can be modified to introduce particular functional groups. Alternatively, the substituents can be modified at any step of the overall synthesis by methods know to one skilled in the art, e.g., as described by R. C. Larock, Comprehensive Organic Transformations: A Guide to Functional Group Preparations (Wiley, 1999); R. C. Larock, et al., Comprehensive Organic Transformations: A Guide to Functional Group Preparations, 3^{rd} Ed. Vols. 1-4 (Wiley, 2018); A. R. Katritzky, et al. (Eds.), Comprehensive Organic Functional Group Transformations, Vols. 1-6 (Pergamon Press, 1995), and A. R. Katritzky et al. (Eds.), Comprehensive Organic Functional Group Transformations II, Vols. 1-6 (Elsevier, 2^{nd} Edition, 2005).

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St. C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### III. Uses of the Compounds

Compounds of the present disclosure, including the compounds of Formula (I), or any of the embodiments thereof, are useful for therapy as described in further detail below.

The present disclosure provides compounds of Formula (I), for use as a medicament, or for use in medicine. The present disclosure provides compounds of Formula (I), for use as a medicament, or for use in treating disease, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure are KRAS inhibitors and, thus, are useful in treating diseases and disorders associated with activity of KRAS. For the uses described herein, any of the compounds of Formula (I), including any of the embodiments thereof, may be used.

In particular, compounds of the invention are KRAS inhibitors having activity against one or more mutant forms of KRAS, and, thus, are useful in treating diseases and disorders associated with the presence or activity of mutant forms of KRAS, such as G12C, G12D, and/or the G12V mutant forms of KRAS.

The Ras family is comprised of three members: KRAS, NRAS and HRAS. RAS mutant cancers account for about 25% of human cancers. KRAS is the most frequently mutated isoform in human cancers: 85% of all RAS mutations are in KRAS, 12% in NRAS, and 3% in HRAS (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). The majority of RAS mutations occur at amino acid residues/codons 12, 13, and 61; Codon 12 mutations are most frequent in KRAS. The frequency of specific mutations varied between RAS genes and G12D mutations are most predominant in KRAS whereas Q61R and G12R mutations are most frequent in NRAS and HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) (A. D. Cox, et al. Nat. Rev. Drug. Discov., 2014, 13(11), 828-51). In contrast, KRAS G12C mutations predominate in non-small cell lung cancer (NSCLC) comprising 11-16% of lung adenocarcinomas (nearly half of mutant KRAS is G12C), as well as 2-5% of pancreatic and colorectal adenocarcinomas, respectively (A. D. Cox, et al. Nat. Rev. Drug. Discov., 2014, 13(11), 828-51). Using shRNA knockdown thousands of genes across hundreds of cancer cell lines, genomic studies have demonstrated that cancer cells exhibiting KRAS mutations are highly dependent on KRAS function for cell growth (R. McDonald, et al., Cell, 2017, 170(3), 577-92).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Diseases that can be treated with the compounds of Formula (I) include cancers. The cancers can include adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. In some embodiments, the cancer can be adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, or Wilms' tumor.

The cancer types in which KRAS harboring G12C, G12V and G12D mutations are implicated and that can be treated using compounds of Formula (I), or any of the embodiments thereof, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (K. Knickelbein, et al., Genes Dis., 2015, 2(1), 4-12). KRAS mutations were found in immunological and inflammatory disorders (Fernandez-Medarde, A. et al. Genes & Cancer, (2011): 344-358) such as Ras-associated lymphoproliferative disorder (RALD) or juvenile myelomonocytic leukemia (JMML) caused by somatic mutations of KRAS or NRAS.

Compounds of the present disclosure, including any of the embodiments thereof, can inhibit the activity of the KRAS protein. For example, compounds of the present disclosure can be used to inhibit activity of KRAS in a cell or in an individual or patient in need of inhibition of the enzyme by administering an inhibiting amount of one or more compounds of the present disclosure to the cell, individual, or patient.

As KRAS inhibitors, the compounds of the present disclosure, or any of the embodiments thereof, are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, the method comprising contacting a compound of the instant disclosure with KRAS. In an embodiment, the contacting comprises administering the compound to a patient. In an embodiment, KRAS is characterized as having a somatic mutation of G12C. In another embodiment, KRAS is characterized as having a somatic mutation of G12D. In another embodiment, KRAS is characterized as having a somatic mutation of G12V.

In an aspect, provided herein is a method of inhibiting a KRAS protein harboring a G12C mutation, the method comprising contacting a compound of Formula (I), or any of the embodiments thereof, with KRAS.

In an aspect, provided herein is a method of inhibiting a KRAS protein harboring a G12D mutation, the method comprising contacting a compound of Formula (I), or any of the embodiments thereof, with KRAS harboring a G12D mutation.

In an aspect, provided herein is a method of inhibiting a KRAS protein harboring a G12V mutation, the method comprising contacting a compound of Formula (I), or any of the embodiments thereof, with KRAS harboring a G12V mutation.

In another aspect, provided herein is a method of treating a disease or disorder associated with inhibition of KRAS interaction, the method comprising administering to a patient in need thereof a therapeutically effective amount of a compound of Formula (I), or any of the embodiments thereof.

In an embodiment, the disease or disorder is an immunological or inflammatory disorder. In another embodiment, the immunological or inflammatory disorder is Ras-associated lymphoproliferative disorder or juvenile myelomonocytic leukemia caused by somatic mutations of KRAS. In an embodiment, the immunological or inflammatory disorder is caused by a somatic mutation of KRAS.

In another embodiment, the somatic mutation of KRAS is G12C. In another embodiment, the somatic mutation of KRAS is G12D. In another embodiment, the somatic mutation of KRAS is G12V.

In another embodiment, the immunological or inflammatory disorder is associated with activity of KRAS having a G12C mutation. In another embodiment, the immunological or inflammatory disorder is associated with activity of KRAS having a G12D mutation. In another embodiment, the immunological or inflammatory disorder is associated with activity of KRAS having a G12V mutation.

In yet another aspect, provided herein is a method of treating a disease or disorder associated with inhibiting a KRAS protein harboring a G12C mutation, the method comprising administering to a patient in need thereof a therapeutically effective amount of a compound of a compound of Formula (I), or any of the embodiments thereof.

In yet another aspect, provided herein is a method of treating a disease or disorder associated with inhibiting a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with inhibiting a KRAS protein harboring a G12V mutation, the method comprising administering to a patient in need thereof a therapeutically effective amount of a compound of Formula (I), or any of the embodiments thereof.

In yet another aspect, provided herein is a method of treating a disease or disorder associated with activity of a KRAS protein harboring a G12C mutation, the method comprising administering to a patient in need thereof a therapeutically effective amount of a compound of a compound of Formula (I), or any of the embodiments thereof.

In yet another aspect, provided herein is a method of treating a disease or disorder associated with activity of a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with activity of a KRAS protein harboring a G12V mutation, the method comprising administering to a patient in need thereof a therapeutically effective amount of a compound of Formula (I), or any of the embodiments thereof.

In another aspect, provided herein is also a method of treating cancer in a patient in need thereof comprising administering to the patient a therapeutically effective amount of a compound of Formula (I), or any of the embodiments thereof.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12C mutation.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12D mutation.

In another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12V mutation.

In yet another aspect, provided herein is a method for treating a cancer in a patient, the method comprising administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12C mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12D mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12V mutation, and administering to the patient a therapeutically effective amount of any one of the compounds disclosed herein, or pharmaceutically acceptable salt thereof.

In an embodiment, the cancer is selected from carcinomas, hematological cancers, sarcomas, and glioblastoma. In another embodiment, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet another embodiment, the carcinoma is selected from pancreatic, colorectal, lung, bladder, gastric, esophageal, breast, head and neck, cervical, skin, and thyroid. In another embodiment, the cancer is colorectal cancer, pancreatic cancer, or lung cancer. In yet another embodiment, the pancreatic cancer is pancreatic ductal adenocarcinoma (PDAC). In still another embodiment, the cancer is non-small cell lung cancer (NSCLC).

In an embodiment, the cancer is metastatic.

In an aspect, provided herein is a method for treating a disease or disorder associated with inhibition of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an aspect, provided herein is a method for treating a disease or disorder associated with activity of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In certain embodiments, the disclosure provides a method for treating a KRAS-mediated disorder in a patient in need thereof, comprising the step of administering to the patient a compound according to the invention, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastasis.

In addition to oncogenic neoplasms, the compounds of the invention are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

The term “8p11 myeloproliferative syndrome” refers to myeloid/lymphoid neoplasms associated with eosinophilia and abnormalities of FGFR1.

The term “cell” refers to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” KRAS with a compound described herein includes the administration of a compound described herein to an individual or patient, such as a human, having KRAS, as well as, for example, introducing a compound described herein into a sample containing a cellular or purified preparation containing KRAS.

The terms “individual,” “subject,” or “patient,” are used interchangeably, and refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” comprises the prevention of at least one symptom associated with or caused by the state, disease or disorder being prevented.

### III. Combination Therapies

Compounds of the present disclosure, including the compounds of Formula (I), or any of the embodiments thereof, may be useful in therapy when used in combination with one or more additional pharmaceutical agents, as described in further detail below.

a. Cancer Therapies

Compounds of the invention, including the compounds of Formula (I), or any of the embodiments thereof, may be useful in treatment of cancer when used in combination with one or more additional pharmaceutical agents, as described in further detail below.

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of KRAS-associated diseases, disorders or conditions. Other agents such as therapeutic antibodies can be used in combination with the compounds of the present disclosure for treatment of KRAS-associated diseases, disorders or conditions. The one or more additional pharmaceutical agents can be administered to a patient simultaneously or sequentially.

In some embodiments, the KRAS inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein.

Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2; e.g., ruxolitinib or baricitinib; or JAK1; e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., zilurgisertib); or combinations thereof.

In some embodiments, the compound or salt described herein is administered with a PI3K6 inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-1 agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Pat. No. 7,745,437.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, B1853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the embodiments thereof as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the embodiments thereof as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

b. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IB1308), AB122 (GLS-010), AMP-224, AMP-514/MEDI-0680, BMS936559, JTX-4014, BGB-108, SHR-1210, MED14736, FAZ053, BCD-100, KN035, CS1001, BAT1306, LZM009, AKi05, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217,149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the PD-L1 inhibitor is selected from the compounds in Table 3, or a pharmaceutically acceptable salt thereof.

TABLE 3

| | US | |
|---|---|---|
| Cmpd | Publication | |
| No. | Appl. No. | Name and Structure |
| 1 | U.S. 2018- 0179197, Example #24 | 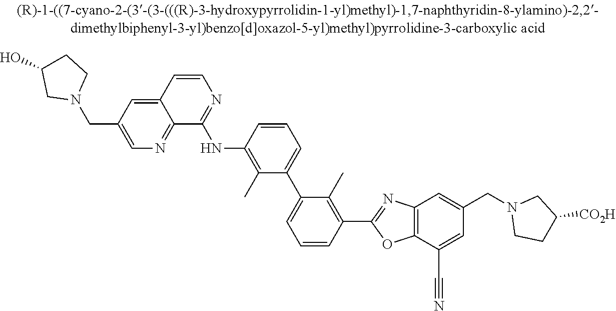 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 2 | U.S. 2018- 0179201, Example #2 | 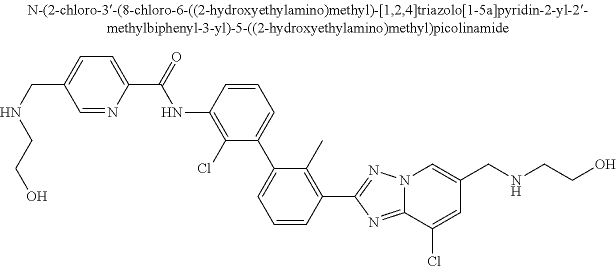 [SMILES: Cc1c(-c2nc3c(Cl)cc(CNCCO)cn3n2)cccc1-c1cccc(NC(=O)c2ccc(CNCCO)cn2)c1Cl] |
| 3 | U.S. 2018- 0179197, Example #25 | 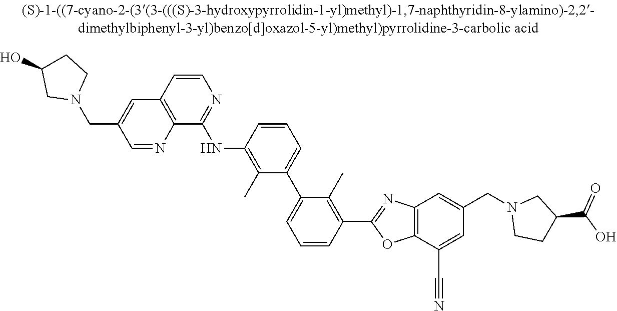 [SMILES: Cc1c(Nc2nccc3cc(CN4CC[C@H](O)C4)cnc23)cccc1-c1cccc(-c2nc3cc(CN4CC[C@H](C(=O)O)C4)cc(C#N)c3o2)c1C] |
| 4 | U.S. 2018- 0179197, Example #26 | 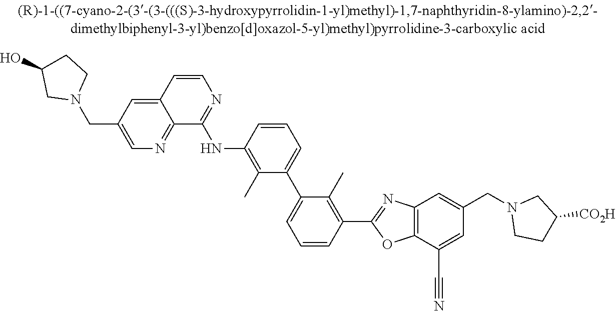 [SMILES: Cc1c(Nc2nccc3cc(CN4CC[C@H](O)C4)cnc23)cccc1-c1cccc(-c2nc3cc(CN4CC[C@@H](C(=O)O)C4)cc(C#N)c3o2)c1C] |
| 5 | U.S. 2018- 0179197, Example #28 | 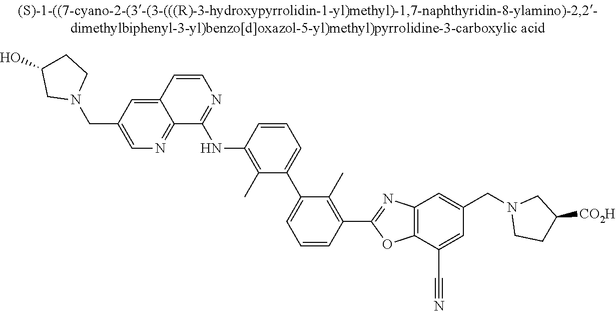 [SMILES: Cc1c(Nc2nccc3cc(CN4CC[C@@H](O)C4)cnc23)cccc1-c1cccc(-c2nc3cc(CN4CC[C@H](C(=O)O)C4)cc(C#N)c3o2)c1C] |
| 6 | U.S. 2018- 0179197, Example #236 | 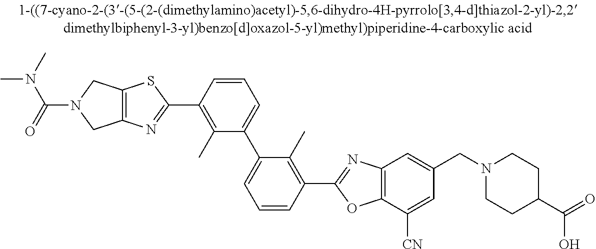 [SMILES: Cc1c(-c2nc3cc(CN4CCC(C(=O)O)CC4)cc(C#N)c3o2)cccc1-c1cccc(-c2nc3c(s2)CN(C(=O)N(C)C)C3)c1C] |
| 7 | U.S. 2018- 0179179, Example #1 | 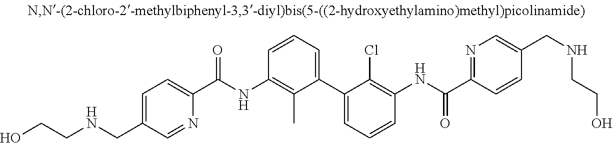 [SMILES: Cc1c(NC(=O)c2ccc(CNCCO)cn2)cccc1-c1cccc(NC(=O)c2ccc(CNCCO)cn2)c1Cl] |
| 8 | U.S. 2018- 0179179, Example #9 | 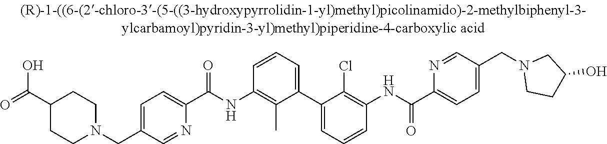 [SMILES: Cc1c(NC(=O)c2ccc(CN3CCC(C(=O)O)CC3)cn2)cccc1-c1cccc(NC(=O)c2ccc(CN3CC[C@@H](O)C3)cn2)c1Cl] |
| 9 | U.S. 2018- 0179179, Example #12 | 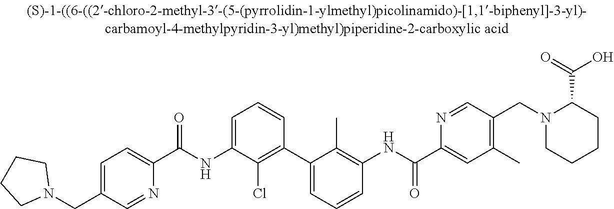 [SMILES: Cc1cc(C(=O)Nc2cccc(-c3cccc(NC(=O)c4ccc(CN5CCCC5)cn4)c3Cl)c2C)ncc1CN1CCCC[C@H]1C(=O)O] |
| 10 | U.S. 2018-0179202, Example #52 | 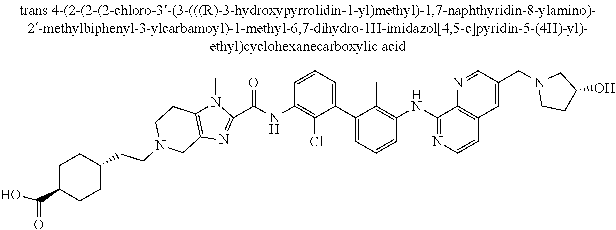 [SMILES: Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl] |
| 11 | U.S. 2018- 0179202, Example #56 | 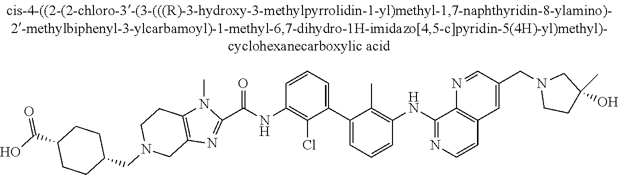 [SMILES: Cc1c(Nc2nccc3cc(CN4CC[C@@](C)(O)C4)cnc23)cccc1-c1cccc(NC(=O)c2nc3c(n2C)CCN(C[C@H]2CC[C@@H](C(=O)O)CC2)C3)c1Cl] |
| 12 | U.S. 2018- 0179202, Example #68 | 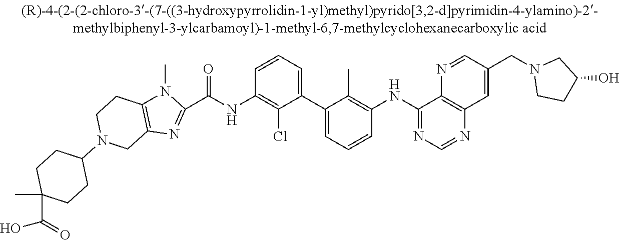 [SMILES: Cc1c(Nc2ncnc3cc(CN4CC[C@@H](O)C4)cnc23)cccc1-c1cccc(NC(=O)c2nc3c(n2C)CCN(C2CCC(C)(C(=O)O)CC2)C3)c1Cl] |
| 13 | U.S. 2018- 0179202, Example #90 | 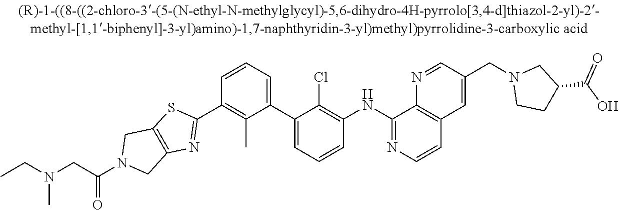 [SMILES: CCN(C)CC(=O)N1Cc2nc(-c3cccc(-c4cccc(Nc5nccc6cc(CN7CC[C@@H](C(=O)O)C7)cnc56)c4Cl)c3C)sc2C1] |
| 14 | U.S. 2018- 0177784, Example #35 | 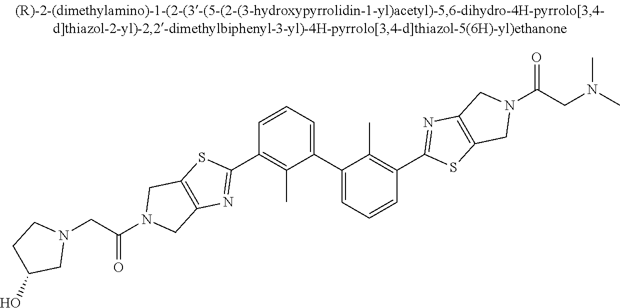 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C] |
| 15 | U.S. 2018- 0177870, Example #37 | 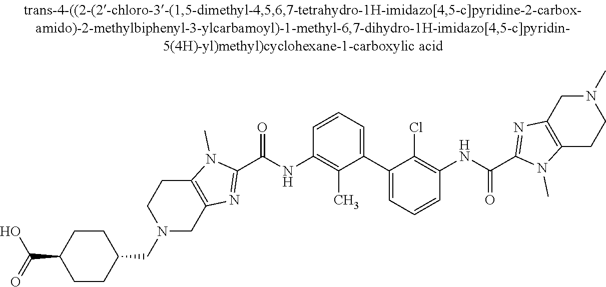 [SMILES: Cc1c(NC(=O)c2nc3c(n2C)CCN(C[C@H]2CC[C@H](C(=O)O)CC2)C3)cccc1-c1cccc(NC(=O)c2nc3c(n2C)CCN(C)C3)c1Cl] |
| 16 | U.S. 2018- 0177870, Example #100 | 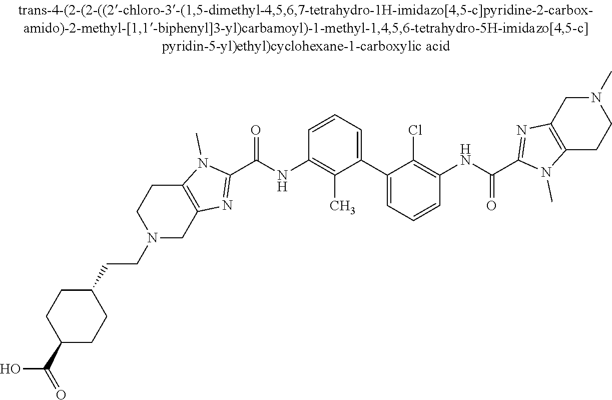 [SMILES: Cc1c(NC(=O)c2nc3c(n2C)CCN(CC[C@H]2CC[C@H](C(=O)O)CC2)C3)cccc1-c1cccc(NC(=O)c2nc3c(n2C)CCN(C)C3)c1Cl] |
| 17 | U.S. 2018- 0177870, Example #114 | 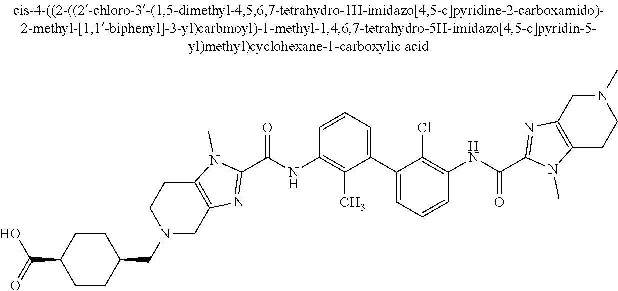 [SMILES: Cc1c(NC(=O)c2nc3c(n2C)CCN(C[C@H]2CC[C@@H](C(=O)O)CC2)C3)cccc1-c1cccc(NC(=O)c2nc3c(n2C)CCN(C)C3)c1Cl] |
| 18 | U.S. 2018- 0177870, Example #135 | 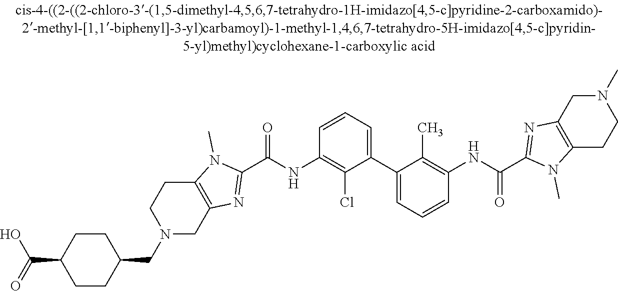 [SMILES: Cc1c(NC(=O)c2nc3c(n2C)CCN(C)C3)cccc1-c1cccc(NC(=O)c2nc3c(n2C)CCN(C[C@H]2CC[C@@H](C(=O)O)CC2)C3)c1Cl] |
| 19 | U.S. 2018- 0177870, Example #148 | 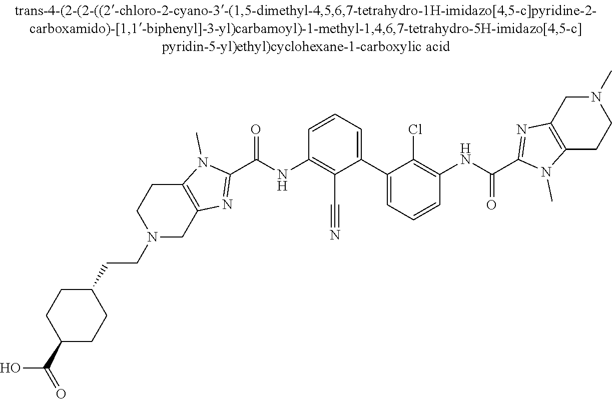 [SMILES: CN1CCc2c(nc(C(=O)Nc3cccc(-c4cccc(NC(=O)c5nc6c(n5C)CCN(CC[C@H]5CC[C@H](C(=O)O)CC5)C6)c4C#N)c3Cl)n2C)C1] |
| 20 | U.S. 2018- 0177870, Example #159 | 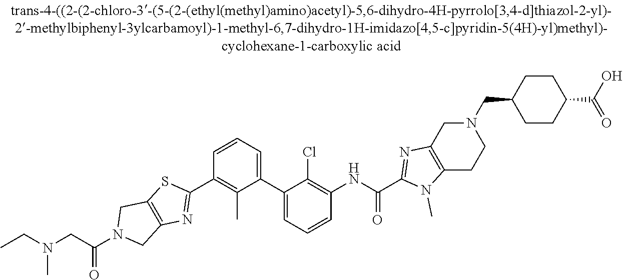 [SMILES: CCN(C)CC(=O)N1Cc2nc(-c3cccc(-c4cccc(NC(=O)c5nc6c(n5C)CCN(C[C@H]5CC[C@H](C(=O)O)CC5)C6)c4Cl)c3C)sc2C1] |
| 21 | U.S. 2018- 0177870, Example #160 | 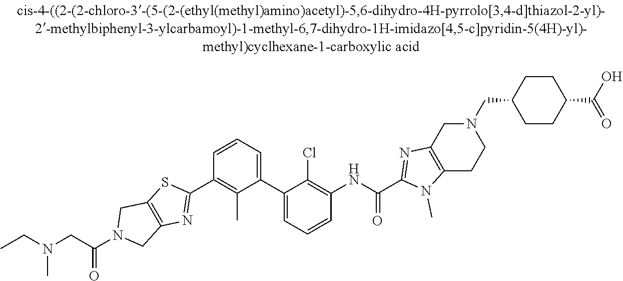 [SMILES: CCN(C)CC(=O)N1Cc2nc(-c3cccc(-c4cccc(NC(=O)c5nc6c(n5C)CCN(C[C@H]5CC[C@@H](C(=O)O)CC5)C6)c4Cl)c3C)sc2C1] |
| 22 | U.S. 2018- 0177870, Example #161 | 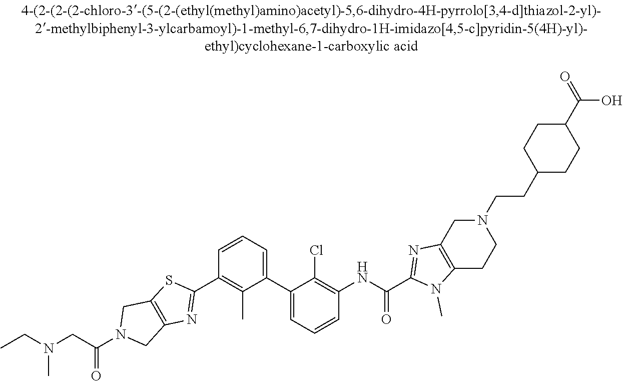 [SMILES: CCN(C)CC(=O)N1Cc2nc(-c3cccc(-c4cccc(NC(=O)c5nc6c(n5C)CCN(CCC5CCC(C(=O)O)CC5)C6)c4Cl)c3C)sc2C1] |
| 23 | U.S. 2018- 0177870, Example #162 | 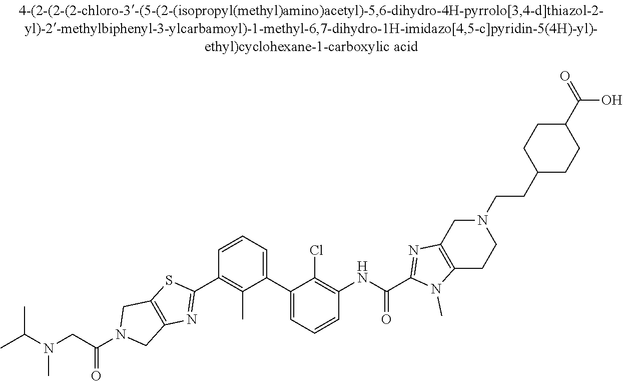 [SMILES: Cc1c(-c2nc3c(s2)CN(C(=O)CN(C)C(C)C)C3)cccc1-c1cccc(NC(=O)c2nc3c(n2C)CCN(CCC2CCC(C(=O)O)CC2)C3)c1Cl] |
| 24 | U.S. 2019- 0300524, Example #16 | 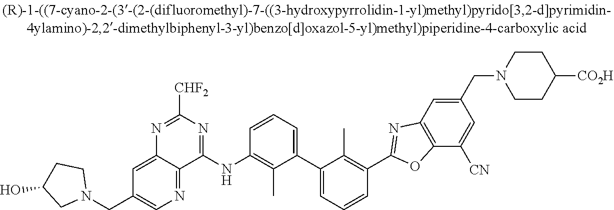 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C] |
| 25 | U.S. 2019- 0300524, Example #17 | 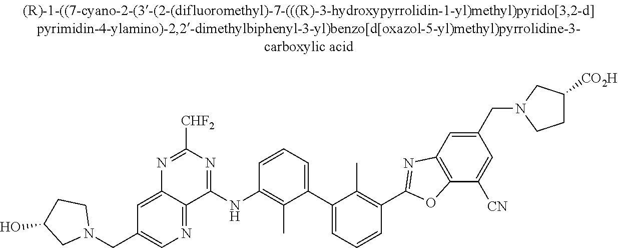 [SMILES: Cc1c(Nc2nc(C(F)F)nc3cc(CN4CC[C@@H](O)C4)cnc23)cccc1-c1cccc(-c2nc3cc(CN4CC[C@@H](C(=O)O)C4)cc(C#N)c3o2)c1C] |
| 26 | U.S. 2019- 0300524, Example #18 | 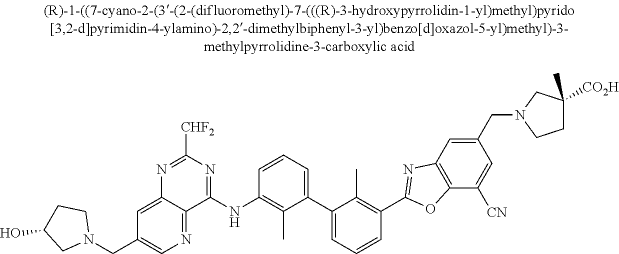 [SMILES: Cc1c(Nc2nc(C(F)F)nc3cc(CN4CC[C@@H](O)C4)cnc23)cccc1-c1cccc(-c2nc3cc(CN4CC[C@@](C)(C(=O)O)C4)cc(C#N)c3o2)c1C] |
| 27 | U.S. 2019- 0300524, Example #30 | 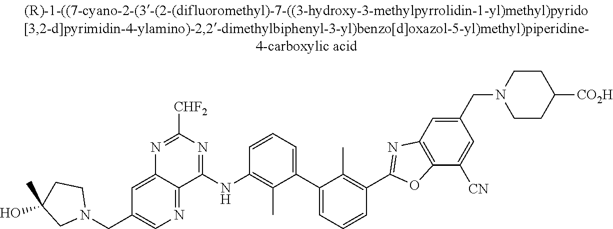 [SMILES: Cc1c(Nc2nc(C(F)F)nc3cc(CN4CC[C@@](C)(O)C4)cnc23)cccc1-c1cccc(-c2nc3cc(CN4CCC(C(=O)O)CC4)cc(C#N)c3o2)c1C] |
| 28 | U.S. 2019- 0300524, Example #31 | 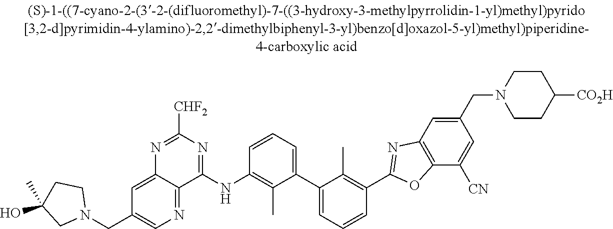 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C] |
| 29 | U.S. 2019- 0345170, Example #13 | 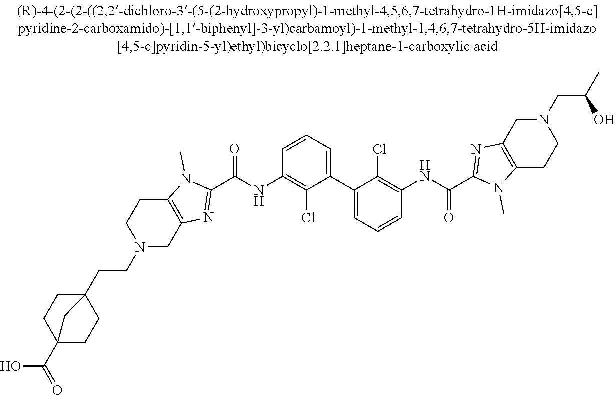 [SMILES: C[C@@H](O)CN1CCc2c(nc(C(=O)Nc3cccc(-c4cccc(NC(=O)c5nc6c(n5C)CCN(CCC57CCC(C(=O)O)(CC5)C7)C6)c4Cl)c3Cl)n2C)C1] |
| 30 | U.S. 2019- 0345170, Example #17 | 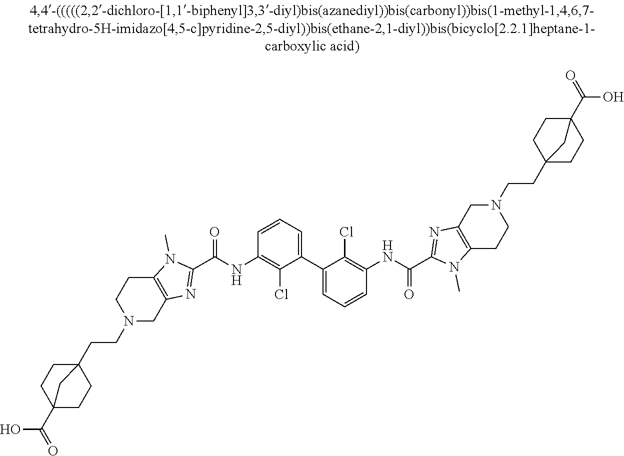 [SMILES: Cn1c(C(=O)Nc2cccc(-c3cccc(NC(=O)c4nc5c(n4C)CCN(CCC46CCC(C(=O)O)(CC4)C6)C5)c3Cl)c2Cl)nc2c1CCN(CCC13CCC(C(=O)O)(CC1)C3)C2] |
| 31 | U.S. 2019- 0345170, Example #18 | 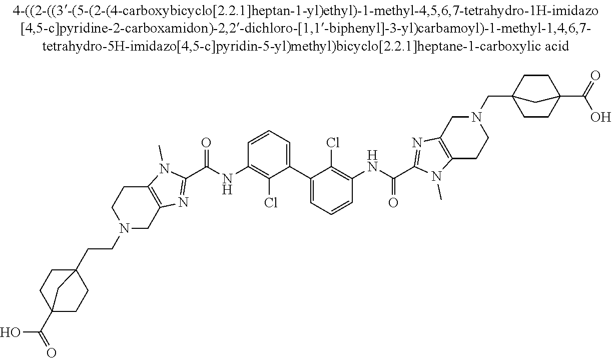 [SMILES: Cn1c(C(=O)Nc2cccc(-c3cccc(NC(=O)c4nc5c(n4C)CCN(CC46CCC(C(=O)O)(CC4)C6)C5)c3Cl)c2Cl)nc2c1CCN(CCC13CCC(C(=O)O)(CC1)C3)C2] |
| 32 | U.S. 2019- 0345170, Example #34 | 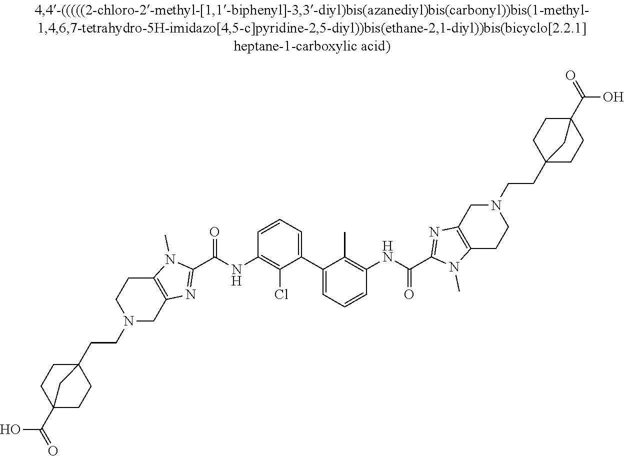 [SMILES: Cc1c(NC(=O)c2nc3c(n2C)CCN(CCC24CCC(C(=O)O)(CC2)C4)C3)cccc1-c1cccc(NC(=O)c2nc3c(n2C)CCN(CCC24CCC(C(=O)O)(CC2)C4)C3)c1Cl] |
| 33 | U.S. 2019- 0345170, Example #51 | 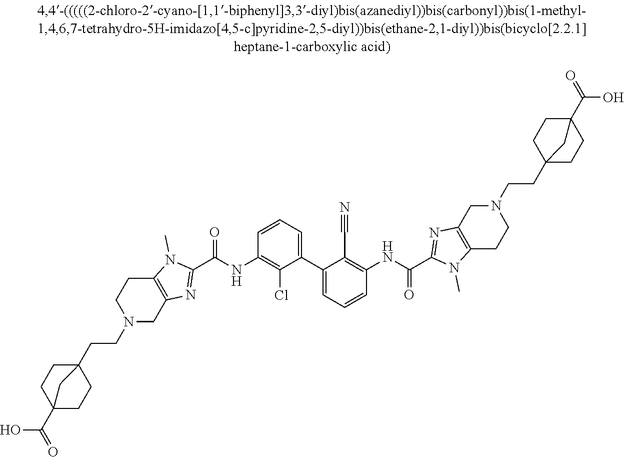 [SMILES: Cn1c(C(=O)Nc2cccc(-c3cccc(NC(=O)c4nc5c(n4C)CCN(CCC46CCC(C(=O)O)(CC4)C6)C5)c3C#N)c2Cl)nc2c1CCN(CCC13CCC(C(=O)O)(CC1)C3)C2] |
| 34 | U.S. 2021- 0094976, Example #1 | 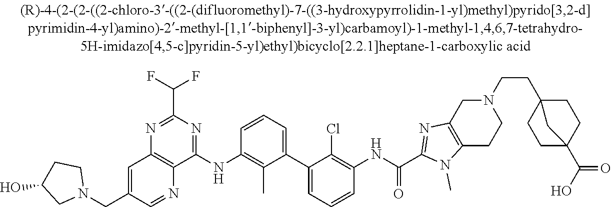 [SMILES: Cc1c(Nc2nc(C(F)F)nc3cc(CN4CC[C@@H](O)C4)cnc23)cccc1-c1cccc(NC(=O)c2nc3c(n2C)CCN(CCC24CCC(C(=O)O)(CC2)C4)C3)c1Cl] |

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MED14736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MED14736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, US 2019/0300524, and US 2019/0345170, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MED19447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MED11873, or MED16469. In some embodiments, the agonist of an immune checkpoint molecule is an agonist of OX40, e.g., OX40 agonist antibody or OX40L fusion protein. In some embodiments, the anti-OX40 antibody is INCAGN01949, MED10562 (tavolimab), MOXR-0916, PF-04518600, GSK3174998, BMS-986178, or 9B12. In some embodiments, the OX40L fusion protein is MED16383.

In some embodiments, the agonist of an immune checkpoint molecule is an agonist of CD40. In some embodiments, the agonist of CD40 is CP-870893, ADC6-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGF3 receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK 04.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### IV. Formulation, Dosage Forms and Administration

When employed as pharmaceuticals, the compounds of the present disclosure can be administered in the form of pharmaceutical compositions. Thus, the present disclosure provides a composition comprising a compound of Formula (I), a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™)

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calc. to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 g/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

Another aspect of the present invention relates to labeled compounds of the disclosure (radio-labeled, fluorescent-labeled, etc.) that would be useful not only in imaging techniques but also in assays, both in vitro and in vivo, for localizing and quantitating KRAS protein in tissue samples, including human, and for identifying KRAS ligands by inhibition binding of a labeled compound. Substitution of one or more of the atoms of the compounds of the present disclosure can also be useful in generating differentiated ADME (Adsorption, Distribution, Metabolism and Excretion). Accordingly, the present invention includes KRAS binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). A “radio-labelled” compound can refer to an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to other forms of hydrogen.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes, et al., J. Med. Chem. 2011, 54(1), 201-10; R. Xu et al., J. Label. Compd. Radiopharm. 2015, 58, 308-12). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the invention can be used in a screening assay to identify and/or evaluate compounds. For example, a newly synthesized or identified compound (i.e., test compound) which is labeled can be evaluated for its ability to bind a KRAS protein by monitoring its concentration variation when contacting with the KRAS, through tracking of the labeling. For example, a test compound (labeled) can be evaluated for its ability to reduce binding of another compound which is known to bind to a KRAS protein (i.e., standard compound). Accordingly, the ability of a test compound to compete with the standard compound for binding to the KRAS protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of KRAS, such as cancer or infections, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

The compounds of the Examples have been found to inhibit the activity of KRAS according to at least one assay described herein.

### EXAMPLES

Experimental procedures for compounds of the invention are provided below. Preparatory LCMS purifications of some of the compounds prepared were performed on Waters mass directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical liquid chromatography mass spectrometry (LCMS) for purity check.

The compounds separated were typically subjected to analytical liquid chromatography mass spectrometry (LCMS) for purity check under the following conditions: Instrument; Agilent 1100 series, LC/MSD, Column: Waters SUNFIRE® C₁₈ 5 μm particle size, 2.1×5.0 mm, Buffers: mobile phase A: 0.025% TFA in water and mobile phase B: MeCN; gradient 2% to 80% of B in 3 min. with flow rate 2.0 mL/min.

Some of the compounds prepared were also separated on a preparative scale by RP-HPLC with MS detection or FCC (silica gel) as indicated in the Examples. Typical preparative RP-HPLC column conditions are as follows:

pH=2 purifications: Waters SUNFIRE® C₁₈ 5 μm particle size, 19×100 mm column, eluting with mobile phase A: 0.1% TFA in water and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with the 30×100 mm column was 60 mL/min.

pH=10 purifications: Waters XBRIDGE® C₁₈ 5 μm particle size, 19×100 mm column, eluting with mobile phase A: 0.15% NH₄OH in water and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with 30×100 mm column was 60 mL/min.

The following abbreviations may be used herein: AcOH (acetic acid); Ac₂O (acetic anhydride); aq. (aqueous); atm. (atmosphere(s)); BF₃·Et₂O (boron trifluoride ethyl etherate); Boc (t-butoxycarbonyl); Boc₂O (di-t-butyl dicarbonate); BOP ((benzotriazol-1-yloxy)tris(dimethylamino)phosphonium hexafluorophosphate); n-BuLi (n-butyllithium); br (broad); Cbz (carboxybenzyl); calc. (calc.); CO₂ (carbion dioxide); Cs₂CO₃ (cesium carbonate); CsF (cesium fluoride); CuI (copper(I) iodide); d (doublet); dd (doublet of doublets); DBU (1,8-diazabicyclo[5.4.0]undec-7-ene); DCM (dichloromethane); DIAD (N,N′diisopropyl azidodicarboxylate); DIPEA (N,N-diisopropylethylamine); DIBAL (diisobutylaluminium hydride); DMF (N,N-dimethylformamide); DMSO (dimethylsulfoxide); eq. (equivalent(s)); Et (ethyl); EtOH (ethanol); EtOAc (ethyl acetate); Ex. (Example); FCC (flash column chromatography); g (gram(s)); h (hour(s)); GPhos PD G6 TES (bromo[dicyclohexyl[3-(1,1-dimethylethoxy)-6-methoxy-2′,6′-bis(1-methylethyl)[1,1′-biphenyl]-2-yl]phosphine](4-((2-(trimethylsilyl)ethoxy)carbonyl)benzen-1-ide)palladium(II)); H₂ (hydrogen); HATU (N,N,N′,N′tetramethyl-O-(7-azabenzotriazol-1-yl)uronium hexafluorophosphate); HCl (hydrochloric acid or hydrogen chloride); HPLC (high performance liquid chromatography); Hz (hertz); J (coupling constant); K₂CO₃ (potassium carbonate); LCMS (liquid chromatography-mass spectrometry); LiAlD₄ (lithium aluminium deuteride); LiBH₄ (lithium borohydride); LiCl (lithium chloride); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); MgSO₄ (magnesium sulfate); min. (minutes(s)); mL (milliliter(s)); mmol (millimole(s)); MTBE (methyl tert-butyl ether); N (normal); NaBH₄ (sodium borohydride); NaBH(OAc)₃ (sodium triacetoxyborohydride); NaCl (sodium chloride); NADPH (nicotinamide adenine dinucleotide phosphate); NaHCO₃ (sodium bicarbonate); NaIO₄ (sodium metaperiodate); NaOH (sodium hydroxide); Na₂SO₄ (sodium sulfate); Na₂S₂O₃ (sodium thiosulfate); NCS (N-chlorosuccinimide); NEt₃ (triethylamine); NH₄OH (ammonium hydroxide); nM (nanomolar); NMP (N-methylpyrrolidinone); NMR (nuclear magnetic resonance spectroscopy); OTf (trifluoromethanesulfonate); Pd(OH)₂/C (palladium hydroxide on carbon); Pd(PPh₃)₄ (tetrakis(triphenylphosphine)palladium(0)); Ph (phenyl); PhI(OAc)₂ (phenyliodonium diacetate); pM (picomolar); PPT (precipitate); PPTS (pyridinium p-toluenesulfonate); prep. (preparative); RP-HPLC (reverse phase high performance liquid chromatography); r.t. (room temperature), RuPhos (2-dicyclohexylphosphino-2′,6′-diisopropoxybiphenyl); RuPhos Pd G4 ([dicyclohexyl(2′,6′-diisopropoxy-2-biphenylyl)phosphine-KP](methanesulfonatato-κO)[2′-(methylamino-κN)-2-biphenylyl-κC²]palladium); s (singlet); sat. (saturated); t (triplet or tertiary); T3P (Propanephosphonic acid anhydride); TBAF (tetrabutylammonium fluoride); TBDPS (tert-butyldiphenylsilyl); TBDPSCI (tert-butylchlorodiphenylsilane); TBS (tert-butyldimethylsilyl); TBSCI (tert-butylchlorodimethylsilane); TEMPO ((2,2,6,6-tetramethylpiperidin-1-yl)oxyl); tert(tertiary); tt (triplet of triplets); TFA (trifluoroacetic acid); THF (tetrahydrofuran); TLC (thin layer chromatography); μg (microgram(s)); μL (microliter(s)); μM (micromolar); wt % (weight percent); xantphos (4,5-bis(diphenylphosphino)-9,9-dimethylxanthene). Brine is sat. aq. NaCl. In vacuo is under vacuum.

### Intermediate 1. Ethyl (Rₐ)-4-Chloro-6-(2-Cyanoethyl)-7-(2,3-Dichlorophenyl)-8-Fluoro-2-methylquinoline-3-carboxylate

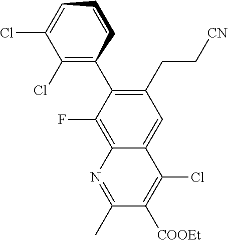

### Step 1. 3-Amino-2′,3′-dichloro-2-fluoro-[1,1′-biphenyl]-4-carboxylic acid

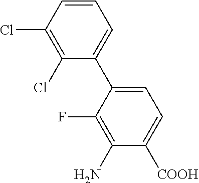 [SMILES: Nc1c(C(=O)O)ccc(-c2cccc(Cl)c2Cl)c1F]

A mixture of 2-amino-4-bromo-3-fluorobenzoic acid (28.0 g, 120 mmol), (2,3-dichlorophenyl)boronic acid (25.1 g, 132 mmol), bis(di-tert-butyl(4-dimethylaminophenyl)phosphine)dichloropalladium(II) (2.12 g, 3.00 mmol) and potassium phosphate (50.8 g, 239 mmol) in 1,4-dioxane (170 mL) and water (30 mL) was sparged with N₂ and heated at 70° C. for 1 h. The mixture was allowed to cool to r.t. and poured into 1 M HCl (200 mL). The mixture was stirred for another 10 min., resulting in precipitation. A precipitate formed that was collected by filtration, washed with water followed by hexanes and dried under reduced pressure to afford the sub-title compound in near quantitative yield. The crude product was used in next step without further purification. LCMS calc. for C₁₃H₉Cl₂FNO₂ (M+H)⁺: m/z=300.0; found 300.0.

### Step 2. 3-Amino-6-bromo-2′,3′-dichloro-2-fluoro-[1,1′-biphenyl]-4-carboxylic acid

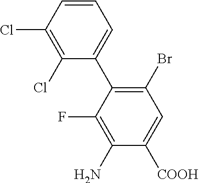 [SMILES: Nc1c(C(=O)O)cc(Br)c(-c2cccc(Cl)c2Cl)c1F]

To a solution of 3-amino-2′,3′-dichloro-2-fluoro-[1,1′-biphenyl]-4-carboxylic acid (35.8 g, 119 mmol) in DMSO (100 mL) was added N-bromosuccinimide (22.3 g, 125 mmol). The resulting mixture was heated at 50° C. for 1 h. The mixture was allowed to cool to r.t. and poured into ice water (400 mL). To the suspension was added sat. aq. Na₂S₂O₃ (20 mL). After stirring for 15 min., the solids were collected by filtration, washed with water followed by hexanes and dried under reduced pressure to afford the sub-title compound (43.0 g, 95% yield). The crude product was used in next step without further purification. LCMS calc. for C₁₃H₈BrCl₂FNO₂ (M+H)⁺: m/z=377.9, 379.9; found 378.0, 380.0.

### Step 3. 6-Bromo-7-(2,3-dichlorophenyl)-8-fluoro-2H-benzo[d][1,3]oxazine-2,4(1H)-dione

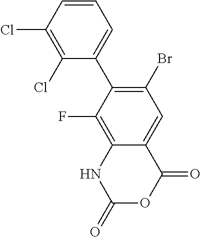 [SMILES: O=c1[nH]c2c(F)c(-c3cccc(Cl)c3Cl)c(Br)cc2c(=O)o1]

To a solution of 3-amino-6-bromo-2′,3′-dichloro-2-fluoro-[1,1′-biphenyl]-4-carboxylic acid (38.6 g, 102 mmol) in THF (300 mL) was added triphosgene (10.6 g, 35.6 mmol) portionwise. After addition, the mixture was heated at 60° C. for 0.5 h. The mixture was allowed to cool to r.t. and poured into heptane (1000 mL). After stirring for 1 h, the solids were collected by filtration, washed with hexanes and dried under reduced pressure to afford the sub-title compound in near quantitative yield. The crude product was used in next step without further purification.

### Step 4. Ethyl 6-bromo-7-(2,3-dichlorophenyl)-8-fluoro-4-hydroxy-2-methylquinoline-3-carboxylate

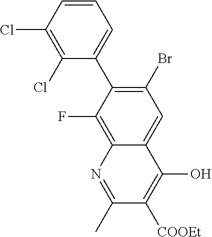 [SMILES: CCOC(=O)c1c(C)nc2c(F)c(-c3cccc(Cl)c3Cl)c(Br)cc2c1O]

To a solution of 6-bromo-7-(2,3-dichlorophenyl)-8-fluoro-2H-benzo[d][1,3]oxazine-2,4(1H)-dione (41.5 g, 102 mmol) in DMSO (200 mL) was added (1-ethoxy-1,3-dioxobutan-2-yl)sodium (18.7 g, 123 mmol) portionwise. After addition, the mixture was heated at 80° C. for 1 h. The mixture was allowed to cool to r.t. and poured into 1 M HCl (400 mL). After stirring for 1 h, the solids were collected by filtration, washed with water followed by hexanes and dried under reduced pressure to afford the sub-title compound (40.0 g, 83% yield). The crude product was used in next step without further purification. LCMS calc. for C₁₉H₁₄BrCl₂FNO₃ (M+H)⁺: m/z=471.9, 473.9; found 471.9, 474.0.

### Step 5. Ethyl (E)-6-(2-cyanovinyl)-7-(2,3-dichlorophenyl)-8-fluoro-4-hydroxy-2-methylquinoline-3-carboxylate

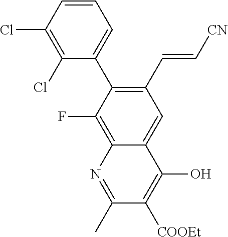 [SMILES: CCOC(=O)c1c(C)nc2c(F)c(-c3cccc(Cl)c3Cl)c(/C=C/C#N)cc2c1O]

To a solution of ethyl 6-bromo-7-(2,3-dichlorophenyl)-8-fluoro-4-hydroxy-2-methylquinoline-3-carboxylate (35.0 g, 74.0 mmol), bis(di-tert-butyl(4-dimethylaminophenyl)phosphine)dichloropalladium(II) (2.62 g, 3.70 mmol) in DMF (100 mL) was added acrylonitrile (12.3 mL, 185 mmol) and NEt₃ (30.9 mL, 222 mmol). The mixture was sparged with N₂ and heated at 85° C. for 1 h. The mixture was allowed to cool to r.t. and poured into 1 M HCl (500 mL). After stirring for 1 h, the solids were collected by filtration, washed with water followed by hexanes and dried under reduced pressure to afford the sub-title compound (19.2 g, 58% yield). The crude product was used in next step without further purification. LCMS calc. for C₂₂H₁₆Cl₂FN₂O₃ (M+H)⁺: m/z=445.0; found 445.0.

### Step 6. Ethyl (E)-4-chloro-6-(2-cyanovinyl)-7-(2,3-dichlorophenyl)-8-fluoro-2-methylquinoline-3-carboxylate

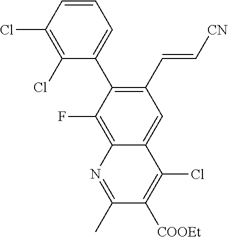 [SMILES: CCOC(=O)c1c(C)nc2c(F)c(-c3cccc(Cl)c3Cl)c(/C=C/C#N)cc2c1Cl]

To a slurry of ethyl (E)-6-(2-cyanovinyl)-7-(2,3-dichlorophenyl)-8-fluoro-4-hydroxy-2-methylquinoline-3-carboxylate (30.0 g, 67.4 mmol) and benzyltriethylammonium chloride (15.4 g, 67.4 mmol) in MeCN (150 mL) at 0° C. was added DIPEA (23.5 mL, 135 mmol). Upon stirring at 0° C., phosphoryl chloride (12.6 mL, 135 mmol) was added dropwise into the mixture. Then the mixture was heated at 60° C. for 1 h. After cooling to r.t., the mixture was slowly poured into ice water (1000 mL). The mixture was extracted with DCM (3×), dried over Na₂SO₄, filtered and concentrated. The crude product was further purified by FCC (0-50% EtOAc/hexanes) to afford the sub-title compound (4.5 g, 14% yield). LCMS calc. for C₂₂H₁₅Cl₃FN₂O₂ (M+H)⁺: m/z=463.0; found 463.0.

### Step 7. Ethyl (Rₐ)-4-chloro-6-(2-cyanoethyl)-7-(2,3-dichlorophenyl)-8-fluoro-2-methylquinoline-3-carboxylate

A mixture of copper(II) acetate monohydrate (0.19 g, 0.97 mmol) and Xantphos (0.56 g, 0.97 mmol) was stirred in toluene (1 mL) and tert-butanol (9 mL) at 60° C. for 0.5 h to afford a homogeneous solution. In a separate vial, to a mixture of ethyl (E)-4-chloro-6-(2-cyanovinyl)-7-(2,3-dichlorophenyl)-8-fluoro-2-methylquinoline-3-carboxylate (4.5 g, 9.70 mmol) and polymethylhydrosiloxane (3.5 g, 58.2 mmol) in toluene (12 mL) at 60° C. was added the previous copper-containing solution. The mixture was stirred at 60° C. for 0.5 h. The mixture was then filtered through diatomaceous earth and concentrated. The crude product was purified by FCC (0-40% EtOAc/DCM) to afford a mixture of two atropisomers (2.0 g, 44% yield). The title compound was separated from its atropisomer (ethyl (Sₐ)-4-chloro-6-(2-cyanoethyl)-7-(2,3-dichlorophenyl)-8-fluoro-2-methylquinoline-3-carboxylate) using chiral supercritical fluid chromatography (ChiralPak IJ column, eluting with 40% MeOH in CO₂ at a flow rate of 70 mL/min.; the title compound eluted after its atropisomer). LCMS calc. for C₂₂H₁₇Cl₃FN₂O₂ (M+H)⁺: m/z=465.0; found 465.0.

### Intermediate 2. tert-Butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

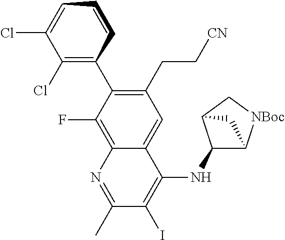

### Step 1. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

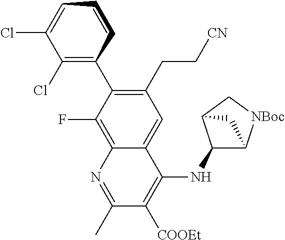

To a solution of ethyl (Rₐ)-4-chloro-6-(2-cyanoethyl)-7-(2,3-dichlorophenyl)-8-fluoro-2-methylquinoline-3-carboxylate (intermediate 1, 7.2 g, 15.5 mmol) in N-methyl-2-pyrrolidone (21 mL) was added tert-butyl (1R,4R,5S)-5-amino-2-azabicyclo[2.1.1]hexane-2-carboxylate (5.52 g, 27.8 mmol) and DIPEA (8.1 mL, 46.4 mmol). The resulting mixture was heated at 80° C. for 18 h. The mixture was allowed to cool to r.t. and poured into 1 M HCl (300 mL) and ice mixture. After stirring for 0.5 h, the solids were collected by filtration, washed with water followed by hexanes and dried under reduced pressure to afford the sub-title compound as a colorless solid (8.2 g, 85% yield). The crude product was used in next step without further purification. LCMS calc. for C₃₂H₃₄Cl₂FN₄O₄ (M+H)⁺: m/z=627.2; found 627.1.

### Step 2. (Rₐ)-4-(((1R,4R,5S)-2-(tert-Butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

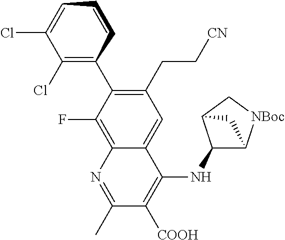

To a solution of tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (4.0 g, 6.37 mmol) in MeCN (13 mL) was added 1 M NaOH (16 mL, 15.94 mmol). The mixture was heated at 50° C. for 2 h. The mixture was then allowed to cool to r.t. and acidified to pH 5 using 1 M HCl. The organic volatiles were removed under reduced pressure. The aqueous phase was extracted with EtOAc (3×). The combined organic layers were dried over Na₂SO₄, filtered and concentrated under reduced pressure to give a yellow solid (3.70 g, 97% yield). The crude material was used in the next step without further purification. LCMS calc. for C₃₀H₃₀Cl₂FN₄O₄ (M+H)⁺: m/z=599.2; found 599.1.

### Step 3. tert-Butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

To a solution of (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (3.70 g, 6.17 mmol) in MeCN (6.2 mL) were added potassium phosphate (2.62 g, 12.34 mmol) and N-iodosuccinimide (2.50 g, 11.1 mmol). The mixture was stirred at r.t. for 1 h. The mixture was then poured into sat. aq. Na₂S₂O₃. After stirring for 10 min., the mixture was extracted with EtOAc (3×). The combined organic layers were dried over Na₂SO₄, filtered and concentrated under reduced pressure. The crude product was further purified by FCC (0-100% EtOAc/hexanes) to afford the title compound as an off-white solid (1.95 g, 46% yield). LCMS calc. for C₂₉H₂₉Cl₂FN₄O₂ (M+H)⁺: m/z=681.1; found 681.0.

The alternative atropisomer tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate can be prepared by an analogous route starting from ethyl (Sₐ)-4-chloro-6-(2-cyanoethyl)-7-(2,3-dichlorophenyl)-8-fluoro-2-methylquinoline-3-carboxylate (the alternative atropisomer from the synthesis of Intermediate 1) instead of ethyl (Rₐ)-4-chloro-6-(2-cyanoethyl)-7-(2,3-dichlorophenyl)-8-fluoro-2-methylquinoline-3-carboxylate.

### Intermediate 3. Methyl (1R,3R,4S)-2-((S)-1-phenylethyl)-2-azabicyclo[2.2.1]hept-5-ene-3-carboxylate

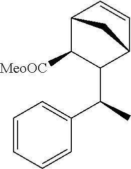

### Step 1. Methyl 2-hydroxy-2-methoxyacetate

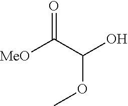

A solution of glyoxylic acid monohydrate (41.4 g, 450 mmol) in anhydrous MeOH (200 mL) was heated at 70° C. overnight. After cooling to r.t., the mixture was stirred with solid NaHCO₃ for 10 min. The resulting mixture was filtered and concentrated under reduced pressure to afford an oily residue. The residue was dissolved in DCM, dried over Na₂SO₄, filtered and concentrated to afford the product (40.0 g, 82% yield). The product was used in next step without further purification.

### Step 2. Methyl (S,E)-2-((1-phenylethyl)imino)acetate

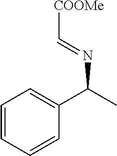

To a solution of methyl 2-hydroxy-2-methoxyacetate (40.0 g, 333 mmol) in toluene (95 mL), (S)-1-phenylethan-1-amine (40.4 g, 333 mmol) was added slowly. The mixture was stirred for 1 h at r.t. and diluted with EtOAc. The organic layer was washed with brine, dried over Na₂SO₄, filtered and concentrated under reduced pressure to a yellow oil. The crude product was used in the next step without further purification.

### Step 3. Methyl (1R,3R,4S)-2-((S)-1-phenylethyl)-2-azabicyclo[2.2.1]hept-5-ene-3-carboxylate

To a solution of methyl (S,E)-2-((1-phenylethyl)imino)acetate (63.7 g, 333 mmol) in 2,2,2-trifluoroethanol (800 mL) at −10° C. was added TFA (25.5 mL, 333 mmol). The mixture was stirred at −10° C. for 1 h before cyclopentadiene (24.2 g, 366 mmol) was added slowly. The mixture was stirred at −10° C. for another 0.5 h and then allowed to warm to r.t. After removal of volatiles, the residue was diluted with 2 M HCl (500 mL) and washed with diethyl ether. The organic layer was extracted with 2 M HCl (100 mL). The combined aqueous layer was neutralized with 28% ammonium hydroxide and extracted with EtOAc (3×). The combined organic layers was dried over Na₂SO₄, filtered and concentrated. The crude product was purified by FCC (0-10% EtOAc/hexanes) in batches to afford a colorless solid. LCMS calc. for C₁₆H₂₀NO₂ (M+H)⁺: m/z=258.1; found 258.2. ¹H NMR (500 MHz, CDCl₃) δ 7.32-7.27 (m, 2H), 7.25 (m, 2H), 7.22-7.16 (m, 1H), 6.44 (ddd, J=5.7, 3.1, 1.2 Hz, 1H), 6.29 (dd, J=5.7, 2.0 Hz, 1H), 4.33 (h, J=1.5 Hz, 1H), 3.37 (s, 3H), 3.06 (q, J=6.5 Hz, 1H), 2.93 (dq, J=3.3, 1.6 Hz, 1H), 2.24 (d, J=0.9 Hz, 1H), 2.13 (dt, J=8.4, 1.7 Hz, 1H), 1.48-1.41 (m, 4H).

### Intermediate 4. 2-(tert-Butyl) 3-methyl (1R,3R,4R,5R,6S)-5,6-dihydroxy-2-azabicyclo[2.2.1]heptane-2,3-dicarboxylate

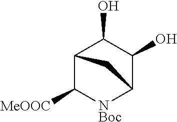 [SMILES: COC(=O)[C@H]1[C@H]2C[C@H]([C@H](O)[C@@H]2O)N1C(=O)OC(C)(C)C]

### Step 1. Methyl (1R,3R,4R,5R,6S)-5,6-dihydroxy-2-((S)-1-phenylethyl)-2-azabicyclo[2.2.1]heptane-3-carboxylate

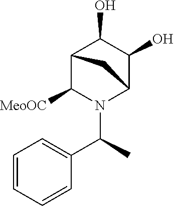

To a solution of methyl (1R,3R,4S)-2-((S)-1-phenylethyl)-2-azabicyclo[2.2.1]hept-5-ene-3-carboxylate (intermediate 3, 8.00 g, 31.1 mmol) in THF/water/tert-butanol (1:0.2:0.15, 62 mL) were added N-methylmorpholine N-oxide (10.9 g, 93.0 mmol) and potassium osmate dihydrate (0.23 g, 0.62 mmol). The mixture was stirred at r.t. for 1 h. Sat. aq. Na₂S₂O₃ was then added to the mixture. After stirring for 10 min., the mixture was extracted with EtOAc (3×). The combined organic layers were dried over Na₂SO₄, filtered and concentrated under reduced pressure. The crude product was further purified by FCC (60-100% EtOAc/hexanes) to afford the sub-title compound as a white solid (7.60 g, 84% yield). LCMS calc. for C₁₆H₂₂NO₄ (M+H)⁺: m/z=292.2; found 292.1.

### Step 2. Methyl (1R,3R,4R,5R,68)-5,6-dihydroxy-2-azabicyclo[2.2.1]heptane-3-carboxylate

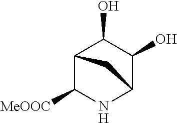 [SMILES: COC(=O)[C@@H]1N[C@@H]2C[C@H]1[C@@H](O)[C@H]2O]

To a solution of methyl (1R,3R,4R,5R,6S)-5,6-dihydroxy-2-((S)-1-phenylethyl)-2-azabicyclo[2.2.1]heptane-3-carboxylate (7.60 g, 26.1 mmol) in EtOH (120 mL) was added 20% Pd(OH)₂/C (3.66 g). The mixture was stirred under H₂ atmosphere for 16 h. The resulting mixture was filtered through diatomaceous earth and concentrated to afford the sub-title compound. The crude material was used for next step without further purification. LCMS calc. for C₈H₁₄NO₄ (M+H)⁺: m/z=188.1; found 188.1.

### Step 3. 2-(tert-Butyl) 3-methyl (1R,3R,4R,5R,6S)-5,6-dihydroxy-2-azabicyclo[2.2.1]heptane-2,3-dicarboxylate

To methyl (1R,3R,4R,5R,6S)-5,6-dihydroxy-2-azabicyclo[2.2.1]heptane-3-carboxylate (4.88 g, 26.1 mmol) dissolved in THF (50 mL) were added DIPEA (13.7 mL, 78.0 mmol) and Boc₂O (11.4 g, 52.2 mmol). The mixture was stirred at r.t. for 0.5 h and diluted with EtOAc. After washing with brine, the organic fraction was dried over Na₂SO₄, filtered and concentrated. The crude product was further purified by FCC (40-100% EtOAc/hexanes) to afford the title compound as a white solid (6.20 g, 83% yield over 2 steps). LCMS calc. for C₉H₁₄NO₆ (M-^{t}Bu+2H)⁺: m/z=232.1; found 232.1.

### Intermediate 5. tert-Butyl (1R,3R,4R,5R,6S)-5,6-bis((tert-butyldiphenylsilyl)oxy)-3-ethynyl-2-azabicyclo[2.2.1]heptane-2-carboxylate

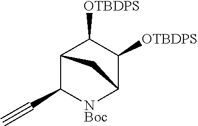

### Step 1. 2-(tert-Butyl) 3-methyl (1R,3R,4R,5R,6S)-5,6-bis((tert-butyldiphenylsilyl)oxy)-2-azabicyclo[2.2.1]heptane-2,3-dicarboxylate

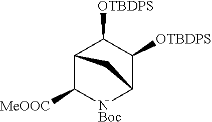

To a solution of 2-(tert-butyl) 3-methyl (1R,3R,4R,5R,6S)-5,6-dihydroxy-2-azabicyclo[2.2.1]heptane-2,3-dicarboxylate (3.83 g, 13.3 mmol) in DMF (27 mL) was added imidazole (5.44 g, 80.0 mmol) and TBDPSCI (7.5 mL, 29.3 mmol). The mixture was stirred at r.t. for 5 days. The mixture was then diluted with EtOAc. After washing with brine 5 times, the organic fraction was dried over Na₂SO₄, filtered and concentrated. The crude product was further purified by FCC (0-20% EtOAc/hexanes) to afford the sub-title compound as a colorless oil (10.0 g, 98% yield). LCMS calc. for C₂₅H₃₂NO₆Si (M-TBDPS-^{t}Bu+3H)⁺: m/z=470.2; found 470.2.

### Step 2. tert-Butyl (1R,3R,4R,5R,6S)-5,6-bis((tert-butyldiphenylsilyl)oxy)-3-(hydroxymethyl)-2-azabicyclo[2.2.1]heptane-2-carboxylate

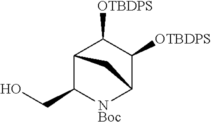

To a solution of 2-(tert-butyl) 3-methyl (1R,3R,4R,5R,6S)-5,6-bis((tert-butyldiphenylsilyl)oxy)-2-azabicyclo[2.2.1]heptane-2,3-dicarboxylate (10.0 g, 13.1 mmol) in THF (65 mL) was added LiBH₄ (2 M THF, 16.4 mL, 32.7 mmol). The mixture was stirred at r.t. for 16 h and then quenched by slow addition of sat. aq. NH₄Cl at 0° C. The mixture was diluted with EtOAc, washed with brine, dried over Na₂SO₄, filtered and concentrated. The crude product was purified by FCC (0-40% EtOAc) to afford the sub-title compound (6.0 g, 62% yield). LCMS calc. for C₂₃H₃₀NO₂Si (M-TBDPS-Boc-H₂O+3H)⁺: m/z=380.2; found 380.1.

### Step 3. tert-Butyl (1R,3R,4R,5R,6S)-5,6-bis((tert-butyldiphenylsilyl)oxy)-3-formyl-2-azabicyclo[2.2.1]heptane-2-carboxylate

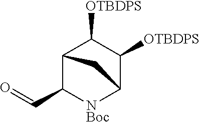

To a solution of tert-butyl (1R,3R,4R,5R,6S)-5,6-bis((tert-butyldiphenylsilyl)oxy)-3-(hydroxymethyl)-2-azabicyclo[2.2.1]heptane-2-carboxylate (6.0 g, 8.2 mmol) in DCM (41 mL) was added Dess-Martin periodinane (4.2 g, 9.8 mmol) portionwise. The resulting mixture was stirred at r.t. for 1 h. Sat. aq. Na₂S₂O₃ (10 mL) was then added to the mixture, which was stirred for an additional 10 min. The organic phase was separated, dried over Na₂SO₄, filtered and concentrated to give crude product. The crude product was used in the next step without further purification. LCMS calc. for C₂₄H₃₀NO₅Si (M-TBDPS-^{t}Bu+3H)⁺: m/z=440.2; found 440.3.

### Step 4. tert-Butyl (1R,3R,4R,5R,6S)-5,6-bis((tert-butyldiphenylsilyl)oxy)-3-ethynyl-2-azabicyclo[2.2.1]heptane-2-carboxylate

To a solution of tert-butyl (1R,3R,4R,5R,6S)-5,6-bis((tert-butyldiphenylsilyl)oxy)-3-formyl-2-azabicyclo[2.2.1]heptane-2-carboxylate (5.98 g, 8.15 mmol) in MeOH (41 mL) were added dimethyl(1-diazo-2-oxopropyl)phosphonate (2.35 g, 12.2 mmol) and K₂CO₃ (3.38 g, 24.5 mmol). After stirring for 16 h, the mixture was filtered through diatomaceous earth. The filtrate was concentrated. The residue was extracted with EtOAc, filtered through diatomaceous earth and concentrated. The crude product was purified by FCC (0-30% EtOAc/hexanes) to afford the title compound as a colorless oil (4.4 g, 74% yield over 2 steps). LCMS calc. for C₂₅H₂₈NO₃Si (M-^{t}Bu-TBDPSOH+2H)⁺: m/z=418.2; found 418.2.

### Intermediate 6. tert-Butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate

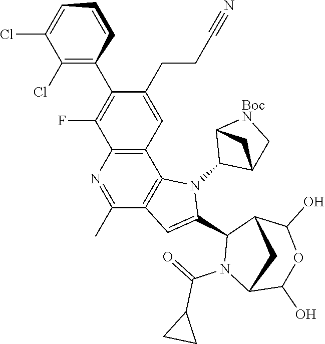

### Step 1. ((1R,3R,4R,5R,6S)-5,6-Bis((tert-butyldiphenylsilyl)oxy)-3-ethynyl-2-azabicyclo[2.2.1]heptan-2-yl)(cyclopropyl)methanone

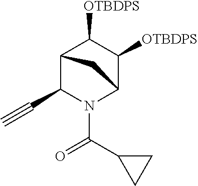

To a solution of tert-butyl (1R,3R,4R,5R,6S)-5,6-bis((tert-butyldiphenylsilyl)oxy)-3-ethynyl-2-azabicyclo[2.2.1]heptane-2-carboxylate (0.88 g, 1.20 mmol) in 1,4-dioxane (4 mL) was added 4 M HCl in 1,4-dioxane (4 mL). The mixture was stirred at r.t. for 0.5 h. The volatiles were then removed under reduced pressure. The residue was dissolved in DCM (6 mL). Upon stirring, DIPEA (10.5 mL, 6.00 mmol) was added followed by cyclopropanecarbonyl chloride (0.19 g, 1.80 mmol). The mixture was stirred for 0.5 h. The mixture was then diluted with DCM, washed with sat. aq. NaHCO₃, dried over Na₂SO₄, filtered and concentrated. The crude material was used for next step without further purification. LCMS calc. for C₂₈H₃₄NO₃Si (M-TBDPS+2H)⁺: m/z=460.2; found 460.3.

### Step 2. tert-Butyl (1R,4R,5S)-5-((Rₐ)-8-(2-cyanoethyl)-2-((1R,3R,4R,5R,6S)-2-(cyclopropanecarbonyl)-5,6-dihydroxy-2-azabicyclo[2.2.1]heptan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

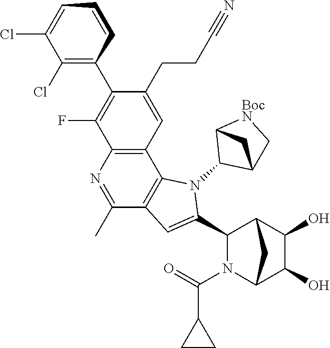

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 0.69 g, 1.01 mmol), ((1R,3R,4R,5R,6S)-5,6-bis((tert-butyldiphenylsilyl)oxy)-3-ethynyl-2-azabicyclo[2.2.1]heptan-2-yl)(cyclopropyl)methanone (0.84 g, 1.21 mmol), CuI (0.077 g, 0.40 mmol), Pd(PPh₃)₄ (0.23 g, 0.20 mmol), CsF (0.76 g, 5.03 mmol) and DIPEA (1.8 mL, 10.1 mmol) in DMF (5.0 mL) was sparged with N₂ and heated at 70° C. for 0.5 h. Then, Cs₂CO₃ (0.98 g, 3.02 mmol) was added to the mixture. The resulting slurry was stirred at 90° C. for another 18 h. The mixture was then allowed to cool to r.t. and poured into water. The solution was extracted with EtOAc (2×). Then the combined organic layers were washed with brine (5×), dried over Na₂SO₄, filtered and concentrated. The crude product was purified by FCC (0-100% EtOAc/hexanes) to afford the sub-title compound as a solid (0.22 g, 28% yield). LCMS calc. for C₄₁H₄₃Cl₂FN₅O₅ (M+H)⁺: m/z=774.3; found 774.2.

### Step 3. tert-Butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate

To a solution of tert-butyl (1R,4R,5S)-5-((Rₐ)-8-(2-cyanoethyl)-2-((1R,3R,4R,5R,6S)-2-(cyclopropanecarbonyl)-5,6-dihydroxy-2-azabicyclo[2.2.1]heptan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (0.22 g, 0.28 mmol) in 1,4-dioxane/water (4:1, 5.7 mL) were added 2,6-lutidine (0.061 g, 0.57 mmol) and NaIO₄ (0.24 g, 1.14 mmol). The mixture was stirred at r.t. for 1 h. The mixture was then diluted with EtOAc and washed with brine. The organic layer was dried over Na₂SO₄, filtered and concentrated under reduced pressure. The crude product was dissolved in DCM to make a stock solution and used directly for further reactions. LCMS calc. for C₄₁H₄₃Cl₂FN₅O₆ (M+H)⁺: m/z=790.3; found 790.2.

The alternative atropisomer tert-butyl (1R,4R,5S)-5-((7Sₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate can be prepared by an analogous route starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Intermediate 7. 2-(Trimethylsilyl)ethyl (1S,5R,7R)-6-(cyclopropanecarbonyl)-7-ethynyl-3,6-diazabicyclo[3.2.1]octane-3-carboxylate

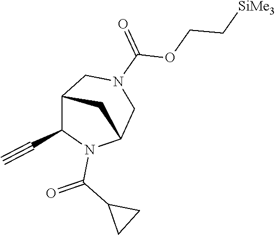 [SMILES: C#C[C@H]1[C@H]2C[C@H](CN(C(=O)OCC[Si](C)(C)C)C2)N1C(=O)C1CC1]

### Step 1. 6-(tert-Butyl) 7-methyl (1R,5R,7R)-2,4-dihydroxy-3-oxa-6-azabicyclo[3.2.1]octane-6,7-dicarboxylate

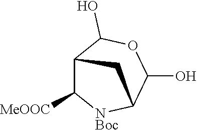 [SMILES: COC(=O)[C@H]1[C@H]2C[C@H](C(O)OC2O)N1C(=O)OC(C)(C)C]

To a solution of 2-(tert-butyl) 3-methyl (1R,3R,4R,5R,6S)-5,6-dihydroxy-2-azabicyclo[2.2.1]heptane-2,3-dicarboxylate (intermediate 4, 1.50 g, 5.22 mmol) in 1,4-dioxane/water (4:1, 26 mL) were added 2,6-lutidine (2.24 g, 20.9 mmol) and NaIO₄ (2.23 g, 10.4 mmol). The mixture was stirred at r.t. for 1 h. The mixture was then diluted with EtOAc and washed with brine. The organic layer was dried over Na₂SO₄, filtered and concentrated under reduced pressure. The crude product was used in the next step without further purification. LCMS calc. for C₉H₁₄NO₇ (M-^{t}Bu+2H)⁺: m/z=248.1; found 248.1.

### Step 2. 6-(tert-Butyl) 7-methyl (1S,5R,7R)-3-benzyl-3,6-diazabicyclo[3.2.1]octane-6,7-dicarboxylate

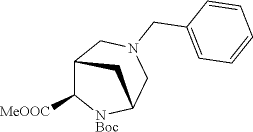 [SMILES: COC(=O)[C@H]1[C@H]2C[C@H](CN(Cc3ccccc3)C2)N1C(=O)OC(C)(C)C]

To a solution of 6-(tert-butyl) 7-methyl (1R,5R,7R)-2,4-dihydroxy-3-oxa-6-azabicyclo[3.2.1]octane-6,7-dicarboxylate (1.58 g, 5.22 mmol) in DCM (26 mL) was added benzylamine (0.59 g, 5.48 mmol) and PPTS (2.62 g, 10.4 mmol). The mixture was stirred for 1 h at r.t. before NaBH(OAc)₃ (11.1 g, 52.2 mmol) was added. After stirring for 0.5 h, the reaction was quenched by slow addition of water. The mixture was extracted with DCM (2×). The combined organic layers were dried over Na₂SO₄, filtered and concentrated. The crude product was further purified by FCC (0-40% EtOAc/hexanes) to afford the sub-title compound as a colorless oil in near quantitative yield over 2 steps. LCMS calc. for C₂₀H₂₉N₂O₄ (M+H)⁺: m/z=361.2; found 361.2.

### Step 3. Methyl (1S,5R,7R)-3-benzyl-6-(cyclopropanecarbonyl)-3,6-diazabicyclo[3.2.1]octane-7-carboxylate

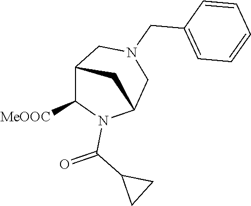 [SMILES: COC(=O)[C@H]1[C@H]2C[C@H](CN(Cc3ccccc3)C2)N1C(=O)C1CC1]

To a solution of 6-(tert-butyl) 7-methyl (1 S,5R,7R)-3-benzyl-3,6-diazabicyclo[3.2.1]octane-6,7-dicarboxylate (1.88 g, 5.22 mmol) in 1,4-dioxane (10 mL) was added 4 M HCl in 1,4-dioxane (10 mL). The mixture was stirred at r.t. for 1 h. The volatiles were then removed under reduced pressure. The residue was dissolved in DCM (21 mL). Upon stirring, DIPEA (4.6 mL, 26.1 mmol) was added followed by cyclopropanecarbonyl chloride (0.82 g, 7.83 mmol). The mixture was stirred for 0.5 h. The mixture was then diluted with DCM, washed with water, dried over Na₂SO₄, filtered and concentrated. The crude product was further purified by FCC (0-60% EtOAc/hexanes) to afford the sub-title compound as a colorless oil (1.27 g, 74% yield). LCMS calc. for C₁₉H₂₅N₂O₃ (M+H)⁺: m/z=329.2; found 329.2.

### Step 4. 7-Methyl 3-(2-(trimethylsilyl)ethyl) (1S,5R,7R)-6-(cyclopropanecarbonyl)-3,6-diazabicyclo[3.2.1]octane-3,7-dicarboxylate

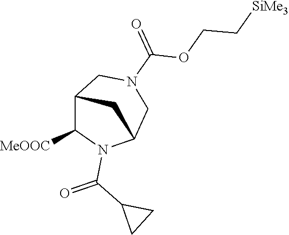 [SMILES: COC(=O)[C@H]1[C@H]2C[C@H](CN(C(=O)OCC[Si](C)(C)C)C2)N1C(=O)C1CC1]

To a solution of methyl (1 S,5R,7R)-3-benzyl-6-(cyclopropanecarbonyl)-3,6-diazabicyclo[3.2.1]octane-7-carboxylate (1.27 g, 3.87 mmol) in EtOH (35 mL) was added 20% Pd(OH)₂/C (0.54 g). The mixture was stirred under H₂ atmosphere for 16 h. The resulting mixture was filtered through diatomaceous earth and concentrated.

The residue was dissolved in THF (12 mL). To the mixture were added DIPEA (2.0 mL, 11.6 mmol) and 1-[2-(trimethylsilyl)ethoxycarbonyloxy]pyrrolidin-2,5-dione (1.25 g, 4.83 mmol). The mixture was stirred for 1 h at r.t. The mixture was then diluted with EtOAc. The organic fraction was washed with brine, dried over Na₂SO₄, filtered and concentrated. The crude product was further purified by FCC (0-60% EtOAc/hexanes) to afford the sub-title compound as a colorless oil (1.19 g, 80% yield). LCMS calc. for C₁₆H₂₇N₂O₅Si (M−2Me+3H)⁺: m/z=355.2; found 355.1.

### Step 5. 2-(Trimethylsilyl)ethyl (1S,5R,7R)-6-(cyclopropanecarbonyl)-7-(hydroxymethyl)-3,6-diazabicyclo[3.2.1]octane-3-carboxylate

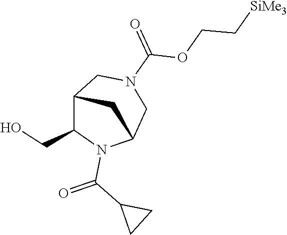 [SMILES: C[Si](C)(C)CCOC(=O)N1C[C@@H]2C[C@H](C1)N(C(=O)C1CC1)[C@H]2CO]

To a solution of 7-methyl 3-(2-(trimethylsilyl)ethyl) (1 S,5R,7R)-6-(cyclopropanecarbonyl)-3,6-diazabicyclo[3.2.1]octane-3,7-dicarboxylate (1.19 g, 3.11 mmol) in THF (15.6 mL) was added LiBH₄ (2 M in THF, 3.9 mL, 7.78 mmol). The mixture was stirred at r.t. for 5 h and then quenched by slow addition of sat. aq. NH₄Cl at 0° C. The mixture was diluted with EtOAc, washed with brine, dried over Na₂SO₄, filtered and concentrated. The crude product was purified by FCC (0-60% EtOAc) to afford the sub-title compound (1.04 g, 94% yield). LCMS calc. for C₁₅H₂₇N₂O₄Si (M−2Me+3H)⁺: m/z=327.2; found 327.2.

### Step 6. 2-(Trimethylsilyl)ethyl (1S,5R,7R)-6-(cyclopropanecarbonyl)-7-formyl-3,6-diazabicyclo[3.2.1]octane-3-carboxylate

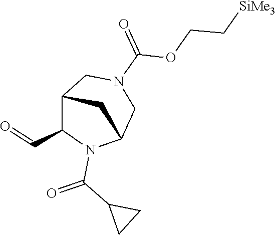 [SMILES: C[Si](C)(C)CCOC(=O)N1C[C@@H]2C[C@H](C1)N(C(=O)C1CC1)[C@H]2C=O]

To a solution of 2-(trimethylsilyl)ethyl (1 S,5R,7R)-6-(cyclopropanecarbonyl)-7-(hydroxymethyl)-3,6-diazabicyclo[3.2.1]octane-3-carboxylate (1.04 g, 2.93 mmol) in DCM (15 mL) was added Dess-Martin periodinane (1.87 g, 4.40 mmol) portionwise. The resulting mixture was stirred at r.t. for 1 h. Sat. aq. Na₂S₂O₃ (5 mL) was added to the mixture, which was then stirred for an additional 10 min. The organic phase was separated, dried over Na₂SO₄, filtered and concentrated to give crude product. The crude product was used in the next step without further purification. LCMS calc. for C₁₅H₂₅N₂O₄Si (M−2Me+3H)⁺: m/z=325.2; found 325.1.

### Step 7. 2-(Trimethylsilyl)ethyl (1S,5R,7R)-6-(cyclopropanecarbonyl)-7-ethynyl-3,6-diazabicyclo[3.2.1]octane-3-carboxylate

To a solution of 2-(trimethylsilyl)ethyl (1 S,5R,7R)-6-(cyclopropanecarbonyl)-7-formyl-3,6-diazabicyclo[3.2.1]octane-3-carboxylate (1.03 g, 2.93 mmol) in MeOH (15 mL) were added dimethyl(1-diazo-2-oxopropyl)phosphonate (0.84 g, 4.40 mmol) and K₂CO₃ (1.22 g, 8.79 mmol). After stirring for 16 h, the mixture was filtered through diatomaceous earth. The filtrate was concentrated. The residue was diluted with water, extracted with EtOAc, filtered through diatomaceous earth and concentrated. The crude product was purified by FCC (0-60% EtOAc/hexanes) to afford the title compound (0.546 g, 54% yield over 2 steps). LCMS calc. for C₁₆H₂₅N₂O₃Si (M−2Me+3H)⁺: m/z=321.2; found 321.1.

### Intermediate 8. tert-Butyl (1R,4R,5S)-5-((Rₐ)-8-(2-cyanoethyl)-2-((1S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate

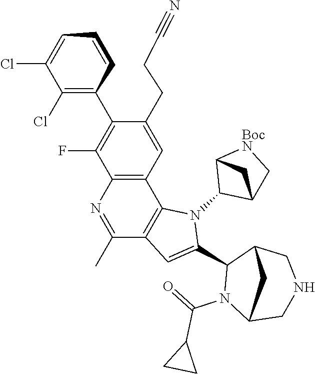

### Step 1. tert-Butyl (1R,4R,5S)-5-((Rₐ)-8-(2-cyanoethyl)-2-((1S,5R,7R)-6-(cyclopropanecarbonyl)-3-((2-(trimethylsilyl)ethoxy)carbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate

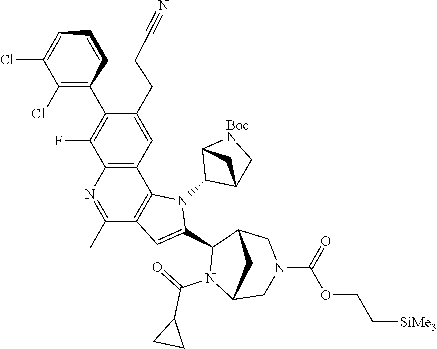

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 0.90 g, 1.32 mmol), 2-(trimethylsilyl)ethyl (1 S,5R,7R)-6-(cyclopropanecarbonyl)-7-ethynyl-3,6-diazabicyclo[3.2.1]octane-3-carboxylate (intermediate 7, 0.55 g, 1.59 mmol), CuI (0.10 g, 0.53 mmol), Pd(PPh₃)₄ (0.31 g, 0.26 mmol) and DIPEA (2.3 mL, 13.2 mmol) in DMF (6.6 mL) was sparged with N₂ and heated at 70° C. for 1 h. Then, Cs₂CO₃ (1.29 g, 3.96 mmol) was added to the mixture. The resulting slurry was stirred at 90° C. for another 18 h. The mixture was then allowed to cool to r.t. and poured into water. The solution was extracted with EtOAc (2×). Then the combined organic layers were washed with brine (5×), dried over Na₂SO₄, filtered and concentrated. The crude product was purified by FCC (0-100% EtOAc/hexanes) to afford the sub-title compound (0.89 g, 75% yield). LCMS calc. for C₄₇H₅₆Cl₂FN₆O₅Si (M+H)⁺: m/z=901.3; found 901.2.

### Step 2. tert-Butyl (1R,4R,5S)-5-((Rₐ)-8-(2-cyanoethyl)-2-((1S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate

To a solution of tert-butyl (1R,4R,5S)-5-((Rₐ)-8-(2-cyanoethyl)-2-((1 S,5R,7R)-6-(cyclopropanecarbonyl)-3-((2-(trimethylsilyl)ethoxy)carbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (0.30 g, 0.33 mmol) in THF (3.3 mL) was added tris(dimethylamino)sulfonium difluorotrimethylsilicate (0.14 g, 0.50 mmol) in one portion. The mixture was heated at 60° C. for 0.5 h. The mixture was then allowed to cool to r.t. and diluted with EtOAc. The organic fraction was washed with brine, dried over Na₂SO₄, filtered and concentrated. The crude solid was dried under reduced pressure and used directly for further reactions. LCMS calc. for C₄₁H₄₄Cl₂FN₆O₃ (M+H)⁺: m/z=757.3; found 757.2.

The alternative atropisomer tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate can be prepared by an analogous route starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Intermediate 9. Cyclopropyl((1R,5R,7R)-7-ethynyl-3-oxa-6-azabicyclo[3.2.1]octan-6-yl)methanone

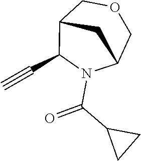

### Step 1. Methyl (1R,5R,7R)-2,4-dihydroxy-6-((S)-1-phenylethyl)-3-oxa-6-azabicyclo[3.2.1]octane-7-carboxylate

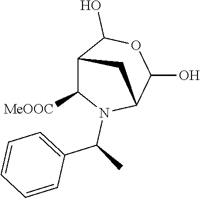 [SMILES: COC(=O)[C@H]1[C@H]2C[C@H](C(O)OC2O)N1[C@@H](C)c1ccccc1]

To a solution of methyl (1R,3R,4S)-2-((S)-1-phenylethyl)-2-azabicyclo[2.2.1]hept-5-ene-3-carboxylate (intermediate 3, 2.00 g, 7.77 mmol) in 1,4-dioxane/water (3:1, 39 mL) were added 2,6-lutidine (1.67 g, 15.5 mmol), sodium periodate (6.65 g, 31.1 mmol) followed by potassium osmate dihydrate (0.086 g, 0.23 mmol). The mixture was stirred at r.t. for 2 h. The mixture was then diluted with EtOAc and washed with brine. The organic layer was dried over Na₂SO₄, filtered and concentrated under reduced pressure. The crude product was used in the next step without further purification. LCMS calc. for C₁₆H₂₂NO₅ (M+H)⁺: m/z=308.1; found 308.1.

### Step 2. Methyl (2R,3R,5R)-3,5-bis(hydroxymethyl)-1-((S)-1-phenylethyl)pyrrolidine-2-carboxylate

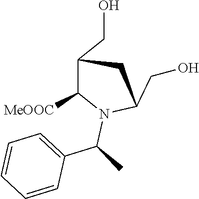 [SMILES: COC(=O)[C@H]1[C@H](CO)C[C@H](CO)N1[C@@H](C)c1ccccc1]

To a solution of methyl (1R,5R,7R)-2,4-dihydroxy-6-((S)-1-phenylethyl)-3-oxa-6-azabicyclo[3.2.1]octane-7-carboxylate (2.39 g, 7.77 mmol) in MeOH (39 mL) cooled at 0° C. was added NABH₄ (1.76 g, 46.6 mmol) portionwise. The mixture was stirred at 0° C. for 1 h. The reaction was quenched by slow addition of sat. aq. NH₄Cl at 0° C. The mixture was diluted with EtOAc and washed with brine. The aqueous layer was extracted by EtOAc (2×). The combined organic layers were dried over Na₂SO₄, filtered and concentrated under reduced pressure. The crude product was further purified by FCC (0-100% EtOAc/hexanes) to afford the sub-title compound (1.12 g, 49% yield over 2 steps). LCMS calc. for C₁₆H₂₄NO₄ (M+H)⁺: m/z=294.2; found 294.2.

### Step 3. Methyl (2R,3R,5R)-3-(((tert-butyldiphenylsilyl)oxy)methyl)-5-(hydroxymethyl)-1-((S)-1-phenylethyl)pyrrolidine-2-carboxylate and methyl (2R,3R,5R)-5-(((tert-butyldiphenylsilyl)oxy)methyl)-3-(hydroxymethyl)-1-((S)-1-phenylethyl)pyrrolidine-2-carboxylate

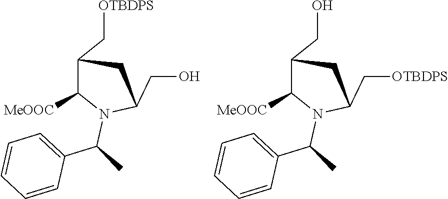

To a solution of methyl (2R,3R,5R)-3,5-bis(hydroxymethyl)-1-((S)-1-phenylethyl)pyrrolidine-2-carboxylate (1.12 g, 3.83 mmol) in DMF (19 mL) was added imidazole (0.78 g, 11.5 mmol) followed by dropwise addition of TBDPSCI (1.0 mL, 4.02 mmol). After stirring for 1 h, the mixture was diluted with EtOAc. After washing with brine 5 times, the organic fraction was dried over Na₂SO₄, filtered and concentrated. The crude product was further purified by FCC (0-100% EtOAc/hexanes) to afford a mixture of two regioisomers (0.83 g, 41% yield). LCMS calc. for C₃₂H₄₂NO₄Si (M+H)⁺: m/z=532.3; found 532.3.

### Step 4. Methyl (1R,5R,7R)-6-((S)-1-phenylethyl)-3-oxa-6-azabicyclo[3.2.1]octane-7-carboxylate

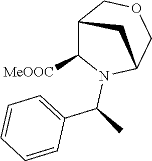 [SMILES: COC(=O)[C@H]1[C@@H]2COC[C@@H](C2)N1[C@@H](C)c1ccccc1]

To a solution of methyl (2R,3R,5R)-3-(((tert-butyldiphenylsilyl)oxy)methyl)-5-(hydroxymethyl)-1-((S)-1-phenylethyl)pyrrolidine-2-carboxylate and methyl (2R,3R,5R)-5-(((tert-butyldiphenylsilyl)oxy)methyl)-3-(hydroxymethyl)-1-((S)-1-phenylethyl)pyrrolidine-2-carboxylate (a mixture of two isomers, 0.83 g, 1.56 mmol) in DCM (7.8 mL) cooled at 0° C. was added DIPEA (0.82 mL, 4.68 mmol) followed by dropwise addition of methanesulfonyl chloride (0.22 g, 1.87 mmol) in DCM (1.2 mL). After stirring at 0° C. for 0.5 h, the mixture was allowed to warm to r.t. and then quenched by sat. aq. NaHCO₃. The mixture was extracted with DCM (2×). The organic layers were combined, dried over Na₂SO₄, filtered and concentrated.

To the residue dissolved in THF (20 mL) was added TBAF (1 M in THF, 4.7 mL, 4.7 mmol). The mixture was stirred at r.t. for 1 h. The mixture was then diluted with EtOAc. The organic layer was washed with brine, dried over Na₂SO₄, filtered and concentrated. The crude product was further purified by FCC (0-40% EtOAc/hexanes) to afford the sub-title compound (0.25 g, 58% yield). LCMS calc. for C₁₆H₂₂NO₃ (M+H)⁺: m/z=276.2; found 276.1.

### Step 5. Methyl (1R,5R,7R)-6-(cyclopropanecarbonyl)-3-oxa-6-azabicyclo[3.2.1]octane-7-carboxylate

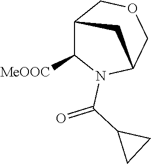 [SMILES: COC(=O)[C@H]1[C@@H]2COC[C@@H](C2)N1C(=O)C1CC1]

To a solution of methyl (1R,5R,7R)-6-((S)-1-phenylethyl)-3-oxa-6-azabicyclo[3.2.1]octane-7-carboxylate (0.25 g, 0.91 mmol) in EtOH (20 mL) was added 20% Pd(OH)₂/C (0.25 g). The mixture was stirred under H₂ atmosphere for 16 h. The resulting mixture was filtered through diatomaceous earth and concentrated.

The residue was dissolved in DCM (4 mL). To the mixture were added DIPEA (0.48 mL, 2.72 mmol) and cyclopropanecarbonyl chloride (0.14 g, 1.36 mmol). The mixture was stirred for 0.5 h and quenched with sat. aq. NaHCO₃. The resulting mixture was extracted with DCM. The organic layers were combined, dried over Na₂SO₄, filtered and concentrated. The crude product was further purified by FCC (0-100% EtOAc/hexanes) to afford the sub-title compound (0.12 g, 56% yield). LCMS calc. for C₁₂H1NO₄ (M+H)⁺: m/z=240.1; found 240.1.

### Step 6. Cyclopropyl((1R,5R,7R)-7-(hydroxymethyl)-3-oxa-6-azabicyclo[3.2.1]octan-6-yl)methanone

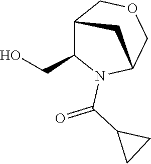 [SMILES: O=C(C1CC1)N1[C@H]2COC[C@H](C2)[C@@H]1CO]

To a solution of methyl (1R,5R,7R)-6-(cyclopropanecarbonyl)-3-oxa-6-azabicyclo[3.2.1]octane-7-carboxylate (0.12 g, 0.51 mmol) in THF (2.5 mL) was added LiBH₄ (2 M in THF, 0.6 mL, 1.28 mmol). The mixture was stirred at r.t. for 16 h and then quenched by slow addition of sat. aq. NH₄Cl at 0° C. The mixture was diluted with EtOAc, washed with brine, dried over Na₂SO₄, filtered and concentrated. The crude product was purified by FCC (0-100% EtOAc/hexanes) to afford the sub-title compound (0.075 g, 70% yield). LCMS calc. for C₁₁H₁₈NO₃ (M+H)⁺: m/z=212.1; found 212.1.

### Step 7. (1R,5R,7R)-6-(Cyclopropanecarbonyl)-3-oxa-6-azabicyclo[3.2.1]octane-7-carbaldehyde

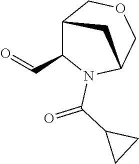 [SMILES: O=C[C@H]1[C@@H]2COC[C@@H](C2)N1C(=O)C1CC1]

To a solution of cyclopropyl((1R,5R,7R)-7-(hydroxymethyl)-3-oxa-6-azabicyclo[3.2.1]octan-6-yl)methanone (0.075 g, 0.36 mmol) in DCM (1.8 mL) was added Dess-Martin periodinane (0.23 g, 0.53 mmol) portionwise. The resulting mixture was stirred at r.t. for 1 h. Sat. aq. Na₂S₂O₃ (1 mL) was then added to the mixture, which was stirred for an additional 0.5 h. The organic phase was separated, dried over Na₂SO₄, filtered and concentrated to give crude product. The crude product was used in the next step without further purification. LCMS calc. for C₁₁H₁₆NO₃ (M+H)⁺: m/z=210.1; found 210.1.

### Step 8. Cyclopropyl((1R,5R,7R)-7-ethynyl-3-oxa-6-azabicyclo[3.2.1]octan-6-yl)methanone

To a solution of (1R,5R,7R)-6-(cyclopropanecarbonyl)-3-oxa-6-azabicyclo[3.2.1]octane-7-carbaldehyde (0.074 g, 0.36 mmol) in MeOH (1.8 mL) were added dimethyl(1-diazo-2-oxopropyl)phosphonate (0.10 g, 0.53 mmol) and K₂CO₃ (0.15 g, 1.07 mmol). After stirring for 8 h, the mixture was filtered through diatomaceous earth. The filtrate was concentrated. The residue was extracted with EtOAc, filtered through diatomaceous earth and concentrated. The crude product was purified by FCC (0-100% EtOAc/hexanes) to afford the title compound (0.024 g, 33% yield over 2 steps). LCMS calc. for C₁₂H₁₆NO₂ (M+H)⁺: m/z=206.1; found 206.1. ¹H NMR (500 MHz, CDCl₃) δ 4.71 (d, J=2.1 Hz, 1H), 4.39 (m, 1H), 3.98-3.84 (m, 2H), 3.73-3.67 (m, 1H), 3.39 (d, J=10.8 Hz, 1H), 2.54 (m, 1H), 2.44 (d, J=2.2 Hz, 1H), 2.39-2.32 (m, 1H), 2.05-1.95 (m, 1H), 1.86 (d, J=11.4 Hz, 1H), 1.15-0.96 (m, 2H), 0.90-0.80 (m, 2H).

### Intermediate 10. Cyclopropyl(7-ethynyl-4-phenyl-1,4-diazepan-1-yl)methanone

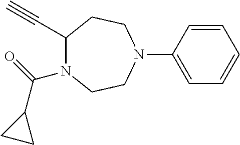 [SMILES: C#CC1CCN(c2ccccc2)CCN1C(=O)C1CC1]

### Step 1: Cyclopropyl(4-phenyl-7-((trimethylsilyl)ethynyl)-1,4-diazepan-1-yl)methanone and cyclopropyl(4-phenyl-2-((trimethylsilyl)ethynyl)-1,4-diazepan-1-yl)methanone

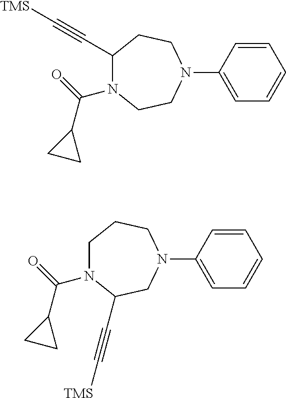 [SMILES: C[Si](C)(C)C#CC1CCN(c2ccccc2)CCN1C(=O)C1CC1 ; C[Si](C)(C)C#CC1CN(c2ccccc2)CCCN1C(=O)C1CC1]

To a mixture of 1-phenyl-1,4-diazepane (687 mg, 3.90 mmol) and Et₂O (8.0 mL), n-BuLi (1.6 mL, 3.90 mmol) was added dropwise at −78° C. The resulting mixture was stirred at −78° C. for 10 min. before 2,2,2-trifluoro-1-phenylethan-1-one (0.602 mL, 4.28 mmol) in diethyl ether (4.0 mL) was added, the resulting mixture was stirred another 10 min. before ((trimethylsilyl)ethynyl)lithium (5.8 mL, 5.8 mmol) and BF₃Et₂O (0.59 mL, 4.7 mmol) were added. The resulting mixture was warmed to r.t. and stirred for 13 h before NEt₃ (2.2 mL, 16 mmol) and cyclopropanecarbonyl chloride (1.1 mL, 12 mmol) were added. The mixture was stirred at the same temperature for 30 min. and quenched with sat. aq. NaHCO₃. The mixture was extracted with EtOAc (3×10 mL). The combined organic layers were washed with brine (30 mL), dried over anhydrous Na₂SO₄, and filtered. The volatiles were removed under vacuum, and the residue was purified by FCC (0-100% EtOAc/hexanes) to give the sub-title compound along with a regio-isomer as an orange oil (267 mg, 20% yield). LCMS calc. for C₂₀H₂₉N₂OSi (M+H)⁺: m/z=341.2; found 341.3.

### Step 2: Cyclopropyl(7-ethynyl-4-phenyl-1,4-diazepan-1-yl)methanone

To a mixture of cyclopropyl(4-phenyl-7-((trimethylsilyl)ethynyl)-1,4-diazepan-1-yl)methanone and cyclopropyl(4-phenyl-2-((trimethylsilyl)ethynyl)-1,4-diazepan-1-yl)methanone (267 mg, 0.785 mmol) were added K₂CO₃ (217 mg, 1.570 mmol) and MeOH (7.8 mL) at r.t. The resulting mixture was stirred at r.t. for 16 h before being diluted with EtOAc (20 mL) and sat. aq. NaHCO₃ (20 mL). The resulting mixture was extracted with EtOAc (3×20 mL). The combined organic layers were washed with brine (50 mL), dried over anhydrous Na₂SO₄, and filtered. The volatiles were removed under vacuum, and the residue was purified by FCC (0-100% EtOAc/hexanes) to give the title compound (eluded prior to its regio-isomer) as a pale yellow solid (65.2 mg, 31% yield). LCMS calc. for C₁₇H₂₁N₂O (M+H)⁺: m/z=269.2; found 269.3.

### Intermediate 11. 2-(Trimethylsilyl)ethyl (1R,3R,5S)-2-(cyclopropanecarbonyl)-3-ethynyl-2,6-diazabicyclo[3.2.1]octane-6-carboxylate

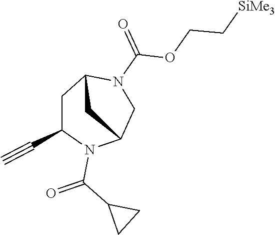 [SMILES: C#C[C@H]1C[C@H]2C[C@H](CN2C(=O)OCC[Si](C)(C)C)N1C(=O)C1CC1]

### Step 1. 1-(tert-Butyl) 2-methyl (2R,4S)-4-((tert-butyldiphenylsilyl)oxy)pyrrolidine-1,2-dicarboxylate

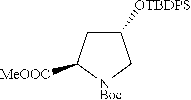

To a stirred solution of 1-(tert-butyl) 2-methyl (2R,4S)-4-hydroxypyrrolidine-1,2-dicarboxylate (39.9 g, 163 mmol) in DCM (407 mL) at 0° C. were added imidazole (18.83 g, 277 mmol) followed by TBDPSCI (44.4 mL, 171 mmol). The mixture was removed from ice bath, allowed to warm to r.t. and stirred for additional 16 h. The mixture was diluted with water (400 mL) and the organic layer was separated. The aqueous layer was further extracted with DCM (3×). The combined organic layers were washed with brine, dried over MgSO₄, and concentrated to afford the sub-title compound as a light yellow oil in near quantitative yield. The crude product was used in the next step without further purification. LCMS calc. for C₂₂H₃₀NO₃Si (M-Boc+2H)⁺: m/z=384.2; found 384.2.

### Step 2. tert-Butyl(2R,4S)-4-((tert-butyldiphenylsilyl)oxy)-2-(hydroxymethyl)pyrrolidine-1-carboxylate

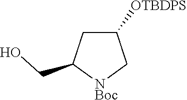

LiBH₄ (2 M in THF, 104 mL, 208 mmol) was slowly added to a stirred solution of 1-(tert-butyl) 2-methyl (2R,4S)-4-((tert-butyldiphenylsilyl)oxy)pyrrolidine-1,2-dicarboxylate (80.3 g, 166 mmol) in THF (415 mL) at 0° C. The mixture was then stirred for another 1 h at 0° C. and then allowed to warm to r.t. After stirring for 16 h, the mixture was allowed to cool to 0° C. and quenched by slow addition of sat. aq. NH₄Cl (caution: gas evolution). The mixture was diluted with EtOAc and brine and separated. The aqueous layer was extracted with EtOAc (3×). The organic layers were combined, washed with brine, dried over MgSO4, and concentrated. The crude product was purified by FCC (0-70% EtOAc/heptane) to give the sub-title compound as colorless oil (71.1 g, 94% yield). LCMS calc. for C₂₂H₃₀NO₄Si (M-^{t}Bu+2H)⁺: m/z=400.2; found 400.2.

### Step 3. tert-Butyl (2R,4S)-4-((tert-butyldiphenylsilyl)oxy)-2-formylpyrrolidine-1-carboxylate

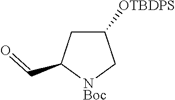

To a stirred solution of tert-butyl (2R,4S)-4-((tert-butyldiphenylsilyl)oxy)-2-(hydroxymethyl)pyrrolidine-1-carboxylate (60.43 g, 133 mmol) in DCM (265 mL) at 0° C. under rapid stirring was added trichloroisocyanuric acid (32.4 g, 139 mmol) followed by TEMPO (0.207 g, 1.326 mmol). The suspension markedly increased in turbidity within a few minutes. The ice bath was removed and the mixture was stirred for an additional 20 min. The mixture was filtered through diatomaceous earth. The diatomaceous earth was washed with DCM and the combined filtrate was concentrated. The crude product was purified by FCC (0-50% EtOAc/heptane) to give the sub-title compound as light yellow oil (51.7 g, 86% yield). LCMS calc. for C₂₁H₂₈NO₂Si (M-Boc+2H)⁺: m/z=354.2; found 354.2.

### Step 4. tert-Butyl (2R,4S)-4-((tert-butyldiphenylsilyl)oxy)-2-ethynylpyrrolidine-1-carboxylate

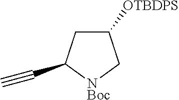

To a solution of tert-butyl (2R,4S)-4-((tert-butyldiphenylsilyl)oxy)-2-formylpyrrolidine-1-carboxylate (32.27 g, 71.1 mmol) in MeOH (356 mL) were added dimethyl (1-diazo-2-oxopropyl)phosphonate (12.28 mL, 82 mmol) and K₂CO₃ (22.12 g, 160 mmol). The mixture was stirred at r.t. for 2 h. The mixture was then diluted with brine and extracted with EtOAc (3×). The combined organic layers were dried over Na₂SO₄, filtered and concentrated. The crude product was purified by FCC (0-50% EtOAc/heptane) to give the sub-title compound as yellow oil (23.9 g, 75% yield).

### Step 5. tert-Butyl (2R,4S)-4-((tert-butyldiphenylsilyl)oxy)-2-(2-oxoethyl)pyrrolidine-1-carboxylate

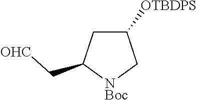

To a solution of tert-butyl (2R,4S)-4-((tert-butyldiphenylsilyl)oxy)-2-ethynylpyrrolidine-1-carboxylate (11.4 g, 25.4 mmol) in THF (50 mL) cooled to 0° C. was added 9-BBN (0.5 M in THF, 63.4 mL, 31.7 mmol) slowly over 15 min. After addition, the mixture was allowed to warm to r.t. and stirred for additional 1 h. Then the mixture was cooled to 0° C. and sodium perborate tetrahydrate (19.5 g, 127.0 mmol) was added portionwise followed by addition of water (50 mL). The mixture was allowed to warm to r.t. and stirred for 2 h. The mixture was then diluted with EtOAc and washed with brine. The organic layer was dried over Na₂SO₄, filtered and concentrated. The crude product was purified by FCC (20% EtOAc/hexanes) to give the sub-title compound as colorless oil (10.98 g, 93% yield). LCMS calc. for C₂₃H₂₉NO₄Si (M-^{t}Bu+2H)⁺: m/z=412.2; found 412.2.

### Step 6. tert-Butyl (2R,4S)-4-((tert-butyldiphenylsilyl)oxy)-2-(2,2-diethoxyethyl)pyrrolidine-1-carboxylate

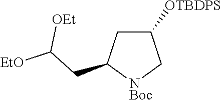

To a solution of tert-butyl (2R,4S)-4-((tert-butyldiphenylsilyl)oxy)-2-(2-oxoethyl)pyrrolidine-1-carboxylate (10.98 g, 23.5 mmol) in EtOH (47 mL) was added triethoxymethane (4.3 mL, 25.8 mmol) followed by tetrabutylammonium tribromide (0.23 g, 0.47 mmol). The mixture was stirred at r.t. for 16 h. The mixture was then poured to sat. aq. NaHCO₃. The resulting mixture was extracted with EtOAc (3×). The combined organic layers were dried over Na₂SO₄, filtered and concentrated. The crude product was used directly for next step. LCMS calc. for C₂₅H₃₄NO₄Si (M-^{t}Bu-EtO+H)⁺: m/z=440.2; found 440.2.

### Step 7. tert-Butyl (2R,4S)-2-(2,2-diethoxyethyl)-4-hydroxypyrrolidine-1-carboxylate

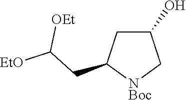 [SMILES: CCOC(C[C@H]1C[C@H](O)CN1C(=O)OC(C)(C)C)OCC]

To a solution of tert-butyl (2R,4S)-4-((tert-butyldiphenylsilyl)oxy)-2-(2,2-diethoxyethyl)pyrrolidine-1-carboxylate (12.7 g, 23.5 mmol) in DMF (45 mL) and water (2.3 mL) was added CsF (10.7 g, 70.4 mmol). The mixture was stirred at 100° C. for 2 h. The mixture was then diluted with EtOAc and washed with brine 5 times. The organic layer was dried over Na₂SO₄, filtered and concentrated. The crude product was purified by FCC (0-100% EtOAc/hexanes) to give the sub-title compound as colorless oil (4.0 g, 56% yield). LCMS calc. for C₉H₁₆NO₄ (M-^{t}Bu-EtO+H)⁺: m/z=202.1; found 202.1.

### Step 8. tert-Butyl (2R,4R)-4-azido-2-(2,2-diethoxyethyl)pyrrolidine-1-carboxylate

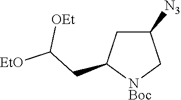 [SMILES: CCOC(C[C@H]1C[C@@H](N=[N+]=[N-])CN1C(=O)OC(C)(C)C)OCC]

To a solution of tert-butyl (2R,4S)-2-(2,2-diethoxyethyl)-4-hydroxypyrrolidine-1-carboxylate (4.80 g, 15.8 mmol) in DCM (40 mL) at 0° C. were added DIPEA (3.3 mL, 19.0 mmol) and methanesulfonyl chloride (1.99 g, 17.4 mmol). The mixture was stirred at 0° C. for 1 h and then quenched with sat. aq. NaHCO₃. The resulting mixture was extracted with DCM. The organic layer was dried over Na₂SO₄, filtered and concentrated.

The residue was dissolved in DMF (40 mL) and sodium azide (2.06 g, 31.6 mmol) was added in one portion. The mixture was stirred at 80° C. for 16 h. The mixture was then diluted with EtOAc and washed with brine 5 times. The organic layer was dried over Na₂SO₄, filtered and concentrated. The crude product was purified by FCC (0-40% EtOAc/hexanes) to give the sub-title compound as colorless oil (4.0 g, 78% yield). LCMS calc. for C₉H₁₅N₄O₄ (M-^{t}Bu-EtO+H)⁺: m/z=227.1; found 227.1.

### Step 9. tert-Butyl (2R,4R)-4-azido-2-(2-oxoethyl)pyrrolidine-1-carboxylate

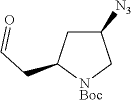 [SMILES: CC(C)(C)OC(=O)N1C[C@H](N=[N+]=[N-])C[C@@H]1CC=O]

To a solution of tert-butyl (2R,4R)-4-azido-2-(2,2-diethoxyethyl)pyrrolidine-1-carboxylate (2.94 g, 8.95 mmol) in acetone (45 mL) were added PPTS (2.25 g, 8.95 mmol) and water (0.32 mL, 17.9 mmol). The mixture was heated at 60° C. for 1 h. Volatiles were then removed under reduced pressure. The residue was diluted with EtOAc and washed with brine and water. The organic layer was dried over Na₂SO₄, filtered and concentrated. The crude product was used in the next step without further purification. LCMS calc. for C₇H₁₁N₄O₃ (M-^{t}Bu+2H)⁺: m/z=199.1; found 199.1.

### Step 10. tert-Butyl (1R,5S)-2-(cyclopropanecarbonyl)-3-((trimethylsilyl)ethynyl)-2,6-diazabicyclo[3.2.1]octane-6-carboxylate

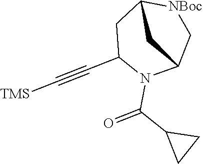 [SMILES: CC(C)(C)OC(=O)N1C[C@H]2C[C@@H]1CC(C#C[Si](C)(C)C)N2C(=O)C1CC1]

To a solution of tert-butyl (2R,4R)-4-azido-2-(2-oxoethyl)pyrrolidine-1-carboxylate (1.27 g, 5.0 mmol) in toluene (25 mL) was added triphenylphosphine (1.31 g, 5.0 mmol). The mixture was stirred at r.t. for 3 h.

To a separate flask containing trimethylsilylacetylene (1.08 g, 11.0 mmol) in THF (12.5 mL) cooled to −78° C. was added n-butyllithium (2.5 M in hexanes, 4.0 mL, 10.0 mmol) slowly. The mixture was stirred at −78° C. for 0.5 h before BF₃Et₂O (1.2 mL, 10.0 mmol) was added. The resulting mixture was stirred at −78° C. for another 15 min. Then, the above toluene solution was added slowly into the mixture at −78° C. After completion of the addition, the mixture was stirred at −78° C. for another 1 h before warming up to r.t. over 1 h. The mixture was then cooled back to −78° C. and quenched with slow addition of MeOH. The mixture was allowed to warm to r.t., diluted with EtOAc and wash with brine and water. The organic layer was dried over Na₂SO₄, filtered and concentrated. The crude product was purified by FCC (0-100% EtOAc/hexanes) to give the sub-title compound as a pair of diastereomers (1.05 g, 56% yield). LCMS calc. for C₁₆H₂₅N₂O₃Si (M-^{t}Bu+2H)⁺: m/z=321.1; found 321.1.

### Step 11. 2-(Trimethylsilyl)ethyl (1R,5S)-2-(cyclopropanecarbonyl)-3-((trimethylsilyl)ethynyl)-2,6-diazabicyclo[3.2.1]octane-6-carboxylate

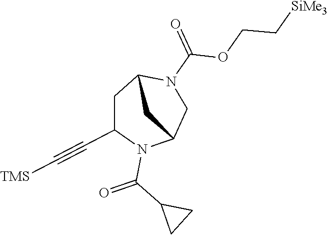 [SMILES: C[Si](C)(C)C#CC1C[C@H]2C[C@H](CN2C(=O)OCC[Si](C)(C)C)N1C(=O)C1CC1]

To a solution of tert-butyl (1R,5S)-2-(cyclopropanecarbonyl)-3-((trimethylsilyl)ethynyl)-2,6-diazabicyclo[3.2.1]octane-6-carboxylate (1.05 g, 2.79 mmol) in DCM (7 mL) was added TFA (7 mL). The mixture was stirred for 0.5 h and concentrated under reduced pressure. The residue was dissolved in THF and DIPEA (2.4 mL, 13.9 mmol) was added followed by 1-[2-(trimethylsilyl)ethoxycarbonyloxy]pyrrolidin-2,5-dione (1.09 g, 4.18 mmol). The mixture was stirred for 0.5 h and diluted with EtOAc. The organics was washed with brine and water, dried over Na₂SO₄, filtered and concentrated. The crude product was purified by FCC (0-40% EtOAc/hexanes) to give the sub-title compound as a pair of diastereomers (1.10 g, 94% yield). LCMS calc. for C₂₁H₃₇N₂O₃Si₂ (M+H)⁺: m/z=421.2; found 421.2.

### Step 12. 2-(Trimethylsilyl)ethyl (1R,3R,5S)-2-(cyclopropanecarbonyl)-3-ethynyl-2,6-diazabicyclo[3.2.1]octane-6-carboxylate

To a solution of 2-(trimethylsilyl)ethyl (1R,5S)-2-(cyclopropanecarbonyl)-3-((trimethylsilyl)ethynyl)-2,6-diazabicyclo[3.2.1]octane-6-carboxylate (0.88 g, 2.1 mmol) in MeOH (11 mL) was added K₂CO₃ (0.87 g, 6.3 mmol). The mixture was stirred at r.t. for 15 min. The mixture was then filtered through diatomaceous earth and concentrated. The residue was dissolved in EtOAc and washed with brine and water. The organic layer was dried over Na₂SO₄, filtered and concentrated. The crude product was purified by FCC (40-100% EtOAc/hexanes) to give the title compound (eluted prior to its diastereomer) as a colorless oil (0.17 g, 23% yield). LCMS calc. for C₁₆H₂₅N₂O₃Si (M−2Me+3H)⁺: m/z=321.2; found 321.1.

### Intermediate 12. tert-Butyl (1R,4R,5S)-5-((Rₐ)-8-(2-cyanoethyl)-2-((1R,3R,5R)-2-(cyclopropanecarbonyl)-2,6-diazabicyclo[3.2.1]octan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

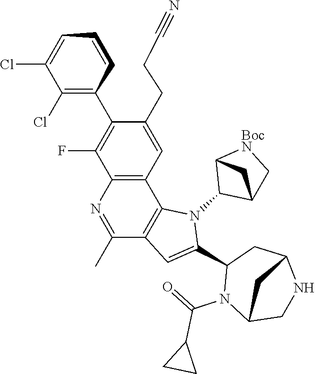

### Step 1. tert-Butyl (1R,4R,5S)-5-((Rₐ)-8-(2-cyanoethyl)-2-((1R,3R,5R)-2-(cyclopropanecarbonyl)-6-((2-(trimethylsilyl)ethoxy)carbonyl)-2,6-diazabicyclo[3.2.1]octan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

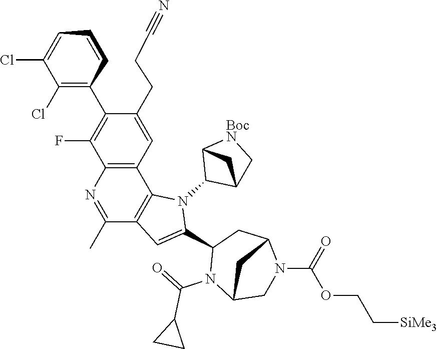

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 0.265 g, 0.389 mmol), 2-(trimethylsilyl)ethyl (1R,3R,5S)-2-(cyclopropanecarbonyl)-3-ethynyl-2,6-diazabicyclo[3.2.1]octane-6-carboxylate (intermediate 11, 0.163 g, 0.467 mmol), CuI (0.030 g, 0.156 mmol), Pd(PPh₃)₄ (0.090 g, 0.078 mmol) and DIPEA (0.7 mL, 3.89 mmol) in DMF (1.9 mL) was sparged with N₂ and heated at 70° C. for 1 h. Then, Cs₂CO₃ (0.380 g, 1.17 mmol) was added to the mixture. The resulting slurry was stirred at 90° C. for another 18 h. The mixture was then allowed to cool to r.t. and poured into water. The solution was extracted with EtOAc (2×). Then the combined organic layers were washed with brine (5×), dried over Na₂SO₄, filtered and concentrated. The crude product was purified by FCC (0-100% EtOAc/hexanes) to afford the sub-title compound (0.250 g, 71% yield). LCMS calc. for C₄₇H₅₆Cl₂FN₆O₅Si (M+H)⁺: m/z=901.3; found 901.5.

### Step 2. tert-Butyl (1R,4R,5S)-5-((Rₐ)-8-(2-cyanoethyl)-2-((1R,3R,5R)-2-(cyclopropanecarbonyl)-2,6-diazabicyclo[3.2.1]octan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

To a solution of tert-butyl (1R,4R,5S)-5-((Rₐ)-8-(2-cyanoethyl)-2-((1R,3R,5R)-2-(cyclopropanecarbonyl)-6-((2-(trimethylsilyl)ethoxy)carbonyl)-2,6-diazabicyclo[3.2.1]octan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (0.250 g, 0.277 mmol) in THF (1.4 mL) was added tris(dimethylamino)sulfonium difluorotrimethylsilicate (0.115 g, 0.416 mmol) in one portion. The mixture was heated at 60° C. for 1 h. The mixture was then allowed to cool to r.t. and diluted with EtOAc. The organic fraction was washed with brine, dried over Na₂SO₄, filtered and concentrated. The crude solid was dried under reduced pressure and used directly for further reactions. LCMS calc. for C₄₁H₄₄Cl₂FN₆O₃ (M+H)⁺: m/z=757.3; found 757.3.

The alternative atropisomer tert-butyl (1R,4R,5S)-5-((Sₐ)-8-(2-cyanoethyl)-2-((1R,3R,5R)-2-(cyclopropanecarbonyl)-2,6-diazabicyclo[3.2.1]octan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate can be prepared by an analogous route starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Intermediate 13. Dimethyl (1 S,5R,7R)-2-methyl-3-((R)-1-phenylethyl)-3,6-diazabicyclo[3.2.1]octane-6,7-dicarboxylate

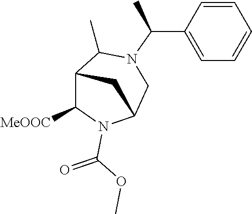 [SMILES: COC(=O)[C@H]1[C@H]2C[C@H](CN([C@@H](C)c3ccccc3)C2C)N1C(=O)OC]

### Step 1. 6-(tert-Butyl) 7-methyl (1S,5R,7R)-3-((R)-1-phenylethyl)-3,6-diazabicyclo[3.2.1]octane-6,7-dicarboxylate

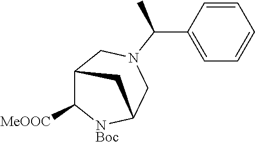 [SMILES: COC(=O)[C@H]1[C@H]2C[C@H](CN([C@@H](C)c3ccccc3)C2)N1C(=O)OC(C)(C)C]

To a solution of 6-(tert-butyl) 7-methyl (1R,5R,7R)-2,4-dihydroxy-3-oxa-6-azabicyclo[3.2.1]octane-6,7-dicarboxylate (intermediate 7, step 1, 2.40 g, 7.91 mmol) in DCM (39.6 mL) was added (S)-1-phenylethan-1-amine (1.071 mL, 8.31 mmol) and PPTS (3.98 g, 15.8 mmol). The mixture was stirred for 1 h at r.t. before NaBH(OAc)₃ (8.39 g, 39.6 mmol) was added. After stirring for 0.5 h, the reaction was quenched by slow addition of water. The mixture was extracted with DCM (2×). The combined organic layers were dried over Na₂SO₄, filtered and concentrated. The crude was further purified by FCC (0-40% EtOAc/hexanes) to afford the sub-title compound as a colorless oil in near quantitative yield. LCMS calc. for C₂₁H₃₁N₂O₄ (M+H)⁺: m/z=375.3; found 375.3

### Step 2. 6-(tert-Butyl) 7-methyl (1R,5R,7R)-2-oxo-3-((R)-1-phenylethyl)-3,6-diazabicyclo[3.2.1]octane-6,7-dicarboxylate

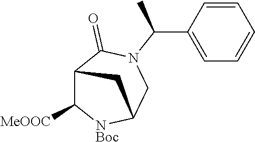 [SMILES: COC(=O)[C@H]1[C@H]2C[C@H](CN([C@@H](C)c3ccccc3)C2=O)N1C(=O)OC(C)(C)C]

Ruthenium(IV) oxide (0.154 g, 1.160 mmol) was added to a solution of NaIO₄ (2.48 g, 11.6 mmol) in water (19 mL) and the mixture was stirred at r.t. for 5 min. To the formed yellow solution was added dropwise a solution of 6-(tert-butyl) 7-methyl (1 S,5R,7R)-3-((R)-1-phenylethyl)-3,6-diazabicyclo[3.2.1]octane-6,7-dicarboxylate (1.45 g, 3.87 mmol) in EtOAc (19 mL), and the resulting mixture was vigorously stirred at r.t. for 1 h. Upon completion, the mixture was quenched by adding isopropyl alcohol, diluted with EtOAc and filtered through diatomaceous earth. The filtrate was washed with brine, dried over Na₂SO₄, filtered and concentrated. The crude was purified by FCC (0-60% EtOAc/hexanes) to afford the sub-title compound as a colorless oil (0.98 g, 65% yield). LCMS calc. for C₂₁H₂₈N₂O₅ (M+H)⁺: m/z=389.3; found 389.3.

### Step 3. 6-(tert-Butyl) 7-methyl (1S,5R,7R)-2-methyl-3-((R)-1-phenylethyl)-3,6-diazabicyclo[3.2.1]octane-6,7-dicarboxylate

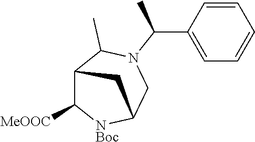 [SMILES: COC(=O)[C@H]1[C@H]2C[C@H](CN([C@@H](C)c3ccccc3)C2C)N1C(=O)OC(C)(C)C]

To a mixture of 6-(tert-butyl) 7-methyl (1R,5R,7R)-2-oxo-3-((R)-1-phenylethyl)-3,6-diazabicyclo[3.2.1]octane-6,7-dicarboxylate (0.98 g, 2.53 mmol) and Ir(CO)Cl(PPh₃)₂ (0.020 g, 0.025 mmol) in anhydrous DCM (25 mL) was added 1,1,3,3-tetramethyldisiloxane (0.68 g, 5.06 mmol). The resulting mixture was stirred at r.t. for 20 min. or until almost clear under an inert atmosphere. After cooling to −78° C., a solution of MeMgCl (3 M in THF, 2.1 mL, 6.33 mmol) was added dropwise. The resulting mixture was stirred at −78° C. for another 5 min. and allowed to warm up to r.t. Upon completion, the reaction mixture was cooled down to 0° C. and quenched by slow addition of sat. aq. NH₄Cl. The organics were extracted with DCM. The combined organic layers were washed with brine, dried over Na₂SO₄, filtered and concentrated. The crude was purified by prep. LCMS (XBRIDGE® C₁₈ column, eluting with a gradient of MeCN/water containing 0.1% NH₄OH, at flow rate of 60 mL/min.) to obtain the sub-title compound (0.21 g, 37% yield). LCMS calc. for C₂₂H₃₃N₂O₄ (M+H)⁺: m/z=389.3; found 389.3.

### Step 4. Dimethyl (1S,5R,7R)-2-methyl-3-((R)-1-phenylethyl)-3,6-diazabicyclo[3.2.1]octane-6,7-dicarboxylate

To a solution of 6-(tert-butyl) 7-methyl (1 S,5R,7R)-2-methyl-3-((R)-1-phenylethyl)-3,6-diazabicyclo[3.2.1]octane-6,7-dicarboxylate (0.212 g, 0.546 mmol) in dioxane (2 mL) was added HCl (4 M in 1,4-dioxane, 1 mL). The mixture was stirred at r.t. for 1 h. The volatiles were removed under reduced pressure. The residue was dissolved in DCM (4 mL). Upon stirring, DIPEA (0.48 mL, 2.73 mmol) was added followed by methyl chloroformate (0.063 mL, 0.819 mmol). The mixture was stirred at r.t. for 0.5 h. The reaction mixture was diluted with DCM, washed with water, dried over Na₂SO₄, filtered and concentrated. The crude was further purified by FCC (0-60% EtOAc/hexanes) to afford the title compound as a colorless oil (0.138 g, 88% yield). LCMS calc. for C₁₉H₂₇N₂O₄ (M+H)⁺: m/z=347.2; found 347.2.

### Intermediate 14. (2R,4R,5R)-1-(tert-Butoxycarbonyl)-4-(((tert-butyidiphenylsilyl)oxy)methyl)-5-ethynylpyrrolidine-2-carboxylic acid

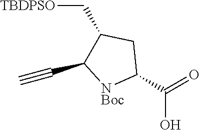

### Step 1. 1-(tert-Butyl) 2-methyl (2R,3R,5R)-3-(hydroxymethyl)-5-((trityloxy)methyl)pyrrolidine-1,2-dicarboxylate

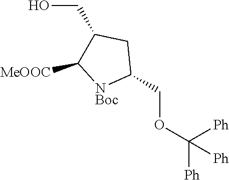 [SMILES: COC(=O)[C@H]1[C@H](CO)C[C@H](COC(c2ccccc2)(c2ccccc2)c2ccccc2)N1C(=O)OC(C)(C)C]

To a solution of 1-(tert-butyl) 2-methyl (2R,3R,5R)-3,5-bis(hydroxymethyl)pyrrolidine-1,2-dicarboxylate (intermediate 9, step 2, 19.6 g, 67.8 mmol) in DCM (226 mL) was added trityl chloride (19.9 g, 71.2 mmol), DMAP (1.16 g, 9.49 mmol) and pyridine (16.5 mL, 203 mmol). The mixture was stirred at r.t. for 16 h. The mixture was diluted with sat. aq. NaHCO₃ and extracted with EtOAc (3×). The organic layers were combined, dried over MgSO₄, filtered and concentrated. The crude product was further purified by FCC (0-40% EtOAc/n-heptane with 0.5% isopropyl alcohol modifier). The fractions containing the major regio-isomer (eluted later) were combined and concentrated to afford sub-title compound (15.4 g, 43% yield). LCMS calc. for C₃₂H₃₇NNaO₆ (M+Na)⁺: m/z=554.3; found 554.4.

### Step 2. 1-(tert-Butyl) 2-methyl (2R,3R,5R)-3-(((tert-butyldiphenylsilyl)oxy)methyl)-5-((trityloxy)methyl)pyrrolidine-1,2-dicarboxylate

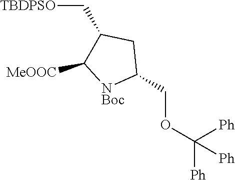

To a solution of 1-(tert-butyl) 2-methyl (2R,3R,5R)-3-(hydroxymethyl)-5-((trityloxy)methyl)pyrrolidine-1,2-dicarboxylate (15.4 g, 28.9 mmol) in DCM (145 mL) was added TBDPSCI (7.8 mL, 30.4 mmol) and imidazole (9.84 g, 145 mmol). The mixture was stirred at r.t. for 16 h. This mixture was diluted with water and extracted with DCM (3×). The organic layers were combined, dried over MgSO₄, filtered and concentrated. The crude product was further purified by FCC (0-30% EtOAc/n-heptane) to afford the sub-title compound (19.3 g, 87% yield) as a viscous, clear oil that solidified upon standing. LCMS calc. for C₄₈H₅₅NNaO₆Si (M+Na)⁺: m/z=792.4; found 792.5.

### Step 3. tert-Butyl (2R,3R,5R)-3-(((tert-butyldiphenylsilyl)oxy)methyl)-2-formyl-5-((trityloxy)methyl)pyrrolidine-1-carboxylate

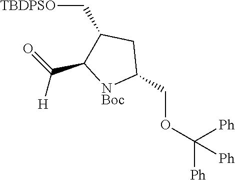

To a solution of 1-(tert-butyl) 2-methyl (2R,3R,5R)-3-(((tert-butyldiphenylsilyl)oxy)methyl)-5-((trityloxy)methyl)pyrrolidine-1,2-dicarboxylate (11.5 g, 14.9 mmol) in DCM (74 mL) cooled to −78° C. was added DIBAL (1 M in toluene, 26.0 mL, 26.0 mmol) over 1 h using a syringe pump. The mixture was stirred at −78° C. for an additional 1 h. The mixture was quenched using a mixture of MeOH (3.8 mL) and DCM (12 mL) over 0.5 h using a syringe pump, maintaining internal temperature at −78° C. The mixture was stirred for another 30 min. at −78° C. before sat. aq. Rochelle's salt (50 mL) was added. Then resulting mixture was allowed to warm up to r.t and diluted with DCM and water. The mixture was stirred vigorously at r.t. for 1 h. The organic layer was separated washed with brine, dried over MgSO₄, filtered and concentrated. The crude product was used in the next step without further purification. LCMS calc. for C₄₇H₅₃NNaO₅Si (M+Na)⁺: m/z=762.4; found 762.5.

### Step 4. tert-Butyl (2R,3R,5R)-3-(((tert-butyldiphenylsilyl)oxy)methyl)-2-ethynyl-5-((trityloxy)methyl)pyrrolidine-1-carboxylate

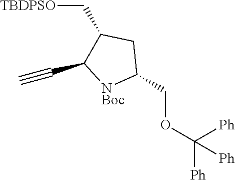

To a solution of tert-butyl (2R,3R,5R)-3-(((tert-butyldiphenylsilyl)oxy)methyl)-2-formyl-5-((trityloxy)methyl)pyrrolidine-1-carboxylate (11.0 g, 14.9 mmol) in MeOH (103 mL) cooled to 0° C. were added K₂CO₃ (7.19 g, 52.0 mmol) and dimethyl (1-diazo-2-oxopropyl)phosphonate (5.4 mL, 35.7 mmol). The mixture was allowed to warm to r.t. and stirred for 2 h. The mixture was diluted with water and extracted with DCM (4×). The organic layers were combined, washed with brine, dried over MgSO₄, filtered and concentrated. The crude product was purified by FCC (0-25% EtOAc/n-heptane) to afford sub-title compound (7.31 g, 67% yield). LCMS calc. for C₄₈H₅₃NNaO₄Si (M+Na)⁺: m/z=758.4; found 758.5.

### Step 5. tert-Butyl (2R,3R,5R)-3-(((tert-butyldiphenylsilyl)oxy)methyl)-2-ethynyl-5-(hydroxymethyl)pyrrolidine-1-carboxylate

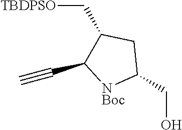

To a solution of tert-butyl (2R,3R,5R)-3-(((tert-butyldiphenylsilyl)oxy)methyl)-2-ethynyl-5-((trityloxy)methyl)pyrrolidine-1-carboxylate (7.31 g, 9.93 mmol) and triisopropylsilane (6.1 mL, 29.8 mmol) in DCM (48 mL) cooled at 0° C. was added dropwise TFA (1.2 mL) over 5 min. The mixture was stirred for 1 h at 0° C. and then quenched with sat. aq. NaHCO₃ (25 mL). The organic phase was separated and the aqueous layer was extracted with DCM (3×). Organic layers were combined, washed with brine, dried over MgSO₄, filtered and concentrated. The crude product was used in the next step without further purification. LCMS calc. for C₂₄H₃₂NO₂Si (M-Boc+2H)⁺: m/z=394.2; found 394.3.

### Step 6. (2R,4R,5R)-1-(tert-Butoxycarbonyl)-4-(((tert-butyldiphenylsilyl)oxy)methyl)-5-ethynylpyrrolidine-2-carboxylic acid

To a solution of tert-butyl (2R,3R,5R)-3-(((tert-butyldiphenylsilyl)oxy)methyl)-2-ethynyl-5-(hydroxymethyl)pyrrolidine-1-carboxylate (3.94 g, 7.98 mmol), Phl(OAc)₂ (5.65 g, 17.6 mmol) and NaHCO₃ (1.34 g, 16.0 mmol) in MeCN (60 mL) and water (20 mL) cooled to 0° C. was added TEMPO (0.13 g, 0.80 mmol). The mixture was stirred at r.t. for 7 h. The mixture was diluted with DCM (50 mL) and cooled to 0° C. Aq. HCl (1 M) was added to acidify the mixture to pH 3. The resulting mixture was extracted with DCM (3×). Organic layers were combined, washed with brine, dried over MgSO₄, filtered and concentrated. The crude product was used in the next step without further purification. LCMS calc. for C₂₄H₃₀NO₃Si (M-Boc+2H)⁺: m/z=408.2; found 408.3.

### Intermediate 15. 2-(Trimethylsilyl)ethyl (1 S,5R,7R)-6-(cyclopropanecarbonyl)-7-ethynyl-3,6-diazabicyclo[3.2.1]octane-3-carboxylate-2,2-d₂

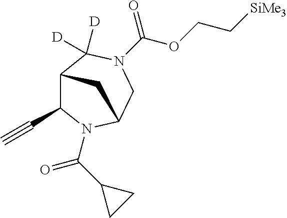 [SMILES: [2H]C1([2H])[C@@H]2C[C@H](CN1C(=O)OCC[Si](C)(C)C)N(C(=O)C1CC1)[C@H]2C#C]

### Step 1. 6-(tert-Butyl) 7-methyl (1R,5R,7R)-2-oxo-3-(1-phenylethyl)-3,6-diazabicyclo[3.2.1]octane-6,7-dicarboxylate

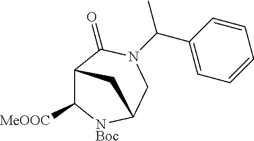 [SMILES: COC(=O)[C@H]1[C@H]2C[C@H](CN(C(C)c3ccccc3)C2=O)N1C(=O)OC(C)(C)C]

The sub-title compound was prepared according to the procedures described for Intermediate 13, steps 1-2, using racemic 1-phenylethan-1-amine instead of the chiral congener. LCMS calc. for C₂₁H₂₉N₂O₅ (M+H)⁺: m/z=389.2; found 389.2.

### Step 2. tert-Butyl (1S,5R,7R)-7-(hydroxymethyl-d₂)-3-(1-phenylethyl)-3,6-diazabicyclo[3.2.1]octane-6-carboxylate-2,2-d₂

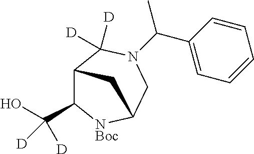 [SMILES: [2H]C([2H])(O)[C@H]1[C@H]2C[C@H](CN(C(C)c3ccccc3)C2([2H])[2H])N1C(=O)OC(C)(C)C]

LiAID₄ (0.137 g, 3.60 mmol) was added to a solution of 6-(tert-butyl) 7-methyl (1R,5R,7R)-2-oxo-3-(1-phenylethyl)-3,6-diazabicyclo[3.2.1]octane-6,7-dicarboxylate (0.35 g, 0.90 mmol) in THF (10 mL) at 0° C. After stirring at 0° C. for 15 min., BF₃Et₂O (0.23 mL, 1.80 mmol) was added dropwise, then the resulting mixture was stirred at 0° C. for 2 h. Upon completion, the mixture was quenched by slow addition of ice, then diluted with DCM. Diatomaceous earth was added and the mixture was stirred for another 20 min. and filtered. The filtrate was washed with brine, dried over Na₂SO₄, and concentrated. The crude product was purified by FCC (0-10% MeOH/DCM) to afford the sub-title compound (0.25 g, 79% yield). LCMS calc. for C₂₀H₂₇D₄N₂O₃ (M+H)⁺: m/z=351.3; found 351.3.

### Step 3. 6-(tert-Butyl) 3-(2-(trimethylsilyl)ethyl) (1S,5R,7R)-7-(hydroxymethyl-d₂)-3,6-diazabicyclo[3.2.1]octane-3,6-dicarboxylate-2,2-d₂

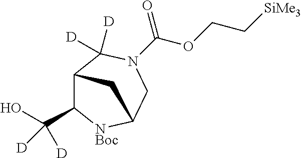 [SMILES: [2H]C([2H])(O)[C@H]1[C@H]2C[C@H](CN(C(=O)OCC[Si](C)(C)C)C2([2H])[2H])N1C(=O)OC(C)(C)C]

To a solution of tert-butyl (1 S,5R,7R)-7-(hydroxymethyl-d₂)-3-(1-phenylethyl)-3,6-diazabicyclo[3.2.1]octane-6-carboxylate-2,2-d₂ (0.25 g, 0.71 mmol) in EtOH (5 mL) was added 20% Pd(OH)₂/C (0.10 g). The mixture was stirred under H₂ atmosphere for 1 h. The reaction mixture was filtered through diatomaceous earth and concentrated.

The residue was dissolved in THF (12 mL). To the solution was added DIPEA (0.37 mL, 2.14 mmol) and 1-[2-(trimethylsilyl)ethoxycarbonyloxy]pyrrolidin-2,5-dione (0.21 g, 0.86 mmol). The mixture was stirred for 1 h at r.t. and then diluted with EtOAc. The organic layer was washed with brine, dried over Na₂SO₄, filtered and concentrated. The crude product was purified by FCC (0-60% EtOAc/hexanes) to afford the sub-title compound (0.25 g, 88% yield). LCMS calc. for C₁₈H₃₀D₄N₂O₅SiNa (M+Na)⁺: m/z=413.3; found 413.3.

### Step 4. 6-(tert-Butyl) 3-(2-(trimethylsilyl)ethyl) (1S,5R,7R)-7-(formyl-d)-3,6-diazabicyclo[3.2.1]octane-3,6-dicarboxylate-2,2-d₂

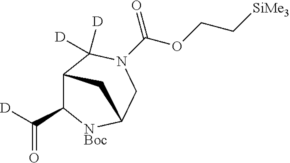 [SMILES: [2H]C(=O)[C@H]1[C@H]2C[C@H](CN(C(=O)OCC[Si](C)(C)C)C2([2H])[2H])N1C(=O)OC(C)(C)C]

To a solution of 6-(tert-butyl) 3-(2-(trimethylsilyl)ethyl) (1 S,5R,7R)-7-(hydroxymethyl-d₂)-3,6-diazabicyclo[3.2.1]octane-3,6-dicarboxylate-2,2-d₂ (0.207 g, 0.53 mmol) in DCM (4 mL) was added Dess-Martin periodinane (0.339 g, 0.80 mmol). The resulting mixture was stirred at r.t. for 1 h. Sat. aq. Na₂S₂O₃ (5 mL) was added to the mixture, which was then stirred for an additional 10 min. The organic phase was separated, dried over Na₂SO₄, filtered and concentrated. The crude product was used in the next step without further purification. LCMS calc. for C₁₄H₂₂D₃N₂O₅Si (M-^{t}Bu+2H)⁺: m/z=332.2; found 332.2.

### Step 5. 6-(tert-Butyl) 3-(2-(trimethylsilyl)ethyl) (1S,5R,7R)-7-ethynyl-3,6-diazabicyclo[3.2.1]octane-3,6-dicarboxylate-2,2-d₂

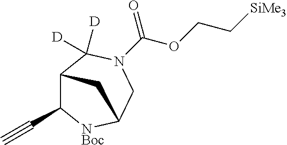 [SMILES: [2H]C1([2H])[C@@H]2C[C@H](CN1C(=O)OCC[Si](C)(C)C)N(C(=O)OC(C)(C)C)[C@H]2C#C]

To a solution of 6-(tert-butyl) 3-(2-(trimethylsilyl)ethyl) (1 S,5R,7R)-7-(formyl-d)-3,6-diazabicyclo[3.2.1]octane-3,6-dicarboxylate-2,2-d₂ (0.207 g, 0.53 mmol) in MeOH (4 mL) were added dimethyl(1-diazo-2-oxopropyl)phosphonate (0.205 g, 1.06 mmol) and K₂CO₃ (0.148 g, 1.06 mmol). After stirring for 1 h, the mixture was filtered through diatomaceous earth. The filtrate was concentrated. The residue was diluted with water, extracted with EtOAc, filtered through diatomaceous earth and concentrated. The crude product was purified by FCC (0-60% EtOAc/hexanes) to afford the sub-title compound (0.169 g, 83% yield over 2 steps). LCMS calc. for C₁₉H₃₀D₂N₂O₄SiNa (M+Na)⁺: m/z=405.2; found 405.2.

### Step 6. 2-(Trimethylsilyl)ethyl (1S,5R,7R)-6-(cyclopropanecarbonyl)-7-ethynyl-3,6-diazabicyclo[3.2.1]octane-3-carboxylate-2,2-d₂

To a solution of 6-(tert-butyl) 3-(2-(trimethylsilyl)ethyl) (1 S,5R,7R)-7-ethynyl-3,6-diazabicyclo[3.2.1]octane-3,6-dicarboxylate-2,2-d₂ (0.110 g, 0.29 mmol) in EtOAc (3 mL) was added HCl (4 M in 1,4-dioxane, 1.4 mL). The mixture was stirred at r.t. for 4 h. The volatiles were then removed under reduced pressure. The residue was dissolved in DCM (4 mL). With stirring, NEt₃ (0.2 mL, 1.4 mmol) was added, followed by cyclopropanecarbonyl chloride (0.060 g, 0.58 mmol), and stirring was continued for 0.5 h. The mixture was diluted with DCM, washed with water, dried over Na₂SO₄, filtered and concentrated. The crude product was purified by FCC (0-60% EtOAc/hexanes) to afford the title compound (0.020 g, 20% yield). LCMS calc. for C₁₈H₂₇D₂N₂O₃Si (M+H)⁺: m/z=351.2; found 351.2.

### Intermediate 16. tert-Butyl (1R,4R,5S)-5-((Rₐ)-8-(2-cyanoethyl)-7-(2,3-dichlorophenyl)-6-fluoro-2-((1S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate

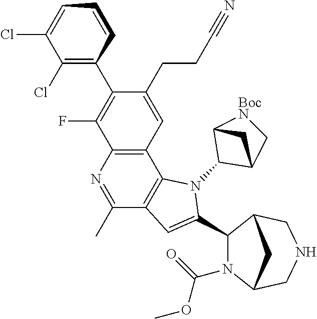

### Step 1. 3-Methyl 2-(2-(trimethylsilyl)ethyl) (1R,3R,4R,5R,6S)-5,6-dihydroxy-2-azabicyclo[2.2.1]heptane-2,3-dicarboxylate

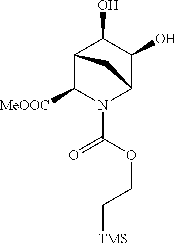 [SMILES: COC(=O)[C@H]1[C@H]2C[C@H]([C@H](O)[C@@H]2O)N1C(=O)OCC[Si](C)(C)C]

To methyl (1R,3R,4R,5R,6S)-5,6-dihydroxy-2-azabicyclo[2.2.1]heptane-3-carboxylate (Intermediate 4, Step 2, 14.8 g, 79 mmol) dissolved in THF (360 mL) were added DIPEA (48 mL, 276 mmol) and 2,5-dioxopyrrolidin-1-yl (2-(trimethylsilyl)ethyl) carbonate (40.9 g, 158 mmol). The mixture was stirred at r.t. for 0.5 h and diluted with EtOAc. After washing with brine, the organic fraction was dried over Na₂SO₄, filtered and concentrated. The crude product was further purified by flash column chromatography (0-100% EtOAc/hexanes) to afford the title compound (22.3 g, 85% yield). LCMS calc. for C₁₂H₂₂NO₆Si (M-(CH₂)₂+H)⁺: m/z=304.1; found 304.1.

### Step 2. 7-Methyl 6-(2-(trimethylsilyl)ethyl) (1R,5R,7R)-2,4-dihydroxy-3-oxa-6-azabicyclo[3.2.1]octane-6,7-dicarboxylate

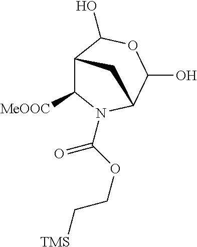 [SMILES: COC(=O)[C@H]1[C@H]2C[C@H](C(O)OC2O)N1C(=O)OCC[Si](C)(C)C]

To a solution of 3-methyl 2-(2-(trimethylsilyl)ethyl) (1R,3R,4R,5R,6S)-5,6-dihydroxy-2-azabicyclo[2.2.1]heptane-2,3-dicarboxylate (22.3 g, 67 mmol) in 1,4-dioxane/water (4:1, 224 mL) were added 2,6-lutidine (31 mL, 269 mmol) and NaIO₄ (28.8 g, 135 mmol). The mixture was stirred at r.t. for 1.5 h. The mixture was then diluted with EtOAc and filtered over Celite. The organics were extracted from the filtrate with EtOAc (3×) and washed with brine. The organic layer was dried over Na₂SO₄, filtered and concentrated under reduced pressure. The crude product was used in the next step without further purification. LCMS calc. for C₁₂H₂₂NO₇Si (M−(CH₂)₂+H)⁺: m/z=320.1; found 320.1.

### Step 3. 7-Methyl 6-(2-(trimethylsilyl)ethyl) (1S,5R,7R)-3-(1-phenylethyl)-3,6-diazabicyclo[3.2.1]octane-6,7-dicarboxylate

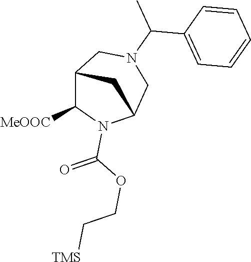 [SMILES: COC(=O)[C@H]1[C@H]2C[C@H](CN(C(C)c3ccccc3)C2)N1C(=O)OCC[Si](C)(C)C]

To a solution of 7-methyl 6-(2-(trimethylsilyl)ethyl) (1R,5R,7R)-2,4-dihydroxy-3-oxa-6-azabicyclo[3.2.1]octane-6,7-dicarboxylate (23.5 g, 67.6 mmol) in DCM (340 mL) was added 1-phenylethan-1-amine (13.1 mL, 101 mmol) and PPTS (34 g, 135 mmol). The mixture was stirred for 1 h at r.t. before NaBH(OAc)₃ (72 g, 338 mmol) was added. After stirring for 2 h, the reaction was quenched by slow addition of water. The mixture was extracted with DCM (3×). The combined organic layers were washed with brine and dried over Na₂SO₄, filtered and concentrated. The crude product was further purified by flash column chromatography (50-100% EtOAc/hexanes) to afford the desired compound (9.37 g, 33% yield). LCMS calc. for C₂₂H₃₅N₂O₄Si (M+H)⁺: m/z=419.2; found 419.2.

### Step 4. 7-Methyl 6-(2-(trimethylsilyl)ethyl) (1S,5R,7R)-3,6-diazabicyclo[3.2.1]octane-6,7-dicarboxylate

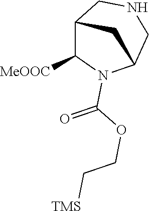 [SMILES: COC(=O)[C@H]1[C@@H]2CNC[C@@H](C2)N1C(=O)OCC[Si](C)(C)C]

To a solution of 7-methyl 6-(2-(trimethylsilyl)ethyl) (1 S,5R,7R)-3-(1-phenylethyl)-3,6-diazabicyclo[3.2.1]octane-6,7-dicarboxylate (7.0 g, 16.7 mmol) in EtOH (110 mL) was added 20% Pd(OH)₂/C (2.35 g). The mixture was stirred under H₂ atmosphere for 16 h. The resulting mixture was filtered through Celite and concentrated under reduced pressure. The crude product was used in the next step without further purification. LCMS calc. for C₁₂H₂₃N₂O₄Si (M−(CH₂)₂+H)⁺: m/z=287.1; found 287.2.

### Step 5. 7-Methyl 6-(2-(trimethylsilyl)ethyl) (1S,5R,7R)-3-((2-nitrophenyl)sulfonyl)-3,6-diazabicyclo[3.2.1]octane-6,7-dicarboxylate

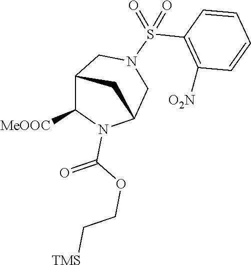 [SMILES: COC(=O)[C@H]1[C@H]2C[C@H](CN(S(=O)(=O)c3ccccc3[N+](=O)[O-])C2)N1C(=O)OCC[Si](C)(C)C]

To a solution of 7-methyl 6-(2-(trimethylsilyl)ethyl) (1 S,5R,7R)-3,6-diazabicyclo[3.2.1]octane-6,7-dicarboxylate (5.26 g, 16.7 mmol) in DCM (85 mL) cooled to 0° C. were added DIPEA (8.76 mL, 50.2 mmol) and 2-nitrobenzenesulfonyl chloride (5.19 g, 23.4 mmol). The mixture was stirred for 3 h and then was diluted with DCM. The organic fraction was washed with water and brine, dried over Na₂SO₄, filtered and concentrated. The crude product was further purified by flash column chromatography (0-100% EtOAc/hexanes) to afford the desired product (6.62 g, 79% yield). LCMS calc. for C₁₈H₂₆N₃O₈SSi (M−(CH₂)₂+H)⁺: m/z=472.1; found 472.1.

### Step 6. 2-(Trimethylsilyl)ethyl (1S,5R,7R)-7-(hydroxymethyl)-3-((2-nitrophenyl)sulfonyl)-3,6-diazabicyclo[3.2.1]octane-6-carboxylate

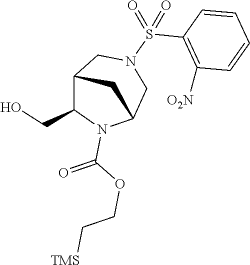 [SMILES: C[Si](C)(C)CCOC(=O)N1[C@@H]2C[C@@H](CN(S(=O)(=O)c3ccccc3[N+](=O)[O-])C2)[C@@H]1CO]

To a solution of 7-methyl 6-(2-(trimethylsilyl)ethyl) (1 S,5R,7R)-3-((2-nitrophenyl)sulfonyl)-3,6-diazabicyclo[3.2.1]octane-6,7-dicarboxylate (6.62 g, 13.3 mmol) in THF (65 mL) cooled to 0° C. was added LiBH₄ (2 M in THF, 7.30 mL, 14.6 mmol). The mixture was stirred at r.t. for 16 h and then quenched by slow addition of sat. aq. NH₄Cl at 0° C. The mixture was diluted with EtOAc, washed with brine, dried over Na₂SO₄, filtered and concentrated. The crude product was purified by flash column chromatography (0-100% EtOAc/hexanes) to afford the desired product (5.26 g, 84% yield). LCMS calc. for C₁₇H₂₆N₃O₇SSi (M−(CH₂)₂+H)⁺: m/z=444.1; found 444.1.

### Step 7. 2-(Trimethylsilyl)ethyl (1S,5R,7R)-7-formyl-3-((2-nitrophenyl)sulfonyl)-3,6-diazabicyclo[3.2.1]octane-6-carboxylate

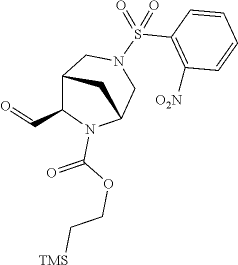 [SMILES: C[Si](C)(C)CCOC(=O)N1[C@@H]2C[C@@H](CN(S(=O)(=O)c3ccccc3[N+](=O)[O-])C2)[C@@H]1C=O]

To a solution of dimethyl sulfoxide (0.95 mL, 13.4 mmol) in DCM (20 mL) at −78° C. was added oxalyl chloride (1.17 mL, 13.4 mmol) dropwise. After 45 min., 2-(trimethylsilyl)ethyl (1 S,5R,7R)-7-(hydroxymethyl)-3-((2-nitrophenyl)sulfonyl)-3,6-diazabicyclo[3.2.1]octane-6-carboxylate (5.26 g, 11.2 mmol) in DCM (20 mL) was added dropwise at −78° C., the mixture was stirred for 3 h. Et₃N (4.66 mL, 33.5 mmol) was then added at −78° C., the resulting mixture was stirred at −78° C. for 45 min., then allowed to warm to r.t. and continued stirring for another 1 h. The reaction was then quenched with the addition of 1 N HCl and the product was extracted with DCM (3×). The combined organics were washed with brine, dried over Na₂SO₄ and concentrated to afford the crude product, which was used in the next step without further purification. LCMS calc. for C₁₇H₂₄N₃O₇SSi (M−(CH₂)₂+H)⁺: m/z=442.1; found 442.1.

### Step 8. 2-(Trimethylsilyl)ethyl (1S,5R,7R)-7-ethynyl-3-((2-nitrophenyl)sulfonyl)-3,6-diazabicyclo[3.2.1]octane-6-carboxylate

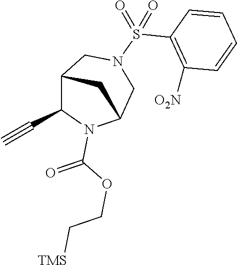 [SMILES: C#C[C@H]1[C@H]2C[C@H](CN(S(=O)(=O)c3ccccc3[N+](=O)[O-])C2)N1C(=O)OCC[Si](C)(C)C]

To a solution of 2-(trimethylsilyl)ethyl (1 S,5R,7R)-7-formyl-3-((2-nitrophenyl)sulfonyl)-3,6-diazabicyclo[3.2.1]octane-6-carboxylate (5.24 g, 11.2 mmol) in MeOH (56 mL) were added dimethyl(1-diazo-2-oxopropyl)phosphonate (3.67 mL, 27.9 mmol) and K₂CO₃ (4.63 g, 33.5 mmol). After stirring for 16 h, the mixture was filtered through Celite and concentrated under reduced pressure. The residue was diluted with EtOAc, washed with brine, dried over Na₂SO₄ and concentrated. The crude product was purified by flash column chromatography (0-100% EtOAc/hexanes) to afford the desired compound (2.87 g, 55% yield). LCMS calc. for C₁₈H₂₄N₃O₆SSi (M−(CH₂)₂+H)⁺: m/z=438.1; found 438.1.

### Step 9. tert-Butyl (1R,4R,5S)-5-((Rₐ)-8-(2-cyanoethyl)-7-(2,3-dichlorophenyl)-6-fluoro-4-methyl-2-((1S,5R,7R)-3-((2-nitrophenyl)sulfonyl)-6-((2-(trimethylsilyl)ethoxy)carbonyl)-3,6-diazabicyclo[3.2.1]octan-7-yl)-1H-pyrrolo[3,2-c]quinolin-1-yl)-2-azabicyclo[2.1.1]hexane-2-carboxylate

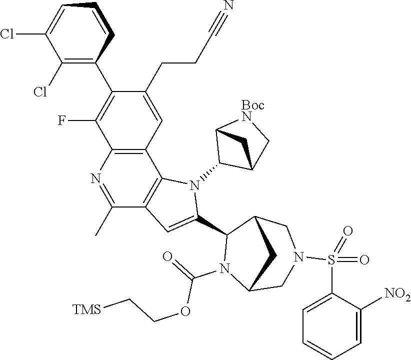

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (Intermediate 2, 4.62 g, 6.78 mmol), 2-(trimethylsilyl)ethyl (1 S,5R,7R)-7-ethynyl-3-((2-nitrophenyl)sulfonyl)-3,6-diazabicyclo[3.2.1]octane-6-carboxylate (2.87 g, 6.16 mmol), CuI (0.235 g, 1.23 mmol), Pd(PPh₃)₄ (0.71 g, 0.61 mmol) and DIPEA (10.8 mL, 61.6 mmol) in DMF (62 mL) was sparged with N₂ and heated at 90° C. for 30 minutes. Then, Cs₂CO₃ (10.0 g, 30.8 mmol) was added to the mixture. The resulting slurry was stirred at 90° C. for 16 h. The mixture was then allowed to cool to r.t. and poured into water. The solution was extracted with EtOAc (3×) and the combined organic layers were washed with brine, dried over Na₂SO₄, filtered and concentrated. The crude product was purified by flash column chromatography (0-30% MeOH/DCM) to afford the desired product (4.5 g, 72% yield). LCMS calc. for C₄₉H₅₅Cl₂FN₇O₈SSi (M+H)⁺: m/z=1018.3/1020.3; found 1018.2/1020.2.

### Step 10. tert-Butyl (1R,4R,5S)-5-((Rₐ)-8-(2-cyanoethyl)-7-(2,3-dichlorophenyl)-6-fluoro-4-methyl-2-((1S,5R,7R)-3-((2-nitrophenyl)sulfonyl)-3,6-diazabicyclo[3.2.1]octan-7-yl)-1H-pyrrolo[3,2-c]quinolin-1-yl)-2-azabicyclo[2.1.1]hexane-2-carboxylate

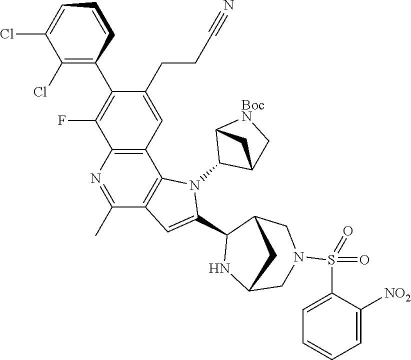

To a solution of tert-butyl (1R,4R,5S)-5-((Rₐ)-8-(2-cyanoethyl)-7-(2,3-dichlorophenyl)-6-fluoro-4-methyl-2-((1 S,5R,7R)-3-((2-nitrophenyl)sulfonyl)-6-((2-(trimethylsilyl)ethoxy)carbonyl)-3,6-diazabicyclo[3.2.1]octan-7-yl)-1H-pyrrolo[3,2-c]quinolin-1-yl)-2-azabicyclo[2.1.1]hexane-2-carboxylate (2.62 g, 2.57 mmol) in DMF (10.3 mL) was added CsF (1.56 g, 10.3 mmol). The mixture was stirred at 90° C. for 1 h. The mixture was then diluted with DCM and washed with 5% aq. LiCl solution (5×) and brine. The organic layer was dried over Na₂SO₄, filtered and concentrated. The crude product was purified by flash column chromatography (0-30% MeOH/DCM) to give the desired product (1.2 g, 53% yield). LCMS calc. for C₄₃H₄₃Cl₂FN₇O₆S (M+H)⁺: m/z=874.2/876.2; found 874.2/876.2.

### Step 11. tert-Butyl (1R,4R,5S)-5-((Rₐ)-8-(2-cyanoethyl)-7-(2,3-dichlorophenyl)-6-fluoro-2-((1S,5R,7R)-6-(methoxycarbonyl)-3-((2-nitrophenyl)sulfonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate

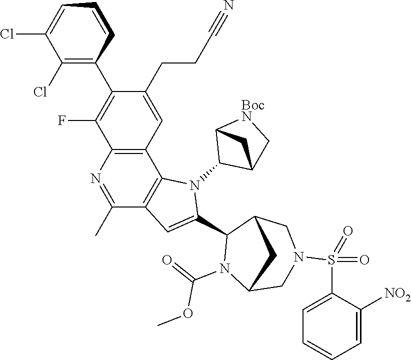

To a solution of tert-butyl (1R,4R,5S)-5-((Rₐ)-8-(2-cyanoethyl)-7-(2,3-dichlorophenyl)-6-fluoro-4-methyl-2-((1 S,5R,7R)-3-((2-nitrophenyl)sulfonyl)-3,6-diazabicyclo[3.2.1]octan-7-yl)-1H-pyrrolo[3,2-c]quinolin-1-yl)-2-azabicyclo[2.1.1]hexane-2-carboxylate (1.2 g, 1.37 mmol) in THF (27 mL) cooled to 0° C. were added DIPEA (1.20 mL, 6.86 mmol) and methyl chloroformate (0.21 mL, 2.74 mmol). The mixture was stirred at r.t. for 30 minutes and was then diluted with EtOAc. The organics were washed with water and brine, dried over Na₂SO₄, filtered and concentrated. The crude product was purified by flash column chromatography (0-100% EtOAc/hexanes) to give the desired product (1.15 g, 90% yield). LCMS calc. for C₄₅H₄₅Cl₂FN₇O₈S (M+H)⁺: m/z=932.2/934.2; found 932.0/934.2.

### Step 12. tert-Butyl (1R,4R,5S)-5-((Rₐ)-8-(2-cyanoethyl)-7-(2,3-dichlorophenyl)-6-fluoro-2-((1S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate

To a solution of tert-butyl (1R,4R,5S)-5-((Rₐ)-8-(2-cyanoethyl)-7-(2,3-dichlorophenyl)-6-fluoro-2-((1 S,5R,7R)-6-(methoxycarbonyl)-3-((2-nitrophenyl)sulfonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate (1.15 g, 1.23 mmol) in DMF (12 mL) were added benzenethiol (0.39 mL, 3.82 mmol) and potassium carbonate (0.53 g, 3.82 mmol). The mixture was stirred at r.t. for 1 h. The mixture was then diluted with DCM and washed with 5% aq. LiCl solution (5×) and brine. The organic layer was dried over Na₂SO₄, filtered and concentrated. The crude product was purified by flash column chromatography (0-40% MeOH/DCM) to give the desired product (0.60 g, 65% yield). LCMS calc. for C₃₉H₄₂Cl₂FN₆O_{4 (M+H)}⁺: m/z=747.3/749.3; found 747.3/749.3.

### Intermediate 17. tert-Butyl (1R,4R,5S)-5-((Rₐ)-8-(2-cyanoethyl)-7-(2,3-dichlorophenyl)-2-((1S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate

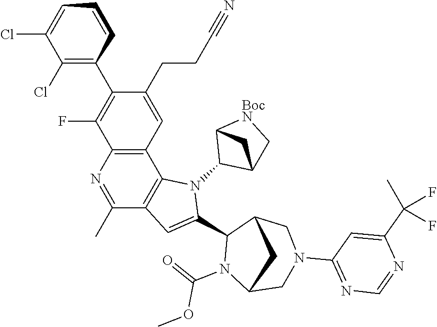

### Step 1. tert-Butyl (1R,4R,5S)-5-((Rₐ)-8-(2-cyanoethyl)-7-(2,3-dichlorophenyl)-6-fluoro-4-methyl-2-((1S,5R,7R)-6-((2-(trimethylsilyl)ethoxy)carbonyl)-3,6-diazabicyclo[3.2.1]octan-7-yl)-1H-pyrrolo[3,2-c]quinolin-1-yl)-2-azabicyclo[2.1.1]hexane-2-carboxylate

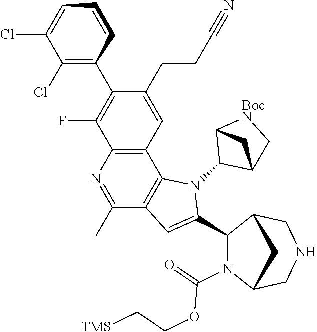

To a solution of tert-butyl (1R,4R,5S)-5-((Rₐ)-8-(2-cyanoethyl)-7-(2,3-dichlorophenyl)-6-fluoro-4-methyl-2-((1 S,5R,7R)-3-((2-nitrophenyl)sulfonyl)-6-((2-(trimethylsilyl)ethoxy)carbonyl)-3,6-diazabicyclo[3.2.1]octan-7-yl)-1H-pyrrolo[3,2-c]quinolin-1-yl)-2-azabicyclo[2.1.1]hexane-2-carboxylate (Intermediate 16, Step 9, 1.3 g, 1.28 mmol) in DMF (13 mL) were added benzenethiol (0.40 mL, 3.95 mmol) and potassium carbonate (0.55 g, 3.95 mmol). The mixture was stirred at r.t. for 1 h. The mixture was then diluted with DCM and washed with 5% aq. LiCl solution (5×) and brine. The organic layer was dried over Na₂SO₄, filtered and concentrated. The crude product was purified by flash column chromatography (0-40% MeOH/DCM) to give the desired product (0.34 g, 32% yield). LCMS calc. for C₄₃H₅₂Cl₂FN₆O₄Si (M+H)⁺: m/z=833.3/835.3; found 833.2/835.2.

### Step 2. tert-Butyl (1R,4R,5S)-5-((Rₐ)-8-(2-cyanoethyl)-7-(2,3-dichlorophenyl)-2-((1S,5R,7R)-3-(6-(1,1-difluoroethyl)pyrimidin-4-yl)-6-((2-(trimethylsilyl)ethoxy)carbonyl)-3,6-diazabicyclo[3.2.1]octan-7-yl)-6-fluoro-4-methyl-1H-pyrrolo[3,2-c]quinolin-1-yl)-2-azabicyclo[2.1. 1]hexane-2-carboxylate

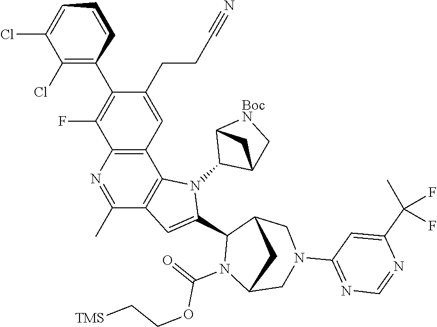

A mixture of tert-butyl (1R,4R,5S)-5-((Rₐ)-8-(2-cyanoethyl)-7-(2,3-dichlorophenyl)-6-fluoro-4-methyl-2-((1 S,5R,7R)-6-((2-(trimethylsilyl)ethoxy)carbonyl)-3,6-diazabicyclo[3.2.1]octan-7-yl)-1H-pyrrolo[3,2-c]quinolin-1-yl)-2-azabicyclo[2.1.1]hexane-2-carboxylate (0.64 g, 0.77 mmol), RuPhos Pd G4 (0.065 g, 0.077 mmol), RuPhos (0.036 g, 0.077 mmol), sodium tert-butoxide (2 M THF, 0.58 mL, 1.15 mmol) and 4-chloro-6-(1,1-difluoroethyl)pyrimidine (0.41 mL, 3.1 mmol) in 1,4-dioxane (15 mL) was sparged with N₂ and heated at 90° C. for 16 h. The mixture was then allowed to cool to r.t. and diluted with EtOAc. The organic fraction was washed with brine, dried over Na₂SO₄, filtered and concentrated. The crude product was purified by flash column chromatography (0-100% EtOAc/hexanes) to give the desired product (0.47 g, 63% yield). LCMS calc. for C₄₉H₅₆Cl₂F₃N₈O₄Si (M+H)⁺: m/z=975.3/977.3; found 975.2/977.2.

### Step 3. tert-Butyl (1R,4R,5S)-5-((Rₐ)-8-(2-cyanoethyl)-7-(2,3-dichlorophenyl)-2-((1S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate

To a solution of tert-butyl (1R,4R,5S)-5-((Rₐ)-8-(2-cyanoethyl)-7-(2,3-dichlorophenyl)-2-((1 S,5R,7R)-3-(6-(1,1-difluoroethyl)pyrimidin-4-yl)-6-((2-(trimethylsilyl)ethoxy)carbonyl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate (0.47 g, 0.49 mmol) in DMF (1.9 mL) was added CsF (0.30 g, 1.94 mmol). The mixture was stirred at 90° C. for 1 h. The mixture was then diluted with DCM and washed with 5% aq. LiCl solution (5×) and brine. The organic layer was dried over Na₂SO₄, filtered and concentrated. The crude product was taken forward without additional purification. LCMS calc. for C₄₃H₄₄Cl₂F₃N₈O₂ (M+H)⁺: m/z=831.3/833.3; found 831.2/833.2.

### Example 1. 3-((Rₐ)-1-((1R,4R,5S)-2-Azabicyclo[2.1.1]hexan-5-yl)-2-((1 S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

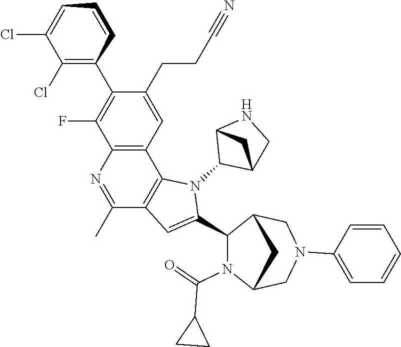

### Step 1. 6-(tert-Butyl) 7-methyl (1S,5R,7R)-3-phenyl-3,6-diazabicyclo[3.2.1]octane-6,7-dicarboxylate

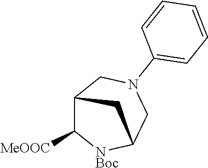 [SMILES: COC(=O)[C@H]1[C@H]2C[C@H](CN(c3ccccc3)C2)N1C(=O)OC(C)(C)C]

To a solution of 2-(tert-butyl) 3-methyl (1R,3R,4R,5R,6S)-5,6-dihydroxy-2-azabicyclo[2.2.1]heptane-2,3-dicarboxylate (intermediate 4, 0.700 g, 2.44 mmol) in 1,4-dioxane/water (4:1, 12 mL) was added 2,6-lutidine (0.522 g, 4.87 mmol) and NaIO₄ (2.08 g, 9.75 mmol). The mixture was stirred at r.t. for 1 h. The mixture was then diluted with EtOAc and washed with brine. The organic layer was dried over Na₂SO₄, filtered and concentrated under reduced pressure.

To the residue dissolved in DCM (12 mL) were added aniline (0.23 g, 2.44 mmol) and PPTS (1.23 g, 4.87 mmol). The mixture was stirred for 1 h at r.t. before NaBH(OAc)₃ (3.10 g, 14.6 mmol) was added. After stirring for 0.5 h, the reaction was quenched by slow addition of water. The mixture was extracted with DCM (2×). The combined organic layers were dried over Na₂SO₄, filtered and concentrated. The crude product was further purified with FCC (0-30% EtOAc/hexanes) to afford the sub-title compound as an oil (0.72 g, 85% yield). LCMS calc. for C₁₅H₁₉N₂O₄ (M-^{t}Bu+2H)⁺: m/z=291.1; found 291.1.

### Step 2. tert-Butyl (1S,5R,7R)-7-(hydroxymethyl)-3-phenyl-3,6-diazabicyclo[3.2.1]octane-6-carboxylate

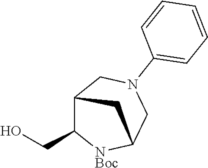 [SMILES: CC(C)(C)OC(=O)N1[C@@H]2C[C@@H](CN(c3ccccc3)C2)[C@@H]1CO]

To a solution of 6-(tert-butyl) 7-methyl (1 S,5R,7R)-3-phenyl-3,6-diazabicyclo[3.2.1]octane-6,7-dicarboxylate (0.72 g, 2.07 mmol) in THF (10.5 mL) was added LiBH₄ (2.6 mL, 2 M in THF, 5.17 mmol). The mixture was stirred at r.t. for 16 h and then quenched by slow addition of sat. aq. NH₄Cl at 0° C. The mixture was diluted with EtOAc, washed with brine, dried over Na₂SO₄, filtered and concentrated. The crude product was purified by FCC (0-60% EtOAc/hexanes) to afford the sub-title compound (0.53 g, 81% yield). LCMS calc. for C₁₄H₁₉N₂O₃ (M-^{t}Bu+2H)⁺: m/z=263.1; found 263.1.

### Step 3. tert-butyl (1S,5R,7R)-7-formyl-3-phenyl-3,6-diazabicyclo[3.2.1]octane-6-carboxylate

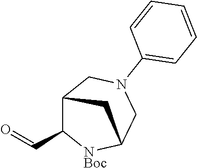 [SMILES: CC(C)(C)OC(=O)N1[C@@H]2C[C@@H](CN(c3ccccc3)C2)[C@@H]1C=O]

To a solution of tert-butyl (1 S,5R,7R)-7-(hydroxymethyl)-3-phenyl-3,6-diazabicyclo[3.2.1]octane-6-carboxylate (0.53 g, 1.67 mmol) in DCM (8.4 mL) was added Dess-Martin periodinane (1.07 g, 2.51 mmol) portionwise. The resulting mixture was stirred at r.t. for 1 h. Sat. aq. Na₂S₂O₃ (1 mL) was then added to the mixture, which was stirred for an additional 0.5 h. The organic phase was separated, dried over Na₂SO₄, filtered and concentrated to give crude product. The crude product was used in the next step without further purification. LCMS calc. for C₁₄H₁₇N₂O₃ (M-^{t}Bu+2H)⁺: m/z=261.1; found 261.1.

### Step 4. tert-Butyl (1S,5R,7R)-7-ethynyl-3-phenyl-3,6-diazabicyclo[3.2.1]octane-6-carboxylate

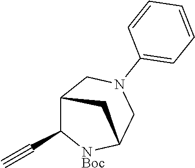 [SMILES: C#C[C@H]1[C@H]2C[C@H](CN(c3ccccc3)C2)N1C(=O)OC(C)(C)C]

To a solution of tert-butyl (1 S,5R,7R)-7-formyl-3-phenyl-3,6-diazabicyclo[3.2.1]octane-6-carboxylate (0.53 g, 1.67 mmol) in MeOH (1.8 mL) were added dimethyl(1-diazo-2-oxopropyl)phosphonate (0.48 g, 2.51 mmol) and K₂CO₃ (0.69 g, 5.02 mmol). After stirring for 16 h, the mixture was filtered through diatomaceous earth. The filtrate was concentrated. The residue was extracted with EtOAc, filtered through diatomaceous earth and concentrated. The crude product was purified by FCC (0-30% EtOAc/hexanes) to afford the sub-title compound (0.23 g, 43% yield over 2 steps). LCMS calc. for C₁₉H₂₅N₂O₂ (M+H)⁺: m/z=313.2; found 313.2

### Step 5. Cyclopropyl((1S,5R,7R)-7-ethynyl-3-phenyl-3,6-diazabicyclo[3.2.1]octan-6-yl)methanone

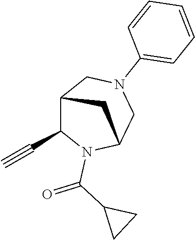 [SMILES: C#C[C@H]1[C@H]2C[C@H](CN(c3ccccc3)C2)N1C(=O)C1CC1]

To a solution of tert-butyl (1 S,5R,7R)-7-ethynyl-3-phenyl-3,6-diazabicyclo[3.2.1]octane-6-carboxylate (0.010 g, 0.033 mmol) in 1,4-dioxane (0.5 mL) was added 4 M HCl in 1,4-dioxane (0.5 mL). The mixture was stirred at r.t. for 0.5 h. The volatiles were then removed under reduced pressure. The residue was dissolved in DCM (21 mL). Upon stirring, DIPEA (0.03 mL, 0.17 mmol) was added followed by cyclopropanecarbonyl chloride (0.005 g, 0.050 mmol). The mixture was stirred for 0.5 h. The mixture was diluted with DCM, washed with water, dried over Na₂SO₄, filtered and concentrated. The crude product was used in the next step without further purification. LCMS calc. for C₁₈H₂₁N₂O (M+H)⁺: m/z=281.2; found 281.2.

### Step 6. 3-((Rₐ)-1-((1R,4R,5S)-2-Azabicyclo[2.1.1]hexan-5-yl)-2-((1S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 0.015 g, 0.022 mmol), cyclopropyl((1 S,5R,7R)-7-ethynyl-3-phenyl-3,6-diazabicyclo[3.2.1]octan-6-yl)methanone (0.009 g, 0.033 mmol), CuI (0.002 g, 0.009 mmol), Pd(PPh₃)₄ (0.005 g, 0.004 mmol) and DIPEA (0.038 mL, 0.22 mmol) in DMF (0.11 mL) was sparged with N₂ and heated at 70° C. for 0.5 h. Then, Cs₂CO₃ (0.022 g, 0.066 mmol) was added to the mixture. The resulting slurry was stirred at 90° C. for another 18 h. The mixture was then allowed to cool to r.t. and poured into water. The solution was extracted with EtOAc (2×). Then the combined organic layers were washed with brine (5×), dried over Na₂SO₄, filtered and concentrated.

To the residue dissolved in DCM (1 mL) were added MeCN (0.1 mL) and TFA (1 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₄₀Cl₂FN₆O (M+H)⁺: m/z=733.3; found 733.2.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing processes analogous to the steps above starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Example 2. 3-((Rₐ)-1-((1R,4R,5S)-2-Azabicyclo[2.1.1]hexan-5-yl)-2-((1 S,5R,7R)-6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

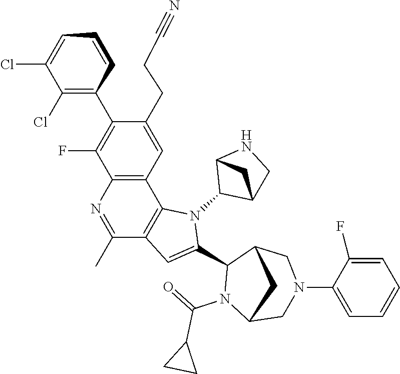

To a solution of tert-butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 6, 0.010 g, 0.013 mmol) in DCM (0.5 mL) were added 2-fluoroaniline (0.002 g, 0.016 mmol) and PPTS (0.007 g, 0.026 mmol). The mixture was stirred for 1 h at r.t. before NaBH(OAc)₃ (0.017 g, 0.078 mmol) was added. After stirring for 0.5 h, the reaction was quenched by slow addition of water. The mixture was extracted with DCM (2×). The combined organic layers were dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₃₉Cl₂F₂N₆O (M+H)⁺: m/z=751.3; found 751.3.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing processes analogous to the step above starting from tert-butyl (1R,4R,5S)-5-((7Sₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Examples 3-29

The following compounds listed in Table 4 were prepared in an analogous fashion to Example 2 using the appropriate substituted aniline and reducing agent (NaBH(OAc)₃ or sodium cyanoborohydride).

TABLE 4
Examples 3-29

| | | | (M + H)⁺ |
|---|---|---|---|
| Ex. | Structure | Name | m/z |
| 3 | 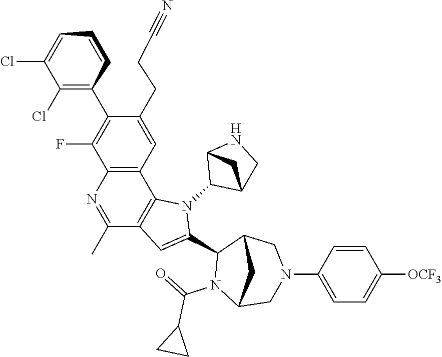 | 3-((Rₐ)-1-((1R,4R,5S)-2- azabicyclo[2.1.1]hexan-5-yl)-2- ((1S,5R,7R)-6- (cyclopropanecarbonyl)-3-(4- (trifluoromethoxy)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 817.2 |
| 4 | 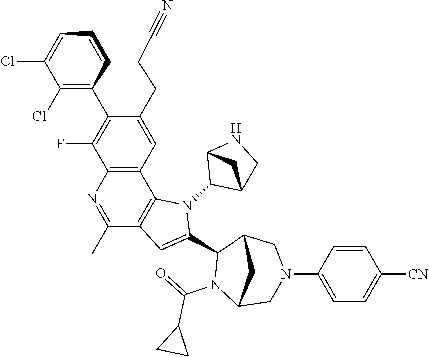 | 4-((1S,5R,7R)-7-((Rₐ)-1- ((1R,4R,5S)-2- azabicyclo[2.1.1]hexan-5-yl)-8-(2- cyanoethyl)-7-(2,3-dichlorophenyl)- 6-fluoro-4-methyl-1H-pyrrolo[3,2- c]quinolin-2-yl)-6- (cyclopropanecarbonyl)-3,6- diazabicyclo[3.2.1]octan-3- yl)benzonitrile | 758.3 |
| 5 | 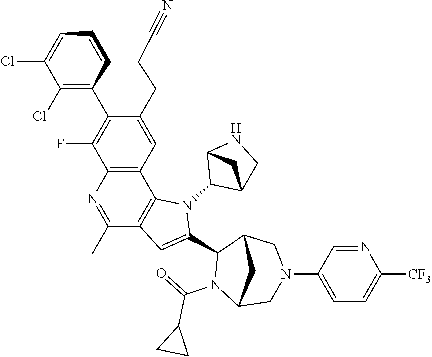 | 3-((Rₐ)-1-((1R,4R,5S)-2- azabicyclo[2.1.1]hexan-5-yl)-2- ((1S,5R,7R)-6- (cyclopropanecarbonyl)-3-(6- (trifluoromethyl)pyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 802.2 |
| 6 | 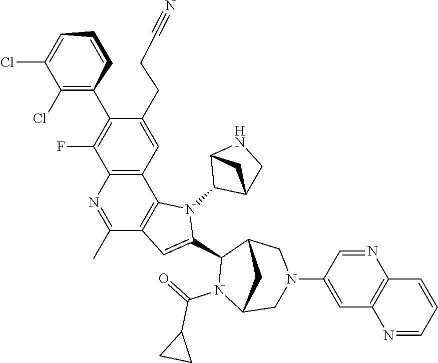 | 3-((Rₐ)-1-((1R,4R,5S)-2- azabicyclo[2.1.1]hexan-5-yl)-2- ((1S,5R,7R)-6- (cyclopropanecarbonyl)-3-(1,5- naphthyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 785.3 |
| 7 | 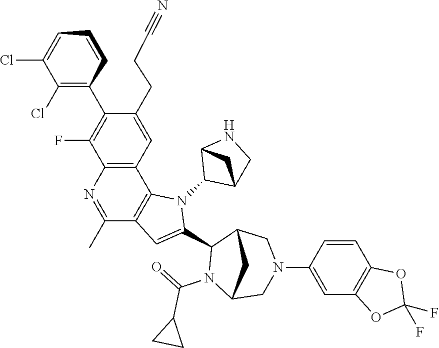 | 3-((Rₐ)-1-((1R,4R,5S)-2- azabicyclo[2.1.1]hexan-5-yl)-2- ((1S,5R,7R)-6- (cyclopropanecarbonyl)-3-(2,2- difluorobenzo[d][1,3]dioxol-5-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 813.2 |
| 8 | 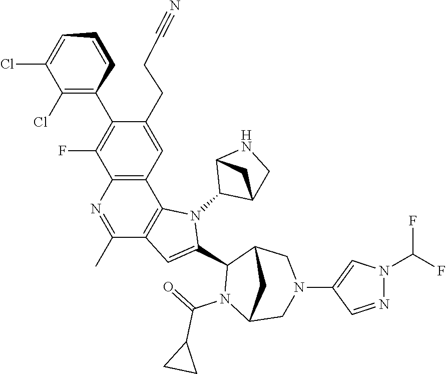 | 3-((Rₐ)-1-((1R,4R,5S)-2- azabicyclo[2.1.1]hexan-5-yl)-2- ((1S,5R,7R)-6- (cyclopropanecarbonyl)-3-(1- (difluoromethyl)-1H-pyrazol-4-yl)- 3,6-diazabicyclo[3.2.1]octan-7-yl)-7- (2,3-dichlorophenyl)-6-fluoro-4- methyl-1H-pyrrolo[3,2-c]quinolin-8- yl)propanenitrile | 773.2 |
| 9 | 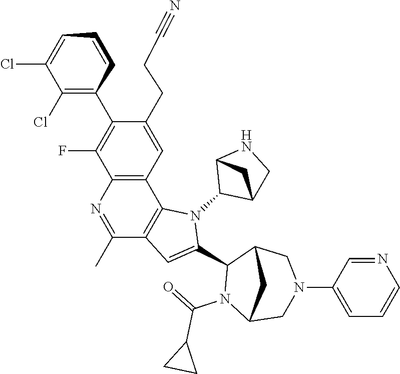 | 3-((Rₐ)-1-((1R,4R,5S)-2- azabicyclo[2.1.1]hexan-5-yl)-2- ((1S,5R,7R)-6- (cyclopropanecarbonyl)-3-(pyridin-3- yl)-3,6-diazabicyclo[3.2.1]octan-7- yl)-7-(2,3-dichlorophenyl)-6-fluoro-4- methyl-1H-pyrrolo[3,2-c]quinolin-8- yl)propanenitrile | 734.3 |
| 10 | 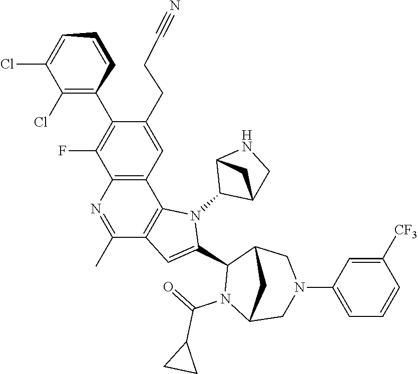 | 3-((Rₐ)-1-((1R,4R,5S)-2- azabicyclo[2.1.1]hexan-5-yl)-2- ((1S,5R,7R)-6- (cyclopropanecarbonyl)-3-(3- (trifluoromethyl)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 801.2 |
| 11 | 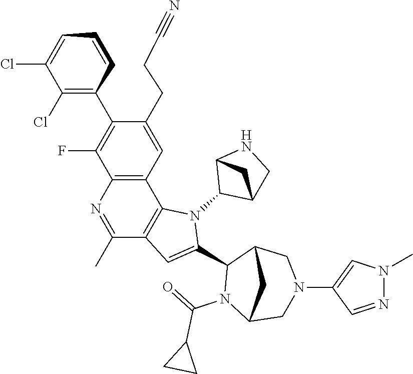 | 3-((Rₐ)-1-((1R,4R,5S)-2- azabicyclo[2.1.1]hexan-5-yl)-2- ((1S,5R,7R)-6- (cyclopropanecarbonyl)-3-(1-methyl- 1H-pyrazol-4-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 737.3 |
| 12 | 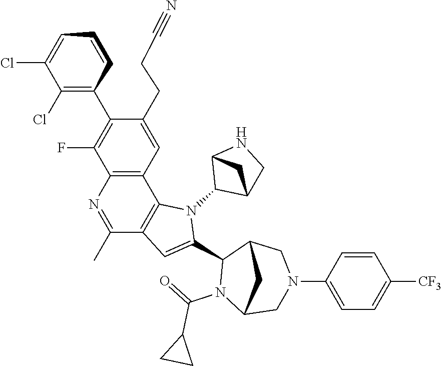 | 3-((Rₐ)-1-((1R,4R,5S)-2- azabicyclo[2.1.1]hexan-5-yl)-2- ((1S,5R,7R)-6- (cyclopropanecarbonyl)-3-(4- (trifluoromethyl)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 801.2 |
| 13 | 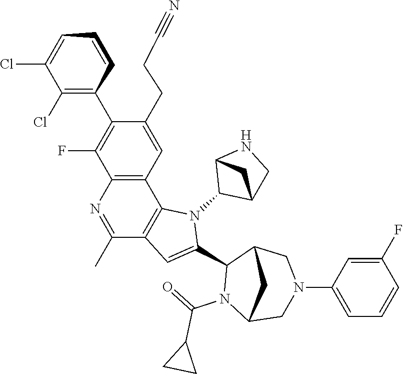 | 3-((Rₐ)-1-((1R,4R,5S)-2- azabicyclo[2.1.1]hexan-5-yl)-2- ((1S,5R,7R)-6- (cyclopropanecarbonyl)-3-(3- fluorophenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 751.3 |
| 14 | 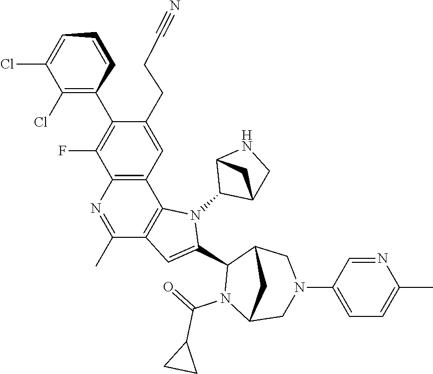 | 3-((Rₐ)-1-((1R,4R,5S)-2- azabicyclo[2.1.1]hexan-5-yl)-2- ((1S,5R,7R)-6- (cyclopropanecarbonyl)-3-(6- methylpyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 748.3 |
| 15 | 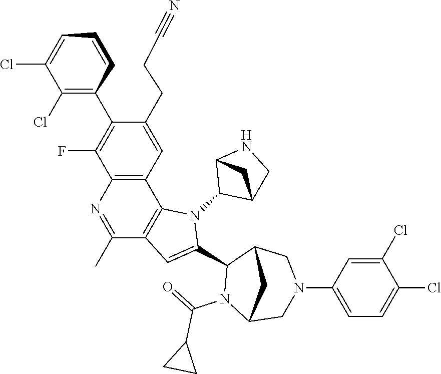 | 3-((Rₐ)-1-((1R,4R,5S)-2- azabicyclo[2.1.1]hexan-5-yl)-2- ((1S,5R,7R)-6- (cyclopropanecarbonyl)-3-(3,4- dichlorophenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 801.2 |
| 16 | 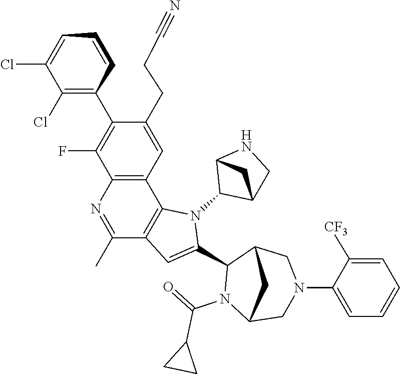 | 3-((Rₐ)-1-((1R,4R,5S)-2- azabicyclo[2.1.1]hexan-5-yl)-2- ((1S,5R,7R)-6- (cyclopropanecarbonyl)-3-(2- (trifluoromethyl)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 801.2 |
| 17 | 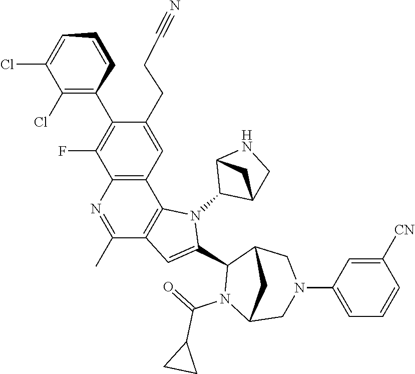 | 3-((1S,5R,7R)-7-((Rₐ)-1- ((1R,4R,5S)-2- azabicyclo[2.1.1]hexan-5-yl)-8-(2- cyanoethyl)-7-(2,3-dichlorophenyl)- 6-fluoro-4-methyl-1H-pyrrolo[3,2- c]quinolin-2-yl)-6- (cyclopropanecarbonyl)-3,6- diazabicyclo[3.2.1]octan-3- yl)benzonitrile | 758.3 |
| 18 | 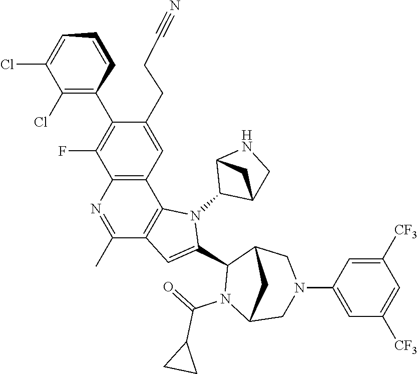 | 3-((Rₐ)-1-((1R,4R,5S)-2- azabicyclo[2.1.1]hexan-5-yl)-2- ((1S,5R,7R)-3-(3,5- bis(trifluoromethyl)phenyl)-6- (cyclopropanecarbonyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 869.2 |
| 19 | 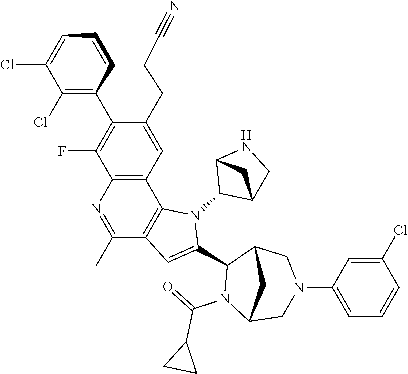 | 3-((Rₐ)-1-((1R,4R,5S)-2- azabicyclo[2.1.1]hexan-5-yl)-2- ((1S,5R,7R)-3-(3-chlorophenyl)-6- (cyclopropanecarbonyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 767.2 |
| 20 | 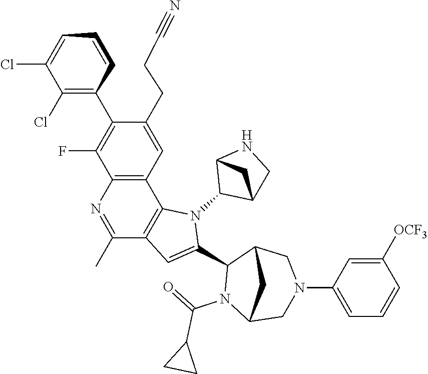 | 3-((Rₐ)-1-((1R,4R,5S)-2- azabicyclo[2.1.1]hexan-5-yl)-2- ((1S,5R,7R)-6- (cyclopropanecarbonyl)-3-(3- (trifluoromethoxy)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 817.2 |
| 21 | 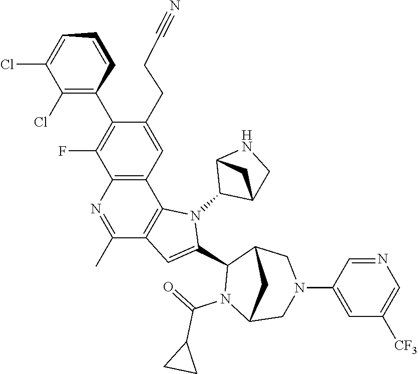 | 3-((Rₐ)-1-((1R,4R,5S)-2- azabicyclo[2.1.1]hexan-5-yl)-2- ((1S,5R,7R)-6- (cyclopropanecarbonyl)-3-(5- (trifluoromethyl)pyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 802.2 |
| 22 | 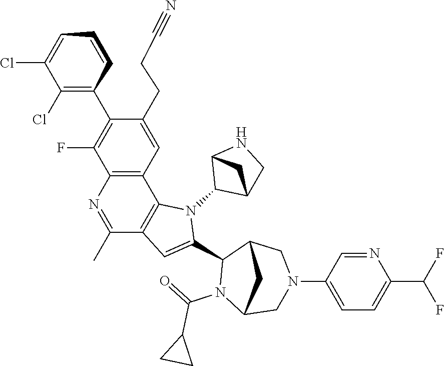 | 3-((Rₐ)-1-((1R,4R,5S)-2- azabicyclo[2.1.1]hexan-5-yl)-2- ((1S,5R,7R)-6- (cyclopropanecarbonyl)-3-(6- (difluoromethyl)pyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 784.3 |
| 23 | 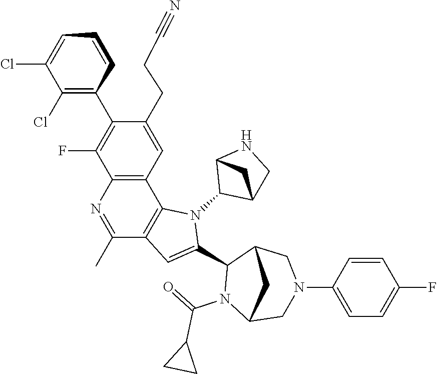 | 3-((Rₐ)-1-((1R,4R,5S)-2- azabicyclo[2.1.1]hexan-5-yl)-2- ((1S,5R,7R)-6- (cyclopropanecarbonyl)-3-(4- fluorophenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 751.3 |
| 24 | 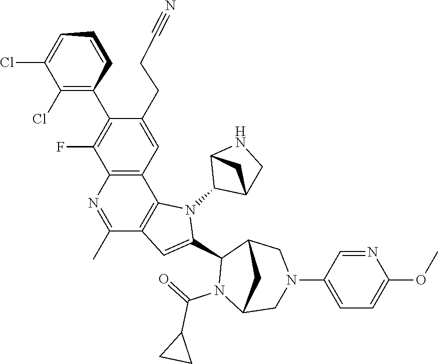 | 3-((Rₐ)-1-((1R,4R,5S)-2- azabicyclo[2.1.1]hexan-5-yl)-2- ((1S,5R,7R)-6- (cyclopropanecarbonyl)-3-(6- methoxypyridin-3-yl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 764.3 |
| 25 | 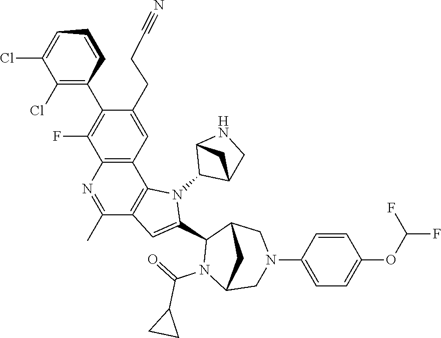 | 3-((Rₐ)-1-((1R,4R,5S)-2- azabicyclo[2.1.1]hexan-5-yl)-2- ((1S,5R,7R)-6- (cyclopropanecarbonyl)-3-(4- (difluoromethoxy)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 799.3 |
| 26 | 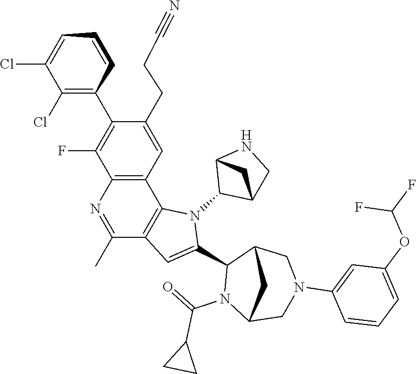 | 3-((Rₐ)-1-((1R,4R,5S)-2- azabicyclo[2.1.1]hexan-5-yl)-2- ((1S,5R,7R)-6- (cyclopropanecarbonyl)-3-(3- (difluoromethoxy)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 799.3 |
| 27 | 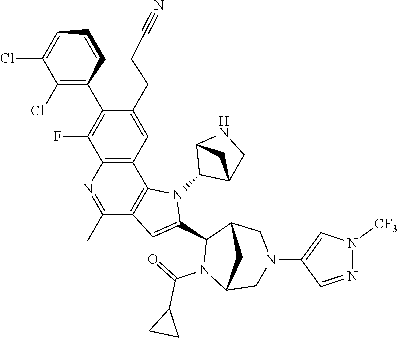 | 3-((Rₐ)-1-((1R,4R,5S)-2- azabicyclo[2.1.1]hexan-5-yl)-2- ((1S,5R,7R)-6- (cyclopropanecarbonyl)-3-(1- (trifluoromethyl)-1H-pyrazol-4-yl)- 3,6-diazabicyclo[3.2.1]octan-7-yl)-7- (2,3-dichlorophenyl)-6-fluoro-4- methyl-1H-pyrrolo[3,2-c]quinolin-8- yl)propanenitrile | 791.2 |
| 28 | 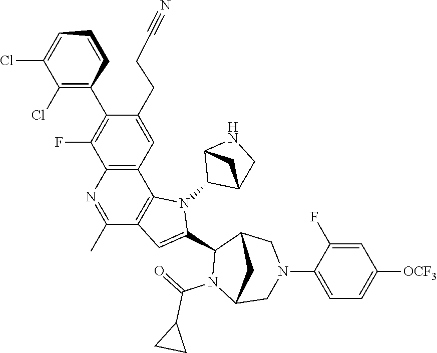 | 3-((Rₐ)-1-((1R,4R,5S)-2- azabicyclo[2.1.1]hexan-5-yl)-2- ((1S,5R,7R)-6- (cyclopropanecarbonyl)-3-(2-fluoro- 4-(trifluoromethoxy)phenyl)-3,6- diazabicyclo[3.2.1]octan-7-yl)-7- (2,3-dichlorophenyl)-6-fluoro-4- methyl-1H-pyrrolo[3,2-c]quinolin-8- yl)propanenitrile | 835.2 |
| 29 | 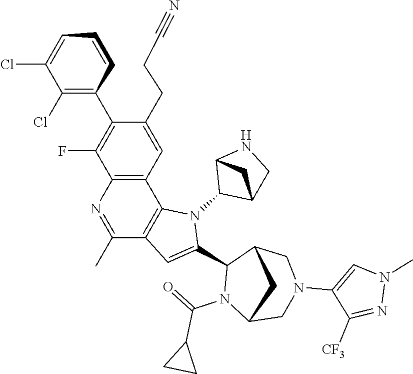 | 3-((Rₐ)-1-((1R,4R,5S)-2- azabicyclo[2.1.1]hexan-5-yl)-2- ((1S,5R,7R)-6- (cyclopropanecarbonyl)-3-(1-methyl- 3-(trifluoromethyl)-1H-pyrazol-4-yl)- 3,6-diazabicyclo[3.2.1]octan-7-yl)-7- (2,3-dichlorophenyl)-6-fluoro-4- methyl-1H-pyrrolo[3,2-c]quinolin-8- yl)propanenitrile | 805.3 |

The alternative atropisomers of the compounds of Table 4 can be prepared by an analogous route by performing processes analogous to Example 2 starting from tert-butyl (1R,4R,5S)-5-((7Sₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 30. 3-((Rₐ)-1-((1R,4R,5S)-2-Azabicyclo[2.1.1]hexan-5-yl)-2-((1 S,5R,7R)-6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

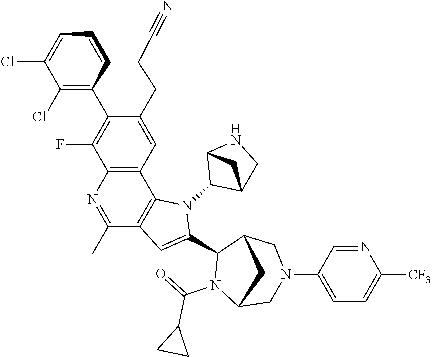

To a solution of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 0.014 g, 0.018 mmol) in MeCN (0.6 mL) were added 2-fluoro-5-(trifluoromethyl)pyridine (0.015 g, 0.092 mmol) and Cs₂CO₃ (0.030 g, 0.092 mmol). The mixture was stirred at 60° C. for 8 h. The mixture was then diluted with EtOAc and washed with brine. The organic layer was dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₃₈Cl₂F₄N₇O (M+H)⁺: m/z=802.2; found 802.2.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 31. 3-((Rₐ)-1-((1R,4R,5S)-2-Azabicyclo[2.1.1]hexan-5-yl)-2-((1 S,5R,7R)-6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

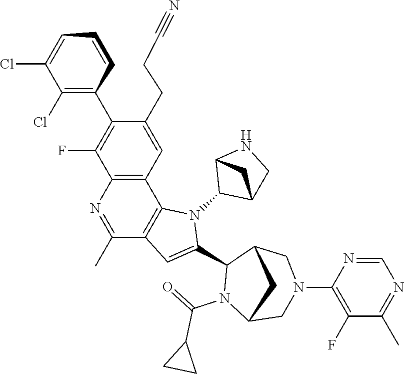

A mixture of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 0.085 g, 0.112 mmol), RuPhos Pd G4 (0.014 g, 0.017 mmol), RuPhos (0.008 g, 0.017 mmol), sodium tert-butoxide (0.013 g, 0.140 mmol) and 4-chloro-5-fluoro-6-methylpyrimidine (0.099 g, 0.673 mmol) in THF (0.6 mL) was sparged with N₂ and heated at 85° C. for 16 h. The mixture was then allowed to cool to r.t. and diluted with EtOAc. The organic fraction was washed with brine, dried over Na₂SO₄, filtered and concentrated.

To the residue dissolved in DCM (2 mL) were added MeCN (0.2 mL) and TFA (2 mL). The mixture was stirred at r.t. for 0.5 h. The volatiles were then removed under reduced pressure and the residue was dissolved in MeCN (12 mL) and water (3 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₁H₃₉Cl₂F₂N₈O (M+H)⁺: m/z=767.3; found 767.2. ¹H NMR was collected on the TFA salt. ¹H NMR (600 MHz, DMSO-d₆) δ 9.37 (br, 1H), 8.38 (d, J=2.3 Hz, 1H), 8.16 (s, 1H), 8.09 (br, 1H), 7.86 (dd, J=8.1, 1.5 Hz, 1H), 7.59 (t, J=7.9 Hz, 1H), 7.47 (dd, J=7.6, 1.6 Hz, 1H), 6.87 (s, 1H), 5.65 (m, 1H), 5.12 (s, 1H), 4.93-4.79 (m, 3H), 4.51 (d, J=13.1 Hz, 1H), 4.08 (m, 1H), 3.84 (m, 1H), 3.48 (d, J=13.0 Hz, 1H), 3.41 (m, 1H), 3.34 (d, J=12.9 Hz, 1H), 3.12-3.04 (m, 1H), 2.90-2.80 (m, 5H), 2.74-2.64 (m, 1H), 2.47 (s, 1H), 2.42-2.38 (m, 1H), 2.36 (d, J=3.4 Hz, 3H), 2.16 (ddd, J=12.7, 7.5, 4.9 Hz, 1H), 2.10-2.05 (m, 1H), 1.92 (d, J=11.4 Hz, 1H), 1.64 (d, J=9.2 Hz, 1H), 0.89 (m, 2H), 0.83 (m, 1H), 0.35 (m, 1H).

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 32. 3-((Rₐ)-1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

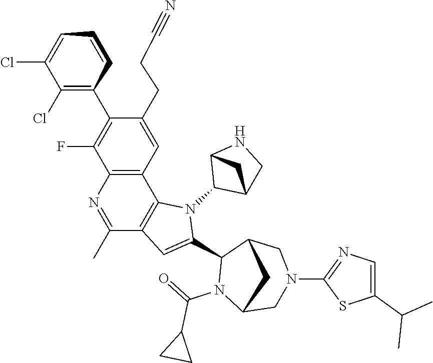

To a 8 mL scintillation vial were added tert-butyl (1R,4R,5S)-5-((Rₐ)-8-(2-cyanoethyl)-2-((1S,5R,7S)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (20 mg, 0.026 mmol) and sodium trimethylsilanolate (14.80 mg, 0.132 mmol).

The vial was evacuated and back-filled with nitrogen (3×). Anhydrous THF (132 μL) was added and the vial was set to stir at r.t. for 5 min. or until complete homogeneity. 2-Bromo-5-isopropylthiazole (6.53 mg, 0.032 mmol) was added followed by GPhos Pd G6 TES (1.246 mg, 1.320 μmol) and the mixture was immediately moved to stir at 90° C. for 3 h. The mixture was then allowed to cool to r.t. and diluted with EtOAc. The organic fraction was washed with brine, dried over Na₂SO₄, filtered and concentrated.

To the residue dissolved in DCM (2 mL) were added MeCN (0.2 mL) and TFA (2 mL). The mixture was stirred at r.t. for 0.5 h. The volatiles were then removed under reduced pressure and the residue was dissolved in MeCN (12 mL) and water (3 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₄₃Cl₂FN₇OS (M+H)⁺: m/z=782.4; found 782.4. ¹H NMR was collected on the TFA salt. ¹H NMR (500 MHz, DMSO-d₆) δ 9.38 (s, 1H), 8.16 (s, 1H), 8.08 (s, 1H), 7.86 (dd, J=8.1, 1.5 Hz, 1H), 7.59 (t, J=7.9 Hz, 1H), 7.47 (dd, J=7.7, 1.5 Hz, 1H), 7.01 (d, J=1.1 Hz, 1H), 6.88 (s, 1H), 5.65-5.60 (m, 1H), 5.02 (s, 1H), 4.93 (t, J=4.6 Hz, 1H), 4.86 (d, J=6.0 Hz, 1H), 4.06 (dd, J=40.2, 11.7 Hz, 2H), 3.86 (s, 1H), 3.81 (p, J=3.1 Hz, 1H), 3.45-3.36 (m, 2H), 3.31 (d, J=11.7 Hz, 1H), 3.07 (tdd, J=8.2, 5.4, 2.5 Hz, 2H), 2.85 (td, J=11.3, 6.6 Hz, 2H), 2.74-2.63 (m, 1H), 2.54 (d, J=4.3 Hz, 1H), 2.39-2.33 (m, 1H), 2.23 (td, J=7.6, 3.9 Hz, 1H), 2.12-2.04 (m, 1H), 1.85 (d, J=11.6 Hz, 1H), 1.58 (d, J=9.2 Hz, 1H), 1.25 (d, J=6.8 Hz, 6H), 1.01-0.92 (m, 2H), 0.89 (q, J=7.4, 6.9 Hz, 1H), 0.69 (qd, J=8.0, 7.3, 3.7 Hz, 1H).

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7S)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7S)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Examples 33-106

The compounds in Table 5 were prepared in an analogous fashion to Example 31 (Examples 33-89) or 32 (Examples 90-106) using the appropriate coupling partner.

TABLE 5
Examples 33-106

| | | | (M + H)⁺ |
|---|---|---|---|
| Ex. | Structure | Name | m/z |
| 33 | 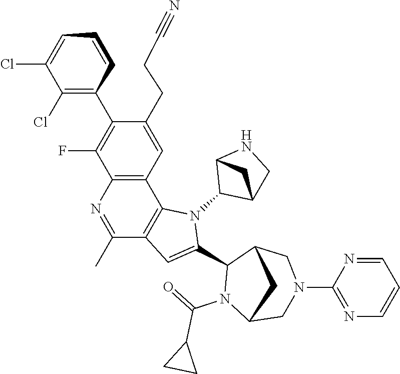 | 3-((Rₐ)-1-((1R,4R,5S)-2- azabicyclo[2.1.1]hexan-5-yl)-2- ((1S,5R,7R)-6-(cyclopropanecarbonyl)-3- (pyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 735.3 |
| 34 | 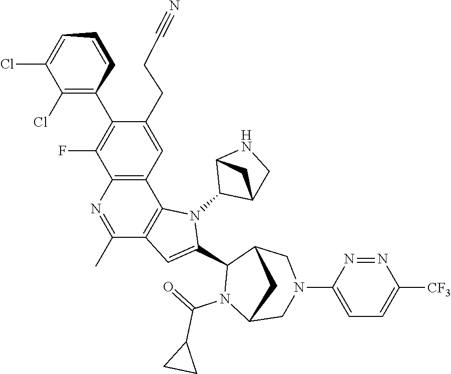 | 3-((Rₐ)-1-((1R,4R,5S)-2- azabicyclo[2.1.1]hexan-5-yl)-2- ((1S,5R,7R)-6-(cyclopropanecarbonyl)-3- (6-(trifluoromethyl)pyridazin-3-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 803.2 |
| 35 | 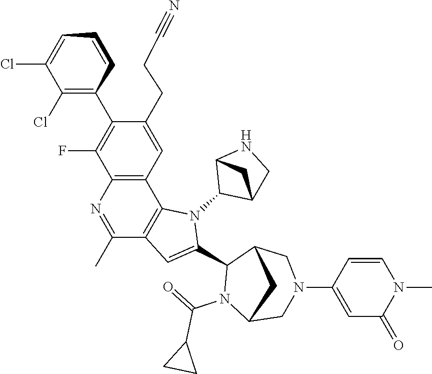 | 3-((Rₐ)-1-((1R,4R,5S)-2- azabicyclo[2.1.1]hexan-5-yl)-2- ((1S,5R,7R)-6-(cyclopropanecarbonyl)-3- (1-methyl-2-oxo-1,2-dihydropyridin-4-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 764.3 |
| 36 | 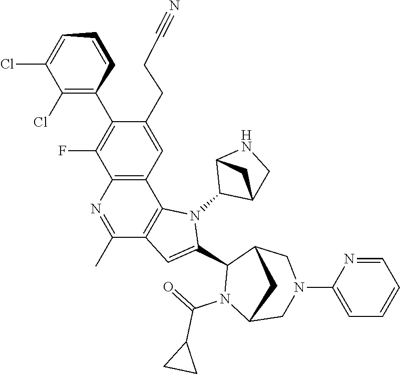 | 3-((Rₐ)-1-((1R,4R,5S)-2- azabicyclo[2.1.1]hexan-5-yl)-2- ((1S,5R,7R)-6-(cyclopropanecarbonyl)-3- (pyridin-2-yl)-3,6-diazabicyclo[3.2.1]octan- 7-yl)-7-(2,3-dichlorophenyl)-6-fluoro-4- methyl-1H-pyrrolo[3,2-c]quinolin-8- yl)propanenitrile | 734.3 |
| 37 | 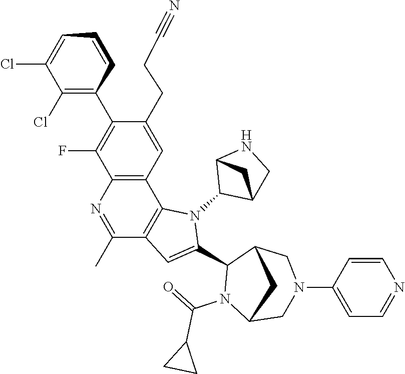 | 3-((Rₐ)-1-((1R,4R,5S)-2- azabicyclo[2.1.1]hexan-5-yl)-2- ((1S,5R,7R)-6-(cyclopropanecarbonyl)-3- (pyridin-4-yl)-3,6-diazabicyclo[3.2.1]octan- 7-yl)-7-(2,3-dichlorophenyl)-6-fluoro-4- methyl-1H-pyrrolo[3,2-c]quinolin-8- yl)propanenitrile | 734.3 |
| 38 | 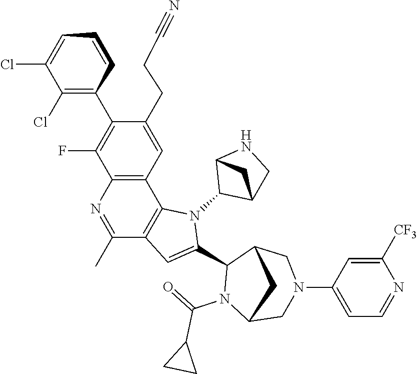 | 3-((Rₐ)-1-((1R,4R,5S)-2- azabicyclo[2.1.1]hexan-5-yl)-2- ((1S,5R,7R)-6-(cyclopropanecarbonyl)-3- (2-(trifluoromethyl)pyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 802.2 |
| 39 | 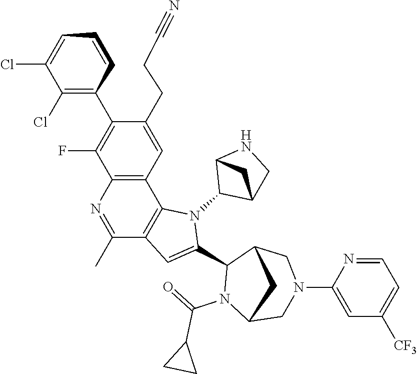 | 3-((Rₐ)-1-((1R,4R,5S)-2- azabicyclo[2.1.1]hexan-5-yl)-2- ((1S,5R,7R)-6-(cyclopropanecarbonyl)-3- (4-(trifluoromethyl)pyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 802.2 |
| 40 | 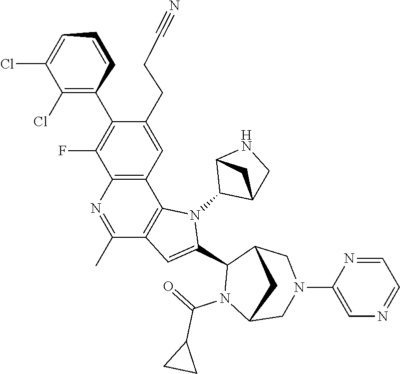 | 3-((Rₐ)-1-((1R,4R,5S)-2- azabicyclo[2.1.1]hexan-5-yl)-2- ((1S,5R,7R)-6-(cyclopropanecarbonyl)-3- (pyrazin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 735.3 |
| 41 | 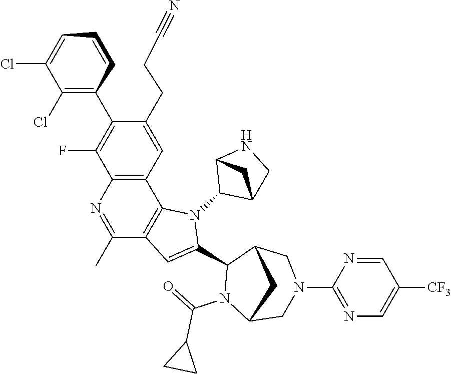 | 3-((Rₐ)-1-((1R,4R,5S)-2- azabicyclo[2.1.1]hexan-5-yl)-2- ((1S,5R,7R)-6-(cyclopropanecarbonyl)-3- (5-(trifluoromethyl)pyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 803.2 |
| 42 | 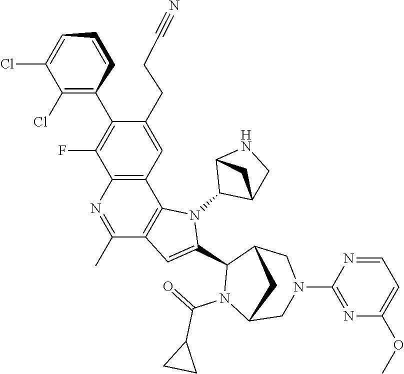 | 3-((Rₐ)-1-((1R,4R,5S)-2- azabicyclo[2.1.1]hexan-5-yl)-2- ((1S,5R,7R)-6-(cyclopropanecarbonyl)-3- (4-methoxypyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 765.3 |
| 43 | 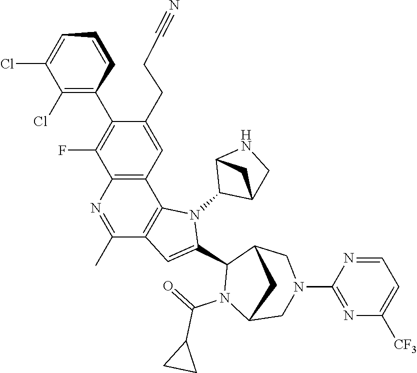 | 3-((Rₐ)-1-((1R,4R,5S)-2- azabicyclo[2.1.1]hexan-5-yl)-2- ((1S,5R,7R)-6-(cyclopropanecarbonyl)-3- (4-(trifluoromethyl)pyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 803.2 |
| 44 | 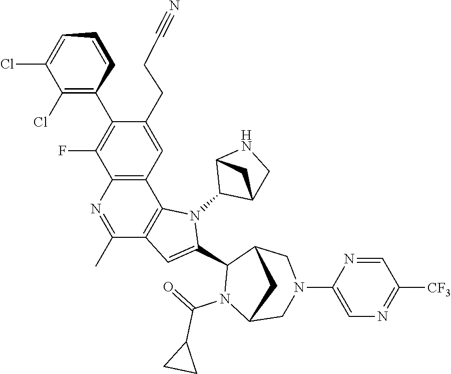 | 3-((Rₐ)-1-((1R,4R,5S)-2- azabicyclo[2.1.1]hexan-5-yl)-2- ((1S,5R,7R)-6-(cyclopropanecarbonyl)-3- (5-(trifluoromethyl)pyrazin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 803.2 |
| 45 | 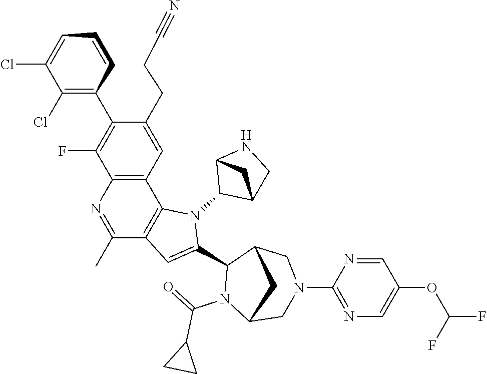 | 3-((Rₐ)-1-((1R,4R,5S)-2- azabicyclo[2.1.1]hexan-5-yl)-2- ((1S,5R,7R)-6-(cyclopropanecarbonyl)-3- (5-(difluoromethoxy)pyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 801.2 |
| 46 | 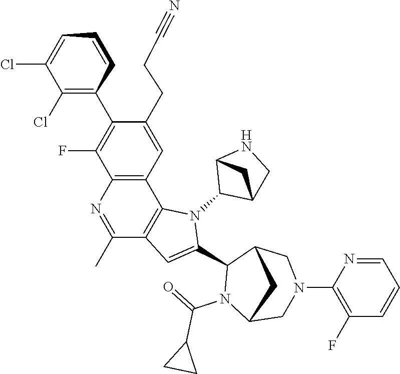 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoropyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 752.2 |
| 47 | 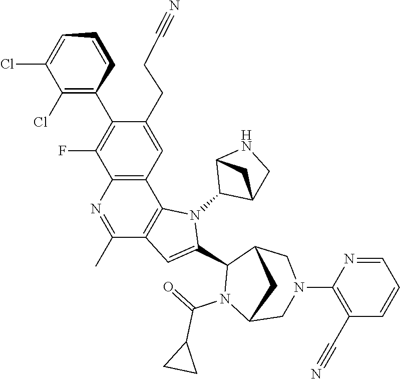 | 2-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3-yl)nicotinonitrile | 759.3 |
| 48 | 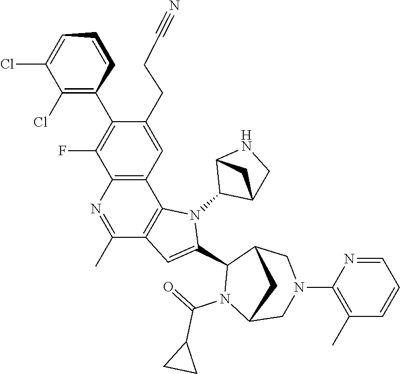 | 3-((Rₐ)-1-((1R,4R,5S)-2- azabicyclo[2.1.1]hexan-5-yl)-2- ((1S,5R,7R)-6-(cyclopropanecarbonyl)-3- (3-methylpyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 748.3 |
| 49 | 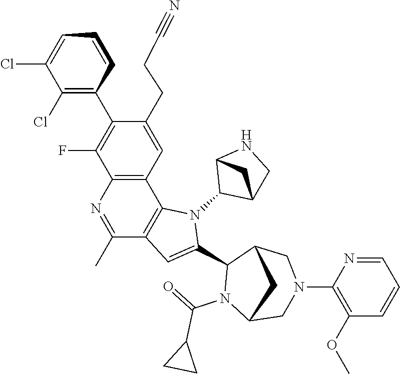 | 3-((Rₐ)-1-((1R,4R,5S)-2- azabicyclo[2.1.1]hexan-5-yl)-2- ((1S,5R,7R)-6-(cyclopropanecarbonyl)-3- (3-methoxypyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 764.3 |
| 50 | 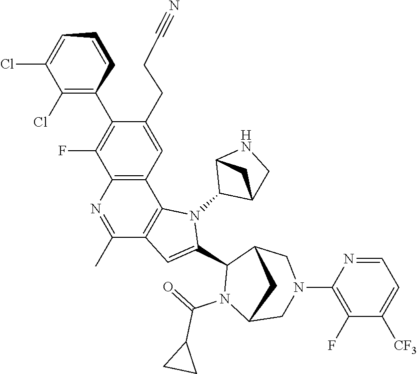 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoro-4-(trifluoromethyl)pyridin-2-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 820.2 |
| 51 | 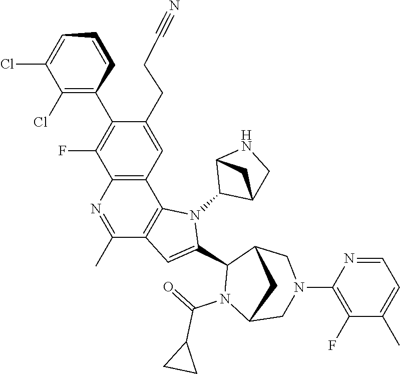 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoro-4-methylpyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 52 | 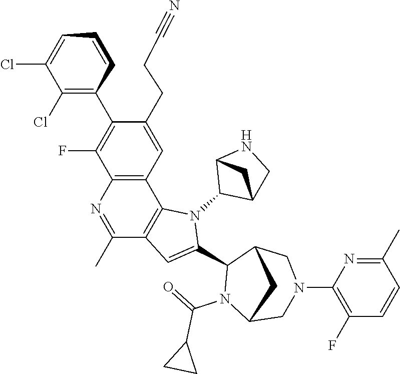 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoro-6-methylpyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 53 | 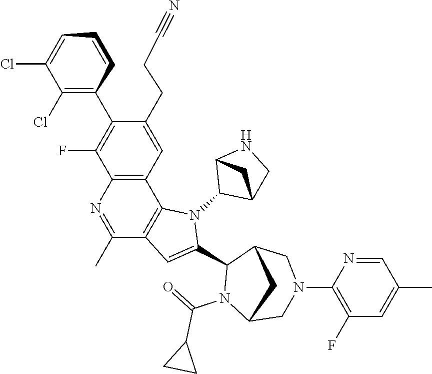 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoro-5-methylpyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 54 | 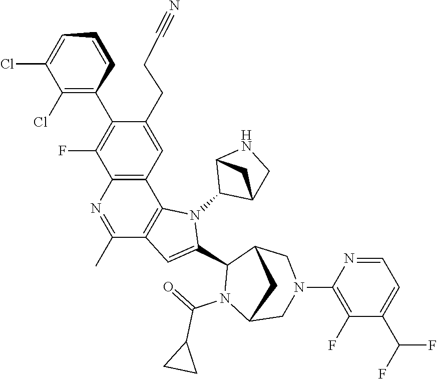 | 3-((Rₐ)-1-((1R,4R,5S)-2- azabicyclo[2.1.1]hexan-5-yl)-2- ((1S,5R,7R)-6-(cyclopropanecarbonyl)-3- (4-(difluoromethyl)-3-fluoropyridin-2-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 802.2 |
| 55 | 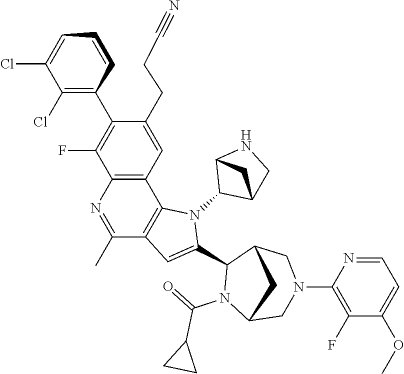 | 3-((Rₐ)-1-((1R,4R,5S)-2- azabicyclo[2.1.1]hexan-5-yl)-2- ((1S,5R,7R)-6-(cyclopropanecarbonyl)-3- (3-fluoro-4-methoxypyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 782.3 |
| 56 | 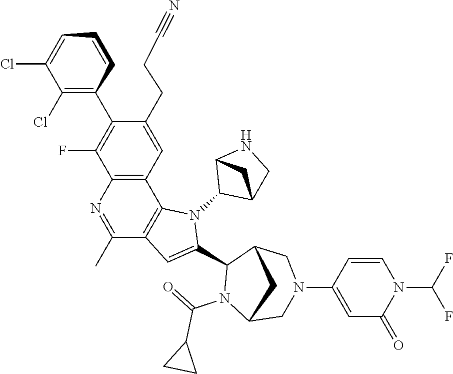 | 3-((Rₐ)-1-((1R,4R,5S)-2- azabicyclo[2.1.1]hexan-5-yl)-2- ((1S,5R,7R)-6-(cyclopropanecarbonyl)-3- (1-(difluoromethyl)-2-oxo-1,2- dihydropyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 800.2 |
| 57 | 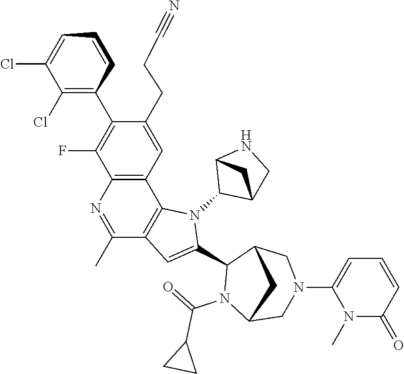 | 3-((Rₐ)-1-((1R,4R,5S)-2- azabicyclo[2.1.1]hexan-5-yl)-2- ((1S,5R,7R)-6-(cyclopropanecarbonyl)-3- (1-methyl-6-oxo-1,6-dihydropyridin-2-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 764.3 |
| 58 | 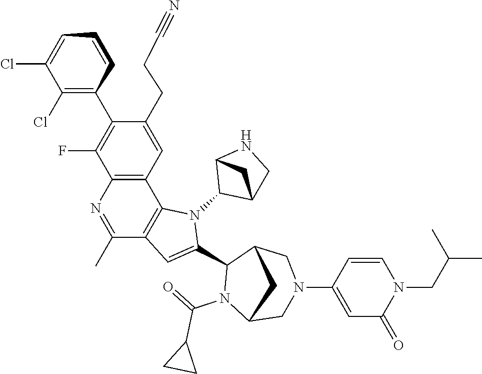 | 3-((Rₐ)-1-((1R,4R,5S)-2- azabicyclo[2.1.1]hexan-5-yl)-2- ((1S,5R,7R)-6-(cyclopropanecarbonyl)-3- (1-isobutyl-2-oxo-1,2-dihydropyridin-4-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 806.3 |
| 59 | 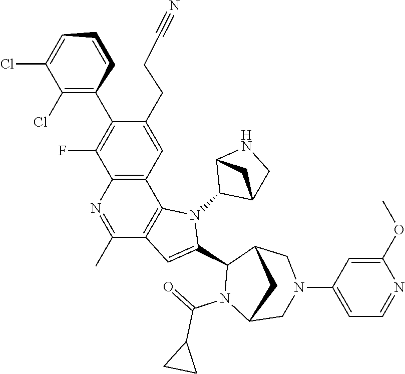 | 3-((Rₐ)-1-((1R,4R,5S)-2- azabicyclo[2.1.1]hexan-5-yl)-2- ((1S,5R,7R)-6-(cyclopropanecarbonyl)-3- (2-methoxypyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 764.3 |
| 60 | 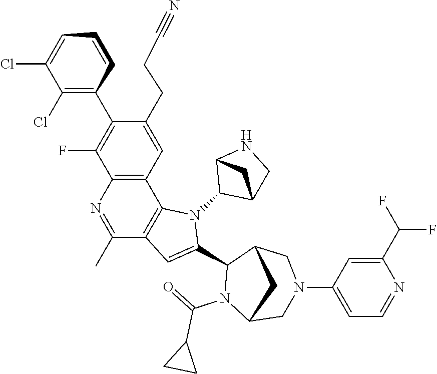 | 3-((Rₐ)-1-((1R,4R,5S)-2- azabicyclo[2.1.1]hexan-5-yl)-2- ((1S,5R,7R)-6-(cyclopropanecarbonyl)-3- (2-(difluoromethyl)pyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 784.3 |
| 61 | 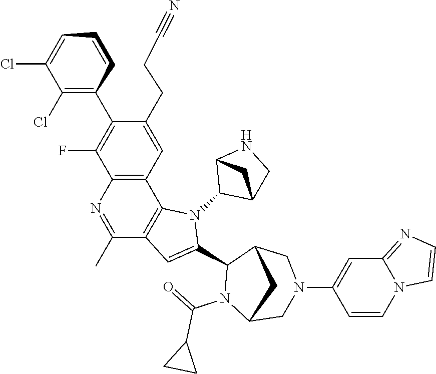 | 3-((Rₐ)-1-((1R,4R,5S)-2- azabicyclo[2.1.1]hexan-5-yl)-2- ((1S,5R,7R)-6-(cyclopropanecarbonyl)-3- (imidazo[1,2-a]pyridin-7-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 773.3 |
| 62 | 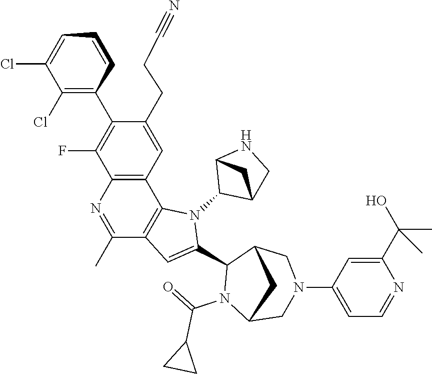 | 3-((Rₐ)-1-((1R,4R,5S)-2- azabicyclo[2.1.1]hexan-5-yl)-2- ((1S,5R,7R)-6-(cyclopropanecarbonyl)-3- (2-(2-hydroxypropan-2-yl)pyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 792.3 |
| 63 | 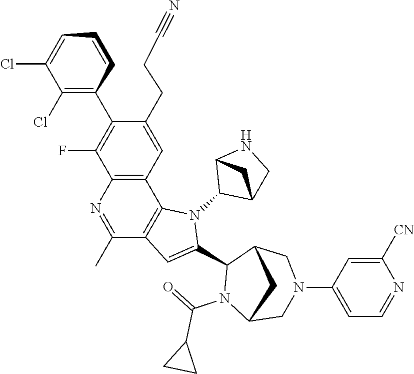 | 4-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3- yl)picolinonitrile | 759.3 |
| 64 | 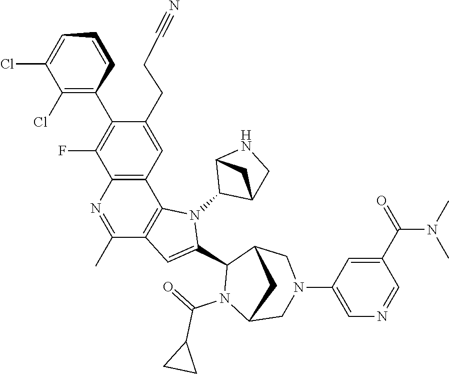 | 4-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3-yl)-N,N- dimethylpicolinamide | 805.3 |
| 65 | 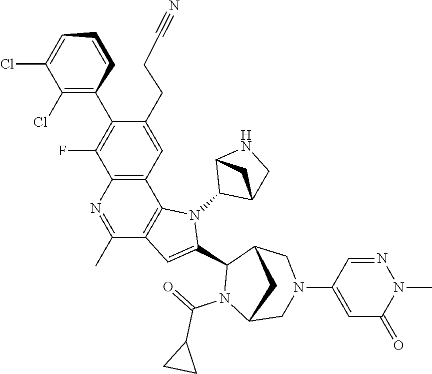 | 3-((Rₐ)-1-((1R,4R,5S)-2- azabicyclo[2.1.1]hexan-5-yl)-2- ((1S,5R,7R)-6-(cyclopropanecarbonyl)-3- (1-methyl-6-oxo-1,6-dihydropyridazin-4- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 765.3 |
| 66 | 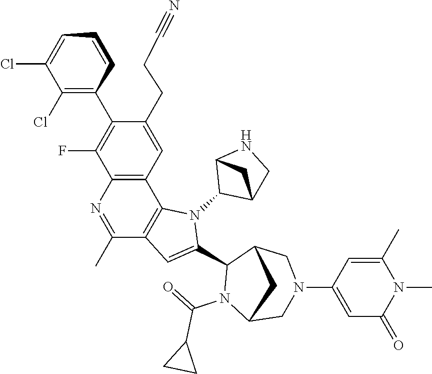 | 3-((Rₐ)-1-((1R,4R,5S)-2- azabicyclo[2.1.1]hexan-5-yl)-2- ((1S,5R,7R)-6-(cyclopropanecarbonyl)-3- (1,6-dimethyl-2-oxo-1,2-dihydropyridin-4- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 778.3 |
| 67 | 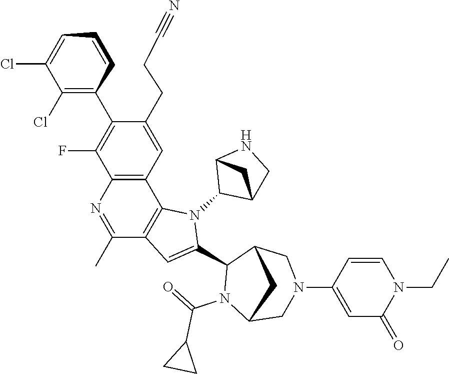 | 3-((Rₐ)-1-((1R,4R,5S)-2- azabicyclo[2.1.1]hexan-5-yl)-2- ((1S,5R,7R)-6-(cyclopropanecarbonyl)-3- (1-ethyl-2-oxo-1,2-dihydropyridin-4-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 778.3 |
| 68 | 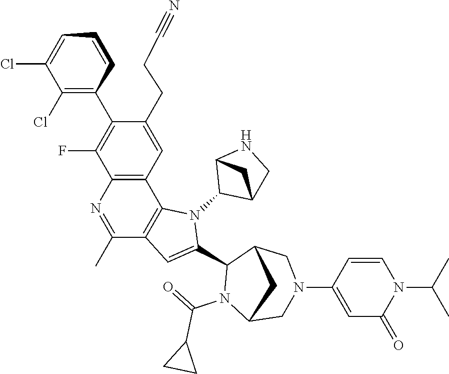 | 3-((Rₐ)-1-((1R,4R,5S)-2- azabicyclo[2.1.1]hexan-5-yl)-2- ((1S,5R,7R)-6-(cyclopropanecarbonyl)-3- (1-isopropyl-2-oxo-1,2-dihydropyridin-4- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 792.3 |
| 69 | 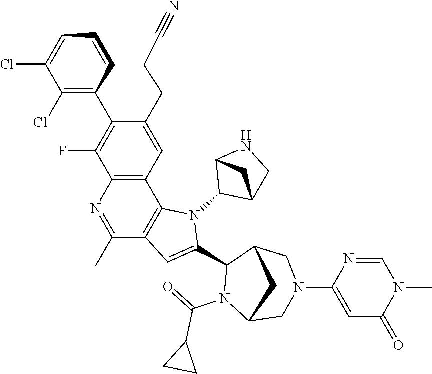 | 3-((Rₐ)-1-((1R,4R,5S)-2- azabicyclo[2.1.1]hexan-5-yl)-2- ((1S,5R,7R)-6-(cyclopropanecarbonyl)-3- (1-methyl-6-oxo-1,6-dihydropyrimidin-4- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 765.3 |
| 70 | 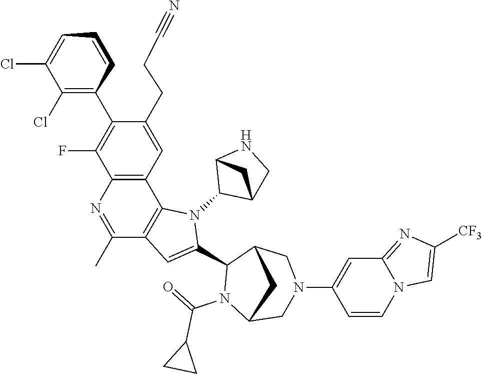 | 3-((Rₐ)-1-((1R,4R,5S)-2- azabicyclo[2.1.1]hexan-5-yl)-2- ((1S,5R,7R)-6-(cyclopropanecarbonyl)-3- (2-(trifluoromethyl)imidazo[1,2-a]pyridin-7- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 841.3 |
| 71 | 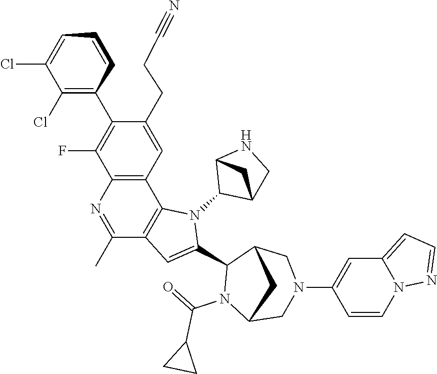 | 3-((Rₐ)-1-((1R,4R,5S)-2- azabicyclo[2.1.1]hexan-5-yl)-2- ((1S,5R,7R)-6-(cyclopropanecarbonyl)-3- (pyrazolo[1,5-a]pyridin-5-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 773.3 |
| 72 | 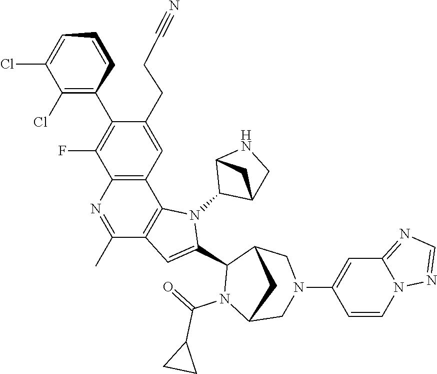 | 3-((Rₐ)-2-((1S,5R,7R)-3- ([1,2,4]triazolo[1,5-a]pyridin-7-yl)-6- (cyclopropanecarbonyl)-3,6- diazabicyclo[3.2.1]octan-7-yl)-1- ((1R,4R,5S)-2-azabicyclo[2.1.1]hexan-5- yl)-7-(2,3-dichlorophenyl)-6-fluoro-4- methyl-1H-pyrrolo[3,2-c]quinolin-8-yl) propanenitrile | 774.3 |
| 73 | 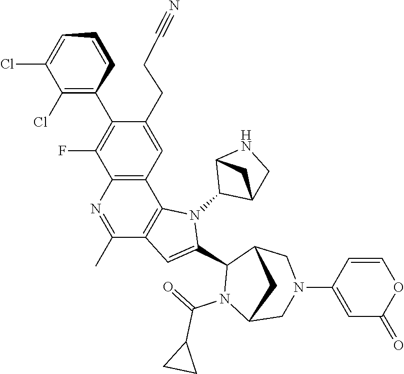 | 3-((Rₐ)-1-((1R,4R,5S)-2- azabicyclo[2.1.1]hexan-5-yl)-2- ((1S,5R,7R)-6-(cyclopropanecarbonyl)-3- (2-oxo-2H-pyran-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 751.2 |
| 74 | 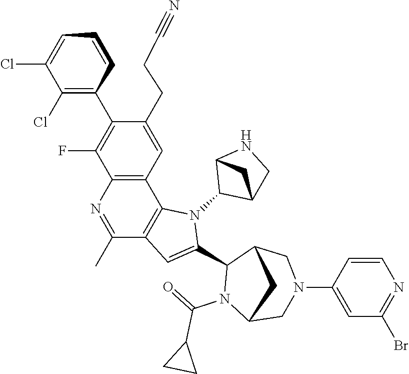 | 3-((Rₐ)-1-((1R,4R,5S)-2- azabicyclo[2.1.1]hexan-5-yl)-2- ((1S,5R,7R)-3-(2-bromopyridin-4-yl)-6- (cyclopropanecarbonyl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 812.2 |
| 75 | 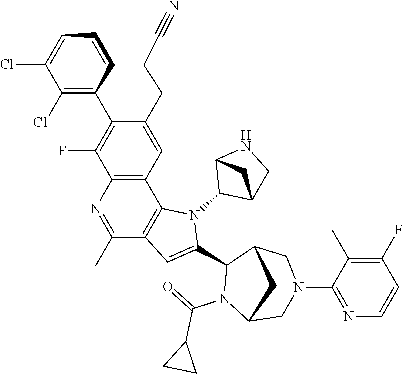 | 3-((Rₐ)-1-((1R,4R,5S)-2- azabicyclo[2.1.1]hexan-5-yl)-2- ((1S,5R,7R)-6-(cyclopropanecarbonyl)-3- (4-fluoro-3-methylpyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 76 | 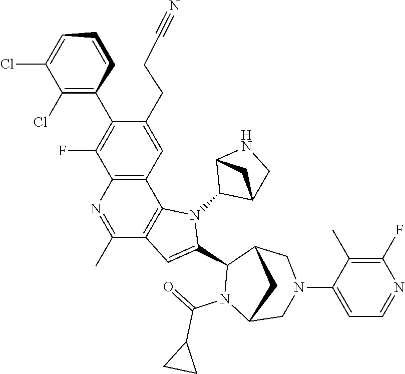 | 3-((Rₐ)-1-((1R,4R,5S)-2- azabicyclo[2.1.1]hexan-5-yl)-2- ((1S,5R,7R)-6-(cyclopropanecarbonyl)-3- (2-fluoro-3-methylpyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 77 | 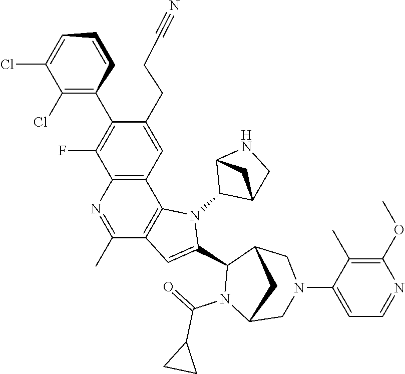 | 3-((Rₐ)-1-((1R,4R,5S)-2- azabicyclo[2.1.1]hexan-5-yl)-2- ((1S,5R,7R)-6-(cyclopropanecarbonyl)-3- (2-methoxy-3-methylpyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 778.3 |
| 78 | 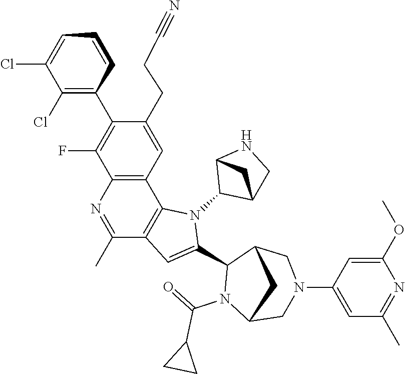 | 3-((Rₐ)-1-((1R,4R,5S)-2- azabicyclo[2.1.1]hexan-5-yl)-2- ((1S,5R,7R)-6-(cyclopropanecarbonyl)-3- (2-methoxy-6-methylpyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 778.3 |
| 79 | 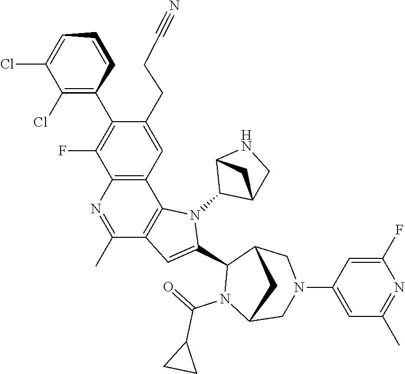 | 3-((Rₐ)-1-((1R,4R,5S)-2- azabicyclo[2.1.1]hexan-5-yl)-2- ((1S,5R,7R)-6-(cyclopropanecarbonyl)-3- (2-fluoro-6-methylpyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 766.3 |
| 80 | 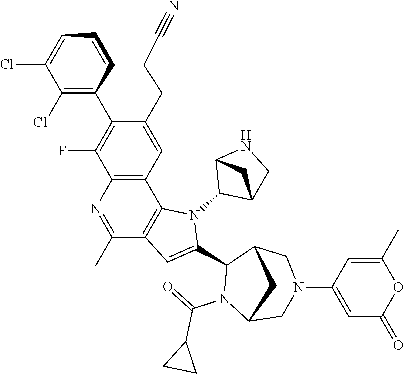 | 3-((Rₐ)-1-((1R,4R,5S)-2- azabicyclo[2.1.1]hexan-5-yl)-2- ((1S,5R,7R)-6-(cyclopropanecarbonyl)-3- (2-fluoro-6-methylpyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 765.2 |
| 81 | 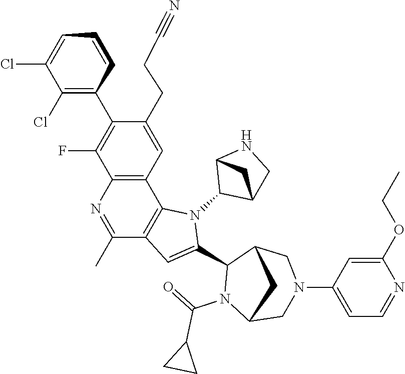 | 3-((Rₐ)-1-((1R,4R,5S)-2- azabicyclo[2.1.1]hexan-5-yl)-2- ((1S,5R,7R)-6-(cyclopropanecarbonyl)-3- (2-ethoxypyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 778.3 |
| 82 | 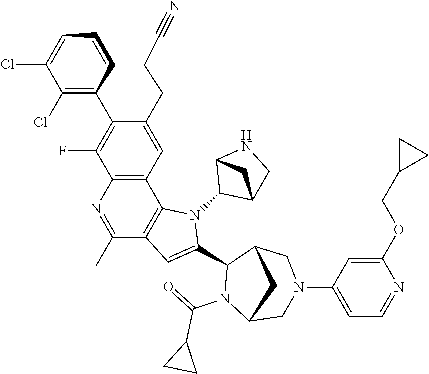 | 3-((Rₐ)-1-((1R,4R,5S)-2- azabicyclo[2.1.1]hexan-5-yl)-2- ((1S,5R,7R)-6-(cyclopropanecarbonyl)-3- (2-(cyclopropylmethoxy)pyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 804.3 |
| 83 | 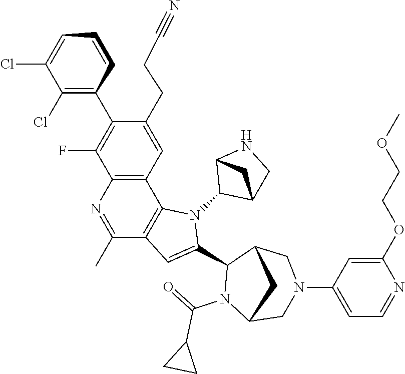 | 3-((Rₐ)-1-((1R,4R,5S)-2- azabicyclo[2.1.1]hexan-5-yl)-2- ((1S,5R,7R)-6-(cyclopropanecarbonyl)-3- (2-(2-methoxyethoxy)pyridin-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 808.3 |
| 84 | 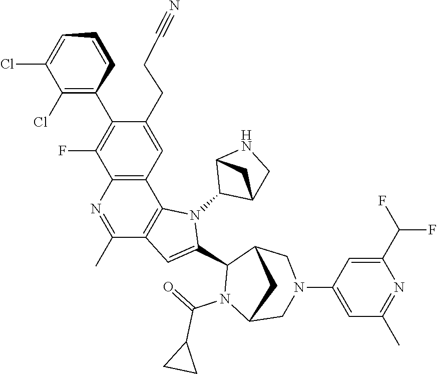 | 3-((Rₐ)-1-((1R,4R,5S)-2- azabicyclo[2.1.1]hexan-5-yl)-2- ((1S,5R,7R)-6-(cyclopropanecarbonyl)-3- (2-(difluoromethyl)-6-methylpyridin-4-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 798.3 |
| 85 | 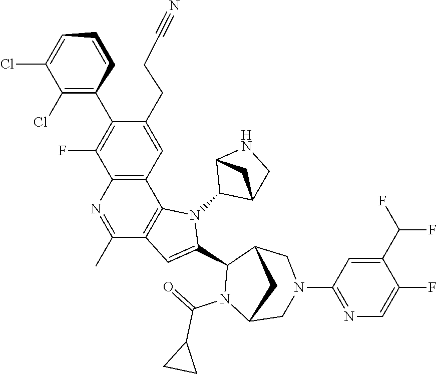 | 3-((Rₐ)-1-((1R,4R,5S)-2- azabicyclo[2.1.1]hexan-5-yl)-2- ((1S,5R,7R)-6-(cyclopropanecarbonyl)-3- (4-(difluoromethyl)-5-fluoropyridin-2-yl)- 3,6-diazabicyclo[3.2.1]octan-7-yl)-7-(2,3- dichlorophenyl)-6-fluoro-4-methyl-1H- pyrrolo[3,2-c]quinolin-8-yl)propanenitrile | 802.2 |
| 86 | 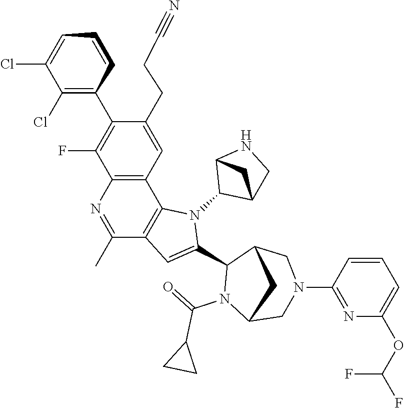 | 3-((Rₐ)-1-((1R,4R,5S)-2- azabicyclo[2.1.1]hexan-5-yl)-2- ((1S,5R,7R)-6-(cyclopropanecarbonyl)-3- (6-(difluoromethoxy)pyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 800.2 |
| 87 | 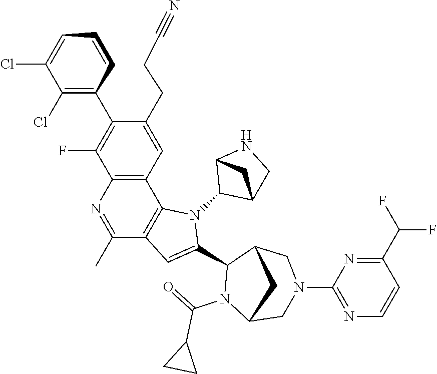 | 3-((Rₐ)-1-((1R,4R,5S)-2- azabicyclo[2.1.1]hexan-5-yl)-2- ((1S,5R,7R)-6-(cyclopropanecarbonyl)-3- (4-(difluoromethyl)pyrimidin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 785.2 |
| 88 | 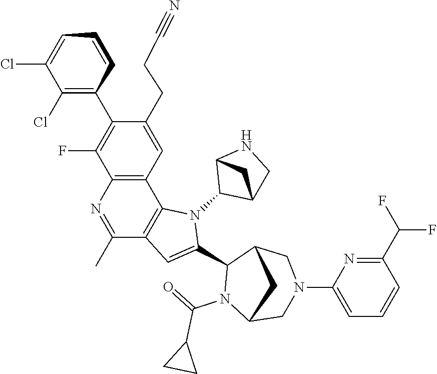 | 3-((Rₐ)-1-((1R,4R,5S)-2- azabicyclo[2.1.1]hexan-5-yl)-2- ((1S,5R,7R)-6-(cyclopropanecarbonyl)-3- (6-(difluoromethyl)pyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 784.3 |
| 89 | 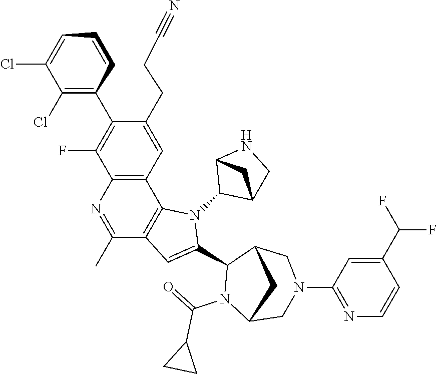 | 3-((Rₐ)-1-((1R,4R,5S)-2- azabicyclo[2.1.1]hexan-5-yl)-2- ((1S,5R,7R)-6-(cyclopropanecarbonyl)-3- (4-(difluoromethyl)pyridin-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 784.3 |
| 90 | 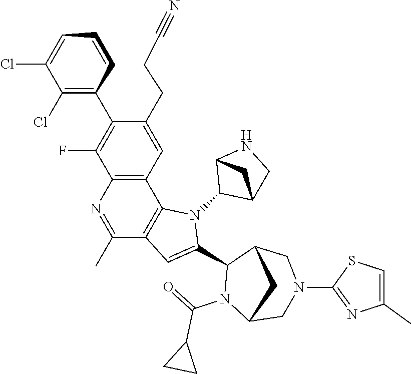 | 3-((Rₐ)-1-((1R,4R,5S)-2- azabicyclo[2.1.1]hexan-5-yl)-2- ((1S,5R,7R)-6-(cyclopropanecarbonyl)-3- (4-methylthiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 754.2 |
| 91 | 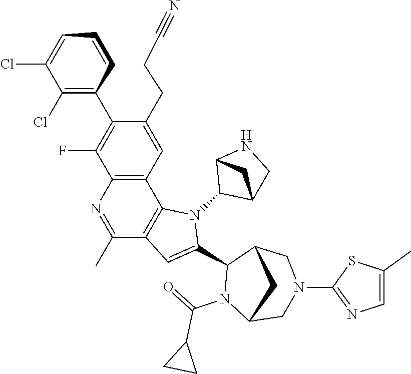 | 3-((Rₐ)-1-((1R,4R,5S)-2- azabicyclo[2.1.1]hexan-5-yl)-2- ((1S,5R,7R)-6-(cyclopropanecarbonyl)-3- (5-methylthiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 754.2 |
| 92 | 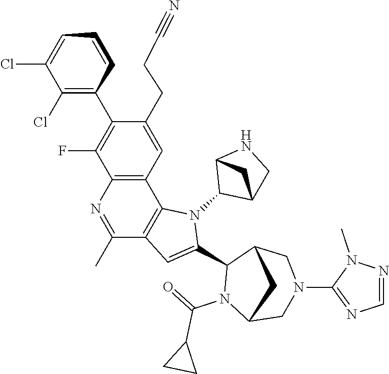 | 3-((Rₐ)-1-((1R,4R,5S)-2- azabicyclo[2.1.1]hexan-5-yl)-2- ((1S,5R,7R)-6-(cyclopropanecarbonyl)-3- (1-methyl-1H-1,2,4-triazol-5-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 738.3 |
| 93 | 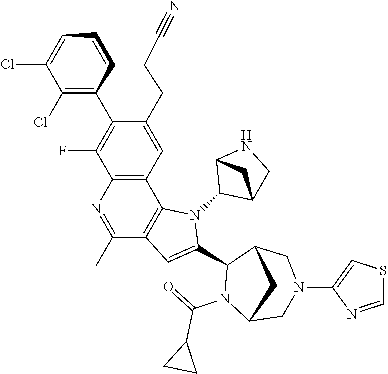 | 3-((Rₐ)-1-((1R,4R,5S)-2- azabicyclo[2.1.1]hexan-5-yl)-2- ((1S,5R,7R)-6-(cyclopropanecarbonyl)-3- (thiazol-4-yl)-3,6-diazabicyclo[3.2.1]octan- 7-yl)-7-(2,3-dichlorophenyl)-6-fluoro-4- methyl-1H-pyrrolo[3,2-c]quinolin-8- yl)propanenitrile | 740.2 |
| 94 | 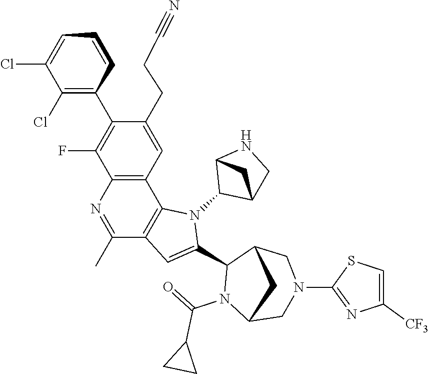 | 3-((Rₐ)-1-((1R,4R,5S)-2- azabicyclo[2.1.1]hexan-5-yl)-2- ((1S,5R,7R)-6-(cyclopropanecarbonyl)-3- (4-(trifluoromethyl)thiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 808.2 |
| 95 | 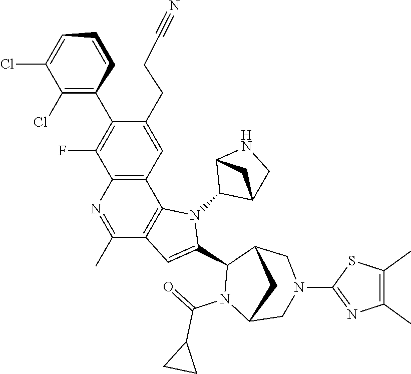 | 3-((Rₐ)-1-((1R,4R,5S)-2- azabicyclo[2.1.1]hexan-5-yl)-2- ((1S,5R,7R)-6-(cyclopropanecarbonyl)-3- (4,5-dimethylthiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 768.2 |
| 96 | 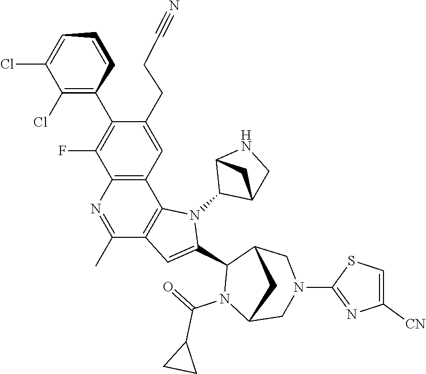 | 2-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3-yl)thiazole-4- carbonitrile | 765.2 |
| 97 | 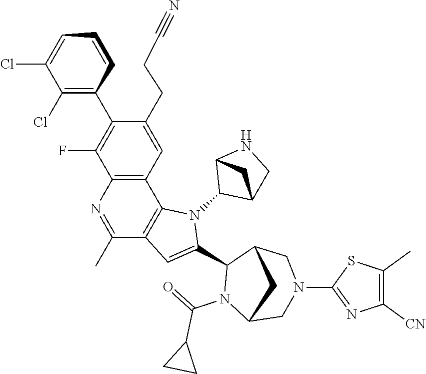 | 2-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3-yl)-5- methylthiazole-4-carbonitrile | 779.2 |
| 98 | 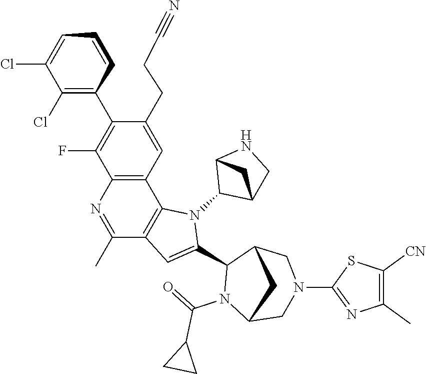 | 2-((1S,5R,7R)-7-((Rₐ)-1-((1R,4R,5S)-2- azabicyclo[2.1.1]hexan-5-yl)-8-(2- cyanoethyl)-7-(2,3-dichlorophenyl)-6- fluoro-4-methyl-1H-pyrrolo[3,2-c]quinolin- 2-yl)-6-(cyclopropanecarbonyl)-3,6- diazabicyclo[3.2.1]octan-3-yl)-4- methylthiazole-5-carbonitrile | 779.2 |
| 99 | 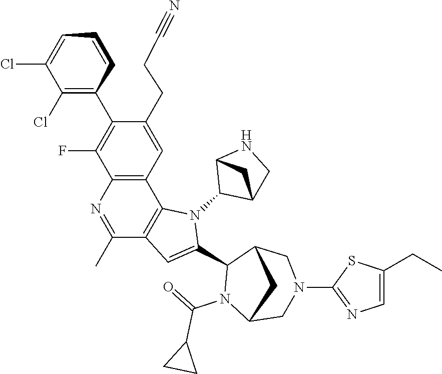 | 3-((Rₐ)-1-((1R,4R,5S)-2- azabicyclo[2.1.1]hexan-5-yl)-2- ((1S,5R,7R)-6-(cyclopropanecarbonyl)-3- (5-ethylthiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 768.2 |
| 100 | 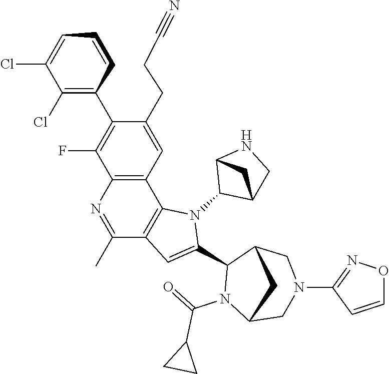 | 3-((Rₐ)-1-((1R,4R,5S)-2- azabicyclo[2.1.1]hexan-5-yl)-2- ((1S,5R,7R)-6-(cyclopropanecarbonyl)-3- (isoxazol-3-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 724.2 |
| 101 | 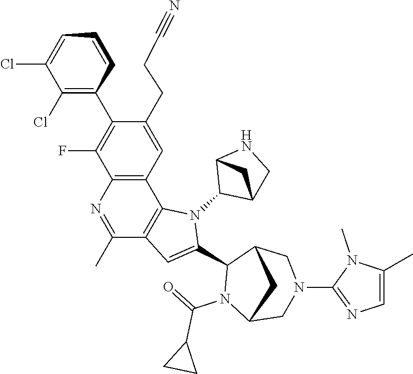 | 3-((Rₐ)-1-((1R,4R,5S)-2- azabicyclo[2.1.1]hexan-5-yl)-2- ((1S,5R,7R)-6-(cyclopropanecarbonyl)-3- (1,5-dimethyl-1H-imidazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 751.3 |
| 102 | 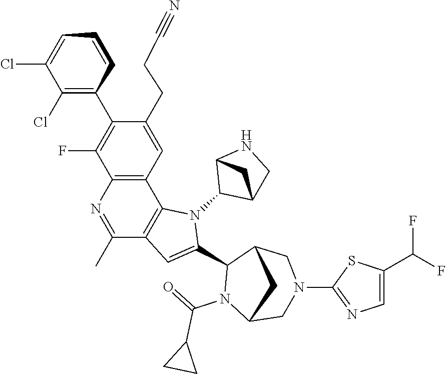 | 3-((Rₐ)-1-((1R,4R,5S)-2- azabicyclo[2.1.1]hexan-5-yl)-2- ((1S,5R,7R)-6-(cyclopropanecarbonyl)-3- (5-(difluoromethyl)thiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 790.2 |
| 103 | 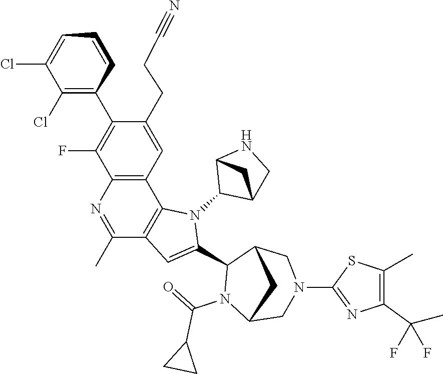 | 3-((Rₐ)-1-((1R,4R,5S)-2- azabicyclo[2.1.1]hexan-5-yl)-2- ((1S,5R,7R)-6-(cyclopropanecarbonyl)-3- (4-(1,1-difluoroethyl)-5-methylthiazol-2- yl)-3,6-diazabicyclo[3.2.1]octan-7-yl)-7- (2,3-dichlorophenyl)-6-fluoro-4-methyl- 1H-pyrrolo[3,2-c]quinolin-8-yl)propanenitrile | 818.2 |
| 104 | 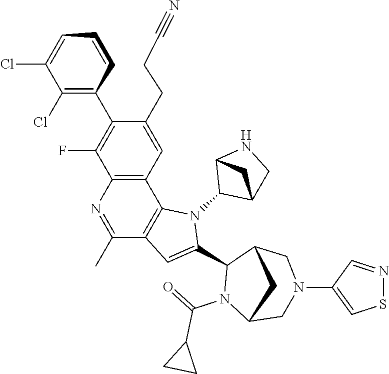 | 3-((Rₐ)-1-((1R,4R,5S)-2- azabicyclo[2.1.1]hexan-5-yl)-2- ((1S,5R,7R)-6-(cyclopropanecarbonyl)-3- (isothiazol-4-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 740.2 |
| 105 | 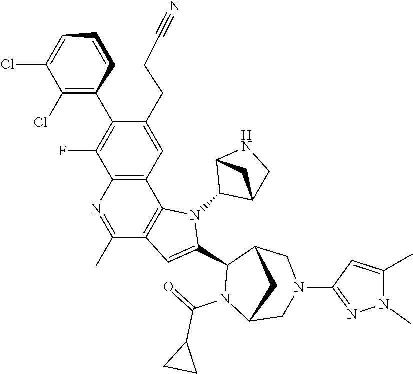 | 3-((Rₐ)-1-((1R,4R,5S)-2- azabicyclo[2.1.1]hexan-5-yl)-2- ((1S,5R,7R)-6-(cyclopropanecarbonyl)-3- (1,5-dimethyl-1H-pyrazol-3-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 751.3 |
| 106 | 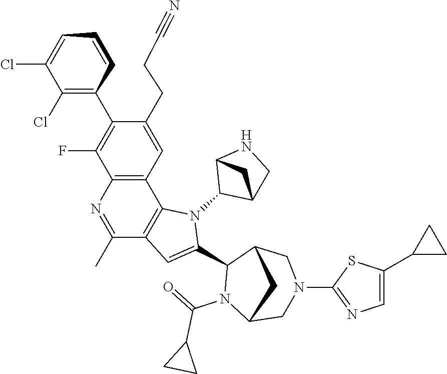 | 3-((Rₐ)-1-((1R,4R,5S)-2- azabicyclo[2.1.1]hexan-5-yl)-2- ((1S,5R,7R)-6-(cyclopropanecarbonyl)-3- (5-cyclopropylthiazol-2-yl)-3,6- diazabicyclo[3.2.1]octan-7-yl)-7-(2,3- dichlorophenyl)-6-fluoro-4-methyl-1H- pyrrolo[3,2-c]quinolin-8-yl)propanenitrile | 780.2 |

The alternative atropisomers of the compounds of Table 5 can be prepared by an analogous route by performing processes analogous to Example 31 starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 107. 3-((Rₐ)-1-((1R,4R,5S)-2-Azabicyclo[2.1.1]hexan-5-yl)-2-((1 S,5R,7R)-3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

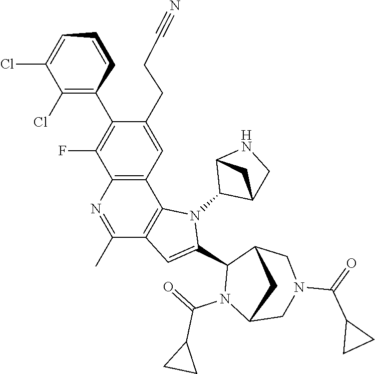

To a solution containing tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 0.009 g, 0.012 mmol) in DCM (0.12 mL) were added DIPEA (0.006 mL, 0.036 mmol) and cyclopropanecarbonyl chloride (0.002 g, 0.024 mmol). The mixture was stirred at r.t. for 0.5 h. The mixture was then diluted with DCM, washed with water, dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₀H₄₀Cl₂FN₆O₂ (M+H)⁺: m/z=725.3; found 725.2.

The alternative atropisomer 3-((Sₐ)-1-((1R,4R,5S)-2-azabicyclo[2.1.1]hexan-5-yl)-2-((1 S,5R,7R)-3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 108. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate

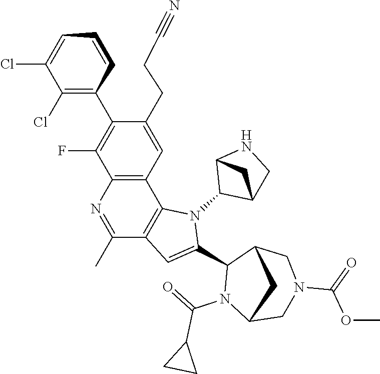

To a solution containing tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 0.009 g, 0.012 mmol) in DCM (0.12 mL) were added DIPEA (0.006 mL, 0.036 mmol) and methyl chloroformate (0.002 g, 0.024 mmol). The mixture was stirred at r.t. for 0.5 h. The mixture was then diluted with DCM, washed with water, dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₃₈H₃₈Cl₂FN₆O₃ (M+H)⁺: m/z=715.2; found 715.2.

The alternative atropisomer methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 109. (1 S,5R,7R)-7-((Rₐ)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide

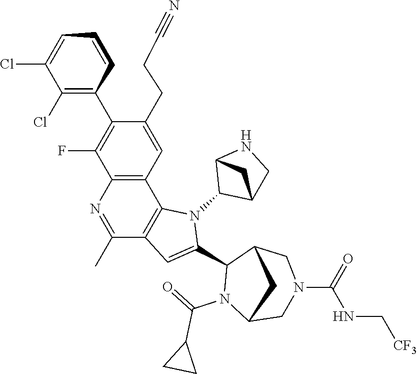

To a solution containing tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 0.008 g, 0.011 mmol) in DCM (0.1 mL) were added DIPEA (0.004 mL, 0.032 mmol) and 1,1,1-trifluoro-2-isocyanatoethane (0.007 g, 0.053 mmol). The mixture was stirred at r.t. for 1 h. The mixture was then diluted with DCM, washed with sat. aq. NaHCO₃, dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₃₉H₃₈Cl₂F₄N₇O₂ (M+H)⁺: m/z=782.2; found 782.2.

The alternative atropisomer (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 110. 3-((Rₐ)-1-((1R,4R,5S)-2-Azabicyclo[2.1.1]hexan-5-yl)-2-((1 S,5R,7R)-6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

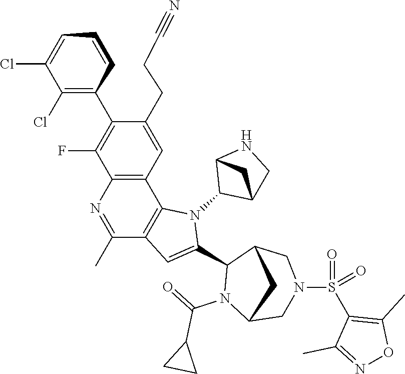

To a 8 mL scintillation vial was added tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 8, 15.0 mg, 0.020 mmol) in THF (198 μL). NEt₃ (22.01 μL, 0.158 mmol) was added and the mixture was allowed to cool to 0° C. After 5 min., 3,5-dimethylisoxazole-4-sulfonyl chloride (11.62 mg, 0.059 mmol) was added in small increments. The mixture was stirred 0° C. and was allowed to warm to r.t. over 16 h. The mixture was diluted with EtOAc and washed with brine. The organic layer was dried over Na₂SO₄, filtered and concentrated.

To the residue dissolved in DCM (0.5 mL) was added TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₁H₄₁Cl₂FN₇O₄S (M+H)⁺: m/z=816.2; found 816.2.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 111. 3-((Rₐ)-1-((1R,4R,5S)-2-Azabicyclo[2.1.1]hexan-5-yl)-2-((1 S,5R,7R)-6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

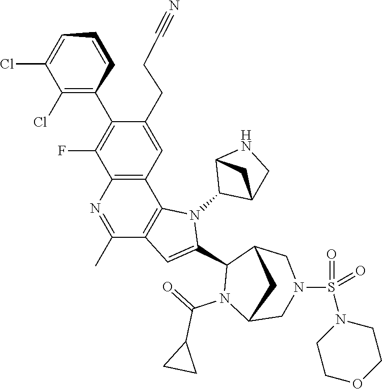

This compound was prepared in an analogous fashion to Example 110, with morpholine-4-sulfonyl chloride replacing 3,5-dimethylisoxazole-4-sulfonyl chloride. LCMS calc. for C₄₀H₄₃Cl₂FN₇O₄S (M+H)⁺: m/z=806.2; found 806.1

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 112. 3-((Rₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Sₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

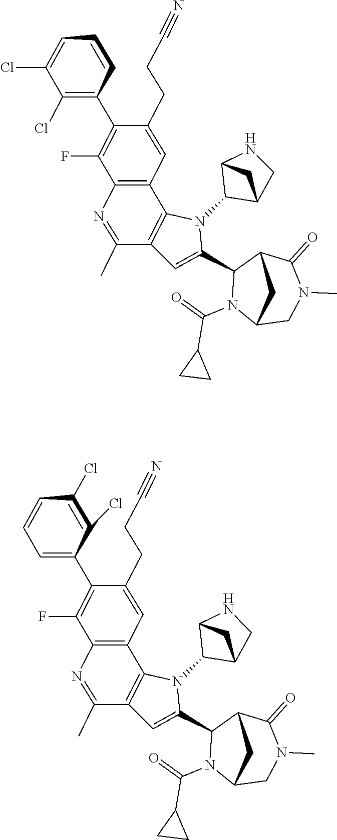

### Step 1. 6-(tert-Butyl) 7-methyl (1S,5R,7R)-3-methyl-3,6-diazabicyclo[3.2.1]octane-6,7-dicarboxylate

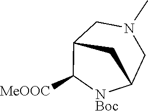 [SMILES: COC(=O)[C@H]1[C@H]2C[C@H](CN(C)C2)N1C(=O)OC(C)(C)C]

To a solution of 2-(tert-butyl) 3-methyl (1R,3R,4R,5R,6S)-5,6-dihydroxy-2-azabicyclo[2.2.1]heptane-2,3-dicarboxylate (intermediate 4, 0.54 g, 1.88 mmol) in 1,4-dioxane/water (8 mL/2 mL) were added 2,6-lutidine (0.88 mL, 7.52 mmol) and NaIO₄ (0.8 g, 3.76 mmol). The mixture was stirred at r.t. for 1 h. The mixture was then diluted with EtOAc and washed with brine. The organic layer was dried over Na₂SO₄, filtered and concentrated under reduced pressure.

To the residue dissolved in DCM (10 mL) were added methanamine (2M in THF, 1.04 mL, 2.08 mmol) and PPTS (1.0 g, 2.0 mmol). The mixture was stirred for 30 min. at r.t. before NaBH(OAc)₃ (1.68 g, 8.0 mmol) was added. After stirring for 0.5 h, the reaction was quenched by slow addition of water. The mixture was diluted with DCM, washed with ammonia and brine subsequently. The combined organic layers were dried over Na₂SO₄, filtered and concentrated. The crude product was further purified by FCC (0-10% MeOH in DCM containing 1% NEts) to afford the sub-title compound as an oil (0.5 g, 89% yield). LCMS calc. for C₁₄H₁₇N₂O₄ (M+H)⁺: m/z=285.2; found 285.2.

### Step 2. 6-(tert-Butyl) 7-methyl (1R,5R,7R)-3-methyl-2-oxo-3,6-diazabicyclo[3.2.1]octane-6,7-dicarboxylate

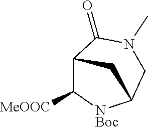 [SMILES: COC(=O)[C@H]1[C@H]2C[C@H](CN(C)C2=O)N1C(=O)OC(C)(C)C]

Ruthenium(IV) oxide (47.7 mg, 0.359 mmol) was added to a solution of NaIO₄ (767 mg, 3.59 mmol) in water (5 mL), which was stirred at r.t. for 5 min. To the formed yellow solution was added a solution of 6-(tert-butyl) 7-methyl (1 S,5R,7R)-3-methyl-3,6-diazabicyclo[3.2.1]octane-6,7-dicarboxylate (340 mg, 1.196 mmol) in EtOAc (5.00 mL), and the resulting mixture was vigorously stirred at r.t. for 2 h. The mixture was then diluted with EtOAc and filtered through diatomaceous earth, the filtrate was washed with brine, dried over Na₂SO₄, filtered and concentrated. The crude product was further purified by FCC (0-100% EtOAc in hexanes) to afford the sub-title compound as an oil (275 mg, 77% yield). LCMS calc. for C₁₄H₂₃N₂O₅ (M+H)⁺: m/z=299.2; found 299.2.

### Step 3. tert-Butyl (1R,5R,7R)-7-(hydroxymethyl)-3-methyl-2-oxo-3,6-diazabicyclo[3.2.1]octane-6-carboxylate

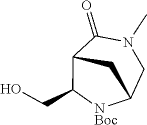 [SMILES: CN1C[C@H]2C[C@@H](C1=O)[C@H](CO)N2C(=O)OC(C)(C)C]

To a solution of 6-(tert-butyl) 7-methyl (1R,5R,7R)-3-methyl-2-oxo-3,6-diazabicyclo[3.2.1]octane-6,7-dicarboxylate (0.2 g, 0.67 mmol) in THF (10 mL) was added LiBH₄ (2 M in THF, 0.67 mL, 1.34 mmol). The mixture was stirred at r.t. for 10 h and then quenched by slow addition of sat. aq. NH₄Cl at 0° C. The mixture was diluted with EtOAc, washed with brine, dried over Na₂SO₄, filtered and concentrated. The crude product was purified by FCC (0-100% EtOAc in hexanes) to afford the sub-title compound (0.13 g, 71% yield). LCMS calc. for C₁₃H₂₃N₂O₄ (M+H)⁺: m/z=271.2; found 271.2.

### Step 4. tert-Butyl (1R,5R,7R)-7-formyl-3-methyl-2-oxo-3,6-diazabicyclo[3.2.1]octane-6-carboxylate

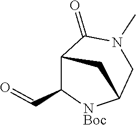 [SMILES: CN1C[C@H]2C[C@@H](C1=O)[C@H](C=O)N2C(=O)OC(C)(C)C]

To a solution of dimethyl sulfoxide (0.101 mL, 1.420 mmol) in DCM (6 mL) at −78° C. was added oxalyl dichloride (0.080 mL, 0.947 mmol) dropwise. After 30 min., tert-butyl (1R,5R,7R)-7-(hydroxymethyl)-3-methyl-2-oxo-3,6-diazabicyclo[3.2.1]octane-6-carboxylate (128 mg, 0.473 mmol) in DCM (1 mL) was added dropwise at −78° C., the mixture was stirred for 1 h. NEt₃ (0.264 mL, 1.894 mmol) was then added dropwise at −78° C., the resulting mixture was stirred at −78° C. for 10 min. then allowed to warm to r.t. and continue stirring for another 1 h. Water was added and the product was extracted with DCM (3×), washed with brine, dried over Na₂SO₄ and concentrated to afford the crude product, which was used in the next step without further purification. LCMS calc. for C₁₃H₂₁N₂O₄ (M+H)⁺: m/z=269.1; found 269.2.

### Step 5. tert-Butyl (1R,5R,7R)-7-ethynyl-3-methyl-2-oxo-3,6-diazabicyclo[3.2.1]octane-6-carboxylate

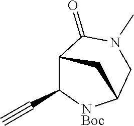 [SMILES: C#C[C@H]1[C@H]2C[C@H](CN(C)C2=O)N1C(=O)OC(C)(C)C]

To a solution of tert-butyl (1R,5R,7R)-7-formyl-3-methyl-2-oxo-3,6-diazabicyclo[3.2.1]octane-6-carboxylate (120 mg, 0.447 mmol) in MeOH (5 mL) were added dimethyl(1-diazo-2-oxopropyl)phosphonate (129 mg, 0.671 mmol) and K₂CO₃ (124 mg, 0.894 mmol). After stirring for 2 h, the mixture was filtered through diatomaceous earth. The filtrate was concentrated. The residue was extracted with EtOAc, washed with brine, dried over Na₂SO₄, and concentrated. The crude product used in next step without further purification. LCMS calc. for C₁₄H₂₁N₂O₃ (M+H)⁺: m/z=265.2; found 265.2.

### Step 6. (1R,5R,7R)-6-(Cyclopropanecarbonyl)-7-ethynyl-3-methyl-3,6-diazabicyclo[3.2.1]octan-2-one

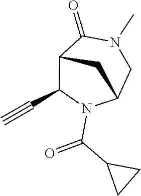 [SMILES: C#C[C@H]1[C@H]2C[C@H](CN(C)C2=O)N1C(=O)C1CC1]

Trimethylsilyl trifluoromethanesulfonate (0.282 mL, 1.562 mmol) was added dropwise to a solution of tert-butyl (1R,5R,7R)-7-ethynyl-3-methyl-2-oxo-3,6-diazabicyclo[3.2.1]octane-6-carboxylate (118 mg, 0.446 mmol) in DCM (6 mL) at 0° C., then the mixture was allowed to warm up to r.t. and stirring for 30 min. The mixture was quenched with sat. aq. NaHCO₃, washed with brine, dried over Na₂SO₄, and concentrated. The residue was dissolved in DCM (5 mL), NEt₃ (0.30 mL, 2.23 mmol) and cyclopropanecarbonyl chloride (93 mg, 0.89 mmol) were added subsequently at 0° C. The mixture was stirred at r.t. for 0.5 h. The mixture was then concentrated under vacuum. The crude product was purified by FCC (0-100% EtOAc in hexanes) to give the sub-title compound (40 mg, 39% yield over three steps). LCMS calc. for C₁₃H₁₇N₂O₂ (M+H)⁺: m/z=233.1; found 233.2.

### Step 7. 3-((Rₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Sₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

A mixture of tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (racemic intermediate 2, 15 mg, 0.022 mmol), (1R,5R,7R)-6-(cyclopropanecarbonyl)-7-ethynyl-3-methyl-3,6-diazabicyclo[3.2.1]octan-2-one (7.7 mg, 0.033 mmol), CuI (4.19 mg, 0.02 mmol), Pd(PPh₃)₄ (5.1 mg, 0.004 mmol) and DIPEA (0.031 mL, 0.18 mmol) in DMF (0.8 mL) was sparged with N₂ and heated at 70° C. for 1 h. After cooling to r.t., Cs₂CO₃ (35.9 mg, 0.11 mmol) was added to the mixture, and the resulting mixture was stirred at 90° C. for 12 h. The mixture was then allowed to cool to r.t. and diluted with EtOAc. The solution was washed with water (3×) and brine, dried over Na₂SO₄, filtered and concentrated.

The reaction residue was dissolved in TFA (1 mL), and stirred for at r.t. for 10 min. The mixture was then diluted with MeCN (4 mL) and purified by prep. LCMS (XBRIDGE® C₁₈ column, eluting with a gradient of MeCN/water containing 0.1% TFA, at flow rate of 60 mL/min.) to afford the two title compounds.

Peak 1: LCMS calc. for C₃₇H₃₆Cl₂FN₆O₂ (M+H)⁺: m/z=685.3; found 685.3. This is the desired peak

Peak 2: LCMS calc. for C₃₇H₃₆Cl₂FN₆O₂ (M+H)⁺: m/z=685.3; found 685.3.

### Example 113. 3-((Rₐ)-1-((1R,4R,5S)-2-Azabicyclo[2.1.1]hexan-5-yl)-2-((1R,5R,7R)-3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Sₐ)-1-((1R,4R,5S)-2-Azabicyclo[2.1.1]hexan-5-yl)-2-((1R,5R,7R)-3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

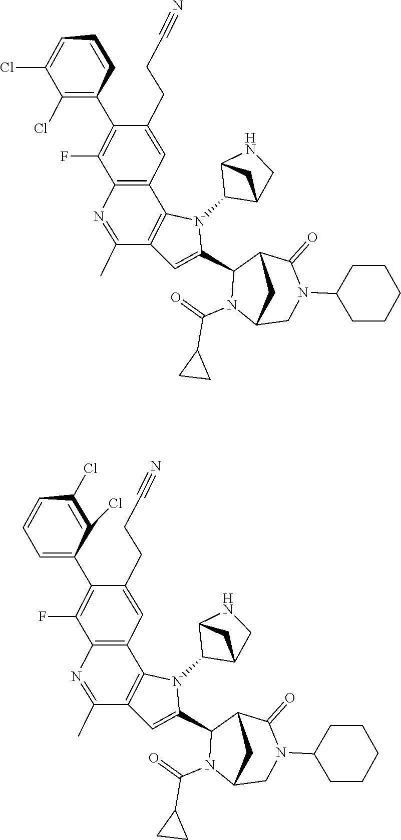

The title compound was prepared according to the procedure described in Example 112, by replacing methanamine with cyclohexylamine in Step 1.

Peak 1: LCMS calc. for C₄₂H₄₄Cl₂FN₆O₂ (M+H)⁺: m/z=753.3; found 753.4. This is the desired peak

Peak 2: LCMS calc. for C₄₂H₄₄Cl₂FN₆O₂ (M+H)⁺: m/z=753.3; found 753.4.

### Example 114. 3-((Rₐ)-1-((1R,4R,5S)-2-Azabicyclo[2.1.1]hexan-5-yl)-2-((1R,5R,7R)-3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

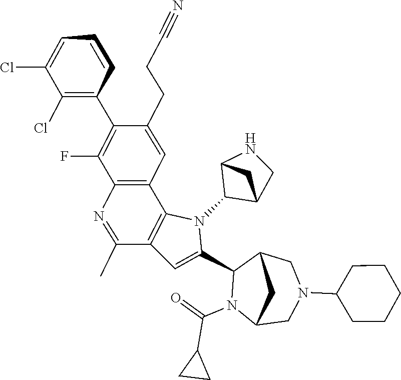

tert-Butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (Intermediate 8, 10 mg, 0.013 mmol) was dissolved in dichloroethane (1 mL). To this solution was added 0.2 mL premixed Et₃N/HOAc (v/v=1:1), followed by addition of cyclohexanone (3.89 mg, 0.040 mmol) and NaBH(OAc)₃ (8.39 mg, 0.040 mmol). The resulting mixture was stirred at r.t. for 1 h. The mixture was then diluted with DCM, washed with water and brine, dried over Na₂SO₄, filtered and concentrated. The reaction residue was dissolved in TFA (1 mL), and stirred for at r.t. for 10 min. The mixture was then diluted with MeCN (4 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₄₆Cl₂FN₆O (M+H)⁺: m/z=739.3 found 739.3.

The alternative atropisomer 3-((Sₐ)-1-((1R,4R,5S)-2-azabicyclo[2.1.1]hexan-5-yl)-2-((1R,5R,7R)-3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 115. 3-((Rₐ)-1-((1R,4R,5S)-2-Azabicyclo[2.1.1]hexan-5-yl)-2-((1R,5R,7R)-6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile

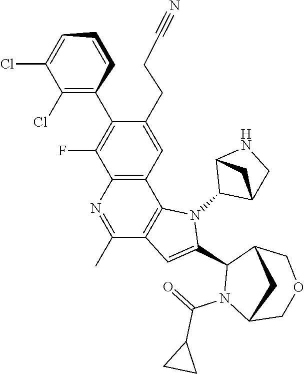

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 0.020 g, 0.029 mmol), cyclopropyl((1R,5R,7R)-7-ethynyl-3-oxa-6-azabicyclo[3.2.1]octan-6-yl)methanone (intermediate 9, 0.009 g, 0.044 mmol), CuI (0.002 g, 0.012 mmol), Pd(PPh₃)₄ (0.007 g, 0.006 mmol) and DIPEA (0.050 mL, 0.29 mmol) in DMF (0.15 mL) was sparged with N₂ and heated at 70° C. for 0.5 h. Then, Cs₂CO₃ (0.029 g, 0.088 mmol) was added to the mixture. The resulting slurry was stirred at 90° C. for another 18 h. The mixture was then allowed to cool to r.t. and poured into water. The solution was extracted with EtOAc (2×). Then the combined organic layers were washed with brine (5×), dried over Na₂SO₄, filtered and concentrated.

To the residue dissolved in DCM (1 mL) were added MeCN (0.1 mL) and TFA (1 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₃₆H₃₅Cl₂FN₅O₂ (M+H)⁺: m/z=658.2; found 658.2.

The alternative atropisomer 3-((Sₐ)-1-((1R,4R,5S)-2-azabicyclo[2.1.1]hexan-5-yl)-2-((1R,5R,7R)-6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Example 116. 3-((Rₐ)-1-((1R,4R,5S)-2-Azabicyclo[2.1.1]hexan-5-yl)-2-((1R,5R,7R)-6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile

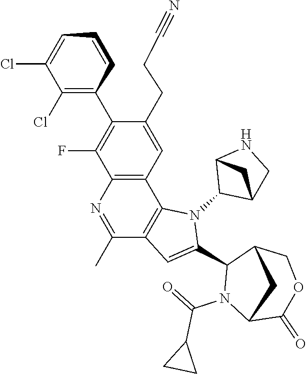

To a solution of tert-butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate (intermediate 6, 0.010 g, 0.013 mmol) in DCM (0.5 mL) was added NaBH(OAc)₃ (0.015 g, 0.066 mmol). After stirring for 0.5 h, the mixture was quenched by slow addition of water. The mixture was extracted with DCM (2×). The combined organic layers were dried over Na₂SO₄, filtered and concentrated.

To the residue dissolve in DCM (0.5 mL) was added Dess-Martin periodinane (0.023 g, 0.055 mmol). The mixture was stirred at r.t. for 0.5 h. The mixture was then diluted with DCM, filtered through diatomaceous earth. The organic fraction was washed with water, dried over Na₂SO₄, filtered and concentrated.

To the residue dissolved in DCM (0.5 mL) were added MeCN (0.05 mL) and TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₃₆H₃₃Cl₂FN₅O₃ (M+H)⁺: m/z=672.2; found 672.1.

The alternative atropisomer 3-((Sₐ)-1-((1R,4R,5S)-2-azabicyclo[2.1.1]hexan-5-yl)-2-((1R,5R,7R)-6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((7Sa)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((7Rₐ)-8-(2-cyanoethyl)-2-((1R,5R)-6-(cyclopropanecarbonyl)-2,4-dihydroxy-3-oxa-6-azabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 117. 3-((Rₐ)-1-((1R,4R,5S)-2-Azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile

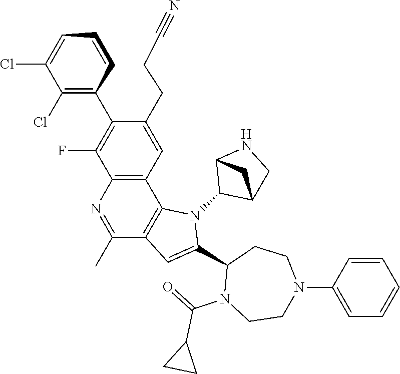

### Step 1. tert-Butyl (1R,4R,5S)-5-(((7Rₐ)-6-(2-cyanoethyl)-3-((4-(cyclopropanecarbonyl)-1-phenyl-1,4-diazepan-5-yl)ethynyl)-7-(2,3-dichlorophenyl)-8-fluoro-2-methylquinolin-4-yl)amino)-2-azabicyclo[2.1.1]hexane-2-carboxylate

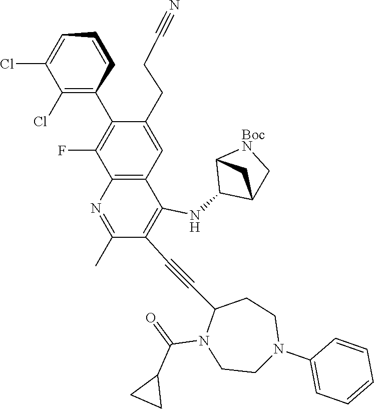

To a 8 mL scintillation vial charged with a magnetic stir bar were added cyclopropyl(7-ethynyl-4-phenyl-1,4-diazepan-1-yl)methanone (intermediate 10, 9.5 mg, 0.035 mmol), tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 20.0 mg, 0.029 mmol), Pd(PPh₃)₄ (6.8 mg, 5.9 μmol) and CuI (2.2 mg, 0.012 mmol), DMF (587 μL), and DIPEA (26 μL, 0.15 mmol). The mixture was sparged with N₂ and heated at 70° C. for 2 h before cooling to r.t. The mixture was diluted with brine (10 mL) and EtOAc (10 mL). The resulting mixture was extracted with EtOAc (3×20 mL). The combined organic phased were washed with brine (30 mL), dried over anhydrous Na₂SO₄, and filtered. The volatiles were removed under vacuum, and the residue was purified by FCC (0-100% EtOAc/hexanes) to give a mixture of two isomers as a light brown oil (22.8 mg, 95% yield). LCMS calc. for C₄₆H₄₈Cl₂FN₆O₃ (M+H)⁺: m/z=821.3; found 821.4.

### Step 2. tert-Butyl (1R,4R,5S)-5-((Rₐ)-8-(2-cyanoethyl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-1-yl)-2-azabicyclo[2.1.1]hexane-2-carboxylate

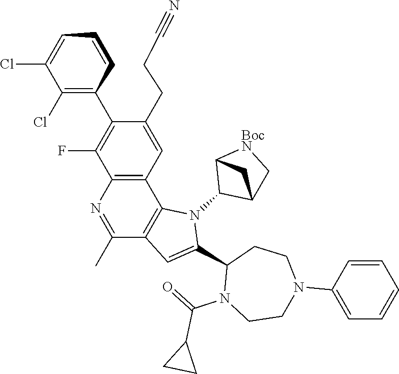

To a 8 mL scintillation vial charged with a magnetic stir bar were added tert-butyl (1R,4R,5S)-5-(((7Rₐ)-6-(2-cyanoethyl)-3-((4-(cyclopropanecarbonyl)-1-phenyl-1,4-diazepan-5-yl)ethynyl)-7-(2,3-dichlorophenyl)-8-fluoro-2-methylquinolin-4-yl)amino)-2-azabicyclo[2.1.1]hexane-2-carboxylate (22.8 mg, 0.028 mmol), Cs₂CO₃ (18.1 mg, 0.055 mmol), and DMF (0.6 mL). The mixture was sparged with N₂ before being heated at 70° C. for 2 h before cooling to r.t. The mixture was diluted with brine (10 mL) and EtOAc (10 mL). The resulting mixture was extracted with EtOAc (3×20 mL). The combined organic phases were washed with brine (30 mL), dried over anhydrous Na₂SO₄, and filtered. The volatiles were removed under vacuum, and the residue was purified by FCC (0-100% EtOAc/hexanes) to give title compound as a single isomer (eluded prior to the other isomer) as a light brown solid (8.4 mg, 35% yield). LCMS calc. for C₄₆H₄₈Cl₂FN₆O₃ (M+H)⁺: m/z=821.3; found 821.4.

### Step 3. 3-((Rₐ)-1-((1R,4R,5S)-2-Azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile

To a 8 mL scintillation vial charged with a magnetic stir bar were added tert-butyl (1R,4R,5S)-5-((Rₐ)-8-(2-cyanoethyl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-1-yl)-2-azabicyclo[2.1.1]hexane-2-carboxylate (8.4 mg, 0.010 mmol), DCM (200 μL), TFA (200 μL), and MeCN (5.0 μL) at r.t. The resulting mixture was allowed to stir at that temperature for 35 min. before the volatiles were removed under vacuum, the residue was dissolved in MeCN (4.5 mL) and water (0.5 mL), and further purified by prep. LCMS (XBRIDGE® C₁₈ column, eluting with a gradient of MeCN/water containing 0.1% TFA, at flow rate of 60 mL/min.) to give the title compound as a white solid (5.1 mg, TFA salt). LCMS calc. for C₄₁H₄₀Cl₂FN₆O₃ (M+H)⁺: m/z=721.3; found 721.4.

The alternative atropisomer 3-((Sₐ)-1-((1R,4R,5S)-2-azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile can be prepared by an analogous route by performing a process analogous to the steps above starting from tert-butyl (1R,4R,5S)-5-((Sₐ)-8-(2-cyanoethyl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 118. 3-((Rₐ)-1-((1R,4R,5S)-2-Azabicyclo[2.1.1]hexan-5-yl)-2-((1R,3R,5R)-2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile

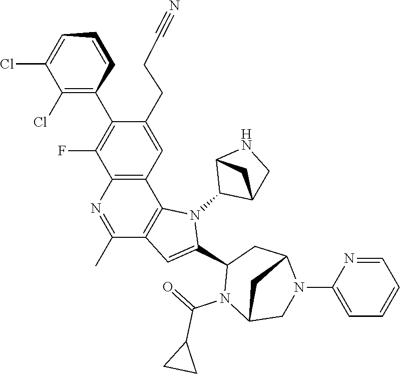

This compound was prepared in an analogous fashion to Example 31 with 2-chloropyridine replacing 4-chloro-5-fluoro-6-methylpyrimidine and Intermediate 12 replacing Intermediate 8. LCMS calc. for C₄₁H₃₉Cl₂FN₇O (M+H)⁺: m/z=734.3; found 734.1.

The alternative atropisomer 3-((Sₐ)-1-((1R,4R,5S)-2-azabicyclo[2.1.1]hexan-5-yl)-2-((1R,3R,5R)-2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 119. 3-((Rₐ)-2-((1R,3R,5R)-6-Benzoyl-2-(cyclopropanecarbonyl)-2,6-diazabicyclo[3.2.1]octan-3-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile

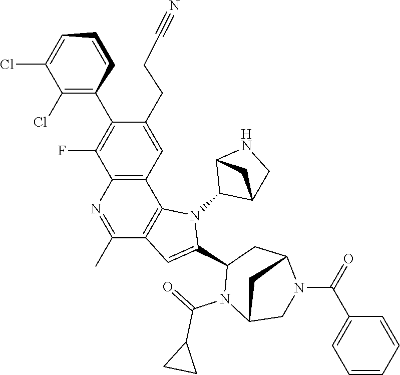

This compound was prepared in an analogous fashion to Example 108 with benzoyl chloride replacing methyl chloroformate and Intermediate 12 replacing Intermediate 8. LCMS calc. for C₄₃H₄₀Cl₂FN₆O₂ (M+H)⁺: m/z=761.3; found 761.2.

The alternative atropisomer 3-((Sₐ)-2-((1R,3R,5R)-6-benzoyl-2-(cyclopropanecarbonyl)-2,6-diazabicyclo[3.2.1]octan-3-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-((Sₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate.

### Example 120a and 120b. 3-((Rₐ)-1-((1R,4R,5S)-2-Azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Rₐ)-1-((1R,4R,5S)-2-Azabicyclo[2.1.1]hexan-5-yl)-2-((1S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

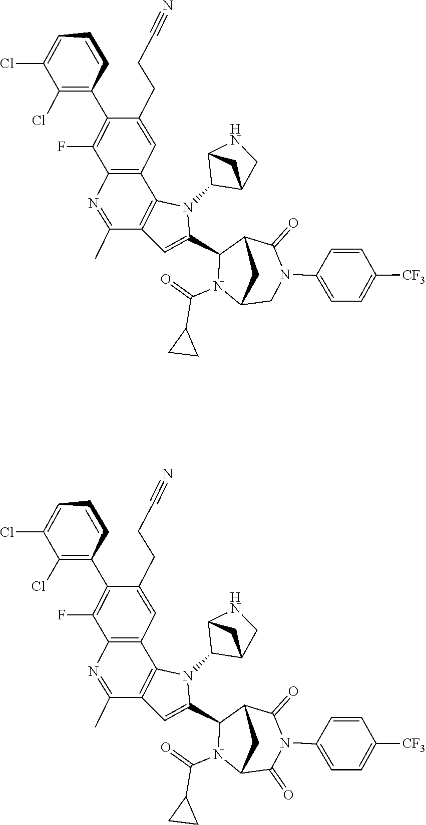

### Step 1. 6-(tert-Butyl) 7-methyl (1S,5R,7R)-3-(4-(trifluoromethyl)phenyl)-3,6-diazabicyclo[3.2.1]octane-6,7-dicarboxylate

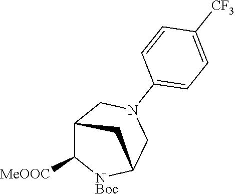 [SMILES: COC(=O)[C@H]1[C@H]2C[C@H](CN(c3ccc(C(F)(F)F)cc3)C2)N1C(=O)OC(C)(C)C]

To a solution of 2-(tert-butyl) 3-methyl (1R,3R,4R,5R,6S)-5,6-dihydroxy-2-azabicyclo[2.2.1]heptane-2,3-dicarboxylate (intermediate 4, 143 mg, 0.50 mmol) in 1,4-dioxane/water (2 mL/0.5 mL) were added 2,6-lutidine (0.23 mL, 2.0 mmol) and NaIO₄ (213 mg, 1.0 mmol). The mixture was stirred at r.t. for 1 h. The mixture was then diluted with EtOAc and washed with brine. The organic layer was dried over Na₂SO₄, filtered and concentrated under reduced pressure.

The crude mixture was dissolved in DCM (5 mL), to which were added 4-(trifluoromethyl)aniline (89 mg, 0.550 mmol) and PPTS (251 mg, 1.000 mmol). The resulting mixture was stirred for 30 min. at r.t. before NaBH(OAc)₃ (424 mg, 2.0 mmol) was added.

After stirring for 3 h, the reaction was quenched by slow addition of water. The mixture was diluted with DCM and washed with brine. The combined organic layers were dried over Na₂SO₄, filtered and concentrated. The crude product was further purified by FCC (0-100% EtOAc in hexanes) to afford the sub-title compound (170 mg, 82% yield). LCMS calc. for C₁₆H₁₈F₃N₂O₄(M-^{t}Bu+2H)⁺: m/z=359.2; found 359.2.

### Step 2. tert-Butyl (1S,5R,7R)-7-(hydroxymethyl)-3-(4-(trifluoromethyl)phenyl)-3,6-diazabicyclo[3.2.1]octane-6-carboxylate

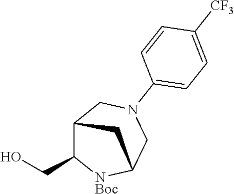 [SMILES: CC(C)(C)OC(=O)N1[C@@H]2C[C@@H](CN(c3ccc(C(F)(F)F)cc3)C2)[C@@H]1CO]

To a solution of 6-(tert-butyl) 7-methyl (1 S,5R,7R)-3-(4-(trifluoromethyl)phenyl)-3,6-diazabicyclo[3.2.1]octane-6,7-dicarboxylate (170 mg, 0.41 mmol) in THF (6 mL) was added LiBH₄ (2 M in THF, 0.41 mL, 0.82 mmol) at 0° C. The mixture was then allowed to warm up to r.t. and continue stirring for 12 h. Upon completion, the reaction was quenched by slow addition of sat. aq. NH₄Cl at 0° C. The mixture was diluted with EtOAc, washed with brine, dried over Na₂SO₄, filtered and concentrated. The crude product was used in the next step without further purification. LCMS calc. for C₁₅H₁₈F₃N₂O₃(M-^{t}Bu+2H)⁺: m/z=331.1; found 331.2.

### Step 3. tert-Butyl (1S,5R,7R)-7-(((tert-butyldimethylsilyl)oxy)methyl)-3-(4-(trifluoromethyl)phenyl)-3,6-diazabicyclo[3.2.1]octane-6-carboxylate

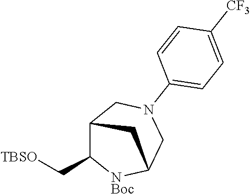 [SMILES: CC(C)(C)OC(=O)N1[C@@H]2C[C@@H](CN(c3ccc(C(F)(F)F)cc3)C2)[C@@H]1CO[Si](C)(C)C(C)(C)C]

TBSCI (86 mg, 0.571 mmol) was added to a solution of tert-butyl (1 S,5R,7R)-7-(hydroxymethyl)-3-(4-(trifluoromethyl)phenyl)-3,6-diazabicyclo[3.2.1]octane-6-carboxylate (147 mg, 0.380 mmol) in DMF (4 mL) at r.t., followed by the addition of imidazole (51.8 mg, 0.761 mmol). The resulting mixture was stirred for 3 h to achieve full conversion. The mixture was then diluted with EtOAc, washed with water (3×) and brine, dried over Na₂SO₄, and concentrated under reduced pressure. The crude product was purified by FCC (0-100% EtOAc in hexanes) to afford the sub-title compound (157 mg, 82% yield). LCMS calc. for C₂₁H₃₂F₃N₂O₃Si (M-^{t}Bu+2H)⁺: m/z=445.3; found 445.3.

### Step 4. tert-Butyl (1R,5R,7R)-7-(((tert-butyldimethylsilyl)oxy)methyl)-2-oxo-3-(4-(trifluoromethyl)phenyl)-3,6-diazabicyclo[3.2.1]octane-6-carboxylate and tert-butyl (1 S,5R,7R)-7-(((tert-butyldimethylsilyl)oxy)methyl)-4-oxo-3-(4-(trifluoromethyl)phenyl)-3,6-diazabicyclo[3.2.1]octane-6-carboxylate

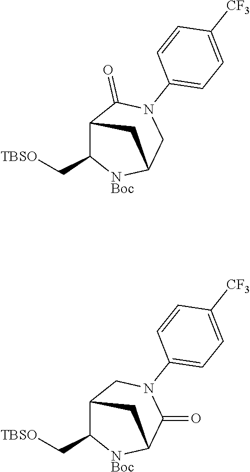 [SMILES: CC(C)(C)OC(=O)N1[C@@H]2C[C@@H](C(=O)N(c3ccc(C(F)(F)F)cc3)C2)[C@@H]1CO[Si](C)(C)C(C)(C)C ; CC(C)(C)OC(=O)N1[C@@H]2C[C@@H](CN(c3ccc(C(F)(F)F)cc3)C2=O)[C@@H]1CO[Si](C)(C)C(C)(C)C]

Ruthenium(IV) oxide (12.5 mg, 0.094 mmol) was added to a solution of NaIO₄ (201 mg, 0.94 mmol) in water (4 mL), which was stirred at r.t. for 5 min. To the formed yellow solution was added a solution of tert-butyl (1 S,5R,7R)-7-(((tert-butyldimethylsilyl)oxy)methyl)-3-(4-(trifluoromethyl)phenyl)-3,6-diazabicyclo[3.2.1]octane-6-carboxylate (157 mg, 0.314 mmol) in EtOAc (4 mL), and the resulting mixture was vigorously stirred at r.t. for 2 h. Upon completion, the reaction was quenched by adding isopropyl alcohol, diluted with EtOAc and filtered through diatomaceous earth. The filtrate was washed with brine, dried over Na₂SO₄, filtered and concentrated. Two lactam regioisomers were formed in this reaction. The crude products was used in the next step without separation.

### Step 5. tert-Butyl (1R,5R,7R)-7-(hydroxymethyl)-2-oxo-3-(4-(trifluoromethyl)phenyl)-3,6-diazabicyclo[3.2.1]octane-6-carboxylate and tert-butyl (1S,5R,7R)-7-(hydroxymethyl)-4-oxo-3-(4-(trifluoromethyl)phenyl)-3,6-diazabicyclo[3.2.1]octane-6-carboxylate

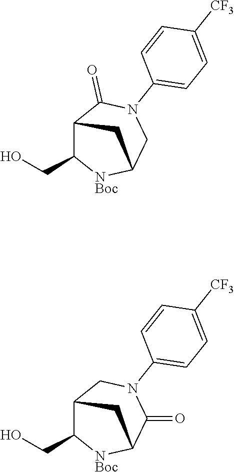 [SMILES: CC(C)(C)OC(=O)N1[C@@H]2C[C@@H](C(=O)N(c3ccc(C(F)(F)F)cc3)C2)[C@@H]1CO ; CC(C)(C)OC(=O)N1[C@@H]2C[C@@H](CN(c3ccc(C(F)(F)F)cc3)C2=O)[C@@H]1CO]

TBAF (1 M in THF, 0.31 mL, 0.31 mmol) was added to a solution of the crude product from step 4 in THF (4.0 mL) at r.t., and the resulting mixture was stirred for 2 h. The volatiles were then removed under reduced pressure, and the residue was purified by FCC (0-100% EtOAc in hexanes) to afford a mixture of the sub-title compounds (80 mg, 64% yield over two steps). LCMS calc. for C₁₅H₁₆F₃N₂O₄(M-^{t}Bu+2H)⁺: m/z=345.1; found 345.1.

### Step 6. tert-Butyl (1R,5R,7R)-7-formyl-2-oxo-3-(4-(trifluoromethyl)phenyl)-3,6-diazabicyclo[3.2.1]octane-6-carboxylate and tert-butyl (1S,5R,7R)-7-formyl-4-oxo-3-(4-(trifluoromethyl)phenyl)-3,6-diazabicyclo[3.2.1]octane-6-carboxylate

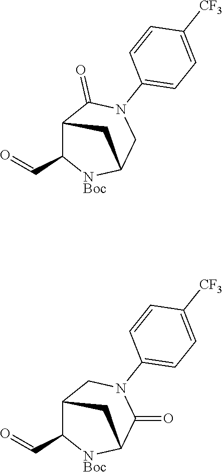 [SMILES: CC(C)(C)OC(=O)N1[C@@H]2C[C@@H](C(=O)N(c3ccc(C(F)(F)F)cc3)C2)[C@@H]1C=O ; CC(C)(C)OC(=O)N1[C@@H]2C[C@@H](CN(c3ccc(C(F)(F)F)cc3)C2=O)[C@@H]1C=O]

Dess-Martin periodinane (127 mg, 0.3 mmol) was added to a solution of tert-butyl (1R,5R,7R)-7-(hydroxymethyl)-2-oxo-3-(4-(trifluoromethyl)phenyl)-3,6-diazabicyclo[3.2.1]octane-6-carboxylate and tert-butyl (1 S,5R,7R)-7-(hydroxymethyl)-4-oxo-3-(4-(trifluoromethyl)phenyl)-3,6-diazabicyclo[3.2.1]octane-6-carboxylate (80 mg, 0.2 mmol) in DCM (4 mL) at 0° C. After stirring at 0° C. for 30 min., the mixture was allowed to warm up to r.t. The reaction was quenched by adding sat. aq. Na₂S₂O₃, then extracted with DCM. The organic phase was washed with brine, dried over Na₂SO₄, and concentrated. The crude product was purified by FCC (0-100% EtOAc in hexanes) to afford a mixture of the sub-title compounds (60 mg, 75% yield). LCMS calc. for C₁₉H₂₂F₃N₂O₄ (M+H)⁺: m/z=399.2; found 399.2.

### Step 7. tert-Butyl (1R,5R,7R)-7-ethynyl-2-oxo-3-(4-(trifluoromethyl)phenyl)-3,6-diazabicyclo[3.2.1]octane-6-carboxylate and tert-butyl (1S,5R,7R)-7-ethynyl-4-oxo-3-(4-(trifluoromethylphenyl)-3,6-diazabicyclo[3.2._1]octane-6-carboxylate

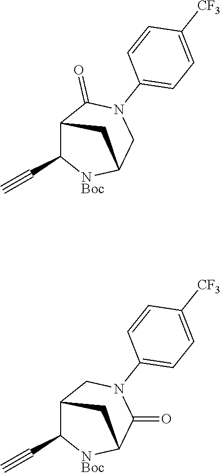 [SMILES: C#C[C@H]1[C@H]2C[C@H](CN(c3ccc(C(F)(F)F)cc3)C2=O)N1C(=O)OC(C)(C)C ; C#C[C@H]1[C@H]2C[C@H](C(=O)N(c3ccc(C(F)(F)F)cc3)C2)N1C(=O)OC(C)(C)C]

To a solution of tert-butyl (1R,5R,7R)-7-formyl-2-oxo-3-(4-(trifluoromethyl)phenyl)-3,6-diazabicyclo[3.2.1]octane-6-carboxylate and tert-butyl (1 S,5R,7R)-7-formyl-4-oxo-3-(4-(trifluoromethyl)phenyl)-3,6-diazabicyclo[3.2.1]octane-6-carboxylate (48 mg, 0.12 mmol) in MeOH (2 mL) were added dimethyl(1-diazo-2-oxopropyl)phosphonate (46.3 mg, 0.24 mmol) and K₂CO₃ (33.3 mg, 0.24 mmol) at 0° C. Then the mixture was gradually allowed to warm to r.t. After stirring for 1 h, the mixture was filtered through diatomaceous earth. The filtrate was diluted with EtOAc, washed with brine, dried over Na₂SO₄, and concentrated. The residue was purified by FCC (0-100% EtOAc in hexanes) to afford a mixture of the sub-title compounds (40 mg, 84% yield). LCMS calc. for C₂₀H₂₂F₃N₂O₃ (M+H)⁺: m/z=395.2; found 395.2.

### Step 8. (1R,5R,7R)-6-(Cyclopropanecarbonyl)-7-ethynyl-3-(4-(trifluoromethyl)phenyl)-3,6-diazabicyclo[3.2.1]octan-2-one and (1S,5R,7R)-6-(cyclopropanecarbonyl)-7-ethynyl-3-(4-(trifluoromethyl)phenyl)-3,6-diazabicyclo[3.2.1]octan-4-one

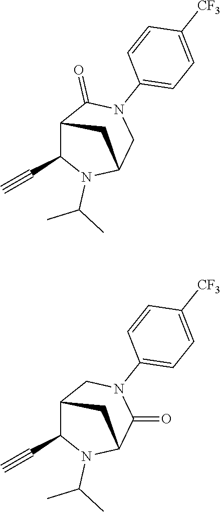 [SMILES: C#C[C@H]1[C@H]2C[C@H](CN(c3ccc(C(F)(F)F)cc3)C2=O)N1C(C)C ; C#C[C@H]1[C@H]2C[C@H](C(=O)N(c3ccc(C(F)(F)F)cc3)C2)N1C(C)C]

HCl (4 M in 1,4-dioxane, 0.5 mL) was added dropwise to a solution of tert-butyl (1R,5R,7R)-7-ethynyl-2-oxo-3-(4-(trifluoromethyl)phenyl)-3,6-diazabicyclo[3.2.1]octane-6-carboxylate and tert-butyl (1S,5R,7R)-7-ethynyl-4-oxo-3-(4-(trifluoromethyl)phenyl)-3,6-diazabicyclo[3.2.1]octane-6-carboxylate (40 mg, 0.10 mmol) in EtOAc (1 mL) at r.t. The resulting mixture was continued stirring for 3 h with LCMS monitoring. The volatiles were then removed under reduced pressure. The residue was re-dissolved in DCM (2 mL), NEt₃ (70 μL, 0.50 mmol) and cyclopropanecarbonyl chloride (21.2 mg, 0.20 mmol) were added at 0° C. The mixture was stirred at r.t. for 0.5 h. The mixture then was concentrated under vacuum. The crude product was purified by FCC (0-100% EtOAc in hexanes) to give the sub-title compounds (33 mg, 90% yield). LCMS calc. for C₁₉H₁₈F₃N₂O₂ (M+H)⁺: m/z=363.1; found 363.1.

### Step 9. 3-((Rₐ)-1-((1R,4R,5S)-2-Azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Rₐ)-1-((1R,4R,5S)-2-azabicyclo[2.1.1]hexan-5-yl)-2-((1S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

A mixture of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate (intermediate 2, 20 mg, 0.029 mmol), (1R,5R,7R)-6-(cyclopropanecarbonyl)-7-ethynyl-3-(4-(trifluoromethyl)phenyl)-3,6-diazabicyclo[3.2.1]octan-2-one and (1 S,5R,7R)-6-(cyclopropanecarbonyl)-7-ethynyl-3-(4-(trifluoromethyl)phenyl)-3,6-diazabicyclo[3.2.1]octan-4-one (18 mg, 0.050 mmol), Pd(PPh₃)₄ (6.8 mg, 0.006 mmol), CuI (5.6 mg, 0.029 mmol) and DIPEA (0.041 mL, 0.235 mmol) in DMF (1 mL) was sparged with N₂ and heated at 80° C. for 2 h. After cooling to r.t., Cs₂CO₃ (47.8 mg, 0.147 mmol) was added to the mixture, and the resulting mixture was stirred at 90° C. for 3 h. The mixture was then allowed to cool to r.t. and diluted with EtOAc. The solution was washed with water (3×) and brine, dried over Na₂SO₄, filtered and concentrated.

The reaction residue was dissolved in TFA (1 mL), and stirred for at r.t. for 10 min. The mixture was then diluted with MeCN (4 mL) and purified by prep. LCMS (XBRIDGE® C₁₈ column, eluting with a gradient of MeCN/water containing 0.1% TFA, at flow rate of 60 mL/min.) to afford the title compounds as two separate peaks.

Example 120a: Peak 1 (major peak). LCMS calc. for C₄₃H₃₇Cl₂F₄N₆O₂ (M+H)⁺: m/z=815.2; found 815.2

Example 120b: Peak 2 (minor peak). LCMS calc. for C₄₃H₃₇Cl₂F4N₆O₂ (M+H)⁺: m/z=815.2; found 815.2

The alternative atropisomers 3-((Sₐ)-1-((1R,4R,5S)-2-azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile and 3-((Sₐ)-1-((1R,4R,5S)-2-azabicyclo[2.1.1]hexan-5-yl)-2-((1 S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above starting from tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate.

### Example 121. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate

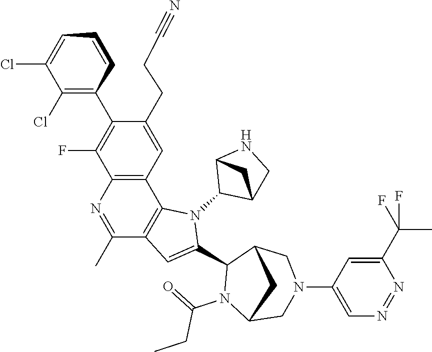

### Step 1. Dimethyl (1S,5R,7R)-2-methyl-3,6-diazabicyclo[3.2.1]octane-6,7-dicarboxylate

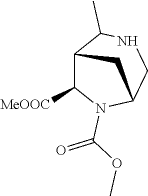 [SMILES: COC(=O)[C@H]1[C@H]2C[C@H](CNC2C)N1C(=O)OC]

To a solution of dimethyl (1 S,5R,7R)-2-methyl-3-(1-phenylethyl)-3,6-diazabicyclo[3.2.1]octane-6,7-dicarboxylate (Intermediate 13, 0.189 g, 0.546 mmol) in MeOH (10 mL) was added Pd(OH)₂/C (0.077 g, 0.109 mmol). The mixture was stirred under a H₂ atmosphere for 3 h. The resulting mixture was filtered through diatomaceous earth and concentrated. The crude material was used for next step without further purification. LCMS calc. for C₁₁H₁₉N₂O₄ (M+H)⁺: m/z=243.2; found 243.2

### Step 2. Dimethyl (1S,5R,7R)-3-(6-(1,1-difluoroethyl)pyrimidin-4-yl)-2-methyl-3,6-diazabicyclo[3.2.1]octane-6,7-dicarboxylate

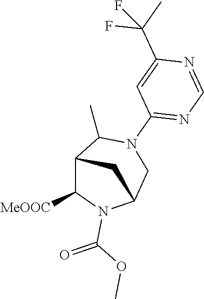 [SMILES: COC(=O)[C@H]1[C@H]2C[C@H](CN(c3cc(C(C)(F)F)ncn3)C2C)N1C(=O)OC]

A mixture of dimethyl (1 S,5R,7R)-2-methyl-3,6-diazabicyclo[3.2.1]octane-6,7-dicarboxylate (0.115 g, 0.475 mmol), RuPhos Pd G4 (0.040 g, 0.047 mmol), RuPhos (0.022 g, 0.047 mmol), sodium tert-butoxide (0.068 g, 0.712 mmol) and 4-chloro-6-(1,1-difluoroethyl)pyrimidine (0.424 g, 2.37 mmol) in THF (2.3 mL) was sparged with N₂ and heated at 85° C. for 3 h. The mixture was then allowed to cool to r.t. and diluted with EtOAc. The organic fraction was washed with brine, dried over Na₂SO₄, filtered and concentrated. The crude product was purified by FCC (0-60% EtOAc/hexanes) to afford the sub-title compound (0.039 g, 21% yield). LCMS calc. for C₁₇H₂₃F₂N₄O₄ (M+H)⁺: m/z=385.2; found 385.2.

### Step 3. Methyl (1S,5R,7R)-3-(6-(1,1-difluoroethyl)pyrimidin-4-yl)-7-(hydroxymethyl)-2-methyl-3,6-diazabicyclo[3.2.1]octane-6-carboxylate

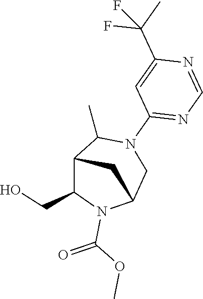 [SMILES: COC(=O)N1[C@@H]2C[C@@H](C(C)N(c3cc(C(C)(F)F)ncn3)C2)[C@@H]1CO]

To a solution of dimethyl (1 S,5R,7R)-3-(6-(1,1-difluoroethyl)pyrimidin-4-yl)-2-methyl-3,6-diazabicyclo[3.2.1]octane-6,7-dicarboxylate (0.039 g, 0.10 mmol) in THF (0.5 mL) was added LiBH₄ (2 M in THF, 0.13 mL, 0.25 mmol). The mixture was stirred at r.t. for 5 h and then quenched by slow addition of sat. aq. NH₄Cl at 0° C. The mixture was diluted with EtOAc, washed with brine, dried over Na₂SO₄, filtered and concentrated. The crude product was used for next step without further purification. LCMS calc. for C₁₆H₂₃F₂N₄O₃ (M+H)⁺: m/z=357.2; found 357.2.

### Step 4. Methyl (1S,5R,7R)-3-(6-(1,1-difluoroethyl)pyrimidin-4-yl)-7-formyl-2-methyl-3,6-diazabicyclo[3.2.1]octane-6-carboxylate

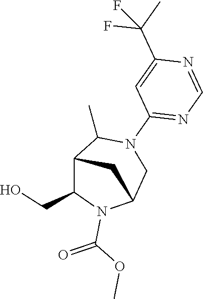 [SMILES: COC(=O)N1[C@@H]2C[C@@H](C(C)N(c3cc(C(C)(F)F)ncn3)C2)[C@@H]1CO]

To a solution of methyl (1 S,5R,7R)-3-(6-(1,1-difluoroethyl)pyrimidin-4-yl)-7-(hydroxymethyl)-2-methyl-3,6-diazabicyclo[3.2.1]octane-6-carboxylate (0.035 g, 0.098 mmol) in DCM (0.5 mL) was added Dess-Martin periodinane (0.063 g, 0.15 mmol) portionwise. The resulting mixture was stirred at r.t. for 1 h. Sat. aq. Na₂S₂O₃ (5 mL) was added to the mixture, which was then stirred for an additional 10 min. The organic phase was separated, dried over Na₂SO₄, filtered and concentrated to give crude product. The crude product was used in the next step without further purification. LCMS calc. for C₁₆H₂₁F₂N₄O₃ (M+H)⁺: m/z=355.2; found 355.2.

### Step 5. Methyl (1S,5R,7R)-3-(6-(1,1-difluoroethyl)pyrimidin-4-yl)-7-ethynyl-2-methyl-3,6-diazabicyclo[3.2.1]octane-6-carboxylate

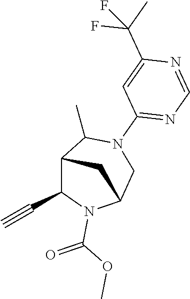 [SMILES: C#C[C@H]1[C@H]2C[C@H](CN(c3cc(C(C)(F)F)ncn3)C2C)N1C(=O)OC]

To a solution of methyl (1 S,5R,7R)-3-(6-(1,1-difluoroethyl)pyrimidin-4-yl)-7-formyl-2-methyl-3,6-diazabicyclo[3.2.1]octane-6-carboxylate (0.035 g, 0.098 mmol) in MeOH (0.5 mL) were added dimethyl(1-diazo-2-oxopropyl)phosphonate (0.047 g, 0.25 mmol) and K₂CO₃ (0.041 g, 0.30 mmol). After stirring for 2 h, the mixture was filtered through diatomaceous earth. The filtrate was concentrated. The residue was diluted with water, extracted with EtOAc, filtered through diatomaceous earth, dried over Na₂SO₄, filtered and concentrated. The crude product was purified by FCC (0-60% EtOAc/hexanes) to afford sub-title compound (0.014 g, 40% yield over 2 steps). LCMS calc. for C₁₇H₂₁F₂N₄O₂ (M+H)⁺: m/z=351.2; found 351.2.

### Step 6. Methyl (1S,5R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate

A mixture of Intermediate 2 (0.021 g, 0.031 mmol), methyl (1 S,5R,7R)-3-(6-(1,1-difluoroethyl)pyrimidin-4-yl)-7-ethynyl-2-methyl-3,6-diazabicyclo[3.2.1]octane-6-carboxylate (0.014 g, 0.039 mmol), CuI (0.002 g, 0.001 mmol), Pd(PPh₃)₄ (0.014 g, 0.012 mmol) and DIPEA (0.054 mL, 0.31 mmol) in DMF (0.15 mL) was sparged with N₂ and heated at 90° C. for 1 h. Then, Cs₂CO₃ (0.060 g, 0.19 mmol) was added to the mixture. The resulting slurry was stirred at 90° C. for another 5 h. The mixture was then allowed to cool to r.t. and poured into water. The solution was extracted with EtOAc (2×). Then the combined organic layers were washed with brine (5×), dried over Na₂SO₄, filtered and concentrated.

To the residue dissolved in DCM (0.5 mL) was added TFA (0.5 mL). The mixture was stirred at r.t. for 0.5 h. The volatiles were then removed under reduced pressure and the residue was dissolved in MeCN (4 mL) and water (1 mL) and purified by RP-HPLC (XBRIDGE® C₁₈ column, eluting with a gradient of MeCN/water containing 0.1% TFA, at flow rate of 60 mL/min.) to afford the title compound. LCMS calc. for C₄₁H₄₀Cl₂F₃N₈O₂ (M+H)⁺: m/z=803.3; found 803.3.

The alternative atropisomer methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate can be prepared by an analogous route by performing a process analogous to the step above using tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate in Step 6.

### Example 122. 3-((Rₐ)-1-((1R,4R,5S)-2-Azabicyclo[2.1.1]hexan-5-yl)-2-((1 S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

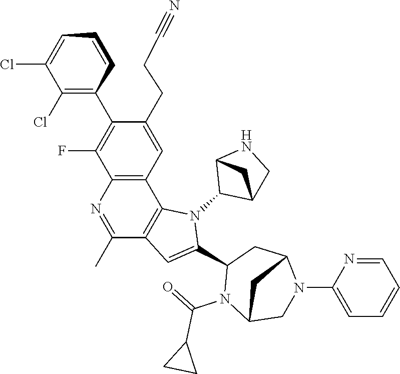

### Step 1. tert-Butyl (2R,3R,5R)-3-(((tert-butyldiphenylsilyl)oxy)methyl)-2-ethynyl-5-(pyridin-2-ylcarbamoyl)pyrrolidine-1-carboxylate

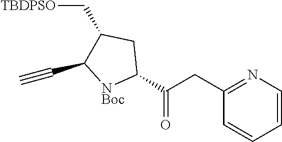

To a solution of (2R,4R,5R)-1-(tert-butoxycarbonyl)-4-(((tert-butyldiphenylsilyl)oxy)methyl)-5-ethynylpyrrolidine-2-carboxylic acid (intermediate 14, 0.480 g, 0.600 mmol) in EtOAc (3 mL) was added T3P (0.813 mL, 1.80 mmol), pyridine (0.194 mL, 2.40 mmol), and pyridin-2-amine (0.113 g, 1.20 mmol). The mixture was stirred at r.t. for 16 h. The mixture was diluted with brine and extracted with DCM (3×). The combined organic layers were washed with brine, dried over MgSO₄, filtered and concentrated. The crude product was used in the next step without further purification. LCMS calc. for C₃₄H₄₂N₃O₄Si (M+H)⁺: m/z=584.3; found 584.3.

### Step 2. tert-Butyl (2R,3R,5R)-2-ethynyl-3-(hydroxymethyl)-5-(pyridin-2-ylcarbamoyl)pyrrolidine-1-carboxylate

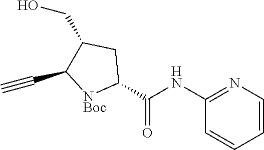 [SMILES: C#C[C@H]1[C@H](CO)C[C@H](C(=O)Nc2ccccn2)N1C(=O)OC(C)(C)C]

To a solution of tert-butyl (2R,3R,5R)-3-(((tert-butyldiphenylsilyl)oxy)methyl)-2-ethynyl-5-(pyridin-2-ylcarbamoyl)pyrrolidine-1-carboxylate (0.275 g, 0.471 mmol) in THF (2.3 mL) was added TBAF (1 M in THF, 0.71 mL, 0.71 mmol). The mixture was stirred at r.t. for 2 h and then diluted with sat. aq. NH₄Cl (10 mL) and extracted with MTBE (3×). The combined organic layers were washed with brine, dried over MgSO₄, filtered and concentrated. The crude product was purified by FCC (0-100% EtOAc/n-heptane) to afford the sub-title compound (0.137 g, 84%). LCMS calc. for C₁₈H₂₄N₃O₄ (M+H)⁺: m/z=346.2; found 346.2.

### Step 3. tert-Butyl (2R,3R,5R)-2-ethynyl-5-(pyridin-2-ylcarbamoyl)-3-((((4-(trifluoromethyl)phenyl)sulfonyl)oxy)methyl)pyrrolidine-1-carboxylate

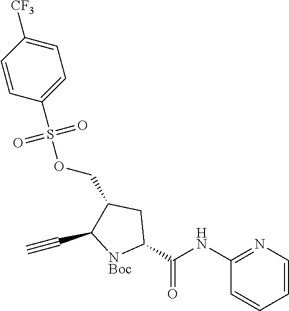 [SMILES: C#C[C@H]1[C@H](COS(=O)(=O)c2ccc(C(F)(F)F)cc2)C[C@H](C(=O)Nc2ccccn2)N1C(=O)OC(C)(C)C]

To a solution of tert-butyl (2R,3R,5R)-2-ethynyl-3-(hydroxymethyl)-5-(pyridin-2-ylcarbamoyl)pyrrolidine-1-carboxylate (0.135 g, 0.391 mmol) in DCM (2 mL) at 0° C. was added NEt₃ (0.27 mL, 1.95 mmol) and 4-(trifluoromethyl)benzenesulfonyl chloride (0.143 g, 0.586 mmol). After stirring at 0° C. for 45 min, the mixture was diluted with DCM and quenched with sat. aq. NaHCO₃. The resulting mixture was extracted with DCM (3×). The combined organic layers were washed with brine, dried over MgSO₄, filtered and concentrated. The crude product was purified by FCC (0-60% EtOAc/n-heptane) to afford sub-title compound (0.207 g, 96%). LCMS calc. for C₂₅H₂₇F₃N₃O₆S+(M+H)⁺: m/z=554.2; found 554.2.

### Step 4. tert-Butyl (1S,5R,7R)-7-ethynyl-4-oxo-3-(pyridin-2-yl)-3,6-diazabicyclo[3.2.1]octane-6-carboxylate

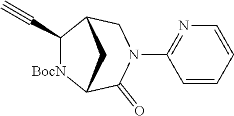 [SMILES: C#C[C@H]1[C@H]2C[C@H](C(=O)N(c3ccccn3)C2)N1C(=O)OC(C)(C)C]

To a solution of tert-butyl (2R,3R,5R)-2-ethynyl-5-(pyridin-2-ylcarbamoyl)-3-((((4-(trifluoromethyl)phenyl)sulfonyl)oxy)methyl)pyrrolidine-1-carboxylate (0.180 g, 0.325 mmol) in DMF (6.5 mL) was added Cs₂CO₃ (0.424 g, 1.30 mmol). The mixture was heated at 80° C. for 30 min. After cooling to r.t., the mixture was diluted with MTBE and 10% aq. LiCl. The mixture was extracted with MTBE (2×) and EtOAc. Combined organic extracts were washed with 10% aq. LiCl, dried over MgSO₄, filtered and concentrated. The crude product was used in the next step without further purification. LCMS calc. for C₁₈H₂₂N₃O₃ (M+H)⁺: m/z=328.2; found 328.2.

### Step 5. (1S,5R,7R)-7-Ethynyl-3-(pyridin-2-yl)-3,6-diazabicyclo[3.2.1]octan-4-one

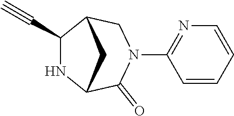 [SMILES: C#C[C@@H]1N[C@@H]2C[C@H]1CN(c1ccccn1)C2=O]

To a solution of tert-butyl (1 S,5R,7R)-7-ethynyl-4-oxo-3-(pyridin-2-yl)-3,6-diazabicyclo[3.2.1]octane-6-carboxylate (0.113 g, 0.345 mmol) in 2-methyltetrahydrofuran (1.1 mL) was added HCl (4 M in 1,4-dioxane, 1.1 mL). The mixture was stirred for 2 h and then slowly added into an oversized flask containing crushed ice and sat. aq. NaHCO₃ with rapid stirring. The resulting mixture was extracted with DCM (3×). The combined organic layers were washed with brine, dried over MgSO₄, filtered and concentrated. The crude product was used in the next step without further purification. LCMS calc. for C₁₃H₁₄N₃O+(M+H)⁺: m/z=228.1; found 228.1.

### Step 6. (1S,5R,7R)-6-(Cyclopropanecarbonyl)-7-ethynyl-3-(pyridin-2-yl)-3,6-diazabicyclo[3.2.1]octan-4-one

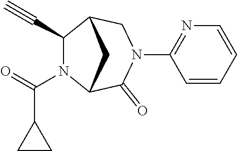 [SMILES: C#C[C@H]1[C@H]2C[C@H](C(=O)N(c3ccccn3)C2)N1C(=O)C1CC1]

To a solution of (1 S,5R,7R)-7-ethynyl-3-(pyridin-2-yl)-3,6-diazabicyclo[3.2.1]octan-4-one (0.026 g, 0.11 mmol) in DCM (2 mL) cooled to 0° C. was added DIPEA (0.40 mL, 2.3 mmol) and cyclopropanecarbonyl chloride (0.06 L mL, 0.57 mmol). The mixture was stirred at 0° C. for 30 min. The reaction was quenched with sat. aq. NaHCO₃ and the mixture was extracted with DCM (3×). The combined organic layers were washed with brine, dried over MgSO₄, filtered and concentrated. The crude product was used in the next step without further purification. LCMS calc. for C₁₇H₁₈N₃O₂ (M+H)⁺: m/z=296.1; found 296.1.

### Step 7. 3-((Rₐ)-1-((1R,4R,5S)-2-Azabicyclo[2.1.1]hexan-5-yl)-2-((1S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile

A mixture of Intermediate 2 (0.045 g, 0.066 mmol), (1 S,5R,7R)-6-(cyclopropanecarbonyl)-7-ethynyl-3-(pyridin-2-yl)-3,6-diazabicyclo[3.2.1]octan-4-one (0.025 g, 0.086 mmol), CuI (0.013 g, 0.066 mmol), Pd(PPh₃)₄ (0.010 g, 0.009 mmol) and DIPEA (0.12 mL, 0.66 mmol) in DMF (1.3 mL) was sparged with N₂ and heated at 85° C. for 1 h. After cooling to r.t., NMP (2.0 mL) and Cs₂CO₃ (0.325 g, 1.00 mmol) were added to the mixture. The resulting slurry was stirred at 95° C. for 1.5 h. The mixture was allowed to cool to r.t. and diluted with EtOAc/MTBE (1:1) and 10% aq. LiCl. The resulting mixture was extracted with EtOAc/MTBE (1:1, 3×). The combined organic layers were washed with 10% aq. LiCl, brine, dried over MgSO₄, filtered and concentrated.

To the residue dissolved in DCM (0.5 mL) was added TFA (0.5 mL). The mixture was stirred at r.t. for 0.5 h. The volatiles were then removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄H₃₇Cl₂FN₇O₂ (M+H)⁺: m/z=748.2; found 748.2.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile can be prepared by an analogous route by performing a process analogous to the step above using tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate in Step 7.

### Example 123. 3-((Rₐ)-1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile

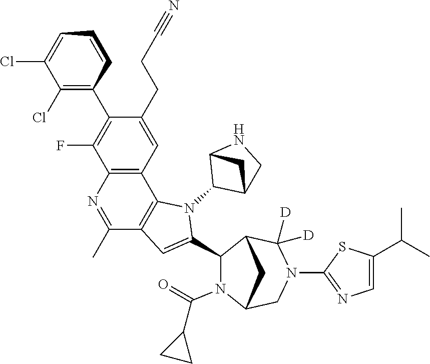

### Step 1. tert-Butyl (1R,4R,5S)-5-((Rₐ)-8-(2-cyanoethyl)-2-((1S,5R,7R)-6-(cyclopropanecarbonyl)-3-((2-(trimethylsilyl)ethoxy)carbonyl)-3,6-diazabicyclo[3.2.1]octan-7-yl-2,2-d2)-7-(2,3-dichlorophenyl)-6-fluoro-4-methyl-1H-pyrrolo[3,2-c]quinolin-1-yl)-2-azabicyclo[2.1.1]hexane-2-carboxylate

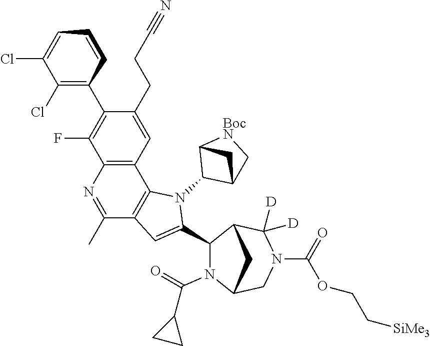

A mixture of Intermediate 2 (0.035 g, 0.050 mmol), Intermediate 15 (0.020 g, 0.057 mmol), CuI (0.004 g, 0.020 mmol), Pd(PPh₃)₄ (0.012 g, 0.010 mmol) and DIPEA (0.072 mL, 0.41 mmol) in DMF (1.5 mL) was sparged with N₂ and heated at 70° C. for 1 h. After cooling to r.t., Cs₂CO₃ (0.084 g, 0.26 mmol) was added to the mixture, and the resulting mixture was stirred at 90° C. for 2 h. The mixture was cooled down to r.t. and diluted with EtOAc. The resulting mixture was washed with brine (3×), dried over Na₂SO₄, filtered and concentrated. The crude product was used in the next step without further purification. LCMS calc. for C₄₇H₅₄D₂Cl₂FN₆O₅Si (M+H)⁺: m/z=903.4; found 903.5.

### Step 2. tert-Butyl (1R,4R,5S)-5-((Rₐ)-8-(2-cyanoethyl)-2-((1S,5R,7R)-6-(cyclopropanecarbonyl)-3,6-diazabicyclo[3.2.1]octan-7-yl-2,2-d2)-7-(2,3-dichlorophenyl)-6-fluoro-4-methyl-1H-pyrrolo[3,2-c]quinolin-1-yl)-2-azabicyclo[2.1.1]hexane-2-carboxylate

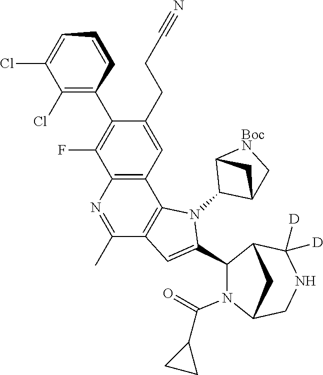

To a solution of tert-butyl (1R,4R,5S)-5-((Rₐ)-8-(2-cyanoethyl)-2-((1 S,5R,7R)-6-(cyclopropanecarbonyl)-3-((2-(trimethylsilyl)ethoxy)carbonyl)-3,6-diazabicyclo[3.2.1]octan-7-yl-2,2-d₂)-7-(2,3-dichlorophenyl)-6-fluoro-4-methyl-1H-pyrrolo[3,2-c]quinolin-1-yl)-2-azabicyclo[2.1.1]hexane-2-carboxylate (0.035 g, 0.039 mmol) in THF (1 mL) was added tris(dimethylamino)sulfonium difluorotrimethylsilicate(IV) (0.016 g, 0.058 mmol). The mixture was stirred at 60° C. for 1 h. After cooling to r.t., the mixture was diluted with EtOAc, washed with brine, dried over Na₂SO₄, filtered and concentrated. The crude product was used in the next step without further purification. LCMS calc. for C₄₁H₄₂D₂Cl₂FN₆O₃ (M+H)⁺: m/z=759.3; found 759.4.

### Step 3. 3-((Rₐ)-1-((1R,4R,5S)-2-Azabicyclo[2.1.1]hexan-5-yl)-2-((1S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile

A mixture of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl-2,2-d₂)-7-(2,3-dichlorophenyl)-6-fluoro-4-methyl-1H-pyrrolo[3,2-c]quinolin-1-yl)-2-azabicyclo[2.1.1]hexane-2-carboxylate (0.010 g, 0.013 mmol), GPhos Pd G6 TES (0.001 g, 0.001 mmol), sodium trimethylsilanolate (0.008 g, 0.066 mmol), 2-bromo-5-isopropylthiazole (0.011 g, 0.053 mmol) in THF (0.8 mL) was sparged with N₂ and heated to 90° C. for 6 h. After cooling the mixture to r.t., the volatiles were removed under reduced pressure.

The residue was dissolved in TFA (1 mL) and stirred for at r.t. for 10 min. The reaction mixture was then diluted with MeCN (4 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₂H₄₁D₂Cl₂FN₇OS (M+H)⁺: m/z=784.3; found 784.4.

The alternative atropisomer 3-((Sₐ)-1-((1R,4R,5S)-2-azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile can be prepared by an analogous route by performing a process analogous to the step above using tert-butyl (1R,4R,5S)-5-(((Sₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate instead of tert-butyl (1R,4R,5S)-5-((Rₐ)-8-(2-cyanoethyl)-2-((1 S,5R,7R)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-1-yl)-2-azabicyclo[2.1.1]hexane-2-carboxylate in Step 1.

### Example 124. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate

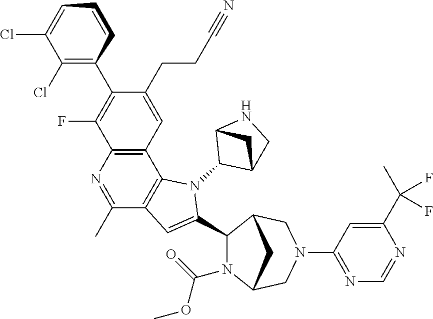

A mixture of tert-butyl (1R,4R,5S)-5-((Rₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate (Intermediate 16, 0.040 g, 0.053 mmol), RuPhos Pd G4 (0.005 g, 10 mol %), RuPhos (0.002 g, 10 mol %), sodium tert-butoxide (2M THF, 7 L, 0.08 mmol) and 4-chloro-6-(1,1-difluoroethyl)pyrimidine (0.048 g, 0.27 mmol) in THF (0.27 mL) was sparged with N₂ and heated at 85° C. for 16 h. The mixture was then allowed to cool to r.t. and diluted with EtOAc. The organic fraction was washed with brine, dried over Na₂SO₄, filtered and concentrated.

The residue was dissolved in DCM (2 mL) were added MeCN (0.2 mL) and TFA (2 mL). The mixture was stirred at r.t. for 0.5 h. The volatiles were then removed under reduced pressure and the residue was dissolved in MeCN (12 mL) and water (3 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₄₀H₃₈Cl₂F₃N₈O₂ (M+H)⁺: m/z=789.2/791.2; found 789.3/791.3. ¹H NMR was collected on the TFA salt. ¹H NMR (500 MHz, DMSO) δ 9.26 (broad s, 1H), 8.69 (s, 1H), 8.15 (s, 1H), 8.10 (broad s, 1H), 7.86 (d, J=8.1 Hz, 1H), 7.59 (t, J=7.9 Hz, 1H), 7.51-7.42 (m, 1H), 7.09-6.89 (m, 2H), 5.60 (s, 1H), 4.91-4.83 (m, 2H), 4.44 (s, 1H), 3.99-3.89 (m, 1H), 3.71 (s, 3H), 3.65-3.49 (m, 2H), 3.47-3.31 (m, 2H), 3.23-3.00 (m, 2H), 2.94-2.78 (m, 5H), 2.72-2.59 (m, 1H), 2.56-2.54 (m, 1H), 2.43-2.32 (m, 1H), 2.04-1.91 (m, 4H), 1.89-1.73 (m, 2H), 1.66-1.58 (m, 1H).

The alternative atropisomer methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate can be prepared by an analogous route by performing a process analogous to the step above using tert-butyl (1R,4R,5S)-5-((Sₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate.

### Example 125. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate

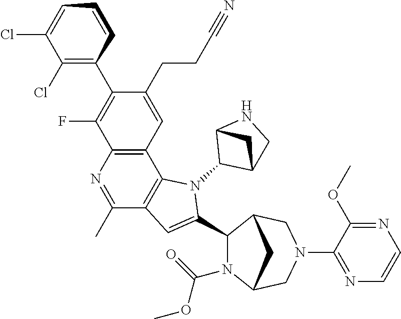

A mixture of tert-butyl (1R,4R,5S)-5-((Rₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate (Intermediate 16, 0.30 g, 0.40 mmol), RuPhos Pd G4 (0.034 g, 10 mol %), sodium tert-butoxide (2M THF, 0.30 mL, 0.60 mmol) and 2-iodo-3-methoxypyrazine (0.47 g, 2.0 mmol) in 1,4-dioxane (5.7 mL) was sparged with N₂ and heated at 30° C. for 2 h. The mixture was diluted with EtOAc and water. The organic fraction was washed with brine, dried over Na₂SO₄, filtered and concentrated. The crude material was purified by prep. LCMS (XBRIDGE® C₁₈ column, eluting with a gradient of MeCN/water containing 0.1% NH₄OH, at flow rate of 60 mL/min.). The resulting product fraction was concentrated under reduced pressure.

The residue was dissolved in DCM (1 mL) and TFA (1 mL) and was stirred at r.t. for 0.5 h. The volatiles were then removed under reduced pressure and the residue was dissolved in MeCN and water and purified by prep. LCMS (XBRIDGE® C₁₈ column, eluting with a gradient of MeCN/water containing 0.1% TFA, at flow rate of 60 mL/min.) to afford the title compound. LCMS calc. for C₃₉H₃₈Cl₂FN₈O₃ (M+H)⁺: m/z=755.2/757.2; found 755.3/757.3. ¹H NMR was collected on the TFA salt. ¹H NMR (600 MHz, DMSO, single rotamer) δ 9.47-9.28 (m, 1H), 8.17-8.12 (m, 2H), 7.86 (dd, J=8.1, 1.5 Hz, 1H), 7.82 (d, J=2.8 Hz, 1H), 7.68-7.63 (m, 1H), 7.59 (t, J=8.0 Hz, 1H), 7.50-7.44 (m, 1H), 6.97 (s, 1H), 5.66 (s, 1H), 5.04 (s, 1H), 4.94-4.88 (m, 1H), 4.55-4.44 (m, 1H), 4.44-4.38 (m, 1H), 4.30 (t, J=4.8 Hz, 1H), 3.98-3.93 (m, 4H), 3.73-3.68 (m, 1H), 3.66 (s, 3H), 3.53-3.47 (m, 1H), 3.19-3.12 (m, 1H), 3.11-3.04 (m, 1H), 3.04-2.96 (m, 1H), 2.92-2.80 (m, 5H), 2.73-2.63 (m, 1H), 2.50-2.44 (m, 1H), 2.43-2.38 (m, 1H), 2.02-1.95 (m, 1H), 1.77-1.67 (m, 1H), 1.64 (d, J=9.2 Hz, 1H).

The alternative atropisomer methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate can be prepared by an analogous route by performing a process analogous to the step above using tert-butyl (1R,4R,5S)-5-((Sₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate.

### Example 126. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate

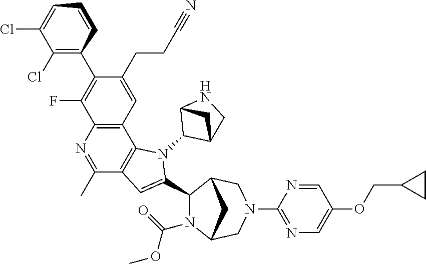

This compound was prepared in an analogous fashion to Example 31, with 2-chloro-5-(cyclopropylmethoxy)pyrimidine replacing 4-chloro-5-fluoro-6-methylpyrimidine and Intermediate 16 replacing Intermediate 8. LCMS calc. for C₄₂H₄₂Cl₂FN₈O₃ (M+H)⁺: m/z=795.3/797.3; found 795.3/797.3.

The alternative atropisomer methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate can be prepared by an analogous route by performing a process analogous to the step above using tert-butyl (1R,4R,5S)-5-((Sₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate.

### Example 127. Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate

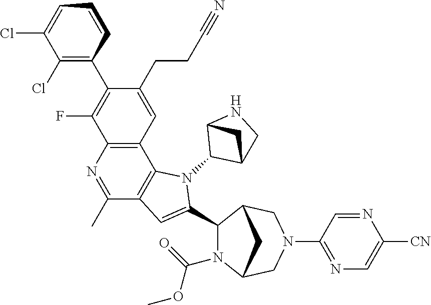

This compound was prepared in an analogous fashion to Example 31, with 5-bromopyrazine-2-carbonitrile replacing 4-chloro-5-fluoro-6-methylpyrimidine and Intermediate 16 replacing Intermediate 8. LCMS calc. for C₃₉H₃₅Cl₂FN₉O₂ (M+H)⁺: m/z=750.2/752.2; found 750.1/752.2.

The alternative atropisomer Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate can be prepared by an analogous route by performing a process analogous to the step above using tert-butyl (1R,4R,5S)-5-((Sₐ)-8-(2-cyanoethyl)-7-(2,3-dichlorophenyl)-6-fluoro-2-((1 S,5R,7R)-6-(methoxycarbonyl)-3,6-diazabicyclo[3.2.1]octan-7-yl)-4-methyl-1H-pyrrolo[3,2-c]quinolin-1-yl)-2-azabicyclo[2.1.1]hexane-2-carboxylate.

### Example 128. 3-((Rₐ)-1-((1R,4R,5S)-2-azabicyclo[2.1.1]hexan-5-yl)-7-(2,3-dichlorophenyl)-2-((1S,5R,7R)-3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile

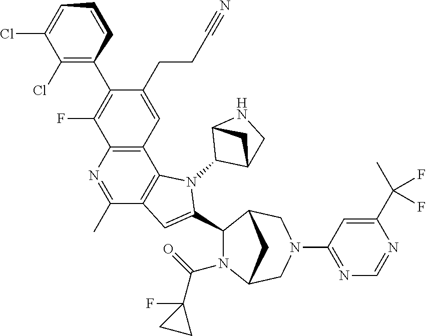

To a 0° C. cooled solution of tert-butyl (1R,4R,5S)-5-((Rₐ)-8-(2-cyanoethyl)-7-(2,3-dichlorophenyl)-2-((1 S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate (Intermediate 17, 0.008 g, 0.0096 mmol), HATU (0.011 g, 0.029 mmol), and DIPEA (8.4 L, 0.048 mmol) in THF (0.5 mL) was added 1-fluorocyclopropane-1-carboxylic acid (0.002 g, 0.019 mmol). The mixture was stirred for 30 min at r.t., at which point, the reaction mixture was diluted with EtOAc, washed with water and then brine. The organics were dried over Na₂SO₄, filtered and concentrated. The residue was dissolved in DCM (1 mL) and TFA (1 mL) and was stirred at r.t. for 0.5 h. The volatiles were then removed under reduced pressure and the crude was purified by preparative LC-MS (XBridge® C18 column, eluting with a gradient of acetonitrile/water containing 0.1% TFA, at flow rate of 60 mL/min) to afford the title compound. LC-MS calc. for C₄₂H₃₉Cl₂F₄N₈O (M+H)⁺: m/z=817.3/819.3; found 817.3/819.3.

The alternative atropisomer 3-((Rₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile can be prepared by an analogous route by performing a process analogous to the step above using tert-butyl (1R,4R,5S)-5-((Sₐ)-8-(2-cyanoethyl)-7-(2,3-dichlorophenyl)-2-((1 S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate.

### Example 129. 3-((Rₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile

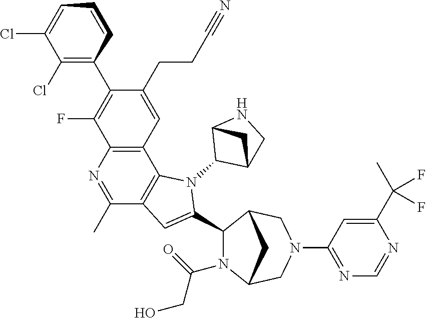

To a solution of tert-butyl (1R,4R,5S)-5-((Rₐ)-8-(2-cyanoethyl)-7-(2,3-dichlorophenyl)-2-((1 S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate (Intermediate 17, 0.008 g, 0.0096 mmol) in THF (0.5 mL) cooled to 0° C. were added DIPEA (8.4 μL, 0.048 mmol) and 2-chloro-2-oxoethyl acetate (2.63 mg, 0.019 mmol). The mixture was stirred at r.t. for 30 minutes and was then diluted with EtOAc. The organics were washed with water and brine, dried over Na₂SO₄, filtered and concentrated. The residue was dissolved in DCM (1 mL) and TFA (1 mL) and was stirred at r.t. for 0.5 h. The volatiles were removed under reduced pressure. The residue was dissolved in MeOH (0.5 mL) and 1 N NaOH (0.48 mL, 0.48 mmol) was added. The solution was stirred at r.t. for 16 h and then neutralized with 1 N HCl. The crude was purified by preparative LC-MS (XBridge® C18 column, eluting with a gradient of acetonitrile/water containing 0.1% TFA, at flow rate of 60 mL/min) to afford the title compound. LC-MS calc. for C₄₀H₃₈Cl₂F₃N₈O₂ (M+H)⁺: m/z=789.2/791.2; found 789.3/791.2.

The alternative atropisomer 3-((Sₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile can be prepared by an analogous route by performing a process analogous to the step above using tert-butyl (1R,4R,5S)-5-((Sₐ)-8-(2-cyanoethyl)-7-(2,3-dichlorophenyl)-2-((1 S,5R,7R)-3-(6-(1,1-difluoroethyl)pyrimidin-4-yl)-3,6-diazabicyclo[3.2.1]octan-7-yl)-6-fluoro-4-methyl-1H-pyrrolo[3,2-c]quinolin-1-yl)-2-azabicyclo[2.1.1]hexane-2-carboxylate.

### Example A. GDP-GTP Exchange Assay

The inhibitor potency of the exemplified compounds was determined in a fluorescence based guanine nucleotide exchange assay, which measures the exchange of bodipy-GDP (fluorescently labeled GDP) for GppNHp (Non-hydrolyzable GTP analog) to generate the active state of KRAS in the presence of SOS1 (guanine nucleotide exchange factor). Inhibitors were serially diluted in DMSO and a volume of 0.1 μL was transferred to the wells of a black low volume 384-well plate. 5 μL/well volume of bodipy-loaded KRAS G12D diluted to 2.5 nM in assay buffer (25 mM Hepes pH 7.5, 50 mM NaCl, 10 mM MgCl₂ and 0.01% Brij-35) was added to the plate and pre-incubated with inhibitor for 4 h at r.t. Appropriate controls (enzyme with no inhibitor or with a G12D inhibitor) were included on the plate. The exchange was initiated by the addition of a 5 μL/well volume containing 1 mM GppNHp and 300 nM SOS1 in assay buffer. The 10 μL/well reaction concentration of the bodipy-loaded KRAS G12D, GppNHp, and SOS1 were 2.5 nM, 500 μM, and 150 nM, respectively. The reaction plates were incubated at r.t. for 2 h, a time estimated for complete GDP-GTP exchange in the absence of inhibitor. For the KRAS G12V mutant, similar guanine nucleotide exchange assays were used with 2.5 nM as final concentration for the bodipy loaded KRAS proteins and 3 h incubation after adding GppNHp-SOS1 mixture. A cyclic peptide described to selectively bind G12D mutant (Sakamoto et al., BBRC 484.3 (2017), 605-611) or internal compounds with confirmed binding were used as positive controls in the assay plates. Fluorescence intensities were measured on a PheraStar plate reader instrument (BMG Labtech) with excitation at 485 nm and emission at 520 nm.

Either GraphPad prism or Genedata Screener SmartFit was used to analyze the data. The IC₅₀ values were derived by fitting the data to a four parameter logistic equation producing a sigmoidal dose-response curve with a variable Hill coefficient.

The KRAS_G12D and KRAS_G12V exchange assay IC₅₀ data are provided in Table 6 below. The symbol “⬆” indicates IC₅₀ 100 nM, “⬆⬆” indicates IC₅₀ >100 nM but ≤1 μM; and “⬆⬆⬆” indicates IC₅₀ is >1 μM but ≤5 μM, “⬆⬆⬆⬆” indicates IC₅₀ is >5 μM but ≤10 μM. “NA” indicates IC₅₀ not available.

TABLE 6

| Ex. No. | G12D_exchange | G12V_exchange |
|---|---|---|
| 1 | †† | † |
| 2 | † | † |
| 3 | †† | † |
| 4 | † | † |
| 5 | † | † |
| 6 | † | † |
| 7 | † | † |
| 8 | † | † |
| 9 | † | † |
| 10 | †† | † |
| 11 | † | † |
| 12 | †† | † |
| 13 | † | † |
| 14 | † | † |
| 15 | ††† | †††† |
| 16 | ††† | ††† |
| 17 | †† | † |
| 18 | †† | † |
| 19 | †† | † |
| 20 | †† | † |
| 21 | † | † |
| 22 | † | † |
| 23 | † | † |
| 24 | †† | † |
| 25 | †† | † |
| 26 | †† | † |
| 27 | † | † |
| 28 | †† | † |
| 29 | † | † |
| 30 | † | † |
| 31 | † | † |
| 32 | † | † |
| 33 | † | † |
| 34 | † | † |
| 35 | † | † |
| 36 | † | † |
| 37 | † | † |
| 38 | † | † |
| 39 | † | † |
| 40 | † | † |
| 41 | † | † |
| 42 | † | † |
| 43 | † | † |
| 44 | † | † |
| 45 | † | † |
| 46 | † | † |
| 47 | †† | † |
| 48 | † | † |
| 49 | † | † |
| 50 | †† | † |
| 51 | † | † |
| 52 | †† | † |
| 53 | † | † |
| 54 | † | † |
| 55 | †† | † |
| 56 | † | † |
| 57 | †† | † |
| 58 | † | † |
| 59 | † | † |
| 60 | † | † |
| 61 | † | † |
| 62 | † | † |
| 63 | † | † |
| 64 | † | † |
| 65 | † | † |
| 66 | † | † |
| 67 | † | † |
| 68 | † | † |
| 69 | † | † |
| 70 | † | † |
| 71 | † | † |
| 72 | † | † |
| 73 | † | † |
| 74 | † | † |
| 75 | †† | † |
| 76 | † | † |
| 77 | †† | † |
| 78 | † | † |
| 79 | † | † |
| 80 | † | † |
| 81 | † | † |
| 82 | † | † |
| 83 | † | † |
| 84 | † | † |
| 85 | † | † |
| 86 | † | † |
| 87 | † | † |
| 88 | † | † |
| 89 | † | † |
| 90 | † | † |
| 91 | † | † |
| 92 | † | † |
| 93 | † | † |
| 94 | † | † |
| 95 | † | † |
| 96 | † | † |
| 96 | † | † |
| 97 | † | † |
| 98 | † | † |
| 99 | † | † |
| 100 | † | † |
| 101 | † | † |
| 102 | † | † |
| 103 | † | † |
| 104 | † | † |
| 105 | † | † |
| 106 | † | † |
| 107 | † | † |
| 108 | † | † |
| 109 | † | † |
| 110 | † | † |
| 111 | †† | † |
| 112 | †† | † |
| 113 | ††† | † |
| 114 | † | † |
| 115 | † | † |
| 116 | † | † |
| 117 | † | † |
| 118 | † | † |
| 119 | † | † |
| 120a | †† | † |
| 120b | † | † |
| 121 | † | † |
| 122 | † | † |
| 123 | †† | † |
| 124 | † | † |
| 125 | NA | † |
| 126 | NA | † |
| 127 | NA | † |
| 128 | NA | † |
| 129 | NA | † |

### Example B: Luminescent Viability Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are cultured in RPMI 1640 media supplemented with 10% FBS (Gibco/Life Technologies). Eight hundred cells per well in RPMI 1640 media supplemented with 2% FBS are seeded into white, clear bottomed 384-well Costar tissue culture plates containing 50 nL dots of test compounds (final concentration is a 1:500 dilution, with a final concentration in 0.2% DMSO). Plates are incubated for 3 days at 37° C., 5% CO₂. At the end of the assay, 25 μL/well of CellTiter-Glo reagent (Promega) is added. Luminescence is read after 15 min. with a PHERAstar (BMG). Data are analyzed in Genedata Screener using SmartFit for IC₅₀ values.

### Example C: Cellular pERK HTRF Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are purchased from ATCC and maintained in RPMI 1640 media supplemented with 10% FBS (Gibco/Life Technologies). The cells are plated at 5000 cells per well (8 μL) into Greiner 384-well low volume, flat-bottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h (G12C and G12V) or 2 h (G12D) at 37° C., 5% CO₂. 4 μL of 4× lysis buffer with blocking reagent (Cisbio) are added to each well and plates are rotated gently (300 rpm) for 30 min. at r.t. 4 μL per well of Cisbio anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example D: Whole Blood pERK1/2 HTRF Assay

MIA PaCa-2 cells (KRAS G12C; ATCC® CRL-1420), HPAF-II (KRAS G12D; ATCC® CRL-1997) and YAPC (KRAS G12V; DSMZ ACC382) are maintained in RPMI 1640 with 10% FBS (Gibco/Life Technologies). For MIA PaCa-2 assay, cells are seeded into 96 well tissue culture plates (Corning #3596) at 25000 cells per well in 100 μL media and cultured for 2 days at 37° C., 5% CO₂ before the assay. For HPAF-II and YAPC assay, cells are seeded in 96 well tissue culture plates at 50000 cells per well in 100 μL media and cultured for 1 day before the assay. Whole Blood are added to the 1 μL dots of compounds (prepared in DMSO) in 96 well plates and mixed gently by pipetting up and down so that the concentration of the compound in blood is 1× of desired concentration, in 0.5% DMSO. The media is aspirated from the cells and 50 μL per well of whole blood with test compound is added and incubated for 4 h for MIA PaCa and YAPC assay; or 2 h for HPAF-II assay, respectively at 37° C., 5% CO₂. After dumping the blood, the plates are gently washed (2×) by adding PBS to the side of the wells and dumping the PBS from the plate onto a paper towel, tapping the plate to drain well. 50 μL/well of 1× lysis buffer #1 (Cisbio) with blocking reagent (Cisbio) and Benzonase nuclease (Sigma Cat #E1014-5KU, 1:10000 final concentration) is then added and incubated at r.t. for 30 min. with shaking (250 rpm). Following lysis, 16 μL of lysate is transferred into 384-well Greiner small volume white plate using an Assist Plus (Integra Biosciences, NH). 4 μL of 1:1 mixture of anti Phospho-ERK 1/2 d2 and anti Phospho-ERK 1/2 Cryptate (Cisbio) is added to the wells using the Assist Plus and incubated at r.t. overnight in the dark. Plates are read on the Pherastar plate reader at 665 nm and 620 nm wavelengths. Data are analyzed in Genedata Screener using SmartFit for IC₅₀ values.

### Example E: Ras Activation ELISA

The 96-Well Ras Activation ELISA Kit (Cell Biolabs Inc; #STA441) uses the Raf1 RBD (Rho binding domain) bound to a 96-well plate to selectively pull down the active form of Ras from cell lysates. The captured GTP-Ras is then detected by a pan-Ras antibody and HRP-conjugated secondary antibody. MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are maintained in RPMI 1640 with 10% FBS (Gibco/Life Technologies). The cells are seeded into 96 well tissue culture plates (Corning #3596) at 25000 cells per well in 100 μL media and cultured for 2 days at 37° C., 5% CO₂ so that they are approximately 80% confluent at the start of the assay. The cells are treated with compounds for either 4 h or overnight at 37° C., 5% CO₂. At the time of harvesting, the cells are washed with PBS, drained well and then lysed with 50 μL of the 1× Lysis buffer (provided by the kit) plus added Halt Protease and Phosphatase inhibitors (1:100) for 1 h on ice.

The Raf-1 RBD is diluted 1:500 in Assay Diluent (provided in kit) and 100 μL of the diluted Raf-1 RBD is added to each well of the Raf-1 RBD Capture Plate. The plate is covered with a plate sealing film and incubated at r.t. for 1 h on an orbital shaker. The plate is washed (3×) with 250 μL 1× Wash Buffer per well with thorough aspiration between each wash. 50 μL of Ras lysate sample (10-100 μg) is added per well in duplicate. A “no cell lysate” control is added in a couple of wells for background determination. 50 μL of Assay Diluent is added to all wells immediately to each well and the plate is incubated at r.t. for 1 h on an orbital shaker. The plate is washed 5 times with 250 μL 1× Wash Buffer per well with thorough aspiration between each wash. 100 μL of the diluted Anti-pan-Ras Antibody is added to each well and the plate is incubated at r.t. for 1 h on an orbital shaker. The plate is washed 5 times as previously. 100 μL of the diluted Secondary Antibody, HRP Conjugate is added to each well and the plate is incubated at r.t. for 1 h on an orbital shaker. The plate is washed 5 times as previously and drained well. 100 μL of Chemiluminescent Reagent (provided in the kit) is added to each well, including the blank wells. The plate is incubated at r.t. for 5 min. on an orbital shaker before the luminescence of each microwell is read on a plate luminometer. The % inhibition is calc. relative to the DMSO control wells after a background level of the “no lysate control” is subtracted from all the values. IC₅₀ determination is performed by fitting the curve of inhibitor percent inhibition versus the log of the inhibitor concentration using the GraphPad Prism 7 software.

### Example F: Inhibition of RAS-RAF and PI3K-AKT Pathways

The cellular potency of compounds is determined by measuring phosphorylation of KRAS downstream effectors extracellular-signal-regulated kinase (ERK), ribosomal S6 kinase (RSK), AKT (also known as protein kinase B, PKB) and downstream substrate S6 ribosomal protein.

To measure phosphorylated extracellular-signal-regulated kinase (ERK), ribosomal S6 kinase (RSK), AKT and S6 ribosomal protein, cells (details regarding the cell lines and types of data produced are further detailed in Table 7) are seeded overnight in Corning 96-well tissue culture treated plates in RPMI medium with 10% FBS at 4×104 cells/well. The following day, cells are incubated in the presence or absence of a concentration range of test compounds for 4 h at 37° C., 5% CO₂. Cells are washed with PBS and lysed with 1× lysis buffer (Cisbio) with protease and phosphatase inhibitors (Thermo Fisher, 78446). 10 or 20 μg of total protein lysates is subjected to SDS-PAGE and immunoblot analysis using following antibodies: phospho-ERK1/2-Thr202/Tyr204 (#9101 L), total-ERK1/2 (#9102L), phosphor-AKT-Ser473 (#4060L), phospho-p90RSK-Ser380 (#11989S) and phospho-S6 ribosomal protein-Ser235/Ser236 (#2211S) are from Cell Signaling Technologies (Danvers, MA).

TABLE 7

| | | KRAS | |
|---|---|---|---|
| Cell Line | Histology | alteration | Readout |
| H358 | Lung | G12C | pERK, pAKT, p-S6, p-p90RSK |
| MIA PaCa-2 | Pancreas | G12C | pERK, pAKT, p-S6, p-p90RSK |
| HPAF II | Pancreas | G12D | pERK, pAKT, p-S6, p-p90RSK |
| A427 | Lung | G12D | pERK, pAKT, p-S6, p-p90RSK |
| AGS | Stomach | G12D | pERK, pAKT, p-S6, p-p90RSK |
| PaTu 8988s | Pancreas | G12V | pERK, pAKT, p-S6, p-p90RSK |
| H441 | Lung | G12V | pERK, pAKT, p-S6, p-p90RSK |
| YAPC | Pancreas | G12V | pERK, pAKT, p-S6, p-p90RSK |
| SW480 | Colorectal | G12V | pERK, pAKT, p-S6, p-p90RSK |

### Example G: In Vivo Efficacy Studies

MIA-PaCa-2 (KRAS G12C), H358 (KRAS G12C), HPAF-II (KRAS G12D), AGS (KRAS G12D), SW480 (KRAS G12V) or YAPC(KRAS G12V) human cancer cells are obtained from the American Type Culture Collection and maintained in RPMI media supplemented with 10% FBS. For efficacy studies experiments, 5×106 cells are inoculated subcutaneously into the right hind flank of 6- to 8-week-old BALB/c nude mice (Charles River Laboratories, Wilmington, MA, USA). When tumor volumes are approximately 150-250 mm3, mice are randomized by tumor volume and compounds are orally administered. Tumor volume is calc. using the formula (L×W2)/2, where L and W refer to the length and width dimensions, respectively. Tumor growth inhibition is calc. using the formula (1−(VT/VC))×100, where VT is the tumor volume of the treatment group on the last day of treatment, and VC is the tumor volume of the control group on the last day of treatment. Two-way analysis of variance with Dunnett's multiple comparisons test is used to determine statistical differences between treatment groups (GraphPad Prism). Mice are housed at 10-12 animals per cage, and are provided enrichment and exposed to 12 h light/dark cycles. Mice whose tumor volumes exceeded limits (10% of body weight) are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example H: Caco2 Assay

Caco-2 cells are grown at 37° C. in an atmosphere of 5% CO₂ in DMEM growth medium supplemented with 10% (v/v) fetal bovine serum, 1% (v/v) nonessential amino acids, penicillin (100 U/mL), and streptomycin (100 μg/mL). Confluent cell monolayers are subcultured every 7 days or 4 days for Caco-2 by treatment with 0.05% trypsin containing 1 μM EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values 300 Ω·cm² are used for transport experiments. To determine the Pₐₚₚ in the absorptive direction (A-B), solution of test compound (50 μM) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

Pₐₚₚ(cm/s)=(F*VD)/(SA*MD),

where the flux rate (F, mass/time) is calc. from the slope of cumulative amounts of compound of interest on the receiver side, SA is the surface area of the cell membrane, VD is the donor volume, and MD is the initial amount of the solution in the donor chamber.

The Caco-2 data are provided in Table 8 below. The symbol “+” indicates a Caco-2 value of ≤0.5, “++” indicates a Caco-2 value of >0.5 but ≤1; and “+++” indicates a Caco-2 value of >1. “NA” indicates Caco-2 value not available.

TABLE 8

| Ex. No. | Caco-2 |
|---|---|
| 1 | + |
| 2 | + |
| 3 | + |
| 4 | + |
| 5 | + |
| 6 | NA |
| 7 | + |
| 8 | + |
| 9 | + |
| 10 | + |
| 11 | NA |
| 12 | + |
| 13 | + |
| 14 | + |
| 15 | + |
| 16 | NA |
| 17 | + |
| 18 | NA |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | +++ |
| 33 | + |
| 34 | + |
| 35 | + |
| 36 | + |
| 37 | NA |
| 38 | + |
| 39 | + |
| 40 | + |
| 41 | + |
| 42 | + |
| 43 | + |
| 44 | + |
| 45 | + |
| 46 | + |
| 47 | + |
| 48 | ++ |
| 49 | + |
| 50 | + |
| 51 | ++ |
| 52 | ++ |
| 53 | ++ |
| 54 | + |
| 55 | ++ |
| 56 | + |
| 57 | NA |
| 58 | + |
| 59 | + |
| 60 | + |
| 61 | + |
| 62 | + |
| 63 | NA |
| 64 | + |
| 65 | + |
| 66 | NA |
| 67 | + |
| 68 | + |
| 69 | + |
| 70 | + |
| 71 | + |
| 72 | + |
| 73 | + |
| 74 | + |
| 75 | NA |
| 76 | ++ |
| 77 | NA |
| 78 | + |
| 79 | + |
| 80 | + |
| 81 | + |
| 82 | NA |
| 83 | + |
| 84 | + |
| 85 | + |
| 86 | + |
| 87 | + |
| 88 | + |
| 89 | + |
| 90 | + |
| 91 | + |
| 92 | + |
| 93 | + |
| 94 | + |
| 95 | + |
| 96 | + |
| 97 | + |
| 98 | + |
| 99 | + |
| 100 | + |
| 101 | NA |
| 102 | NA |
| 103 | + |
| 104 | + |
| 105 | + |
| 106 | + |
| 107 | + |
| 108 | + |
| 109 | NA |
| 110 | + |
| 111 | + |
| 112 | + |
| 113 | + |
| 114 | NA |
| 115 | + |
| 116 | + |
| 117 | +++ |
| 118 | + |
| 119 | + |
| 120a | + |
| 120b | + |
| 124 | + |
| 125 | +++ |
| 126 | + |
| 127 | + |
| 128 | + |

### Example I: Human Whole Blood Stability

The whole blood stability of the exemplified compounds is determined by LCMS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. A 96-deep well analysis plate is prepared with the addition of 100 μL ultrapure water/well. 50 μL chilled ultrapure water/well is added to 96-deep well sample collection plate and covered with a sealing mat. 1 μL of 0.5 M compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4 h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LCMS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example J: In Vitro Intrinsic Clearance Protocol

For in vitro metabolic stability experiments, test compounds are incubated with human liver microsomes at 37° C. The incubation mixture contains test compounds (1 μM), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LCMS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard is achieved using a Hypersil Gold C₁₈ column (50×2.1 mm, 5 μM, 175 Å) from ThermoFisher Scientific (Waltham, MA). Mobile phase A consists of 0.1% formic acid in water, and mobile phase B consists of 0.1% formic acid in MeCN. The total LCMS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calc. from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln 2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) is used. The in vivo hepatic clearance (CL_{H}) is then calc. by using CLᵢₙₜ and hepatic blood flow, Q (20 mL·min.⁻¹·kg⁻¹ in humans) in the well-stirred liver model disregarding all binding from CL_{H}=(Q×CLᵢₙₜ)/(Q+CLᵢₙₜ). The hepatic extraction ratio was calc. as CL_{H} divided by Q.

### Example K: In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LCMS methods.

The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example L: Time Dependent Inhibition (TDI) of CyP Protocol

This assay is designed to characterize an increase in CyP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CyP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8 min. in the presence of the substrate cocktail with the addition of a fresh aliquot of NADPH regenerating system. A calibration curve of metabolite standards can be used to quantitatively measure the enzyme activity using LCMS/MS. In addition, incubations with known time dependent inhibitors, tienilic acid (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LCMS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calc. based on 0 min. as 100%. A linear regression plot of the natural log of % activity remaining versus time for each isozyme is used to calculate the slope. The −slope is equal to the rate of enzyme loss, or the K_{obs}.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound having Formula (I): 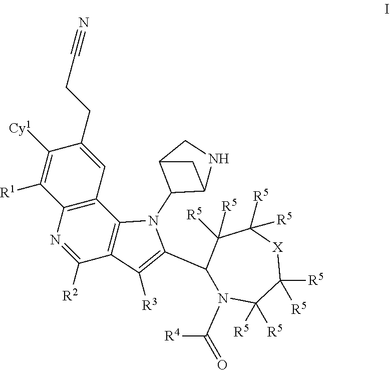
or a pharmaceutically acceptable salt thereof, wherein:
Cy¹ is C₆₋₁₀ aryl or 6-10 membered heteroaryl; wherein the C₆₋₁₀ aryl and 6-10 membered heteroaryl forming Cy¹ are each optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, C(O)R^{bCy1}, C(O)NR^{cCy1}R^{dCy1}, C(O)OR^{aCy1}, NR^{cCy1}R^{dCy1}, and S(O)₂R^{bCy1},
each R^{aCy1}, R^{bCy1}, R^{cCy1}, and R^{dCy1} is independently selected from H, C₁₋₃ alkyl, and C₁₋₃ haloalkyl;
R¹ is selected from H, D, C₁₋₃ alkyl, C₁₋₃ haloalkyl, C₂₋₃ alkenyl, C₂₋₃ alkynyl, cyclopropyl, halo, CN, OH, C₁₋₃ alkoxy, and C₁₋₃ haloalkoxy; wherein the C₁₋₃ alkyl, C₂₋₃ alkenyl, and C₂₋₃ alkynyl forming R¹ are each optionally substituted with 1 or 2 substituents independently selected from R^{1A}; and wherein the cyclopropyl forming R¹ is optionally substituted with 1 or 2 substituents independently selected from C₁₋₃ alkyl, C₁₋₃ haloalkyl, C₂₋₃ alkenyl, C₂₋₃ alkynyl, and R^{1A}; and wherein the C₁₋₃ alkyl, C₂₋₃ alkenyl, and C₂₋₃ alkynyl forming R¹ is optionally substituted with 1 or 2 substituents independently selected from R^{1B};
each R^{1A} is independently selected from C₁₋₃ alkyl, C₂₋₃ alkenyl, 2-3 alkynyl and R^{1B}, wherein the C₁₋₃ alkyl, C₂₋₃ alkenyl, and C₂₋₃ alkynyl forming R^{1A} is optionally substituted with 1 or 2 substituents independently selected from R^{1B};
each R^{1B} is independently selected from D, halo, CN, OH, C₁₋₃ alkoxy, C₁₋₃ haloalkoxy, amino, C₁₋₃ alkylamino, and di(C₁₋₃ alkyl)amino;
R² is selected from H, D, C₁₋₃ alkyl, C₂₋₃ alkenyl, C₂₋₃ alkynyl, C₁₋₃ haloalkyl, Cy^{R2}, halo, CN, OR^{a2}, C(O)R^{b2}, C(O)NR^{c2}R^{d2}, NR^{c2}R^{e2}, and NR^{c2}C(O)R^{b2}; wherein the C₁₋₃ alkyl, C₂₋₃ alkenyl, and C₂₋₃ alkynyl forming R² are each optionally substituted with Cy^{R2}, and are also optionally substituted with 1, 2, or 3 substituents independently selected from R^{2B};
Cy^{R2} is selected from C₃₋₅ cycloalkyl, 4-6 membered heterocycloalkyl, C₆₋₁₀ aryl, and 5-6 membered heteroaryl, wherein the C₃₋₅ cycloalkyl, 4-6 membered heterocycloalkyl, C₆₋₁₀ aryl, and 5-6 membered heteroaryl forming Cy^{R2} are each optionally substituted with 1, 2, or 3 substituents independently selected from R^{2A};
each R^{2A} is independently selected from C₁₋₃ alkyl, C₂₋₃ alkenyl, C₂₋₃ alkynyl, C₁₋₃ haloalkyl, and R^{2B}, wherein the C₁₋₃ alkyl, C₂₋₃ alkenyl, and C₂₋₃ alkynyl, forming R^{2A} are each optionally substituted with 1, 2 or 3 substituents independently selected from R^{2B};
each R^{2B} is independently selected from C₃₋₆cycloalkyl, 4-10 membered heterocycloalkyl, C₆₋₁₀ aryl, 5-6 membered heteroaryl, halo, D, CN, OR^{a2B}C(O)R^{b2B}, C(O)NR^{c2B}R^{d2B}, C(O)OR^{a2B}, NR^{c2B}R^{d2B}, and S(O)₂R^{b2B}; wherein the C₁₋₃ alkyl, C₃₋₆ cycloalkyl, 4-6 membered heterocycloalkyl, C₆₋₁₀ aryl, and 5-6 membered heteroaryl forming R^{2B} are each optionally substituted with 1, 2, or 3 substituents independently selected from R^{2C};
each R^{2C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, 5-6 membered heteroaryl, halo, D, CN, OR^{a2C}, C(O)R^{b2C}, C(O)NR^{c2C}R^{d2C}, C(O)OR^{a2}C, NR^{c2C}R^{d2C}, and S(O)₂R^{b2C};
each R^{a2} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming R^{a2} are each optionally substituted with 1, 2, or 3 substituents independently selected from R^{2A}; and wherein the C₁₋₆ alkyl, C₂₋₆ alkenyl, and C₂₋₆ alkynyl forming R^{a2} are each optionally substituted with 1, 2, or 3 substituents independently selected from R^{2B};
each R^{b2}, R^{c2}, and R^{d2} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming R^{b2}, R^{c2}, and R^{d2} are each optionally substituted with 1, 2, or 3 substituents independently selected from R^{2A}; and wherein the 1-3 alkyl, 2-3 alkenyl, and 2-3 alkynyl forming R^{b2}, R^{c2}, and R^{d2} are each optionally substituted with 1, 2, or 3 substituents independently selected from R^{2B}; or
any R^{c2} and R^{d2} attached to the same N atom, together with the N atom to which they are attached, form a 4-, 5-, or 6-membered heterocycloalkyl group optionally substituted with 1, 2, or 3 substituents independently selected from R^{2B};
each R^{e2} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, C₆₋₁₀ aryl, and 5-6 membered heteroaryl; wherein the C₃₋₆ cycloalkyl, 4-6 membered heterocycloalkyl, C₆₋₁₀ aryl, and 5-6 membered heteroaryl forming Re² are each optionally substituted with 1, 2, or 3 substituents independently selected from R^{2A}; and wherein the C₁-3 alkyl, C₂₋₃ alkenyl, and C₂₋₃ alkynyl, forming R^{e2} are each optionally substituted with 1, 2, or 3 substituents independently selected from R^{2B}; or
R^{c2} and R^{e2} attached to the same N atom, together with the N atom to which they are attached, form a 4-, 5-, or 6-membered heterocycloalkyl group optionally substituted with 1, 2, or 3 substituents independently selected from R^{2B};
each R^{a2B}, R^{b2B}, R^{c2B}, and R^{d2B} is independently selected from H, C₁₋₃ alkyl, and C₁₋₃ haloalkyl;
each R^{a2C}, R^{b2C}, R^{c2C} and R^{d2C} is independently selected from H, C₁₋₃ alkyl, and C₁₋₃ haloalkyl;
R³ is selected from H, D, C₁₋₃ alkyl, C₁₋₃ haloalkyl, C₂₋₃ alkenyl, C₂₋₃ alkynyl, C₃₋₆ cycloalkyl, halo, CN, and OR^{a3}; wherein the C₃₋₆ cycloalkyl forming R³ is optionally substituted with 1, 2 or 3 substituents independently selected from R^{3A}; and wherein the C₁₋₃ alkyl, C₂₋₃ alkenyl and 02-3 alkynyl forming R³ are each optionally substituted with 1, 2, or 3 substituents independently selected from R^{3B};
each R^{3A} is independently selected from C₁₋₃ alkyl, 2-3 alkenyl, 2-3 alkynyl and R^{3B}, wherein the C₁₋₃ alkyl, C₂₋₃ alkenyl, and C₂₋₃ alkynyl forming R^{3A} are each optionally substituted by 1, 2 or 3 substituents each independently selected from R^{3B};
each R^{3B} is independently selected from D, OH, CN, halo, C₁₋₃ alkoxy, C₁₋₃ haloalkoxy, amino, C₁₋₃ alkylamino, and di(C₁₋₃ alkyl)amino;
R^{a3} is selected from H, C₁₋₃ alkyl, C₁₋₃ haloalkyl, C₂₋₃ alkenyl, and C₂₋₃ alkynyl;
R⁴ is selected from C₁₋₃ alkyl, C₂₋₃ alkenyl, C₂₋₃ alkynyl, C₁₋₃ haloalkyl, Cy^{R4}, OR^{4A}, and NR^{4B}R^{4C}; wherein the C₁-3 alkyl, C₂₋₃ alkenyl, and C₂₋₃ alkynyl forming R⁴ are each optionally substituted with Cy^{R4} and also optionally substituted with 1, 2, or 3 substituents independently selected from R^{4E}Cy^{R4} is selected from C₃₋₁₀ cycloalkyl, 4-10 membered heterocycloalkyl, C₆₋₁₀ aryl, 5-10 membered heteroaryl, each optionally substituted with 1, 2, 3, 4, or 5 substituents independently selected from R^{4D};
R^{4A} is selected from C₁-3 alkyl, C₂₋₃ alkenyl, C₂₋₃ alkynyl, and Cy^{R4}; wherein the C₁₋₃ alkyl, C₂₋₃ alkenyl, and C₂₋₃ alkynyl forming R^{4A} are each optionally substituted with Cy^{R4} and also optionally substituted with 1, 2, or 3 substituents independently selected from R^{4E};
R^{4B} is selected from H, C₁₋₃ alkyl, C₂₋₃ alkenyl, C₂₋₃ alkynyl, and Cy^{R4}; wherein the C₁₋₃ alkyl, C₂₋₃ alkenyl, and C₂₋₃ alkynyl forming R^{4B} are each optionally substituted with Cy^{R4} and also optionally substituted with 1, 2, or 3 substituents independently selected from R^{4E};
R^{4C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{4C} are each optionally substituted with 1, 2, or 3 substituents independently selected from R^{4E}; or
R^{4B} and R^{4C}, together with the N atom to which they are both attached, optionally form a 4-, 5-, 6-, or 7-membered heterocycloalkyl group that is optionally substituted with 1, 2, or 3 substituents independently selected from independently selected from R^{4D};
each R^{4D} is independently selected from C₁₋₃ alkyl, C₁₋₃ haloalkyl, C₂₋₃ alkenyl, C₂₋₃ alkynyl, and R^{4E}; wherein each of said C₁₋₃ alkyl, C₂₋₃ alkenyl, and C₂₋₃ alkynyl forming R^{4D} is optionally substituted with 1, 2, or 3 substituents independently selected from R^{4E};
each R^{4E} is independently selected from D, halo, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, and S(O)₂NR^{c4}R^{d4};
R^{a4}, R^{b4}, R^{c4}, and R^{d4} are each independently selected from H, C₁₋₃ alkyl, C₂₋₃ alkenyl, C₂₋₃ alkynyl, and Cy^{Ra4} wherein the C₁₋₃ alkyl, C₂₋₃ alkenyl, and C₂₋₃ alkynyl forming R^{a4}, R^{b4}, R^{c4}, and R^{d4} are each optionally substituted by Cy^{Ra4};
Cy^{Ra4} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₃ alkyl, C₂₋₃ alkynyl, C₁₋₃ haloalkyl, C₂₋₃ alkenyl, halo, CN, OR^{a4A}SR^{a4A}, C(O)R^{b4A}, C(O)NR^{c4A}R^{d4A}, C(O)OR^{a4A}, OC(O)R^{b4A}, OC(O)NR^{c4A}R^{d4A}, NR^{c4A}R^{d4A}, NR^{c4A}C(O)R^{b4A}, NR^{c4A}C(O)NR^{c4A}R^{d4A}, NR^{c4A}C(O)OR^{a4A}, C(═NR^{e4A})NR^{c4A}R^{d4A}, NR^{c4A}C(═NR^{e4A})NR^{c4A}R^{d4A}, S(O)R^{b4A}, S(O)NR^{c4A}R^{d4A}, S(O)₂R^{b4A}, NR^{c4A}S(O)₂R^{b4A}, and S(O)₂NR^{c4A}R^{d4A}; or
R^{c4} and R^{d4} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₂₋₃ alkenyl, C₂₋₃ alkynyl, C₁₋₃ haloalkyl, halo, CN, OR^{a4A}, SR^{a4A}, C(O)R^{b4A}, C(O)NR^{c4A}R^{d4A}, C(O)OR^{a4A}, OC(O)R^{b4A}, OC(O)NR^{c4A}R^{d4A}, NR^{c4A}R^{d4A}, NR^{c4A}C(O)R^{b4A}, NR^{c4A}C(O)NR^{c4A}R^{d4A}, NR^{c4A}C(O)OR^{a4A}, C(═NR^{e4A})NR^{c4A}R^{d4A}, NR^{c4A}C(═NR^{e4A})NR^{c4A}R^{d4A}, S(O)R^{b4A}, S(O)NR^{c4A}R^{d4A}, S(O)₂R^{b4A}, NR^{c4A}S(O)₂R^{b4A}, and S(O)₂NR^{c4A}R^{d4A};
R^{a4A}, R^{b4A}, R^{c4A}, and R^{d4A} are each independently selected from H, C₁₋₃ alkyl, C₂₋₃ alkenyl, C₂₋₃ alkynyl, C₁₋₃ haloalkyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a4A}, R^{b4A}, R^{c4A}, and R^{d4A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy;
R^{c4A} and R^{d4A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆alkyl, C₂₋₆ alkenyl, C₂₋₆ alkynyl, C₁₋₆ alkoxy, C₁₋₆ haloalkyl, and C₁₋₆ haloalkoxy;
R^{e4} and R^{e4A} are each, independently, H, CN or NO₂;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl;
R⁶ is selected from H, C₁₋₆ alkyl, C₂₋₆ alkenyl, C₂₋₆ alkynyl, C₁₋₆ haloalkyl, Cy^{R6}, C(O)R^{6A}C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from C₃₋₆ cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is selected from H, C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6C} are each optionally substituted with Cy^{R6} and also optionally substituted with 1, 2, 3, or 4 substituents independently selected from R^{6F};
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E}; or
R^{6C} and R^{6D}, together with the N atom to which they are both attached, optionally form a 4-, 5-, 6-, or 7-membered heterocycloalkyl group or 5-membered heteroaryl group, that is optionally substituted with 1, 2, 3, or 4 substituents independently selected from independently selected from R^{6E};
each R^{6E} is independently selected from C₁₋₆ alkyl, C₁₋₆ haloalkyl, C₂₋₆ alkenyl, C₂₋₆ alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6};
Cy^{Ra6} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₆ alkyl, C₁₋₆ haloalkyl, C₂₋₆ alkenyl, C₂₋₆ alkynyl, halo, CN, OR^{a6A}, SR^{a6A}, OC(O)R^{b6A}, C(O)NR^{c6A}R^{d6A}, C(O)OR^{a6}A, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6}A, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A}; or
R^{c6} and R^{d6} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₆ alkyl, C₁₋₆ haloalkyl, C₂₋₆ alkenyl, C₂₋₆ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6}A, C(═NR^{e6A})NR^{c6A}R^{d6A}, NR^{c6A}C(═NR^{e6A})NR^{c6A}R^{d6A} S(O)R^{b6A}, S(O)NR^{c6A}R^{d6A}, S(O)₂R^{b6A}, NR^{c6A}S(O)₂R^{b6A}, and S(O)₂NR^{c6A}R^{d6A};
R^{a6A}, R^{b6A}, R^{c6A}, and R^{d6A} are each independently selected from H, C₁₋₆ alkyl, C₁₋₆ haloalkyl, C₂₋₆ alkenyl, C₂₋₆ alkynyl, aryl, C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl; wherein the C₁₋₆ alkyl, C₁₋₆ haloalkyl, C₂₋₆ alkenyl, C₂₋₆ alkynyl C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy; or
R^{c6A} and R^{d6A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆alkyl, C₂₋₆ alkenyl, C₂₋₆ alkynyl, C₁₋₆ alkoxy, C₁₋₆ haloalkyl, and C₁₋₆ haloalkoxy; and
R^{e6} and R^{e6A} are each, independently, H, CN or NO₂.

2. The compound of claim 1, wherein the compound of Formula (I) is a compound of any one of the Formulae (I-A), (I-B), (I-C), (I-D), (I-E), (I-F), (I-G), (I-H), (I-I), and (I-J): 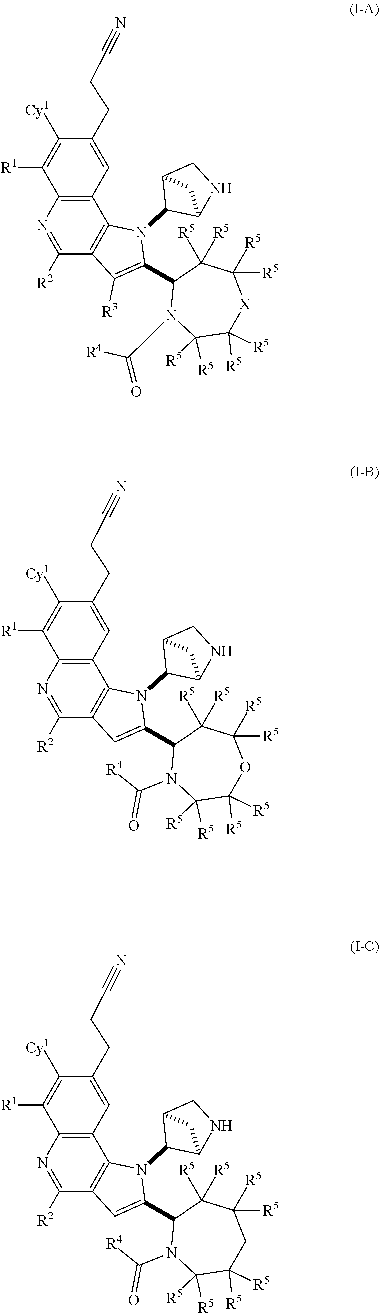 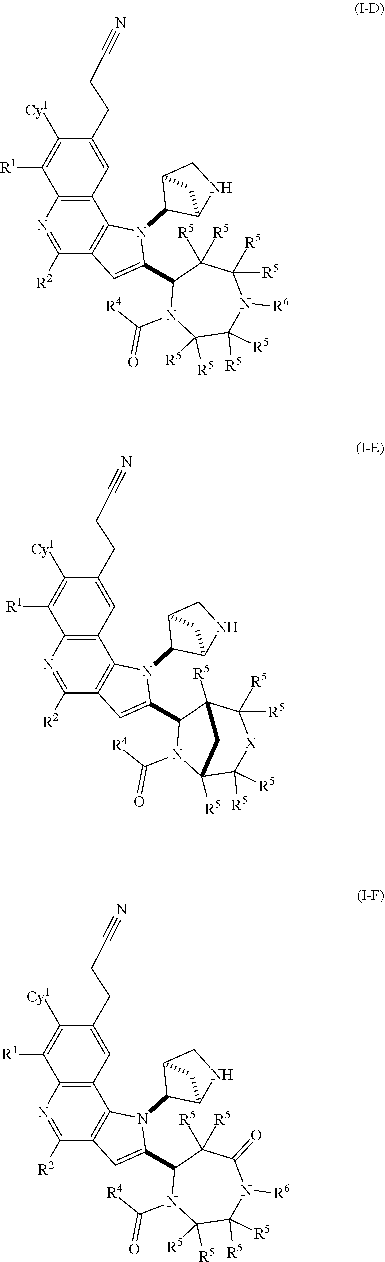 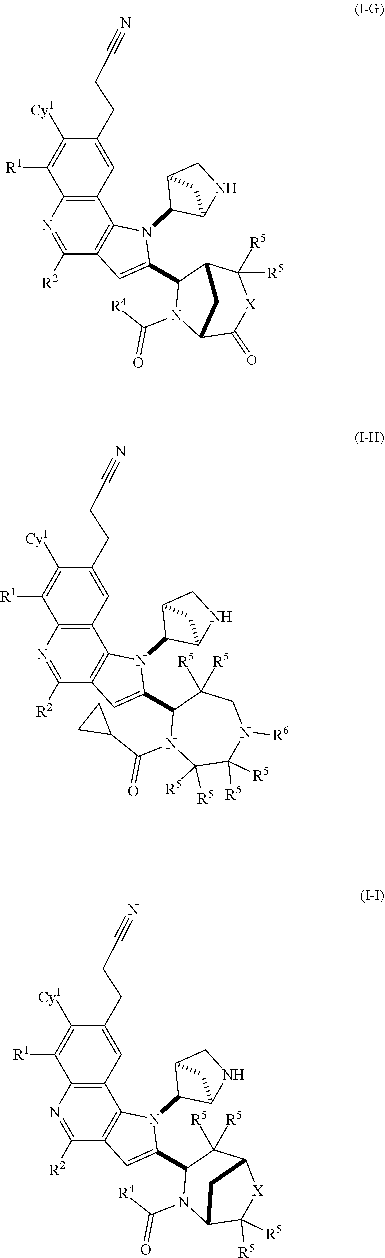 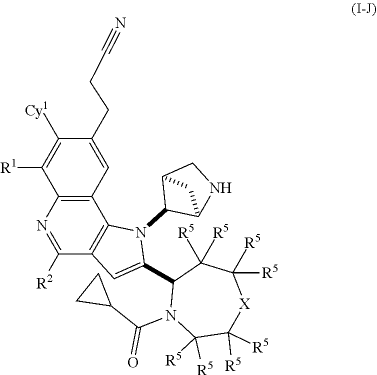
or a pharmaceutically acceptable salt thereof.

3. The compound of claim 1, or a pharmaceutically acceptable salt thereof, wherein
Cy¹ is C₆₋₁₀ aryl optionally substituted with 1, 2, 3, or 4 substituents independently selected from R^{Cy1};
each R^{Cy1} is independently selected from D, C₁₋₃ alkyl, C₁₋₃ haloalkyl, C₂₋₃ alkenyl, C₂₋₃ alkynyl, halo, CN, OR^{aCy1}, C(O)R^{bCy1}, C(O)NR^{cCy}1R^{dCy1}, C(O)OR^{aCy1}, NR^{cCy1}R^{dCy1}, and S(O)₂R^{bCy1}, each R^{aCy1}, R^{bCy1}, R^{cCy1}, and R^{dCy1} is independently selected from H, C₁₋₃ alkyl, and C₁₋₃ haloalkyl;
R¹ is selected from H, D, C₁₋₃ alkyl, C₁₋₃ haloalkyl, C₂₋₃ alkenyl, C₂₋₃ alkynyl, cyclopropyl, halo, CN, OH, C₁₋₃ alkoxy, and C₁₋₃ haloalkoxy;
R² is selected from H, D, C₁₋₃ alkyl, C₂₋₃ alkenyl, C₂₋₃ alkynyl, C₁₋₃ haloalkyl, halo, and CN;
R³ is selected from H, D, C₁₋₃ alkyl, C₁₋₃ haloalkyl, C₂₋₃ alkenyl, C₂₋₃ alkynyl, C₃₋₆ cycloalkyl, halo, CN, and OR^{a3};
R^{a3} is H or C₁₋₃ alkyl;
R⁴ is selected from C₁₋₃ alkyl, C₂₋₃ alkenyl, C₂₋₃ alkynyl, C₁₋₃ haloalkyl, and Cy^{R4};
Cy^{R4} is selected from C₃₋₁₀ cycloalkyl and 4-10 membered heterocycloalkyl;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl;
R⁶ is selected from H, C₁₋₆ alkyl, C₂₋₆ alkenyl, C₂₋₆ alkynyl, C₁₋₆ haloalkyl, Cy^{R6}, C(O)R^{6A}C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from C₃₋₆ cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is selected from H, C₁₋₆ alkyl, C₂₋₆ alkenyl, C₂₋₆ alkynyl, C₁₋₆ haloalkyl, and Cy^{R6}; wherein the C₁₋₆ alkyl, C₂₋₆ alkenyl, and C₂₋₆ alkynyl forming R^{6C} are each optionally substituted with Cy^{R6} and also optionally substituted with 1, 2, 3, or 4 substituents independently selected from R^{6F};
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E}; or
R^{6C} and R^{6D}, together with the N atom to which they are both attached, optionally form a 4-, 5-, 6-, or 7-membered heterocycloalkyl group or 5-membered heteroaryl group, that is optionally substituted with 1, 2, 3, or 4 substituents independently selected from independently selected from R^{6E};
each R^{6E} is independently selected from C₁₋₆ alkyl, C₁₋₆ haloalkyl, C₂₋₆ alkenyl, 02-6 alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6};
Cy^{Ra6} is selected from C₆₋₁₀ aryl, C₃₋₇ cycloalkyl, 5-10 membered heteroaryl, 4-10 membered heterocycloalkyl, each optionally substituted with 1, 2, 3, 4, or 5 substituents independently selected from C₁₋₆ alkyl, C₁₋₆ haloalkyl, C₂₋₆ alkenyl, C₂₋₆ alkynyl, halo, CN, OR^{a6A}, SR^{a6A}, C(O)R^{b6A}, C(O)NR^{c6A}R^{d6A}, C(O)OR^{a6}A, C(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6}A S(O)R^{b6A}, S(O)NR^{c6A}R^{d6A}, S(O)₂R^{b6A}, NR^{c6A}S(O)₂R^{b6A}, and S(O)₂NR^{c6A}R^{d6A}; and
R^{a6A}, R^{b6A}, R^{c6A}, and R^{d6A} are each independently selected from H, C₁₋₆ alkyl, C₁₋₆ haloalkyl, C₂₋₆ alkenyl, C₂₋₆ alkynyl, aryl, C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl; wherein the C₁₋₆ alkyl, C₁₋₆ haloalkyl, C₂₋₆ alkenyl, C₂₋₆ alkynyl C₆₋₁₀ aryl-C₁₋₆ alkyl, 5-10 membered heteroaryl-C₁₋₆ alkyl, C₃₋₇ cycloalkyl-C₁₋₆ alkyl, and 4-10 membered heterocycloalkyl-C₁₋₆ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₂₋₆ alkenyl, C₂₋₆ alkynyl, C₁₋₆ alkoxy, C₁₋₆ haloalkyl, and C₁₋₆ haloalkoxy.

4. The compound of claim 1, or a pharmaceutically acceptable salt thereof, wherein
Cy¹ is C₆₋₁₀ aryl optionally substituted with 1, 2, 3, or 4 substituents independently selected from halo;
R¹ is halo;
R² is C₁₋₃ alkyl;
R³ is H;
R⁴ is Cy^{R4};
Cy^{R4} is C₃₋₁₀ cycloalkyl;
X is C(R⁵)₂, O, or N—R⁶;
each R⁵ is independently selected from H, D, halo, C₁₋₃ alkyl, OR^{a5}, C₁₋₃ haloalkyl, C₂₋₃ alkenyl, and C₂₋₃ alkynyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
each R^{a5} is independently selected from H, C₁₋₃ alkyl, and C₁₋₃ haloalkyl;
R⁶ is selected from H, C₁₋₆ alkyl, C₂₋₆ alkenyl, C₂₋₆ alkynyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl, C₂₋₆ alkenyl, and C₂₋₆ alkynyl forming R⁶ are each optionally substituted with Cy^{R6}, and also optionally substituted with 1, 2, or 3 substituents independently selected from R^{6E};
Cy^{R6} is selected from C₃₋₆ cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6B} are each optionally substituted with Cy^{R6} and also optionally substituted with 1, 2, 3, or 4 substituents independently selected from R^{6F};
R^{6C} is H;
R^{6D} is selected from H, C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{6D} are each optionally substituted with 1, 2, 3, or 4 substituents independently selected from R^{6E};
each R^{6E} is independently selected from C₁₋₆ alkyl, C₁₋₆ haloalkyl, C₂₋₆ alkenyl, 02-6 alkynyl, and R^{6F}; wherein each of said C₁₋₆ alkyl, C₂₋₆ alkenyl, and C₂₋₆ alkynyl forming R^{6E} is optionally substituted with 1, 2, 3, or 4 substituents independently selected from R^{6F};
each R^{6F} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, C₂₋₆ alkenyl, C₂₋₆ alkynyl, and Cy^{Ra6} wherein the C₁₋₆ alkyl, C₂₋₆ alkenyl, and C₂₋₆ alkynyl forming R^{a6}, R^{b6}, R^{c6}, and R^{d6} are each optionally substituted by Cy^{Ra6}; and
Cy^{Ra6} is selected from C₆₋₁₀ aryl, C₃₋₇ cycloalkyl, 5-10 membered heteroaryl, 4-10 membered heterocycloalkyl.

5. The compound of claim 1, or a pharmaceutically acceptable salt thereof, wherein
Cy¹ is C₆₋₁₀ aryl optionally substituted with 1, 2, or 3 substituents independently selected from halo;
R¹ is halo;
R² is C₁₋₃ alkyl;
R³ is H;
R⁴ is Cy^{R4};
Cy^{R4} is C₃₋₁₀ cycloalkyl;
X is C(R⁵)₂, O, or N—R⁶;
each R⁵ is independently selected from H, D, halo, C₁₋₃ alkyl, and C₁₋₃ haloalkyl; optionally, two R⁵ attached to the same carbon atom, together with the carbon atom to which they are both attached, form a carbonyl; or, optionally, two R⁵ together form a C₁₋₄ alkylene that forms a spiro ring when both R⁵ forming the C₁₋₄ alkylene are attached to the same carbon atom, a fused ring when both R⁵ forming the C₁₋₄ alkylene are attached to adjacent carbon atoms and a bridged ring when both R⁵ forming the C₁₋₄ alkylene are attached to different, non-adjacent carbon atoms;
R⁶ is selected from C₁₋₆ alkyl, C₁₋₆ haloalkyl, Cy^{R6}, C(O)R^{6A}, C(O)OR^{6B}, C(O)NR^{6C}R^{6D}, SO₂R^{6A}, and SO₂NR^{6C}R^{6D} wherein the C₁₋₆ alkyl forming R⁶ is optionally substituted with Cy^{R6}, and also optionally substituted with 1, 2, or 3 substituents independently selected from R^{6E};
Cy^{R6} is selected from C₃₋₆ cycloalkyl, 4-10 membered heterocycloalkyl, C₆₋₁₀ aryl, and 5-10 membered heteroaryl, wherein the C₃₋₅ cycloalkyl, 4-10 membered heterocycloalkyl, C₆₋₁₀ aryl, and 5-10 membered heteroaryl forming Cy^{R6} are each optionally substituted with 1, 2, 3, 4 or 5 substituents independently selected from R^{6E};
R^{6A} is selected from C₁₋₆ alkyl, C₂₋₆ alkenyl, C₂₋₆ alkynyl, 1-6 haloalkyl, and Cy^{R6}; wherein the C₁₋₆ alkyl, C₂₋₆ alkenyl, and C₂₋₆ alkynyl forming R^{6A} are each optionally substituted with Cy^{R6} and also optionally substituted with 1, 2, 3, or 4 substituents independently selected from R^{6F};
R^{6B} is selected from C₁₋₆ alkyl, C₁₋₆ haloalkyl, and Cy^{R6}; wherein the C₁₋₆ alkyl forming R^{6B} is optionally substituted with Cy^{R6} and also optionally substituted with 1 or 2 substituents independently selected from R^{6F};
R^{6C} is H;
R^{6D} is selected from H, C₁₋₆ alkyl, and C₁₋₆ haloalkyl; wherein the C₁₋₆ alkyl forming R^{6D} is optionally substituted with 1 or 2 substituents independently selected from R^{6E};
each R^{6E} is independently selected from C₁₋₆ alkyl, 1-6 haloalkyl, and R^{6F}; wherein each of said C₁₋₆ alkyl forming R^{6E} is optionally substituted with 1, 2, or 3 substituents independently selected from R^{6F};
each R^{6F} is independently selected from D, halo, CN, OR^{a6}, C(O)R¹⁶, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₆ alkyl, C₁₋₆ alkyl-O—C₁₋₆ alkyl, C₁₋₆ haloalkyl, and Cy^{Ra6} wherein the C₁₋₆ alkyl forming R^{a6}, R^{b6}, R^{c6}, and R^{d6} is optionally substituted by Cy^{Ra6}; and
Cy^{Ra6} is C₃₋₇ cycloalkyl.

6. The compound of claim 1, or a pharmaceutically acceptable salt thereof, wherein X is N—R⁶.

7. (canceled)

8. The compound of claim 1, or a pharmaceutically acceptable salt thereof, wherein X is O.

9. The compound of claim 1, or a pharmaceutically acceptable salt thereof, wherein Cy¹ is phenyl optionally substituted with 1, 2, or 3 substituents independently selected from halo.

10-11. (canceled)

12. The compound of claim 1, or a pharmaceutically acceptable salt thereof, wherein R¹ is halo.

13. (canceled)

14. The compound of claim 1, or a pharmaceutically acceptable salt thereof, wherein R² is C₁₋₃ alkyl.

15. (canceled)

16. The compound of claim 1, or a pharmaceutically acceptable salt thereof, wherein R³ is H.

17. The compound of claim 1, or a pharmaceutically acceptable salt thereof, wherein R⁴ is Cy^{R4}; and
Cy^{R4} is C₃₋₆ cycloalkyl.

18-19. (canceled)

20. The compound of claim 1, or a pharmaceutically acceptable salt thereof, wherein each R⁵ is selected from H; optionally, two R⁵ attached to the same carbon atom, together with the carbon atom to which they are both attached, form a carbonyl; or, optionally, two R⁵ together form a bridged ring when both R⁵ forming the C₁₋₄ alkylene are attached to different, non-adjacent carbon atoms.

21. (canceled)

22. The compound of claim 1, or a pharmaceutically acceptable salt thereof, wherein R⁶ is selected from Cy^{R6}, C(O)R^{6A}, C(O)OR^{6B}, C(O)NR^{6C}R^{6D}, and SO₂R^{6A};
R^{6B} is C₁₋₆ alkyl;
R^{6C} is H; and
R^{6D} is selected from H, C₁₋₄ alkyl, and C₁₋₄ haloalkyl.

23. The compound of claim 1, or a pharmaceutically acceptable salt thereof, wherein R^{6A} is Cy^{R6}; and
Cy^{R6} is selected from C₃₋₆ cycloalkyl, 4-6 membered heterocycloalkyl, C₆₋₁₀ aryl, and 5-10 membered heteroaryl, wherein the C₃₋₅ cycloalkyl, 4-6 membered heterocycloalkyl, C₆₋₁₀ aryl, and 5-10 membered heteroaryl forming Cy^{R6} are each optionally substituted with 1, 2, or 3 substituents independently selected from R^{6E};
each R^{6E} is independently selected from C₁₋₆ alkyl, C₁₋₆ haloalkyl, halo, CN, OR^{a6}, and C(O)NR^{c6}R^{d6}; and
each R^{a6}, R^{b6}, R^{c6}, and R^{d6} are each independently selected from H, C₁₋₆ alkyl, C₁₋₆ alkyl-O—C₁₋₆ alkyl, and C₁₋₆ haloalkyl, wherein alkyl is optionally substituted with C₃₋₇ cycloalkyl.

24-30. (canceled)

31. The compound of claim 1, wherein the compound of Formula (I) is selected from
3-(1-(2-azabicyclo[2.1.1]hexan-5-yl)-2-(6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
4-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-(1-(2-azabicyclo[2.1.1]hexan-5-yl)-2-(6-(cyclopropanecarbonyl)-3-(6-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1,5-naphthyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2,2-difluorobenzo[d][1,3]dioxol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-(difluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-methyl-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(6-methylpyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3,4-dichlorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-(1-(2-azabicyclo[2.1.1]hexan-5-yl)-2-(3-(3,5-bis(trifluoromethyl)phenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(3-(3-chlorophenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(6-(difluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(6-methoxypyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-fluoro-4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-methyl-3-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(6-(trifluoromethyl)pyridazin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-methyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-(trifluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-methoxypyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(trifluoromethyl)pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(difluoromethoxy)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
2-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)nicotinonitrile;
3-(1-(2-azabicyclo[2.1.1]hexan-5-yl)-2-(6-(cyclopropanecarbonyl)-3-(3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluoro-4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluoro-4-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluoro-6-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(3-fluoro-5-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(4-(difluoromethyl)-3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(3-fluoro-4-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-(difluoromethyl)-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-methyl-6-oxo-1,6-dihydropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(1-isobutyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-methoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-(difluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(2-(2-hydroxypropan-2-yl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
4-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)picolinonitrile;
4-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-N,N-dimethylpicolinamide;
3-((R)-1-((1R,4R,5S)-2-azabicyclo[2.1.1]hexan-5-yl)-2-((1S,5R,7R)-6-(cyclopropanecarbonyl)-3-(1-methyl-6-oxo-1,6-dihydropyridazin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1,6-dimethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-ethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-isopropyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1-methyl-6-oxo-1,6-dihydropyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-(trifluoromethyl)imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(pyrazolo[1,5-a]pyridin-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(2-(3-([1,2,4]triazolo[1,5-a]pyridin-7-yl)-6-(cyclopropanecarbonyl)-3,6-diazabicyclo[3.2.1]octan-7-yl)-1-(2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-oxo-2H-pyran-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(3-(2-bromopyridin-4-yl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-fluoro-3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-fluoro-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-methoxy-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-methoxy-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-ethoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(2-(cyclopropylmethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(2-(2-methoxyethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(2-(difluoromethyl)-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(difluoromethyl)-5-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(6-(difluoromethoxy)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(difluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(6-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(1-methyl-1H-1,2,4-triazol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile:
3-(1-(2-azabicyclo[2.1.1]hexan-5-yl)-2-(6-(cyclopropanecarbonyl)-3-(thiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(trifluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4,5-dimethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
2-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)thiazole-4-carbonitrile;
2-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-5-methylthiazole-4-carbonitrile;
2-(7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-4-methylthiazole-5-carbonitrile;
3-(1-(2-azabicyclo[2.1.1]hexan-5-yl)-2-(6-(cyclopropanecarbonyl)-3-(5-ethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(isoxazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1,5-dimethyl-1H-imidazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-(difluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(4-(1,1-difluoroethyl)-5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(isothiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(1,5-dimethyl-1H-pyrazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-3-(5-cyclopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate:
7-(1-(2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide;
3-(1-(2-Azabicyclo[2.1.1]hexan-5-yl)-2-(6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile;
3-(2-(6-Benzoyl-2-(cyclopropanecarbonyl)-2,6-diazabicyclo[3.2.1]octan-3-yl)-1-(2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate;
3-(1-(2-Azabicyclo[2.1.1]hexan-5-yl)-2-(6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-(2-Azabicyclo[2.1.1]hexan-5-yl)-2-(6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate;
Methyl 7-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate;
3-(1-(2-azabicyclo[2.1.1]hexan-5-yl)-7-(2,3-dichlorophenyl)-2-(3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile; and
3-(1-(2-azabicyclo[2.1.1]hexan-5-yl)-7-(2,3-dichlorophenyl)-2-(3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile; and pharmaceutically acceptable salts thereof.

32. A pharmaceutical composition comprising a compound or pharmaceutically acceptable salt thereof according to claim 1, and at least one pharmaceutically acceptable carrier or excipient.

33. A method of inhibiting KRAS activity, the method comprising contacting the compound or pharmaceutically acceptable salt thereof according to claim 1, with KRAS.

34. (canceled)

35. The method of claim 33, wherein KRAS is characterized as having a somatic mutation of G12C, G12D, or G12V.

36-37. (canceled)

38. A method of treating a disease or disorder associated with activity of KRAS, the method comprising administering to a patient in need thereof a therapeutically effective amount of the compound or pharmaceutically acceptable salt thereof according to claim 1.

39. A method of treating a disease or disorder associated with activity of a KRAS protein harboring a G12C, G12D, or G12V mutation, the method comprising administering to a patient in need thereof a therapeutically effective amount of the compound or pharmaceutically acceptable salt thereof according to claim 1.

40-41. (canceled)

42. A method for treating a cancer in a patient, the method comprising administering to the patient a therapeutically effective amount of the compound or pharmaceutically acceptable salt thereof according to claim 1.

43. The method of claim 42, wherein the cancer is selected from carcinomas, hematological cancers, sarcomas, and glioblastoma.

44. The method of claim 43, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma; or
the cancer is a carcinoma selected from pancreatic, colorectal, lung, bladder, gastric, esophageal, breast, head and neck, cervical, skin, and thyroid cancers.

45. (canceled)

46. The method of claim 42, wherein abnormally proliferating cells of the cancer comprise KRAS having a G12C, G12D, or G12V mutation.

47-48. (canceled)

49. The method of claim 42, wherein the cancer is colorectal cancer, pancreatic cancer, or lung cancer.

50. (canceled)

51. The method of claim 42, wherein the cancer is pancreatic ductal cancer.

52. (canceled)

53. The method of claim 42, wherein the cancer is non-small cell lung cancer (NCSLC).

54. The method of claim 42, wherein the cancer is metastatic.

55. A method of treating an immunological or inflammatory disorder comprising administering to a patient in need thereof a therapeutically effective amount of the compound or pharmaceutically acceptable salt thereof according to claim 1.

56. The method of claim 55, wherein the immunological or inflammatory disorder is associated with activity of KRAS.

57. The method of claim 55, wherein the immunological or inflammatory disorder is associated with activity of KRAS having a G12C, G12D, or G12V mutation.

58-59. (canceled)

60. The method of claim 55, wherein the immunological or inflammatory disorder is Ras-associated lymphoproliferative disorder or juvenile myelomonocytic leukemia caused by a somatic mutation of KRAS.

61. The method of claim 60, wherein the somatic mutation of KRAS is G12C, G12D, or G12V.

62-66. (canceled)

67. The compound of claim 1, wherein the compound of Formula (I) is selected from
3-(1-((1R,4R,5S)-2-azabicyclo[2.1.1]hexan-5-yl)-2-((1 S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-phenyl-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
4-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
4-((1 S,5R,7R)-7-((Rₐ)—1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
4-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-(1-((1R,4R,5S)-2-azabicyclo[2.1.1]hexan-5-yl)-2-((1 S,5R,7R)-6-(cyclopropanecarbonyl)-3-(6-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1,5-naphthyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1,5-naphthyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1,5-naphthyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2,2-difluorobenzo[d][1,3]dioxol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2,2-difluorobenzo[d][1,3]dioxol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2,2-difluorobenzo[d][1,3]dioxol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-(difluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-(difluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-(difluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-methyl-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-methylpyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-methylpyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-methylpyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3,4-dichlorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3,4-dichlorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3,4-dichlorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-((1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)benzonitrile;
3-(1-((1R,4R,5S)-2-azabicyclo[2.1.1]hexan-5-yl)-2-((1 S,5R,7R)-3-(3,5-bis(trifluoromethyl)phenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-3-(3,5-bis(trifluoromethyl)phenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-3-(3,5-bis(trifluoromethyl)phenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-3-(3-chlorophenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-3-(3-chlorophenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-3-(3-chlorophenyl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-(trifluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-(difluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-(difluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-(difluoromethyl)pyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-fluorophenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-methoxypyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-methoxypyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-methoxypyridin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(4-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(3-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-(difluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-fluoro-4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-fluoro-4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-fluoro-4-(trifluoromethoxy)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-3-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-methyl-3-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-3-(trifluoromethyl)-1H-pyrazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1S,5R,7R)-6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-(5-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1S,5R,7R)-6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-(5-fluoro-6-methylpyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1S,5R,7R)-6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-(trifluoromethyl)pyridazin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(6-(trifluoromethyl)pyridazin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-(trifluoromethyl)pyridazin-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(1-methyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(trifluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-(trifluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(trifluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-methoxypyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(4-methoxypyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-methoxypyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(4-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(trifluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-(trifluoromethyl)pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-(trifluoromethyl)pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-(trifluoromethyl)pyrazin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-(difluoromethoxy)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-(difluoromethoxy)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-(difluoromethoxy)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
2-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)nicotinonitrile;
2-((1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)nicotinonitrile;
2-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)nicotinonitrile;
3-(1-((1R,4R,5S)-2-azabicyclo[2.1.1]hexan-5-yl)-2-((1 S,5R,7R)-6-(cyclopropanecarbonyl)-3-(3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoro-4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(3-fluoro-4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoro-4-(trifluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoro-4-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(3-fluoro-4-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoro-4-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoro-6-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-fluoro-6-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoro-6-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoro-5-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-fluoro-5-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoro-5-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(difluoromethyl)-3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(difluoromethyl)-3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(difluoromethyl)-3-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(3-fluoro-4-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(3-fluoro-4-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(3-fluoro-4-methoxypyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-(difluoromethyl)-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-(difluoromethyl)-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-(difluoromethyl)-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-methyl-6-oxo-1,6-dihydropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-isobutyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(1-isobutyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-isobutyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-methoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-methoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-methoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(difluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-(difluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(difluoromethyl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1S,5R,7R)-6-(cyclopropanecarbonyl)-3-(imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1S,5R,7R)-6-(cyclopropanecarbonyl)-3-(imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(2-hydroxypropan-2-yl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-(2-hydroxypropan-2-yl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(2-hydroxypropan-2-yl)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
4-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)picolinonitrile;
4-((1 S,5R,7R)-7-((Rₐ)—1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)picolinonitrile;
4-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)picolinonitrile;
4-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-N,N-dimethylpicolinamide;
4-((1 S,5R,7R)-7-((Rₐ)—1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-N,N-dimethylpicolinamide;
4-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-N,N-dimethylpicolinamide;
3-(1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyridazin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyridazin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyridazin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1,6-dimethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(1,6-dimethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1,6-dimethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-ethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-ethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-ethyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-isopropyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-isopropyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-isopropyl-2-oxo-1,2-dihydropyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-6-oxo-1,6-dihydropyrimidin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1S,5R,7R)-6-(cyclopropanecarbonyl)-3-(2-(trifluoromethyl)imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-(trifluoromethyl)imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1S,5R,7R)-6-(cyclopropanecarbonyl)-3-(2-(trifluoromethyl)imidazo[1,2-a]pyridin-7-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(pyrazolo[1,5-a]pyridin-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(pyrazolo[1,5-a]pyridin-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(pyrazolo[1,5-a]pyridin-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(2-((1S,5R,7R)-3-([1,2,4]triazolo[1,5-a]pyridin-7-yl)-6-(cyclopropanecarbonyl)-3,6-diazabicyclo[3.2.1]octan-7-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-((Rₐ)-2-((1 S,5R,7R)-3-([1,2,4]triazolo[1,5-a]pyridin-7-yl)-6-(cyclopropanecarbonyl)-3,6-diazabicyclo[3.2.1]octan-7-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-((Sₐ)-2-((1 S,5R,7R)-3-([1,2,4]triazolo[1,5-a]pyridin-7-yl)-6-(cyclopropanecarbonyl)-3,6-diazabicyclo[3.2.1]octan-7-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-oxo-2H-pyran-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-oxo-2H-pyran-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-oxo-2H-pyran-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-3-(2-bromopyridin-4-yl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-3-(2-bromopyridin-4-yl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-3-(2-bromopyridin-4-yl)-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-fluoro-3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-fluoro-3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-fluoro-3-methylpyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-fluoro-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-fluoro-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-fluoro-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-methoxy-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile 3-((Rₐ)-1-((1R,4R,5S)-2-azabicyclo[2.1.1]hexan-5-yl)-2-((1 S,5R,7R)-6-(cyclopropanecarbonyl)-3-(2-methoxy-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-methoxy-3-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-methoxy-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(2-methoxy-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-methoxy-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-fluoro-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-ethoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-ethoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-ethoxypyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(cyclopropylmethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-(cyclopropylmethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(cyclopropylmethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(2-methoxyethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-(2-methoxyethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(2-methoxyethoxy)pyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(2-(difluoromethyl)-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(2-(difluoromethyl)-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(2-(difluoromethyl)-6-methylpyridin-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(difluoromethyl)-5-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(difluoromethyl)-5-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(difluoromethyl)-5-fluoropyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-(difluoromethoxy)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-(difluoromethoxy)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-(difluoromethoxy)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(difluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(difluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(difluoromethyl)pyrimidin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(6-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(6-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(6-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(difluoromethyl)pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(4-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1 S,5R,7R)-6-(cyclopropanecarbonyl)-3-(5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1-methyl-1H-1,2,4-triazol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1-methyl-1H-1,2,4-triazol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1-methyl-1H-1,2,4-triazol-5-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(thiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(thiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(thiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(trifluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(trifluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(trifluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4,5-dimethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4,5-dimethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4,5-dimethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
2-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)thiazole-4-carbonitrile;
2-((1 S,5R,7R)-7-((Rₐ)—1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)thiazole-4-carbonitrile;
2-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)thiazole-4-carbonitrile;
2-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-5-methylthiazole-4-carbonitrile;
2-((1 S,5R,7R)-7-((Rₐ)—1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-5-methylthiazole-4-carbonitrile;
2-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-5-methylthiazole-4-carbonitrile;
2-((1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-4-methylthiazole-5-carbonitrile;
2-((1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-4-methylthiazole-5-carbonitrile;
2-((1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octan-3-yl)-4-methylthiazole-5-carbonitrile;
3-(1-((1R,4R,5S)-2-azabicyclo[2.1.1]hexan-5-yl)-2-((1 S,5R,7R)-6-(cyclopropanecarbonyl)-3-(5-ethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-ethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-ethylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(isoxazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(isoxazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(isoxazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1,5-dimethyl-1H-imidazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1,5-dimethyl-1H-imidazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1,5-dimethyl-1H-imidazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-(difluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-(difluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-(difluoromethyl)thiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(4-(1,1-difluoroethyl)-5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(4-(1,1-difluoroethyl)-5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(4-(1,1-difluoroethyl)-5-methylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(isothiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile 3-((Rₐ)-1-((1R,4R,5S)-2-azabicyclo[2.1.1]hexan-5-yl)-2-((1S,5R,7R)-6-(cyclopropanecarbonyl)-3-(isothiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(isothiazol-4-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(1,5-dimethyl-1H-pyrazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(1,5-dimethyl-1H-pyrazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(1,5-dimethyl-1H-pyrazol-3-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-2-((1 S,5R,7R)-6-(cyclopropanecarbonyl)-3-(5-cyclopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-2-((1S,5R,7R)-6-(cyclopropanecarbonyl)-3-(5-cyclopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-2-((1 S,5R,7R)-6-(cyclopropanecarbonyl)-3-(5-cyclopropylthiazol-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1S,5R,7R)-3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-3,6-di(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-3,6-diazabicyclo[3.2.1]octane-3-carboxylate;
(1 S,5R,7R)-7-(1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide;
(1 S,5R,7R)-7-((Rₐ)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide;
(1 S,5R,7R)-7-((Sₐ)-1-((1R,4R,5S)-2-Azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-6-(cyclopropanecarbonyl)-N-(2,2,2-trifluoroethyl)-3,6-diazabicyclo[3.2.1]octane-3-carboxamide;
3-(1-((1R,4R,5S)-2-Azabicyclo[2.1.1]hexan-5-yl)-2-((1 S,5R,7R)-6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1 S,5R,7R)-6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-((3,5-dimethylisoxazol-4-yl)sulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1 S,5R,7R)-6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-(morpholinosulfonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-6-(cyclopropanecarbonyl)-3-methyl-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-3-cyclohexyl-6-(cyclopropanecarbonyl)-2-oxo-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-3-cyclohexyl-6-(cyclopropanecarbonyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-6-(cyclopropanecarbonyl)-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-6-(cyclopropanecarbonyl)-4-oxo-3-oxa-6-azabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((R)-4-(cyclopropanecarbonyl)-1-phenyl-1,4-diazepan-5-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,3R,5R)-2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,3R,5R)-2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,3R,5R)-2-(cyclopropanecarbonyl)-6-(pyridin-2-yl)-2,6-diazabicyclo[3.2.1]octan-3-yl)-7-(2,3-dichlorophenyl)-6-fluoro-4-methyl-1H-pyrrolo[3,2-c]quinolin-8-yl)propanenitrile;
3-(2-((1R,3R,5R)-6-Benzoyl-2-(cyclopropanecarbonyl)-2,6-diazabicyclo[3.2.1]octan-3-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-((Rₐ)-2-((1R,3R,5R)-6-Benzoyl-2-(cyclopropanecarbonyl)-2,6-diazabicyclo[3.2.1]octan-3-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-((Sₐ)-2-((1R,3R,5R)-6-Benzoyl-2-(cyclopropanecarbonyl)-2,6-diazabicyclo[3.2.1]octan-3-yl)-1-((1R,4R,5S)-2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1R,5R,7R)-6-(cyclopropanecarbonyl)-2-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1 S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-4-oxo-3-(4-(trifluoromethyl)phenyl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-2-methyl-3,6-diazabicyclo[3.2.1]octane-6-carboxylate;
3-(1-((1R,4R,5S)-2-Azabicyclo[2.1.1]hexan-5-yl)-2-((1 S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1 S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-4-oxo-3-(pyridin-2-yl)-3,6-diazabicyclo[3.2.1]octan-7-yl)-7-(2,3-dichlorophenyl)-6-fluoro-4-methyl-1H-pyrrolo[3,2-c]quinolin-8-yl)propanenitrile;
3-(1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile;
3-((Rₐ)-1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile;
3-((Sₐ)-1-((1R,4R,5S)-2-Azabicyclo[2.1.1]hexan-5-yl)-2-((1 S,5R,7R)-6-(cyclopropanecarbonyl)-3-(5-isopropylthiazol-2-yl)-3,6-diazabicyclo[3.2.1]octan-7-yl-2,2-d2)-7-(2,3-dichlorophenyl)-6-fluoro-4-methyl-1H-pyrrolo[3,2-c]quinolin-8-yl)propanenitrile;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(6-(1,1-difluoroethyl)pyrimidin-4-yl)-3,6-diazabicyclo[3.2.1]octane-6-carboxylate;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(3-methoxypyrazin-2-yl)-3,6-diazabicyclo[3.2.1]octane-6-carboxylate;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-(cyclopropylmethoxy)pyrimidin-2-yl)-3,6-diazabicyclo[3.2.1]octane-6-carboxylate;
Methyl (1 S,5R,7R)-7-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate;
Methyl (1 S,5R,7R)-7-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate;
Methyl (1 S,5R,7R)-7-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-3-(5-cyanopyrazin-2-yl)-3,6-diazabicyclo[3.2.1]octane-6-carboxylate;
3-(1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile;
3-((Sₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(1-fluorocyclopropane-1-carbonyl)-3,6-diazabicyclo[3.2.1]octan-7-yl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile;
3-((Rₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile; and
3-((Sₐ)-1-((1R,4R,5S)-2-azabicyclo[2.1.1]hexan-5-yl)-7-(2,3-dichlorophenyl)-2-((1 S,5R,7R)-3-(6-(1,1-difluoroethyl)pyrimidin-4-yl)-6-(2-hydroxyacetyl)-3,6-diazabicyclo[3.2.1]octan-7-yl)-6-fluoro-4-methyl-1H-pyrrolo[3,2-c]quinolin-8-yl)propanenitrile;
and pharmaceutically acceptable salts thereof.
